(19) United States
(12) Patent Application Publication
(10) Pub. No.: US 2023/0193329 A1
(43) Pub. Date: Jun. 22, 2023

(54) Compositions and Methods for Recombinant Biosynthesis of Cannabinoids

(71) Applicant: Willow Biosciences, Inc., Calgary (CA)

(72) Inventor: Mathias Schuetz, Langley (CA)

(73) Assignee: Willow Biosciences, Inc., Calgary, AB (CA)

(21) Appl. No.: 17/935,491

(22) Filed: Sep. 26, 2022

Related U.S. Application Data
(63) Continuation of application No. PCT/US21/24390, filed on Mar. 26, 2021.
(60) Provisional application No. 63/001,202, filed on Mar. 27, 2020.

(51) Int. Cl. C12P 7/42; C12N 15/52; C12N 9/10

(52) U.S. Cl. CPC C12P 7/42; C12N 15/52; C12N 9/1085; C12Y 203/01207; C12Y 404/01026; C12Y 602/01002

(57) ABSTRACT

The present disclosure provides recombinant host cells comprising a pathway capable of producing a cannabinoid and a nucleic acid derived from a Cannabis trichome mRNA that that does not encode an enzyme in the pathway but enhances the host cells' ability to produce the cannabinoid. The disclosure also provides methods of using host cells to produce cannabinoids.

### CROSS-REFERENCE TO RELATED APPLICATIONS

This application is a continuation of International Appl. No. PCT/US2021/024390, filed Mar. 26, 2021, which claims priority benefit to U.S. Provisional Appl. No. 63/001,202, filed Mar. 27, 2020, each of which is hereby incorporated by reference herein.

### FIELD

The present disclosure relates generally to recombinant host cells comprising nucleic acids derived from Cannabis trichome mRNA that enhance the ability of the host cells to produce cannabinoids, associated nucleic acid compositions, and methods for using the recombinant host cells for cannabinoid production.

### REFERENCE TO SEQUENCE LISTING

The official copy of the Sequence Listing is submitted concurrently with the specification via USPTO Patent Center as an WIPO Standard ST.26 formatted XML file with file name “13421-001WO1_SeqList_ST26.xml”, a creation date of Sep. 26, 2022, and a size of 747,339 bytes. This Sequence Listing filed via USPTO Patent Center is part of the specification and is incorporated in its entirety by reference herein. This sequence listing corresponds to the ST.26 formatted version of the ST.25 formatted sequence listing file, “13421-001WO1_SeqList_ST25.txt” that was filed with the parent application International Appl. No. PCT/US2021/024390 on Mar. 26, 2021.

### BACKGROUND

Cannabinoids are a class of compounds that act on endocannabinoid receptors and include the phytocannabinoids naturally produced by Cannabis sativa. Cannabinoids include Δ⁹-tetrahydrocannabinol (THC), cannabidiol (CBD) and more than 80 related metabolites and synthetically produced compounds. Cannabinoids are increasingly used to treat a range of diseases and conditions such as multiple sclerosis and chronic pain. Current large-scale production of cannabinoids for pharmaceutical or other use is through extraction from plants. These plant-based production processes, however, have several challenges including susceptibility of the plants to inconsistent production caused by variance in biotic and abiotic factors, difficulty reproducing identical cannabinoid accumulation profiles, and difficulty in producing a single cannabinoid compound with purity high enough for pharmaceutical applications. While some cannabinoids can be produced as a single pure product via chemical synthesis, these processes have proven very costly and too costly for large-scale production.

More economical biosynthetic approaches to cannabinoid production are being developed using microbial hosts. These processes have the potential to be robust, scalable, and capable of producing single cannabinoid compound with higher purity compared to other current processes. Several biosynthetic systems for cannabinoid compound have been reported (see e.g., WO2019071000, WO2018200888, WO2018148849, WO2019014490, US20180073043, US20180334692, and WO2019046941). However, these biosynthetic systems still are not efficient in the biosynthesis of cannabinoids. The possible reasons for the low-yield conversion of these systems include poor protein expression, stressed cell growth, suboptimal metabolite transport, and lack of accessory genes required for optimal cannabinoid biosynthesis.

There exists a need for improved methods for the production of cannabinoid compounds. In particular, there is a need to improve the performance of microbial hosts in carrying out the recombinant biosynthesis of cannabinoid compounds.

### SUMMARY

The present disclosure relates generally to recombinant host cells comprising nucleic acids derived from Cannabis trichome mRNA that enhance the ability of the host cells to produce cannabinoids, associated nucleic acid compositions, and methods for using the recombinant host cells for cannabinoid production.

This summary is intended to introduce the subject matter of the present disclosure, but does not cover each and every embodiment, combination, or variation that is contemplated and described within the present disclosure. Further embodiments are contemplated and described by the disclosure of the detailed description, drawings, and claims.

In at least one embodiment, the present disclosure provides recombinant host cell comprising a pathway capable of producing a cannabinoid and a nucleic acid derived from a Cannabis trichome mRNA that does not encode an enzyme in the pathway.

In at least one embodiment, the nucleic acid derived from a Cannabis trichome mRNA encodes a polypeptide having a protein function selected from: amino acid biosynthesis, amino acid degradation, carbohydrate metabolism, carrier-mediated transport, cell cycle organization, cellular respiration, chromatin organization, circadian clock system regulation, coenzyme metabolism, oxidoreductase, transferase, hydrolase, lyase, effector-triggered immunity network co-regulatory protein, fatty acid biosynthesis, lipid degradation, lipid transfer type protein, messenger ribonucleoparticle (mRNP) export, mulatexin-like, organelle machinery ribonuclease activity, phytohormone action, protein folding, protein phosphorylation, protein S-glutathionylation, protein translocation, redox homeostasis, RNA biosynthesis, RNA processing, solute transport, terpenoid metabolism, and/or vesicle trafficking.

In at least one embodiment, the nucleic acid derived from a Cannabis trichome mRNA encodes a polypeptide selected from: 1-acyl-sn-glycerol-3-phosphate acyltransferase 2; 1-aminocyclopropane-1-carboxylate oxidase homolog 1-like; acyl-acyl carrier protein thioesterase ATL3, chloroplastic-like; barwin-like; beta-adaptin-like protein B; BURP domain protein RD22-like; cationic peroxidase 2-like; cell division control protein 2 homolog 2; chloroplast stem-loop binding protein of 41 kDa b, chloroplastic; cysteine-rich receptor-like protein kinase 19; cytochrome B5-like protein; delta(12)-fatty-acid desaturase FAD2-like; desiccation-related protein PCC13-62-like; dormancy-associated protein 2-like; E3 ubiquitin-protein ligase SDIR1; gamma-carbonic anhydrase-like 2, mitochondrial; germacrene-A synthase-like; glucan endo-1,3-beta-glucosidase 12; glucose-6-phosphate 1-dehydrogenase 6, cytoplasmic-like; glycine-rich RNA-binding protein-like; major allergen Pru av 1-like; malonate--CoA ligase; mannose-1-phosphate guanylyltransferase 1; mediator of RNA polymerase II transcription subunit 11; MLP-like protein 423; NADH dehydrogenase [ubiquinone] flavoprotein 2, mitochondrial; NADP-dependent glyceraldehyde-3-phosphate dehydrogenase; NDR1/HIN1-like protein 1; non-specific lipid-transfer protein 1-like; non-specific lipid-transfer protein 2-like; ornithine aminotransferase, mitochondrial; peptidyl-prolyl cis-trans isomerase CYP19-3; peroxidase 12-like; phosphoinositide phosphatase SAC1; probable gamma-secretase subunit PEN-2; probable protein phosphatase 2C 60; programmed cell death protein 2-like; protein CASP; protein ELF4-LIKE 4-like; protein REVEILLE 8; protein SRC1; protein TIC 56, chloroplastic; pyruvate decarboxylase 1-like; small acidic protein 1; sphingoid long-chain bases kinase 1; structural maintenance of chromosomes protein 1; translationally-controlled tumor protein homolog; tubulin beta-2 chain; ubiquitin-conjugating enzyme E2-17 kDa-like; U-box domain-containing protein 34; upstream activation factor subunit UAF30; uridine kinase-like protein 1, chloroplastic; V-type proton ATPase subunit B 1; and YTH domain-containing protein ECT4-like.

In at least one embodiment, the nucleic acid derived from Cannabis trichome mRNA encodes a polypeptide comprising (a) an amino acid sequence having at least 90% identity to any one of even-numbered SEQ ID NOs: 16-540; (b) an amino acid sequence sequence having at least 90%, at least 95%, at least 96%, at least 97%, at least 98%, or at least 99% identity to any one of SEQ ID NOs: 36, 74, 88, 94, 104, 106, 108, 110, 112, 114, 116, 120, 122, 124, 126, 128, 130, 132, 134, 136, 138, 140, 142, 144, 146, 14, 150, 168, 174, 184, 188, 206, 210, 232, 270, 274, and 396; or (c) an amino acid sequence having at least 90% identity to any one of even-numbered SEQ ID NOs:

In at least one embodiment, the nucleic acid derived from Cannabis trichome mRNA comprises: (a) a nucleotide sequence having at least 90% identity to any one of odd-numbered SEQ ID NOs: 15-539; (b) a nucleotide sequence having at least 90%, at least 95%, at least 96%, at least 97%, at least 98%, or at least 99% identity to any one of SEQ ID NOs: 35, 73, 87, 93, 103, 105, 107, 109, 111, 113, 115, 119, 121, 123, 125, 127, 129, 131, 133, 135, 137, 139, 141, 143, 145, 147, 149, 167, 173, 183, 187, 205, 209, 231, 269, 273, and 395; or (c) a nucleotide sequence having at least 90% identity to any one of odd-numbered SEQ ID NOs: 121-149.

In at least one embodiment, the pathway capable of producing a cannabinoid comprises enzymes capable of converting hexanoic acid to CBGA. In at least one embodiment, the pathway capable of producing a cannabinoid comprises at least the following enzymes: AAE, OLS, OAC, and PT4; optionally, wherein AAE has an amino acid sequence of at least 90% identity to SEQ ID NO: 2, OLS has an amino acid sequence of at least 90% identity to SEQ ID NO: 4, OAC has an amino acid sequence of at least 90% identity to SEQ ID NO: 6, and PT4 has an amino acid sequence of at least 90% identity to SEQ ID NO: 8 or 10.

In at least one embodiment, the pathway further comprises an enzyme capable of catalyzing the conversion of CBGA to Δ⁹-THCA, CBDA, and/or CBCA. In at least one embodiment, the pathway comprising AAE, OLS, OAC, and PT4, further comprises: THCA synthase, CBDA synthase, and/or CBCA synthase; optionally, wherein the pathway further comprises (a) a CBDA synthase having an amino acid sequence of at least 90% identity to SEQ ID NO: 12 or 14, and/or (b) a THCA synthase having an amino acid sequence of at least 90% identity to SEQ ID NO: 542 or 544.

In at least one embodiment, the cannabinoid produced by the host cell is selected from cannabigerolic acid (CBGA), cannabigerol (CBG), cannabidiolic acid (CBDA), cannabidiol (CBD), Δ⁹-tetrahydrocannabinolic acid (Δ⁹-THCA), Δ⁹-tetrahydrocannabinol (Δ⁹-THC), Δ⁸-tetrahydrocannabinolic acid (Δ⁸-THCA), Δ⁸-tetrahydrocannabinol (Δ⁸-THC), cannabichromenic acid (CBCA), cannabichromene (CBC), cannabinolic acid (CBNA), cannabinol (CBN), cannabidivarinic acid (CBDVA), cannabidivarin (CBDV), Δ⁹-tetrahydrocannabivarinic acid (Δ⁹-THCVA), Δ⁹-tetrahydrocannabivann (Δ⁹-THCV), cannabidibutolic acid (CBDBA), cannabidibutol (CBDB), Δ⁹-tetrahydrocannabutolic acid (Δ⁹-THCBA), Δ⁹-tetrahydrocannabutol (Δ⁹-THCB), cannabidiphorolic acid (CBDPA), cannabidiphorol (CBDP), Δ⁹-tetrahydrocannabiphorolic acid (Δ⁹-THCPA), Δ⁹-tetrahydrocannabiphorol (Δ⁹-THCP), cannabichromevarinic acid (CBCVA), cannabichromevarin (CBCV), cannabigerovarinic acid (CBGVA), cannabigerovarin (CBGV), cannabicyclolic acid (CBLA), cannabicyclol (CBL), cannabielsoinic acid (CBEA), cannabielsoin (CBE), cannabicitranic acid (CBTA), cannabicitran (CBT), and any combination thereof.

In at least one embodiment, the recombinant host cell produces the cannabinoid with a titer that is increased at least 1.2-fold, at least 2-fold, at least 3-fold, at least 4-fold, at least 5-fold, or more relative to a control recombinant host cell comprising the pathway and not the nucleic acid derived from a Cannabis trichome mRNA.

In at least one embodiment, the present disclosure also provides a method for producing a cannabinoid comprising: (a) culturing in a suitable medium a recombinant host cell of the present disclosure (e.g., a host comprising a pathway of enzymes capable of converting hexanoic acid to CBGA and a nucleic acid derived from a Cannabis trichome mRNA that does not encode an enzyme in the pathway); and (b) recovering the produced cannabinoid. In at least one embodiment, the method further comprises contacting a cell-free extract of the culture with a biocatalytic reagent or chemical reagent.

In at least one embodiment, the present disclosure also provides method for making a recombinant host cell for producing a cannabinoid comprising introducing into a host cell: (a) a first set of nucleic acids that encode a pathway of enzymes capable of producing a cannabinoid; and (b) a nucleic acid derived from a Cannabis trichome mRNA that does not encode an enzyme in the pathway. In at least one embodiment, the nucleic acid derived from a Cannabis trichome mRNA encodes a polypeptide having a protein function selected from: amino acid biosynthesis, amino acid degradation, carbohydrate metabolism, carrier-mediated transport, cell cycle organization, cellular respiration, chromatin organization, circadian clock system regulation, coenzyme metabolism, oxidoreductase, transferase, hydrolase, lyase, effector-triggered immunity network co-regulatory protein, fatty acid biosynthesis, lipid degradation, lipid transfer type protein, messenger ribonucleoparticle (mRNP) export, mulatexin-like, organelle machinery ribonuclease activity, phytohormone action, protein folding, protein phosphorylation, protein S-glutathionylation, protein translocation, redox homeostasis, RNA biosynthesis, RNA processing, solute transport, terpenoid metabolism, and/or vesicle trafficking. In at least one embodiment, the nucleic acid derived from a Cannabis trichome mRNA encodes a polypeptide comprising an amino acid sequence having at least 90% identity to any one of even-numbered SEQ ID NOs: 16-540. In at least one embodiment, the nucleic acid derived from Cannabis trichome mRNA comprises a nucleotide sequence having at least 90% identity to any one of odd-numbered SEQ ID NOs: 15-539.

### BRIEF DESCRIPTION OF THE DRAWINGS

A better understanding of the novel features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 depicts an exemplary pathway capable of converting hexanoic acid to CBGA.

FIG. 2 depicts an exemplary pathway capable of catalyzing the conversion of CBGA to Δ⁹-THCA, CBDA, and/or CBCA.

FIG. 3 depicts a yeast expression vector with auxotrophic marker (LEU2 gene) into which the cDNA library derived from Cannabis trichome mRNA was sub-cloned for screening as described in Example 1.

### DETAILED DESCRIPTION

For the descriptions herein and the appended claims, the singular forms “a”, and “an” include plural referents unless the context clearly indicates otherwise. Thus, for example, reference to “a protein” includes more than one protein, and reference to “a compound” refers to more than one compound. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. The use of “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.”

Where a range of values is provided, unless the context clearly dictates otherwise, it is understood that each intervening integer of the value, and each tenth of each intervening integer of the value, unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of these limits, ranges excluding (i) either or (ii) both of those included limits are also included in the invention. For example, “1 to 50,” includes “2 to 25,” “5 to 20,” “25 to 50,” “1 to 10,” etc.

Generally, the nomenclature used herein and the techniques and procedures described herein include those that are well understood and commonly employed by those of ordinary skill in the art, such as the common techniques and methodologies described in e.g., Green and Sambrook, Molecular Cloning: A Laboratory Manual (Fourth Edition), Vols. 1-3, Cold Spring Harbor Laboratory, Cold Spring Harbor, N.Y., 2012 (hereinafter “Sambrook”); and Current Protocols in Molecular Biology, F. M. Ausubel et al., eds., originally published in 1987 in book form by Greene Publishing Associates, Inc. and John Wiley & Sons, Inc., and regularly supplemented through 2011, and now available in journal format online as Current Protocols in Molecular Biology, Vols. 00 -130, (1987-2020), published by Wiley & Sons, Inc. in the Wiley Online Library (hereinafter “Ausubel”).

All publications, patents, patent applications, and other documents referenced in this disclosure are hereby incorporated by reference in their entireties for all purposes to the same extent as if each individual publication, patent, patent application or other document were individually indicated to be incorporated by reference herein for all purposes.

Unless defined otherwise, all technical and scientific terms used herein have the same meaning as commonly understood by one of ordinary skill in the art to which the present invention pertains. It is to be understood that the terminology used herein is for describing particular embodiments only and is not intended to be limiting. For purposes of interpreting this disclosure, the following description of terms will apply and, where appropriate, a term used in the singular form will also include the plural form and vice versa.

Definitions

“Cannabis trichome mRNA” refers to an mRNA molecule produced in the glandular trichome tissue of a cannabis plant, e.g., Cannabis sativa.

“Cannabinoid” refers to a compound that acts on cannabinoid receptor, and is intended to include the endocannabinoid compounds that are produced naturally in animals, the phytocannabinoid compounds produced naturally in cannabis plants, and the synthetic cannabinoids compounds. Exemplary cannabinoids of the present disclosure include those compounds listed in Table 3 (below).

“Pathway” refers an ordered sequence of enzymes that act in a linked series to convert an initial substrate molecule into final product molecule. As used herein, “pathway” is intended to encompass naturally-occurring pathways and non-naturally occurring, recombinant pathways. Accordingly, a pathway of the present disclosure can include a series of enzymes that are naturally-occurring and/or non-naturally occurring, and can include a series of enzymes that act in vivo or in vitro.

“Pathway capable of producing a cannabinoid” refers to a pathway that can convert an initial substrate compound into a cannabinoid. For example, the four enzymes AAE, OLS, OAC, and PT4 which convert hexanoic acid (HA) to cannabigerolic acid (CBGA) form a pathway capable of producing a cannabinoid.

“Conversion” as used herein refers to the enzymatic conversion of the substrate(s) to the corresponding product(s). “Percent conversion” refers to the percent of the substrate that is converted to the product within a period of time under specified conditions. Thus, the “enzymatic activity” or “activity” of an enzymatic conversion can be expressed as “percent conversion” of the substrate to the product.

“Substrate” as used herein in the context of an enzyme mediated process refers to the compound or molecule acted on by the enzyme.

“Product” as used herein in the context of an enzyme mediated process refers to the compound or molecule resulting from the activity of the enzyme.

“Host cell” as used herein refers to a cell capable of being functionally modified with recombinant nucleic acids and functioning to express recombinant products, including polypeptides and compounds produced by activity of the polypeptides.

“Nucleic acid,” or “polynucleotide” as used herein refer to two or more nucleosides that are covalently linked together. The nucleic acid may be wholly comprised ribonucleosides (e.g., RNA), wholly comprised of 2′-deoxyribonucleotides (e.g., DNA) or mixtures of ribo- and 2′-deoxyribonucleosides. The nucleoside units of the nucleic acid can be linked together via phosphodiester linkages (e.g., as in naturally occurring nucleic acids), or the nucleic acid can include one or more non-natural linkages (e.g., phosphorothioester linkage). Nucleic acid or polynucleotide is intended to include single-stranded or double-stranded molecules, or molecules having both single-stranded regions and double-stranded regions. Nucleic acid or polynucleotide is intended to include molecules composed of the naturally occurring nucleobases (i.e., adenine, guanine, uracil, thymine and cytosine), or molecules comprising that include one or more modified and/or synthetic nucleobases, such as, for example, inosine, xanthine, hypoxanthine, etc.

“Protein,” “polypeptide,” and “peptide” are used herein interchangeably to denote a polymer of at least two amino acids covalently linked by an amide bond, regardless of length or post-translational modification (e.g., glycosylation, phosphorylation, lipidation, myristilation, ubiquitination, etc.). As used herein “protein” or “polypeptide” or “peptide” polymer can include D- and L-amino acids, and mixtures of D- and L-amino acids.

“Naturally-occurring” or “wild-type” as used herein refers to the form as found in nature. For example, a naturally occurring nucleic acid sequence is the sequence present in an organism that can be isolated from a source in nature and which has not been intentionally modified by human manipulation.

“Recombinant,” “engineered,” or “non-naturally occurring” when used herein with reference to, e.g., a cell, nucleic acid, or polypeptide, refers to a material, or a material corresponding to the natural or native form of the material, that has been modified in a manner that would not otherwise exist in nature, or is identical thereto but is produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

“Nucleic acid derived from Cannabis trichome mRNA” as used herein refers to a nucleic acid having a sequence at least substantially identical to a sequence of an mRNA found in Cannabis trichome cells or tissue. For example, cDNA molecules prepared by reverse transcription of mRNA isolated from Cannabis trichome cells, or nucleic acid molecules prepared synthetically to have a sequence at least substantially identical to, or which hybridizes to a sequence at least substantially identical to, a Cannabis trichome mRNA sequence.

“Coding sequence” refers to that portion of a nucleic acid (e.g., a gene) that encodes an amino acid sequence of a protein.

“Heterologous” as used herein refers to any polynucleotide that is introduced into a host cell by laboratory techniques, and includes polynucleotides that are removed from a host cell, subjected to laboratory manipulation, and then reintroduced into a host cell.

“Codon optimized” refers to changes in the codons of the polynucleotide encoding a protein to those preferentially used in a particular organism such that the encoded protein is efficiently expressed in the organism of interest. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, the polynucleotides encoding the imine reductase enzymes may be codon optimized for optimal production from the host organism selected for expression.

“Preferred, optimal, high codon usage bias codons” refers to codons that are used at higher frequency in the protein coding regions than other codons that code for the same amino acid. The preferred codons may be determined in relation to codon usage in a single gene, a set of genes of common function or origin, highly expressed genes, the codon frequency in the aggregate protein coding regions of the whole organism, codon frequency in the aggregate protein coding regions of related organisms, or combinations thereof. Codons whose frequency increases with the level of gene expression are typically optimal codons for expression. A variety of methods are known for determining the codon frequency (e.g., codon usage, relative synonymous codon usage) and codon preference in specific organisms, including multivariate analysis, for example, using cluster analysis or correspondence analysis, and the effective number of codons used in a gene (see GCG CodonPreference, Genetics Computer Group Wisconsin Package; CodonW, John Peden, University of Nottingham; McInerney, J. O, 1998, Bioinformatics 14:372-73; Stenico et al., 1994, Nucleic Acids Res. 222437-46; Wright, F., 1990, Gene 87:23-29). Codon usage tables are available for a growing list of organisms (see for example, Wada et al., 1992, Nucleic Acids Res. 20:2111-2118; Nakamura et al., 2000, Nucl. Acids Res. 28:292; Duret, et al., supra; Henaut and Danchin, “Escherichia coli and Salmonella,” 1996, Neidhardt, et al. Eds., ASM Press, Washington D.C., p. 2047-2066. The data source for obtaining codon usage may rely on any available nucleotide sequence capable of coding for a protein. These data sets include nucleic acid sequences actually known to encode expressed proteins (e.g., complete protein coding sequences-CDS), expressed sequence tags (ESTS), or predicted coding regions of genomic sequences (see for example, Mount, D., Bioinformatics: Sequence and Genome Analysis, Chapter 8, Cold Spring Harbor Laboratory Press, Cold Spring Harbor, N.Y., 2001; Uberbacher, E. C., 1996, Methods Enzymol. 266:259-281; Tiwari et al., 1997, Comput. Appl. Biosci. 13:263-270).

“Control sequence” as used herein refers to all sequences, which are necessary or advantageous for the expression of a polynucleotide and/or polypeptide as used in the present disclosure. Each control sequence may be native or foreign to the nucleic acid sequence encoding a polypeptide. Such control sequences include, but are not limited to, a leader, a promoter, a polyadenylation sequence, a pro-peptide sequence, a signal peptide sequence, and a transcription terminator. At a minimum, control sequences typically include a promoter, and transcriptional and translational stop signals. The control sequences may be provided with linkers for the purpose of introducing specific restriction sites facilitating ligation of the control sequences with the coding region of the nucleic acid sequence encoding a polypeptide.

“Operably linked” as used herein refers to a configuration in which a control sequence is appropriately placed (e.g., in a functional relationship) at a position relative to a polynucleotide sequence or polypeptide sequence of interest such that the control sequence directs or regulates the expression of the sequence of interest.

“Promoter sequence” refers to a nucleic acid sequence that is recognized by a host cell for expression of a polynucleotide of interest, such as a coding sequence. The promoter sequence contains transcriptional control sequences, which mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

“Percentage of sequence identity,” “percent sequence identity,” “percentage homology,” or “percent homology” are used interchangeably herein to refer to values quantifying comparisons of the sequences of polynucleotides or polypeptides, and are determined by comparing two optimally aligned sequences over a comparison window, wherein the portion of the polynucleotide or polypeptide sequence in the comparison window may comprise additions or deletions (or gaps) as compared to the reference sequence for optimal alignment of the two sequences. The percentage values may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman, 1981, Adv. Appl. Math. 2:482, by the homology alignment algorithm of Needleman and Wunsch, 1970, J. Mol. Biol. 48:443, by the search for similarity method of Pearson and Lipman, 1988, Proc. Natl. Acad. Sci. USA 85:2444, by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)). Examples of algorithms that are suitable for determining percent sequence identity and sequence similarity are the BLAST and BLAST 2.0 algorithms, which are described in Altschul et al., 1990, J. Mol. Biol. 215: 403-410 and Altschul et al., 1977, Nucleic Acids Res. 3389-3402, respectively. Software for performing BLAST analyses is publicly available through the National Center for Biotechnology Information website. This algorithm involves first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff and Henikoff, 1989, Proc Natl Acad Sci USA 89:10915). Exemplary determination of sequence alignment and % sequence identity can employ the BESTFIT or GAP programs in the GCG Wisconsin Software package (Accelrys, Madison Wis.), using default parameters provided.

“Reference sequence” refers to a defined sequence used as a basis for a sequence comparison. A reference sequence may be a subset of a larger sequence, for example, a segment of a full-length nucleic acid or polypeptide sequence. A reference sequence typically is at least 20 nucleotide or amino acid residue units in length, but can also be the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. “Comparison window” refers to a conceptual segment of at least about 20 contiguous nucleotide positions or amino acids residues wherein a sequence may be compared to a reference sequence of at least 20 contiguous nucleotides or amino acids and wherein the portion of the sequence in the comparison window may comprise additions or deletions (or gaps) of 20 percent or less as compared to the reference sequence (which does not comprise additions or deletions) for optimal alignment of the two sequences.

“Substantial identity” or “substantially identical” refers to a polynucleotide or polypeptide sequence that has at least 70% sequence identity, at least 80% sequence identity, at least 85% sequence identity, at least 90% sequence identity, at least 95 % sequence identity, or at least 99% sequence identity, as compared to a reference sequence over a comparison window of at least 20 nucleoside or amino acid residue positions, frequently over a window of at least 30-50 positions, wherein the percentage of sequence identity is calculated by comparing the reference sequence to a sequence that includes deletions or additions which total 20 percent or less of the reference sequence over the window of comparison.

“Corresponding to,” “reference to,” or “relative to” when used in the context of the numbering of a given amino acid or polynucleotide sequence refers to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered imine reductase, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

“Isolated” as used herein in reference to a molecule means that the molecule (e.g., cannabinoid, polynucleotide, polypeptide) is substantially separated from other compounds that naturally accompany it, e.g., protein, lipids, and polynucleotides. The term embraces nucleic acids which have been removed or purified from their naturally-occurring environment or expression system (e.g., host cell or in vitro synthesis).

“Substantially pure” refers to a composition in which a desired molecule is the predominant species present (i.e., on a molar or weight basis it is more abundant than any other individual macromolecular species in the composition), and is generally a substantially purified composition when the object species comprises at least about 50 percent of the macromolecular species present by mole or % weight.

“Recovered” as used herein in relation to an enzyme, protein, or cannabinoid compound, refers to a more or less pure form of the enzyme, protein, or cannabinoid.

Recombinant Host Cells with Enhanced Cannabinoid Production

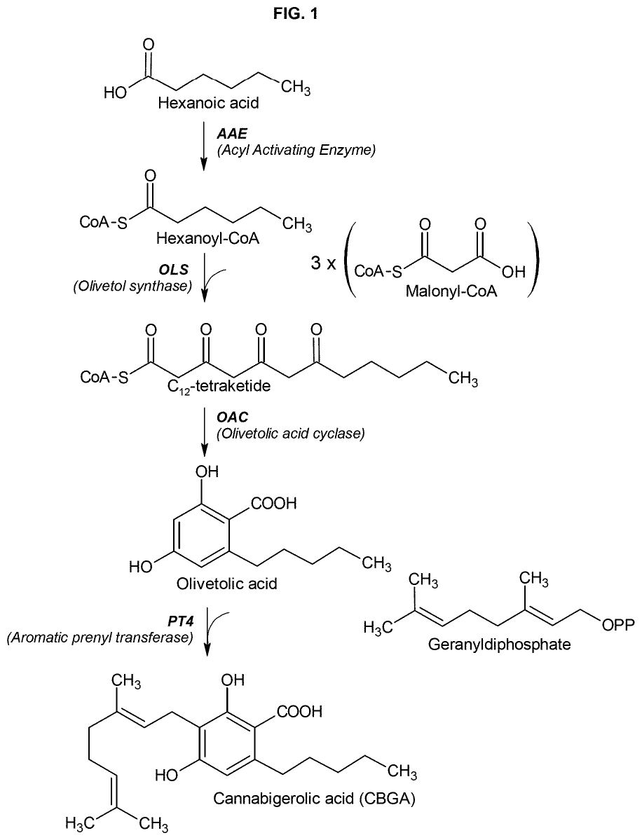

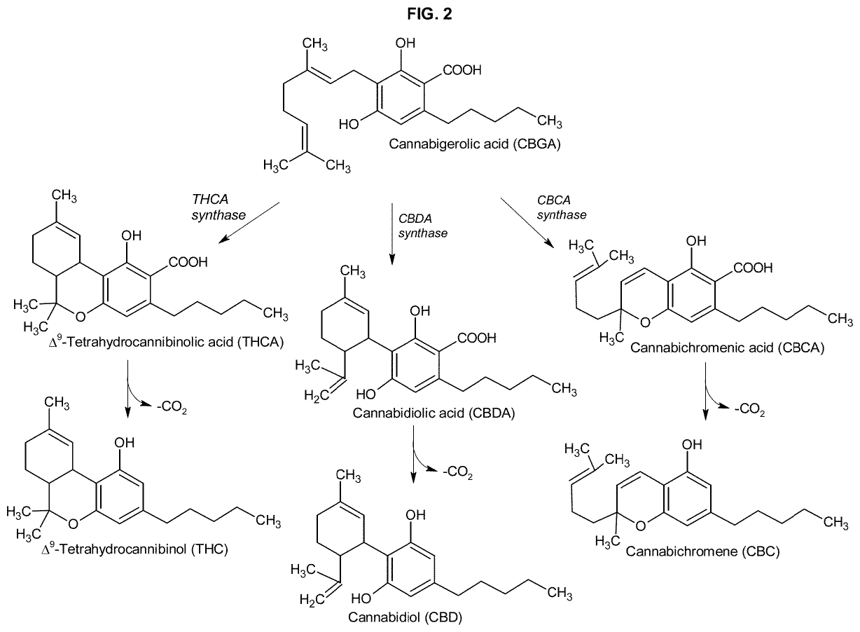

The present disclosure provides recombinant host cells (e.g., S. cerevisiae) already transformed with a cannabinoid biosynthesis pathway, which further comprise an introduced heterologous nucleic acid derived from Cannabis trichome mRNA. In the context of the recombinant host cells already transformed with a cannabinoid biosynthesis pathway, an exemplary cannabinoid biosynthesis pathway is one capable of converting hexanoic acid (HA) cannabigerolic acid (CBGA) as depicted in FIG. 1. The biosynthetic conversion of HA to CBGA is carried out by the sequence of enzymes, Acyl Activating Enzyme (AAE), Olivetol Synthase (OLS), Olivetolic Acid Cyclase (OAC), and a prenyltransferase (PT4). Although FIG. 1 depicts a four enzyme cannabinoid pathway from HA to CBGA, it is contemplated that shorter pathways comprising only the three enzyme, AAE, OLS, and OAC, could be incorporated into a host cell for the biosynthetic production of the cannabinoid precursor olivetolic acid (OA) from HA, or a pathway of PT4 and a cannabinoid synthase could be incorporated in a host cell for biosynthetic production of a cannabinoid from OA. As shown in FIG. 2, an extension of the four enzyme exemplary pathway of FIG. 1 with a cannabinoid synthase (e.g., CBDAS, THCAS, and/or CBCAS) allows for the biosynthetic production of one or more of the cannabinoids, Δ⁹-THCA (or “THCA”), CBDA, and/or CBCA. These cannabinoids are capable of further conversion by decarboxylation to provide the cannabinoids, Δ⁹-THC (or “THC”), CBD, and/or CBC. It is contemplated, that in some embodiments this further decarboxylation reaction can be carried out under in vitro reaction conditions using the cannabinoid acids separated and/or isolated from the recombinant host cells.

The presence of the additional nucleic acid derived from Cannabis trichome mRNA integrated in the recombinant host cells results in substantially enhanced yields of the cannabinoid (e.g., 2-fold or more increased) relative to a recombinant host cell comprising the same cannabinoid synthesis pathway but without a nucleic acid derived from Cannabis trichome mRNA. Furthermore, the additional nucleic acid derived from Cannabis trichome mRNA does not encode an enzyme in the cannabinoid biosynthesis pathway already present in the recombinant host cell. Indeed, the nucleic acids derived from Cannabis trichome mRNA that exhibit this effect on the cannabinoid biosynthesis do not encode enzymes or proteins that would be predicted a priori to enhance cannabinoid production in a recombinant host cell. For example, as disclosed elsewhere herein, nucleic acid derived from a Cannabis trichome mRNA capable of enhancing cannabinoid biosynthesis can include a nucleic acid encoding a polypeptide having a protein function selected from: amino acid biosynthesis, amino acid degradation, carbohydrate metabolism, carrier-mediated transport, cell cycle organization, cellular respiration, chromatin organization, circadian clock system regulation, coenzyme metabolism, oxidoreductase, transferase, hydrolase, lyase, effector-triggered immunity network co-regulatory protein, fatty acid biosynthesis, lipid degradation, lipid transfer type protein, messenger ribonucleoparticle (mRNP) export, mulatexin-like, organelle machinery ribonuclease activity, phytohormone action, protein folding, protein phosphorylation, protein S-glutathionylation, protein translocation, redox homeostasis, RNA biosynthesis, RNA processing, solute transport, terpenoid metabolism, and/or vesicle trafficking.

The unexpected and surprising technical effect of enhanced cannabinoid biosynthesis associated with the introduction of one or more such nucleic acids into a recombinant host cell already capable of biosynthesizing a cannabinoid provides an improved recombinant host cell, and associated improved methods of cannabinoid production. Accordingly, in at least one embodiment, the present disclosure provides a recombinant host cell comprising a pathway capable of producing a cannabinoid and a nucleic acid derived from a Cannabis trichome mRNA that does not encode an enzyme in the pathway. As described further below, an exemplary pathway capable of biosynthesizing a cannabinoid can include four enzymes that convert hexanoic acid to cannabigerolic acid (CBGA).

In at least one embodiment, the recombinant host cell comprising a nucleic acid derived from a Cannabis trichome mRNA is capable of producing the cannabinoid with a titer that is increased relative to a control recombinant host cell comprising the pathway and not the nucleic acid derived from a Cannabis trichome mRNA. In at least one embodiment, the titer of cannabinoid produced is increased by at least 1.1-fold. 1.2-fold, 1.3-fold, 1.4-fold, 1.5-fold, 2-fold, 3-fold, 4-fold, 5-fold, 6-fold, 10-fold, or more relative to a control recombinant host cell comprising the pathway and not the nucleic acid derived from a Cannabis trichome mRNA.

Without intending to be bound by theory, it believed that the nucleic acids derived from a Cannabis trichome mRNA disclosed herein facilitate a range of metabolic mechanisms that can enhance cannabinoid biosynthesis across a range of recombinant host cells that are engineered with cannabinoid pathways. For example, it is believed that these nucleic acids contribute to higher cannabinoid production through the following off-pathway functions: increase heterologous protein expression, improve host stress response, stabilize heterologous proteins, mediate cannabinoid or precursor molecule transport, modify host metabolic regulation, facilitating protein-protein interaction (metabolon formation). In at least one embodiment, the recombinant host cell comprises a cannabinoid producing pathway comprising the enzymes capable of converting hexanoic acid to cannabigerolic acid (CBGA). One such a pathway capable of converting hexanoic acid to CBGA is illustrated in FIG. 1. Accordingly, in at least one embodiment of the recombinant host cell, the pathway capable of producing a cannabinoid comprises enzymes capable of catalyzing reactions (i) — (iv):

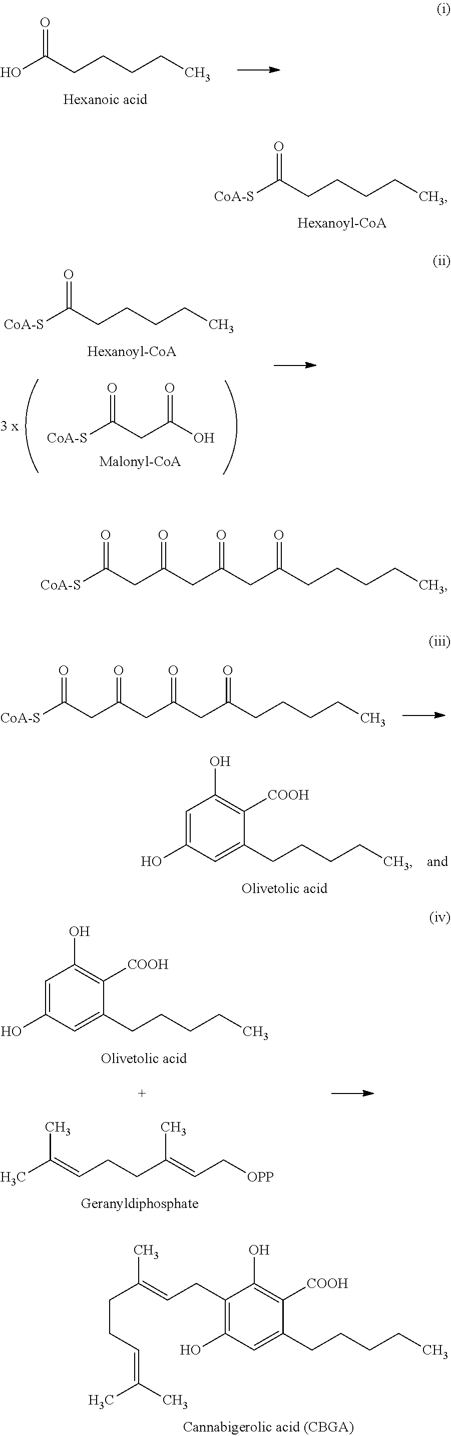

As shown in FIG. 1, exemplary enzymes capable of catalyzing reactions (i)-(iv) are: (i) acyl activating enzyme (AAE); (ii) olivetol synthase (OLS); (iii) olivetolic acid cyclase (OLA); and (iv) aromatic prenyl transferase (PT4).

As shown in FIG. 2, the cannabinoid compound, CBGA, that is produced by the pathway of FIG. 1, can be further converted to at least three other different cannabinoid compounds, Δ⁹-tetrahydrocannabinolic acid (THCA), cannabidiolic acid (CBDA), and/or cannabichromenic acid (CBCA). Accordingly, in at least one embodiment, the present disclosure provides a recombinant host cell comprising a pathway capable of converting hexanoic acid to CBGA and further comprising an enzyme capable of catalyzing the conversion of (v) CBGA to Δ⁹-THCA; (vi) CBGA to CBDA; and/or (vii) CBGA to CBCA. Thus, in at least one embodiment, the recombinant host cell comprises pathway capable of converting hexanoic acid to CBGA further comprises further comprises enzymes capable of catalyzing a reaction (v), (vi), and/or (vii):

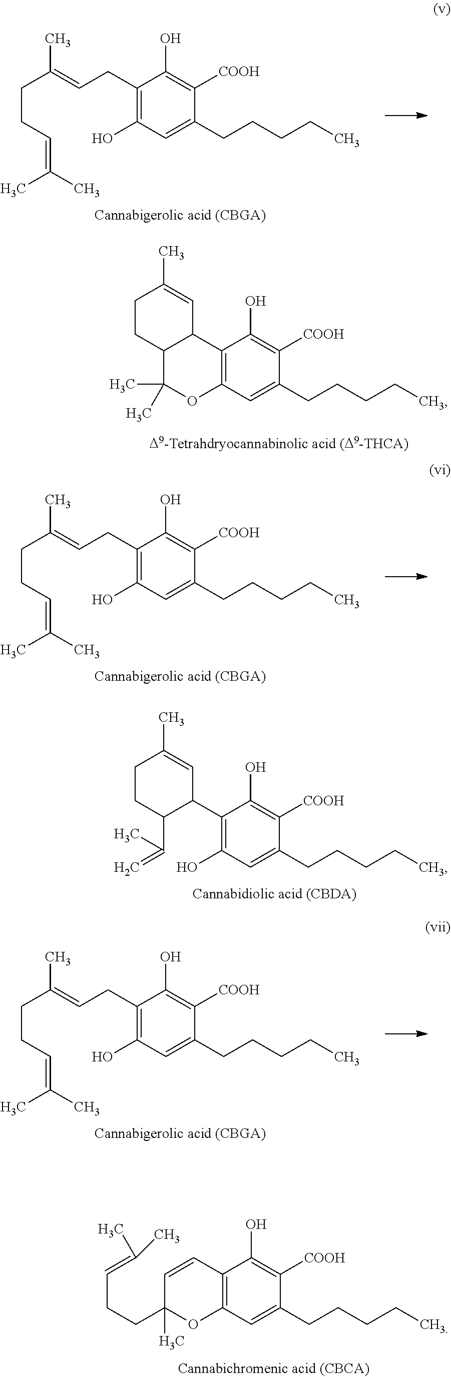

As shown in FIG. 2, exemplary enzymes capable of catalyzing reaction (v)-(vii) are: (v) THCA synthase (THCAS); (vi) CBDA synthase (CBDAS); and (vii) CBCA synthase (CBCAS).

Cannabinoid pathway enzymes that can be introduced into a recombinant host cell to provide the pathways illustrated in FIGS. 1 and 2 include, but are not limited to, the cannabinoid pathway enzymes from Cannabis sativa described in Table 1 (below).

TABLE 1
Cannabinoid pathway enzymes

| | | SEQ | SEQ |
|---|---|---|---|
| | | ID | ID |
| Enzyme Name | | NO: | NO: |
| (abbreviation) | GenBank Identifier | (nt) | (aa) |
| Acyl activating | >AFD33345.1 acyl-activating enzyme 1 | 1 | 2 |
| enzyme (AAE) | [Cannabis sativa] | | |
| Olivetol synthase | >BAG14339.1 olivetol synthase | 3 | 4 |
| (OLS) | [Cannabis sativa] | | |
| Olivetolic acid | >AFN42527.1 olivetolic acid cyclase | 5 | 6 |
| cyclase (OAC) | [Cannabis sativa] | | |
| Aromatic prenyl | >DAC76710.1 prenyltransferase 4, | 7 | 8 |
| transferase (PT4) | [Cannabis sativa] | | |
| CBDA synthase | >BAF65033.1 cannabidiolic acid | 11 | 12 |
| (CBDAS) | synthase [Cannabis sativa] | | |
| THCA synthase | >BAC41356.1 tetrahydrocannabinolic | 543 | 542 |
| (THCAS) | acid synthase [Cannabis sativa] | | |

In at least one embodiment, the recombinant host cell the pathway capable of producing a cannabinoid comprises at least the exemplary enzymes, wherein the enzymes have the amino acid sequences of SEQ ID NO: 2 (AAE), SEQ ID NO: 4 (OLS), SEQ ID NO: 6 (OAC), and SEQ ID NO: 8 (PT4). In at least one embodiment, the recombinant host cell the pathway capable of producing a cannabinoid further comprises the enzyme of SEQ ID NO: 12 (CBDAS).

The exemplary cannabinoid pathway enzymes listed in Table 1 are the naturally occurring sequences from C. sativa, it also is contemplated, however, that cannabinoid pathway enzymes used in the recombinant host can include naturally occurring sequence homologs of these enzymes and/or enzymes having non-naturally occurring sequences. For example, enzymes with amino acid sequences engineered to function optimally in a particular enzyme pathway, and/or optimally for production of particular cannabinoid, and/or optimally in a particular host. Methods for preparing such non-naturally occurring enzyme sequences are known in the art and include methods for enzyme engineering such as directed evolution. In at least one embodiment, the amino acid sequence of non-naturally occurring enzyme can be modified at either its N- or C-terminus by truncation, or fusion. For example, in at least one embodiment of the pathway of producing a cannabinoid, the naturally occurring amino acid sequence of the PT4 enzyme of SEQ ID NO: 8 can be truncated at the N-terminus by up to 82 amino acids to provide the PT4 of SEQ ID NO: 10 (also referred to herein as “d82_PT4”), which is capable of functioning to produce the cannabinoid CBGA in a recombinant host cell. Accordingly, in at least one embodiment of the recombinant host cell, the pathway capable of producing a cannabinoid comprises at least enzymes having an amino acid sequence at least 90% identity to SEQ ID NO: 2 (AAE), SEQ ID NO: 4 (OLS), SEQ ID NO: 6 (OAC), and SEQ ID NOs: 10 (PT4). Similarly, it is contemplated that engineered versions of the AAE, OLS, OAC, and CBDAS enzymes can be prepared using methods known in the art, and used in the compositions and methods of the present disclosure. For example, the CBDAS enzyme of SEQ ID NO: 12 or the THCAS enzyme of SEQ ID NO: 542 can be truncated at the N-terminus by up to 28 amino acids to provide the d28_CBDAS enzyme of SEQ ID NO: 14 and the d28_THCAS enzyme of SEQ ID NO: 544. Accordingly, in at least one embodiment of the recombinant host cell, the pathway capable of producing a cannabinoid comprises at least enzymes having an amino acid sequence at least 90% identity to SEQ ID NO: 2 (AAE), SEQ ID NO: 4 (OLS), SEQ ID NO: 6 (OAC), SEQ ID NOs: 10 (d82_PT4), and SEQ ID NO: 14 (d28_CBDAS). Or in at least one embodiment of the recombinant host cell, the pathway capable of producing a cannabinoid comprises at least enzymes having an amino acid sequence at least 90% identity to SEQ ID NO: 2 (AAE), SEQ ID NO: 4 (OLS), SEQ ID NO: 6 (OAC), SEQ ID NOs: 10 (d82_PT4), and SEQ ID NO: 544 (d28_THCAS).

Other cannabinoid pathway enzymes useful in the recombinant host cells and associated methods of the present disclosure are known in the art, and can include naturally occurring enzymes obtained or derived from cannabis plants, (e.g., Cannabis sativa, and its sub-species, sativa, indica, and ruderalis), or non-naturally occurring enzymes that have been engineered based on the naturally occurring cannabis plant sequences. It is also contemplated that enzymes obtained or derived from other organisms (e.g., microorganisms) having a catalytic activity related to a desired conversion activity useful in a cannabinoid pathway can be engineered for use in a recombinant host cell of the present disclosure.

The heterologous nucleic acids derived from C. sativa trichome mRNA of the present disclosure can be incorporated (e.g., by recombinant transformation, or Cas9 integration) into a range of host cells already comprising a cannabinoid biosynthesis pathway to provide a system for enhanced production of cannabinoids (e.g., CBGA, CBDA, THCA, CBCA) or cannabinoid precursor compounds. Generally, the host cell used for the recombinant host cells of the present disclosure can be any cell that can be recombinantly modified with nucleic acids and then cultured to express the recombinant products of those nucleic acids, including polypeptides and metabolites produced by the activity of the recombinant polypeptides. A wide range of suitable sources of host cells are known in the art, and exemplary host cell sources useful as recombinant host cells of the present disclosure include, but are not limited to, Saccharomyces cerevisiae, Yarrowia lipolytica, Pichia pastoris, and Escherichia coli. It is also contemplated that the host cell source for a recombinant host cell of the present disclosure can include a non-naturally occurring cell source, e.g., an engineered host cell. For example, a non-naturally occurring source host cell, such as a yeast cell previously engineered for improved production of recombinant genes, may be used to prepare the recombinant host cell of the present disclosure. Accordingly, in at least one embodiment, the present disclosure provides a recombinant host cell previously engineered with an integrated functional cannabinoid biosynthesis pathway and a heterologous nucleic acid encoding a protein that is not part of the pathway, wherein the host cell source is selected from Saccharomyces cerevisiae, Yarrowia lipolytica, Pichia pastoris, Escherichia coli, or an engineered cell derived from Saccharomyces cerevisiae, Yarrowia lipolytica, Pichia pastoris, Escherichia coli.

The recombinant host cells of the present disclosure comprise heterologous nucleic acids encoding a pathway of enzymes capable of producing a cannabinoid, such as CBGA, and a heterologous nucleic acid derived from C. sativa trichome mRNA that is “off-pathway” (does not encode a pathway enzyme). As described elsewhere herein, cannabinoid pathway enzymes and the nucleic acid sequences encoding them are known in the art and provided herein, and can readily be used in accordance with the present disclosure. Typically, the nucleic acid sequence encoding enzymes in the cannabinoid pathway further include one or more nucleic acid sequences controlling expression of these pathway enzymes. These one or more additional nucleic acid sequences together with the nucleic acid sequences encoding the enzymes which form a part of an cannabinoid biosynthetic pathway can be considered a heterologous nucleic acid sequence. A variety of techniques and methodologies are available and well known in the art for introducing such heterologous nucleic acid sequences encoding the cannabinoid pathway enzymes into a host cell so as to attain expression in the host cell. Techniques well known to the skilled artisan include, for example, those techniques found in the well known Sambrook and Ausubel references cited elsewhere herein.

One of ordinary skill will recognize that the heterologous nucleic acids encoding the cannabinoid pathway enzymes and/or the nucleic acids derived from C. sativa mRNA can further comprise transcriptional promoters capable of controlling expression of the enzymes in the recombinant host cell. Generally, the transcriptional promoters are selected to be compatible with the host cell, so that promoters obtained from bacterial cells are used when a bacterial host cell is selected in accordance herewith, while a fungal promoter is used when a fungal host cell is selected, a plant promoter is used when a plant cell is selected, and so on.

Promoters useful in the recombinant host cells of the present disclosure may be constitutive or inducible, provided such promoters are operable in the host cells. Promoters that may be used to control expression in fungal host cells, such as Saccharomyces cerevisiae, are well known in the art and include, but are not limited to: inducible promoters, such as a Gall promoter or Gal10 promoter, a constitutive promoter, such as an alcohol dehydrogenase (ADH) promoter, a glyceraldehyde-3-phosphate dehydrogenase (GPD) promoter, or an S. pombe Nmt, or ADH promoter. Exemplary promoters that may be used to control expression in bacterial cells can include the Escherichia coli promoters lac, tac, trc, trp or the T7 promoter. Exemplary promoters that may be used to control expression in plant cells include, for example, a Cauliflower Mosaic Virus 35S promoter (Odell et al. (1985) Nature 313:810-812), a ubiquitin promoter (U.S. Pat. No. 5,510,474; Christensen et al. (1989)), or a rice actin promoter (McElroy et al. (1990) Plant Cell 2:163-171). Exemplary promoters that can be used in mammalian cells include, a viral promoter such as an SV40 promoter or a metallothionine promoter. All of these host cell promoters are well known by and readily available to one of ordinary skill in the art. Further nucleic acid control elements useful for controlling expression in a recombinant host cell can include transcriptional terminators, enhancers and the like, all of which may be used with the heterologous nucleic acids incorporate in the recombinant host cells of the present disclosure.

A wide variety of techniques are well known in the art for linking transcriptional promoters and other control elements to heterologous nucleic acid sequences encoding pathway genes or other heterologous off-pathway nucleic acids. Such techniques are described in e.g., the Sambrook and Ausubel references cited elsewhere herein. Accordingly, in at least one embodiment, the heterologous nucleic acid sequences of the present disclosure comprise a promoter capable of controlling expression in a host cell, wherein the promoter is linked to a heterologous nucleic acid sequence derived from C. sativa trichome mRNA, and/or an enzyme of a cannabinoid pathway (e.g., AAE, OLS, OAC, PT4, or CBDAS). Such heterologous nucleic acid sequences can be integrated into a recombinant expression vector which ensures good expression in the desired host cell, wherein the expression vector is suitable for expression in a host cell, meaning that the recombinant expression vector comprises the heterologous nucleic acid sequence linked to any genetic elements required to achieve expression in the host cell. Genetic elements that may be included in the expression vector in this regard include a transcriptional termination region, one or more nucleic acid sequences encoding marker genes, one or more origins of replication, and the like. In some embodiments, the expression vector further comprises genetic elements required for the integration of the vector or a portion thereof in the host cell's genome.

It is also contemplated that in some embodiments an expression vector comprising a heterologous nucleic acid derived from C. sativa trichome mRNA of the present disclosure may further contain a marker gene. Marker genes useful in accordance with the present disclosure include any genes that allow the distinction of transformed cells from non-transformed cells, including all selectable and screenable marker genes. A marker gene may be a resistance marker such as an antibiotic resistance marker against, for example, kanamycin or ampicillin. Screenable markers that may be employed to identify transformants through visual inspection include β-glucuronidase (GUS) (U.S. Pat. Nos. 5,268,463 and 5,599,670) and green fluorescent protein (GFP) (Niedz et al., 1995, Plant Cell Rep., 14: 403).

Nucleic Acids Derived from Cannabis trichome mRNA that Enhance Recombinant Cannabinoid Biosynthesis

The glandular trichome tissue of C. sativa plants are known to produce and secrete high quantities of metabolites including cannabinoid compounds. Although heterologous enzymes capable of acting as a functional cannabinoid biosynthesis pathway have been transformed recombinantly into host cells to produce cannabinoid compounds, the effect, if any, of other “off-pathway” genes that are expressed in the C. sativa trichome tissue on cannabinoid production has remained unknown. As described elsewhere herein (including the Examples), high-throughput screening of cDNA derived C. sativa trichome mRNA sequences in a recombinant host system containing a biosynthetic pathway that converts hexanoic acid (HA) to the cannabinoid, cannabigerolic acid (CBGA), has identified numerous off-pathway genes that substantially enhance production of CBGA in transformed host cells.

Table 2 below provides a summary description of exemplary off-pathway Cannabis sativa genes derived from C. sativa trichome mRNA screening that result in at least 2-fold increased CBGA yield in a recombinant host cell system, and their associated sequence identifiers. The nucleotide and amino acid sequences are included in the accompanying Sequence Listing.

TABLE 2
C. sativa trichome genes that enhance cannabinoid biosynthesis

| | | | SEQ | SEQ |
|---|---|---|---|---|
| | | | ID | ID |
| | | | NO: | NO: |
| Protein Function¹ | Gene Annotation² | FIOPC³ | (nt) | (aa) |
| Amino acid | 1,2-dihydroxy-3-keto-5- | + | 15 | 16 |
| metabolism. | methylthiopentene dioxygenase 2-like | | | |
| biosynthesis | (LOC115722194) | | | |
| | glutamate decarboxylase-like | + | 17 | 18 |
| | (LOC115711993) | | | |
| | ornithine aminotransferase, | ++ | 19 | 20 |
| | mitochondrial (LOC115697770) | | | |
| | phospho-2-dehydro-3-deoxyheptonate | + | 21 | 22 |
| | aldolase 2, chloroplastic-like | | | |
| | (LOC115724690) | | | |
| Amino acid | fumarylacetoacetase (LOC115707370) | + | 23 | 24 |
| metabolism. | methylcrotonoyl-CoA carboxylase beta | + | 25 | 26 |
| degradation | chain, mitochondrial (LOC115702935) | | | |
| | thiosulfate/3-mercaptopyruvate | + | 27 | 28 |
| | sulfurtransferase 1, mitochondrial | | | |
| | (LOC115709268) | | | |
| Carbohydrate | Glucose-6-phosphate 1-dehydrogenase | ++ | 29 | 30 |
| metabolism. | 6, cytoplasmic-like (LOC115699660) | | | |
| | glyceraldehyde-3-phosphate | + | 31 | 32 |
| | dehydrogenase GAPCP1, chloroplastic- | | | |
| | like (LOC115705493) | | | |
| | probable UDP-arabinopyranose mutase | + | 33 | 34 |
| | 1 (LOC115720163) | | | |
| | pyruvate decarboxylase 1-like | ++ | 35 | 36 |
| | (LOC115718871) | | | |
| Cell cycle | 65-kDa microtubule-associated protein | + | 37 | 38 |
| organization. | 1-like (LOC115724533) | | | |
| | callose synthase 1-like | + | 39 | 40 |
| | (LOC115725331), transcript variant X4 | | | |
| | cell division control protein 2 homolog 2 | ++++ | 41 | 42 |
| | (LOC115708347), transcript variant X1 | | | |
| | expansin-A8-like (LOC115720977) | + | 43 | 44 |
| | uncharacterized LOC115708756 | + | 45 | 46 |
| | (LOC115708756), ncRNA | | | |
| Cellular respiration. | Gamma carbonic anhydrase-like 2, | +++ | 47 | 48 |
| | mitochondrial (LOC115710160) | | | |
| | NADP-dependent glyceraldehyde-3- | ++ | 49 | 50 |
| | phosphate dehydrogenase | | | |
| | (LOC115724840) | | | |
| | probable lactoylglutathione lyase, | + | 51 | 52 |
| | chloroplastic (LOC115707793) | | | |
| | probable mitochondrial-processing | + | 53 | 54 |
| | peptidase subunit beta, mitochondrial | | | |
| | (LOC115698552), transcript variant X1 | | | |
| | ubiquitin carboxyl-terminal hydrolase 16 | + | 55 | 56 |
| | (LOC115717510), transcript variant X2 | | | |
| Chromatin | DNA (cytosine-5)-methyltransferase 1- | + | 57 | 58 |
| organization | like (LOC115707860) | | | |
| | DNA (cytosine-5)-methyltransferase | + | 59 | 60 |
| | DRM2 (LOC115699580), transcript | | | |
| | variant X2 | | | |
| | FACT complex subunit SSRP1 | + | 61 | 62 |
| | (LOC115698505) | | | |
| | histone H4 (LOC115719770) | + | 63 | 64 |
| | nucleosome assembly protein 1; 2 | + | 65 | 66 |
| | (LOC115705230) | | | |
| Coenzyme | pyridoxal 5′-phosphate synthase-like | + | 67 | 68 |
| metabolism | subunit PDX1.2 (LOC115707858) | | | |
| | 1-aminocyclopropane-1-carboxylate | +++ | 69 | 70 |
| | oxidase homolog 1-like (LOC115722336) | | | |
| Enzyme | formate dehydrogenase, mitochondrial | + | 71 | 72 |
| classification. EC_1 | (LOC115698580) | | | |
| oxidoreductase | probable 2-oxoglutarate-dependent | + | 73 | 74 |
| | dioxygenase At3g111800 | | | |
| | (LOC115703220) | | | |
| | trans-cinnamate 4-monooxygenase | + | 75 | 76 |
| | (LOC115719463) | | | |
| Enzyme | 7-deoxyloganetin glucosyltransferase- | + | 77 | 78 |
| classification. EC_2 | like (LOC115701442) | | | |
| transferase | cysteine-rich receptor-like protein kinase | ++++ | 79 | 80 |
| | 19 (LOC115724835) | | | |
| | probable alpha, alpha-trehalose- | + | 81 | 82 |
| | phosphate synthase [UDP-forming] 7 | | | |
| | (LOC115712065) | | | |
| | probable xyloglucan | + | 83 | 84 |
| | endotransglucosylase/hydrolase protein | | | |
| | 28 (LOC115722305) | | | |
| | probable xyloglucan | + | 85 | 86 |
| | endotransglucosylase/hydrolase protein | | | |
| | 6 (LOC115712234) | | | |
| | protein ECERIFERUM 26-like | + | 87 | 88 |
| | (LOC115721023) | | | |
| | scopoletin glucosyltransferase-like | + | 89 | 90 |
| | (LOC115713325) | | | |
| | serine/threonine-protein kinase STY13- | + | 91 | 92 |
| | like (LOC115699359) | | | |
| | stemmadenine O-acetyltransferase-like | + | 93 | 94 |
| | (LOC115705983) | | | |
| Enzyme | 3-hydroxyisobutyryl-CoA hydrolase-like | + | 95 | 96 |
| classification. EC_3 | protein 3, mitochondrial | | | |
| hydrolase | (LOC115706202), transcript variant X1 | | | |
| | glucan endo-1,3-beta-glucosidase 12 | ++ | 97 | 98 |
| | (LOC115698667), transcript variant X1 | | | |
| Enzyme | myrcene synthase, chloroplastic-like | + | 99 | 100 |
| classification. EC_4 | (LOC115716405) | | | |
| lyase | | | | |
| External stimuli | cysteine and histidine-rich domain- | + | 101 | 102 |
| response. | containing protein RAR1 | | | |
| pathogen.effector- | (LOC115716870), transcript variant X1 | | | |
| triggered immunity | | | | |
| (ETI) network.co- | | | | |
| regulatory protein | | | | |
| (RAR1) | | | | |
| Lipid metabolism. | 3-ketoacyl-CoA synthase 6 | + | 103 | 104 |
| fatty acid | (LOC115712453) | | | |
| biosynthesis | acyl carrier protein 1, chloroplastic-like | + | 105 | 106 |
| | (LOC115719263) | | | |
| | delta(12)-fatty-acid desaturase FAD2-like | ++ | 107 | 108 |
| | (LOC115719329) | | | |
| | delta(12)-fatty-acid desaturase FAD2-like | ++ | 109 | 110 |
| | (LOC115719329) | | | |
| | delta(12)-fatty-acid desaturase FAD2-like | + | 111 | 112 |
| | (LOC115719329) | | | |
| | malonate-CoA ligase (LOC115707826) | ++ | 113 | 114 |
| Lipid metabolism. | acyl-acyl carrier protein thioesterase | ++++ | 115 | 116 |
| degradation | ATL3, chloroplastic-like | | | |
| | (LOC115697587) | | | |
| | enoyl-CoA hydratase 2, peroxisomal | + | 117 | 118 |
| | (LOC115702272) | | | |
| | patatin-like protein 1 (LOC115715123) | + | 119 | 120 |
| Lipid transfer type | major allergen Pru av 1-like | +++ | 121 | 122 |
| protein | (LOC115723029) | | | |
| | major allergen Pru av 1-like | +++ | 123 | 124 |
| | (LOC115723029) | | | |
| | major allergen Pru av 1-like | + | 125 | 126 |
| | (LOC115723029) | | | |
| | non-specific lipid-transfer protein 1-like | ++++ | 127 | 128 |
| | (LOC115698126) | | | |
| | non-specific lipid-transfer protein 1-like | ++ | 129 | 130 |
| | (LOC115698127) | | | |
| | non-specific lipid-transfer protein 1-like | + | 131 | 132 |
| | (LOC115698170) | | | |
| | non-specific lipid-transfer protein 1-like | + | 133 | 134 |
| | (LOC115698181) | | | |
| | non-specific lipid-transfer protein 2-like | + | 135 | 136 |
| | (LOC115722949) | | | |
| | non-specific lipid-transfer protein 2-like | ++++ | 137 | 138 |
| | (LOC115722949) | | | |
| | non-specific lipid-transfer protein 2-like | + | 139 | 140 |
| | (LOC115722949) | | | |
| Mulatexin-like | mulatexin-like (LOC115712540) | + | 141 | 142 |
| | mulatexin-like (LOC115712540) | + | 143 | 144 |
| | mulatexin-like (LOC115712540) | + | 145 | 146 |
| | mulatexin-like (LOC115712540) | + | 147 | 148 |
| | mulatexin-like (LOC115712540) | + | 149 | 150 |
| Multi-process | protein ELF4-LIKE 4-like | ++++ | 151 | 152 |
| regulation. | (LOC115707067), transcript variant X2 | | | |
| circadian clock | | | | |
| system.evening | | | | |
| element | | | | |
| regulation.Evening | | | | |
| Complex | | | | |
| (EC).component | | | | |
| ELF4 | | | | |
| N/A | auxin response factor 19-like | + | 153 | 154 |
| | (LOC115709608) | | | |
| N/A | auxin-responsive protein IAA1-like | + | 155 | 156 |
| | (LOC115719320) | | | |
| N/A | barwin-like (LOC115721107) | ++ | 157 | 158 |
| N/A | barwin-like (LOC115721107) | + | 159 | 160 |
| N/A | beta-adaptin-like protein B | + | 161 | 162 |
| | (LOC115716271) | | | |
| N/A | beta-glucuronosyltransferase GlcAT14B- | + | 163 | 164 |
| | like (LOC115723207) | | | |
| N/A | BTB/POZ domain-containing protein | + | 165 | 166 |
| | At2g30600 (LOC115713817), transcript | | | |
| | variant X3 | | | |
| N/A | BURP domain protein RD22-like | + | 167 | 168 |
| | (LOC115702309) | | | |
| N/A | calcium-binding protein CBP-like | + | 169 | 170 |
| | (LOC115710108) | | | |
| N/A | calvin cycle protein CP12-1, | + | 171 | 172 |
| | chloroplastic-like (LOC115705832) | | | |
| N/A | cationic peroxidase 2-like | ++++ | 173 | 174 |
| | (LOC115725319) | | | |
| N/A | cationic peroxidase 2-like | + | 175 | 176 |
| | (LOC115725319) | | | |
| N/A | cyclopropane-fatty-acyl-phospholipid | + | 177 | 178 |
| | synthase-like (LOC115716432), | | | |
| | transcript variant X2 | | | |
| N/A | cytochrome B5-like protein | ++ | 179 | 180 |
| | (LOC115697002) | | | |
| N/A | developmentally-regulated G-protein 3 | + | 181 | 182 |
| | (LOC115713495) | | | |
| N/A | disease resistance protein RGA2-like | + | 183 | 184 |
| | (LOC115697928) | | | |
| N/A | DNA-directed RNA polymerase II subunit | + | 185 | 186 |
| | RPB2 (LOC115697994) | | | |
| N/A | dormancy-associated protein homolog 3- | + | 187 | 188 |
| | like (LOC115705623), transcript variant | | | |
| | X1 | | | |
| N/A | E3 ubiquitin-protein ligase At3g02290- | + | 189 | 190 |
| | like (LOC115699479) | | | |
| N/A | E3 ubiquitin-protein ligase RDUF2 | + | 191 | 192 |
| | (LOC115719629) | | | |
| N/A | elongation factor 1-alpha | + | 193 | 194 |
| | (LOC115719034), transcript variant X2 | | | |
| N/A | elongation factor 2 (LOC115709092) | + | 195 | 196 |
| N/A | FIP1[V]-like protein (LOC115724951), | + | 197 | 198 |
| | transcript variant X2 | | | |
| N/A | GTP-binding protein At2g22870 | + | 199 | 200 |
| | (LOC115723277) | | | |
| N/A | KH domain-containing protein HEN4 | + | 201 | 202 |
| | (LOC115714344) | | | |
| N/A | MLO-like protein 1 (LOC115697297) | + | 203 | 204 |
| N/A | MLP-like protein 423 (LOC115712860) | +++ | 205 | 206 |
| N/A | NDR1/HIN1-like protein 1 | +++ | 207 | 208 |
| | (LOC115699613) | | | |
| N/A | peroxidase 12-like (LOC115708240) | +++ | 209 | 210 |
| N/A | peroxidase 42 (LOC115707759) | + | 211 | 212 |
| N/A | phosphoinositide phosphatase SAC1 | ++ | 213 | 214 |
| | (LOC115714680) | | | |
| N/A | polyphenol oxidase, chloroplastic-like | + | 215 | 216 |
| | (LOC115707591) | | | |
| N/A | probable BOI-related E3 ubiquitin-protein | + | 217 | 218 |
| | ligase 3 (LOC115709298) | | | |
| N/A | probable methyltransferase PMT21 | + | 219 | 220 |
| | (LOC115716678) | | | |
| N/A | probable NAD(P)H dehydrogenase | + | 221 | 222 |
| | (quinone) FQR1-like 3 (LOC115718897), | | | |
| | transcript variant X4 | | | |
| N/A | protein BOBBER 1 (LOC115716844) | + | 223 | 224 |
| N/A | protein LURP-one-related 8-like | + | 225 | 226 |
| | (LOC115716623) | | | |
| N/A | protein OBERON 4-like | + | 227 | 228 |
| | (LOC115717225) | | | |
| N/A | putative DEAD-box ATP-dependent RNA | + | 229 | 230 |
| | helicase 29 (LOC115711108) | | | |
| N/A | pyruvate decarboxylase 1-like | + | 231 | 232 |
| | (LOC115718871) | | | |
| N/A | remorin 4.1 (LOC115709911) | + | 233 | 234 |
| N/A | serine/threonine-protein kinase VPS15- | + | 235 | 236 |
| | like (LOC115705223), transcript variant | | | |
| | X2 | | | |
| N/A | stromal 70 kDa heat shock-related | + | 237 | 238 |
| | protein, chloroplastic (LOC115725252) | | | |
| N/A | structural maintenance of chromosomes | +++ | 239 | 240 |
| | protein 1 (LOC115714502) | | | |
| N/A | transcription factor GTE3, chloroplastic- | + | 241 | 242 |
| | like (LOC115697388) | | | |
| N/A | translationally-controlled tumor protein | ++ | 243 | 244 |
| | homolog (LOC115722265) | | | |
| N/A | translationally-controlled tumor protein | + | 245 | 246 |
| | homolog (LOC115722265) | | | |
| N/A | tubulin beta-2 chain (LOC115717306) | ++ | 247 | 248 |
| N/A | uncharacterized LOC115698826 | + | 249 | 250 |
| | (LOC115698826), transcript variant X6 | | | |
| N/A | uncharacterized LOC115698826 | + | 251 | 252 |
| | (LOC115698826), transcript variant X6 | | | |
| N/A | wound-induced protein 1 | + | 253 | 254 |
| | (LOC115705480) | | | |
| N/A | 1-acyl-sn-glycerol-3-phosphate | +++ | 255 | 256 |
| | acyltransferase 2 (LOC115709728) | | | |
| N/A | auxin-repressed 12.5 kDa protein | + | 257 | 258 |
| | (LOC115697432) | | | |
| N/A | beta-galactosidase 3 (LOC115715122) | + | 259 | 260 |
| N/A | BURP domain protein RD22-like | ++ | 261 | 262 |
| | (LOC115702309) | | | |
| N/A | chaperone protein dnaJ 11, | + | 263 | 264 |
| | chloroplastic-like (LOC115714010) | | | |
| N/A | coiled-coil domain-containing protein 12 | + | 265 | 266 |
| | (LOC115698197) | | | |
| N/A | cysteine-rich receptor-like protein kinase | + | 267 | 268 |
| | 42 (LOC115709148), transcript variant | | | |
| | X2 | | | |
| N/A | desiccation-related protein PCC13-62- | ++++ | 269 | 270 |
| | like (LOC115722674) | | | |
| N/A | dnaJ homolog subfamily B member 3- | + | 271 | 272 |
| | like (LOC115707149), transcript variant | | | |
| | X2 | | | |
| N/A | dormancy-associated protein 2-like | ++++ | 273 | 274 |
| | (LOC115701261) | | | |
| N/A | double-stranded RNA-binding protein 3- | + | 275 | 276 |
| | like (LOC115701449), transcript variant | | | |
| | X2 | | | |
| N/A | dymeclin (LOC115721706), transcript | + | 277 | 278 |
| | variant X2 | | | |
| N/A | early nodulin-75-like (LOC115722790) | + | 279 | 280 |
| N/A | EIN3-binding F-box protein 1 | + | 281 | 282 |
| | (LOC115720532) | | | |
| N/A | ELL-associated factor 2 | + | 283 | 284 |
| | (LOC115713953), transcript variant X4 | | | |
| N/A | F-box/kelch-repeat protein At1g51550- | + | 285 | 286 |
| | like (LOC115713416) | | | |
| N/A | formamidase-like (LOC115713064), | + | 287 | 288 |
| | transcript variant X2 | | | |
| N/A | glycine-rich cell wall structural protein 2- | + | 289 | 290 |
| | like (LOC115706861) | | | |
| N/A | glycine-rich protein 2-like | + | 291 | 292 |
| | (LOC115702777) | | | |
| N/A | guanine nucleotide-binding protein-like | + | 293 | 294 |
| | NSN1 (LOC115713023) | | | |
| N/A | HMG1/2-like protein (LOC115702610), | + | 295 | 296 |
| | transcript variant X2 | | | |
| N/A | late embryogenesis abundant protein, | + | 297 | 298 |
| | group 3-like (LOC115696873) | | | |
| N/A | mannose-1-phosphate | +++ | 299 | 300 |
| | guanylyltransferase 1 (LOC115704726), | | | |
| | transcript variant X2 | | | |
| N/A | metallothionein-like protein 2 | + | 301 | 302 |
| | (LOC115719445) | | | |
| N/A | methyl-CpG-binding domain-containing | + | 303 | 304 |
| | protein 11-like (LOC115720689) | | | |
| N/A | NADH dehydrogenase [ubiquinone] | ++ | 305 | 306 |
| | flavoprotein 2, mitochondrial | | | |
| | (LOC115707882) | | | |
| N/A | NDR1/HIN1-like protein 1 | + | 307 | 308 |
| | (LOC115699613) | | | |
| N/A | polyubiquitin (LOC115709395) | + | 309 | 310 |
| N/A | probable E3 ubiquitin-protein ligase | + | 311 | 312 |
| | RHC2A (LOC115697688), transcript | | | |
| | variant X1 | | | |
| N/A | probable gamma-secretase subunit | +++ | 313 | 314 |
| | PEN-2 (LOC115714842), transcript | | | |
| | variant X4 | | | |
| N/A | probable transmembrane ascorbate | + | 315 | 316 |
| | ferrireductase 4 (LOC115699328) | | | |
| N/A | programmed cell death protein 2-like | +++ | 317 | 318 |
| | (LOC115710273) | | | |
| N/A | protein MEMO1 (LOC115698915) | + | 319 | 320 |
| N/A | protein ROOT HAIR DEFECTIVE 3 | + | 321 | 322 |
| | (LOC115706947), transcript variant X2 | | | |
| N/A | protein SPIRAL1-like 1 (LOC115699427) | + | 323 | 324 |
| N/A | protein SRC1 (LOC115722177) | ++ | 325 | 326 |
| N/A | putative methyltransferase | + | 327 | 328 |
| | DDB_G0268948 (LOC115715148) | | | |
| N/A | rac-like GTP-binding protein ARAC1 | + | 329 | 330 |
| | (LOC115702674), transcript variant X2 | | | |
| N/A | scarecrow-like transcription factor PAT1 | + | 331 | 332 |
| | (LOC115724919), transcript variant X2 | | | |
| N/A | small acidic protein 1 (LOC115721791) | ++++ | 333 | 334 |
| N/A | sorcin-like (LOC115701373), transcript | + | 335 | 336 |
| | variant X1 | | | |
| N/A | sphingoid long-chain bases kinase 1 | ++++ | 337 | 338 |
| | (LOC115723288), transcript variant X2 | | | |
| N/A | stress-associated endoplasmic reticulum | + | 339 | 340 |
| | protein 2-like (LOC115712498) | | | |
| N/A | transcription factor bHLH69-like | + | 341 | 342 |
| | (LOC115707361), transcript variant X3 | | | |
| N/A | transcription factor MYB1R1 | + | 343 | 344 |
| | (LOC115713616) | | | |
| N/A | transmembrane protein 128 | + | 345 | 346 |
| | (LOC115695749) | | | |
| N/A | TVP38/TMEM64 family membrane | + | 347 | 348 |
| | protein slr0305-like (LOC115716862) | | | |
| N/A | ubiquitin-conjugating enzyme E2-17 | +++ | 349 | 350 |
| | kDa-like (LOC115717294) | | | |
| N/A | ubiquitin-like domain-containing protein | + | 351 | 352 |
| | CIP73 (LOC115724769), transcript | | | |
| | variant X4 | | | |
| N/A | uncharacterized LOC115695809 | + | 353 | 354 |
| | (LOC115695809) | | | |
| N/A | uncharacterized LOC115695899 | ++++ | 355 | 356 |
| | (LOC115695899) | | | |
| N/A | uncharacterized LOC115697356 | + | 357 | 358 |
| | (LOC115697356) | | | |
| N/A | uncharacterized LOC115697715 | + | 359 | 360 |
| | (LOC115697715) | | | |
| N/A | uncharacterized LOC115697907 | + | 361 | 362 |
| | (LOC115697907), transcript variant X1, | | | |
| | ncRNA | | | |
| N/A | uncharacterized LOC115697972 | + | 363 | 364 |
| | (LOC115697972) | | | |
| N/A | uncharacterized LOC115698769 | + | 365 | 366 |
| | (LOC115698769) | | | |
| N/A | uncharacterized LOC115700000 | ++ | 367 | 368 |
| | (LOC115700000), transcript variant X7 | | | |
| N/A | uncharacterized LOC115700496 | + | 369 | 370 |
| | (LOC115700496) | | | |
| N/A | uncharacterized LOC115702105 | ++++ | 371 | 372 |
| | (LOC115702105), transcript variant X5 | | | |
| N/A | uncharacterized LOC115705265 | ++ | 373 | 374 |
| | (LOC115705265) | | | |
| N/A | uncharacterized LOC115705385 | + | 375 | 376 |
| | (LOC115705385) | | | |
| N/A | uncharacterized LOC115705912 | + | 377 | 378 |
| | (LOC115705912), transcript variant X4 | | | |
| N/A | uncharacterized LOC115706141 | + | 379 | 380 |
| | (LOC115706141) | | | |
| N/A | uncharacterized LOC115707705 | + | 381 | 382 |
| | (LOC115707705), ncRNA | | | |
| N/A | uncharacterized LOC115708684 | + | 383 | 384 |
| | (LOC115708684) | | | |
| N/A | uncharacterized LOC115709037 | + | 385 | 386 |
| | (LOC115709037), ncRNA | | | |
| N/A | uncharacterized LOC115713062 | + | 387 | 388 |
| | (LOC115713062), transcript variant X2 | | | |
| N/A | uncharacterized LOC115714545 | + | 389 | 390 |
| | (LOC115714545), transcript variant X2 | | | |
| N/A | uncharacterized LOC115715077 | + | 391 | 392 |
| | (LOC115715077) | | | |
| N/A | uncharacterized LOC115716362 | + | 393 | 394 |
| | (LOC115716362), transcript variant X7, | | | |
| | misc_RNA | | | |
| N/A | uncharacterized LOC115716703 | + | 395 | 396 |
| | (LOC115716703) | | | |
| N/A | uncharacterized LOC115717625 | ++++ | 397 | 398 |
| | (LOC115717625) | | | |
| N/A | uncharacterized LOC115718492 | + | 399 | 400 |
| | (LOC115718492), transcript variant X3, | | | |
| | ncRNA | | | |
| N/A | uncharacterized LOC115718913 | + | 401 | 402 |
| | (LOC115718913) | | | |
| N/A | uncharacterized LOC115719070 | + | 403 | 404 |
| | (LOC115719070), transcript variant X2 | | | |
| N/A | uncharacterized LOC115720642 | ++ | 405 | 406 |
| | (LOC115720642) | | | |
| N/A | uncharacterized LOC115721294 | + | 407 | 408 |
| | (LOC115721294) | | | |
| N/A | uncharacterized LOC115721758 | ++++ | 409 | 410 |
| | (LOC115721758) | | | |
| N/A | uncharacterized LOC115722211 | + | 411 | 412 |
| | (LOC115722211) | | | |
| N/A | uncharacterized LOC115722982 | + | 413 | 414 |
| | (LOC115722982) | | | |
| N/A | uncharacterized LOC115724511 | + | 415 | 416 |
| | (LOC115724511), transcript variant X3 | | | |
| N/A | uncharacterized protein At5g39570 | + | 417 | 418 |
| | (LOC115701425) | | | |
| N/A | universal stress protein PHOS32 | + | 419 | 420 |
| | (LOC115722867) | | | |
| N/A | universal stress protein PHOS34-like | + | 421 | 422 |
| | (LOC115697473) | | | |
| N/A | upstream activation factor subunit | ++++ | 423 | 424 |
| | UAF30 (LOC115704517), transcript | | | |
| | variant X2 | | | |
| N/A | uridine kinase-like protein 1, | +++ | 425 | 426 |
| | chloroplastic (LOC115699666) | | | |
| N/A | wiskott-Aldrich syndrome protein family | + | 427 | 428 |
| | member 2-like (LOC115707029) | | | |
| N/A | YTH domain-containing protein ECT4- | ++ | 429 | 430 |
| | like (LOC115725203), transcript variant | | | |
| | X2 | | | |
| Nucleotide | adenosine kinase 2 (LOC115705417) | + | 431 | 432 |
| metabolism | dihydroorotase, mitochondrial-like | + | 433 | 434 |
| | (LOC115700571), transcript variant X2 | | | |
| | guanosine deaminase-like | + | 435 | 436 |
| | (LOC115723428), transcript variant X1 | | | |
| Phytohormone | E3 ubiquitin-protein ligase SDIR1 | ++ | 437 | 438 |
| action | (LOC115706206), transcript variant X7 | | | |
| | gibberellin-regulated protein 6 | + | 439 | 440 |
| | (LOC115708697) | | | |
| | protein phosphatase 2C 16-like | + | 441 | 442 |
| | (LOC115707022), transcript variant X2 | | | |
| Protein | probable protein phosphatase 2C 60 | ++ | 443 | 444 |
| modification. | (LOC115707957), transcript variant X2 | | | |
| phosphorylation | probable protein phosphatase 2C 9 | + | 445 | 446 |
| | (LOC115724962) | | | |
| | probable serine/threonine-protein kinase | + | 447 | 448 |
| | WNK9 (LOC115722879) | | | |
| | serine/threonine-protein kinase STY13- | + | 449 | 450 |
| | like (LOC115699359) | | | |
| | serine/threonine-protein phosphatase | + | 451 | 452 |
| | PP1 isozyme 2 (LOC115718952), | | | |
| | transcript variant X2 | | | |
| | U-box domain-containing protein 34 | ++ | 453 | 454 |
| | (LOC115706965) | | | |
| Protein | peptidyl-prolyl cis-trans isomerase | + | 455 | 456 |
| modification. | CYP19-3 (LOC115720741) | | | |
| protein folding | peptidyl-prolyl cis-trans isomerase | +++ | 457 | 458 |
| | CYP19-3 (LOC115720741) | | | |
| | peptidyl-prolyl cis-trans isomerase | + | 459 | 460 |
| | FKBP12 (LOC115695624) | | | |
| Protein | probable glutathione S-transferase | + | 461 | 462 |
| modification. | (LOC115698237) | | | |
| S-glutathionylation | | | | |
| Protein | mitochondrial import inner membrane | + | 463 | 464 |
| translocation | translocase subunit TIM23-3 | | | |
| | (LOC115719112) | | | |
| | protein TIC 56, chloroplastic | ++++ | 465 | 466 |
| | (LOC115697383) | | | |
| Redox homeostasis | probable nucleoredoxin 1 | + | 467 | 468 |
| | (LOC115712631) | | | |
| | probable phytol kinase 3, chloroplastic | + | 469 | 470 |
| | (LOC115719607) | | | |
| RNA biosynthesis | ethylene-responsive transcription factor | + | 471 | 472 |
| | 4-like (LOC115697318) | | | |
| | GATA transcription factor 28-like | + | 473 | 474 |
| | (LOC115719706), transcript variant X2 | | | |
| | mediator of RNA polymerase II | ++ | 475 | 476 |
| | transcription subunit 11 | | | |
| | (LOC115713950) | | | |
| | myb-related protein 308-like | + | 477 | 478 |
| | (LOC115704719) | | | |
| | myb-related protein 308-like | + | 479 | 480 |
| | (LOC115704719) | | | |
| | PHD finger protein ALFIN-LIKE 2-like | + | 481 | 482 |
| | (LOC115697427) | | | |
| | probable WRKY transcription factor 17 | + | 483 | 484 |
| | (LOC115700154) | | | |
| | protein Dr1 homolog (LOC115709923), | + | 485 | 486 |
| | transcript variant X1 | | | |
| | protein METHYLENE BLUE | + | 487 | 488 |
| | SENSITIVITY 1-like (LOC115697944) | | | |
| | protein METHYLENE BLUE | + | 489 | 490 |
| | SENSITIVITY 1-like (LOC115719535) | | | |
| | protein REVEILLE 8 (LOC115709988), | ++ | 491 | 492 |
| | transcript variant X3 | | | |
| | putative homeobox-leucine zipper | + | 493 | 494 |
| | protein ATHB-51 (LOC115708712) | | | |
| | TATA box-binding protein-associated | + | 495 | 496 |
| | factor RNA polymerase I subunit B-like | | | |
| | (LOC115719006), transcript variant X2 | | | |
| RNA processing | 110 kDa U5 small nuclear | + | 497 | 498 |
| | ribonucleoprotein component CLO | | | |
| | (LOC115712844) | | | |
| | glycine-rich RNA-binding protein-like | ++++ | 499 | 500 |
| | (LOC115714982) | | | |
| | probable CCR4-associated factor 1 | + | 501 | 502 |
| | homolog 11 (LOC115699398) | | | |
| | probable CCR4-associated factor 1 | + | 503 | 504 |
| | homolog 7 (LOC115707916) | | | |
| | probable CCR4-associated factor 1 | + | 505 | 506 |
| | homolog 7 (LOC115707916) | | | |
| | protein BUD31 homolog 2 | + | 507 | 508 |
| | (LOC115701272), transcript variant X2 | | | |
| | serine/arginine-rich-splicing factor SR34- | + | 509 | 510 |
| | like (LOC115696009), transcript variant | | | |
| | X1 | | | |
| | splicing factor-like protein 1 | + | 511 | 512 |
| | (LOC115712402) | | | |
| | U1 small nuclear ribonucleoprotein C | + | 513 | 514 |
| | (LOC115723141), transcript variant X2 | | | |
| RNA processing. | THO complex subunit 4D | + | 515 | 516 |
| messenger | (LOC115699271) | | | |
| ribonucleoparticle | | | | |
| (mRNP) | | | | |
| export.TREX/THO | | | | |
| mRNP trafficking | | | | |
| complex. mRNA- | | | | |
| binding adaptor | | | | |
| component | | | | |
| ALY/Tho4 | | | | |
| RNA processing. | Chloroplast stem-loop binding protein of | ++ | 517 | 518 |
| organelle | 41 kDa b, chloroplastic (LOC115720648) | | | |
| machinery. | | | | |
| ribonuclease | | | | |
| activities. | | | | |
| Secondary | 4-hydroxy-3-methylbut-2-en-1-yl | + | 519 | 520 |
| metabolism. | diphosphate synthase (ferredoxin), | | | |
| terpenoids. | chloroplastic (LOC115720893), transcript | | | |
| | variant X2 | | | |
| | acetyl-CoA acetyltransferase, cytosolic 1 | + | 521 | 522 |
| | (LOC115699135) | | | |
| | germacrene-A synthase-like | ++ | 523 | 524 |
| | (LOC115695866), transcript variant X1 | | | |
| | protein CHUP1, chloroplastic | + | 525 | 526 |
| | (LOC115707563), transcript variant X3 | | | |
| Solute transport | ABC transporter D family member 1 | + | 527 | 528 |
| | (LOC115718469) | | | |
| | mitochondrial outer membrane protein | + | 529 | 530 |
| | porin of 36 kDa (LOC115700790) | | | |
| | V-type proton ATPase subunit B 1 | ++ | 531 | 532 |
| | (LOC115702897), transcript variant X2 | | | |
| Solute transport. | Mitochondrial uncoupling protein 5 | + | 533 | 534 |
| carrier-mediated | (LOC115712714) | | | |
| transport. | protein NRT1/PTR FAMILY 2.13-like | + | 535 | 536 |
| | (LOC115699121) | | | |
| Vesicle trafficking | protein CASP (LOC115708935) | ++++ | 537 | 538 |
| | uncharacterized LOC115725103 | + | 539 | 540 |
| | (LOC115725103) | | | |

¹“Protein Function” correspond to plant protein BIN Descriptions described in Schwacke et al., (2019). “MapMan4: A Refined Protein Classification and Annotation Framework Applicable to Multi-Omics Data Analysis.” Mol. Plant. 12, 879-892.
²“Annotation” correspond to known annotations of the C. sativa genome based on the cs10 cannabis assembly (see at: www.ncbi.nlm.nih.gov/assembly/GCF_900626175.1/) with those specific locations of the genome not yet annotated indicated as “uncharacterized.”
³“FIOPC” is “fold-improvement relative to positive control” which was determined by screening as described in Example 1. FIOPC ranges are indicated as follows: + > 2.0; ++ > 3.0; +++ > 4.0; ++++ > 5.0

All of the C. sativa genes derived from trichome mRNAs shown in Table 2 were capable of providing at least 2-fold improvement of CBGA production in a recombinant host cell system that converts hexanoic acid to CBGA via a pathway comprising the enzymes AAE, OLS, OAC and PT4 (see Table 1). The trichome mRNA derived genes of Table 2, however, encode a wide range of proteins that do not appear to be directly involved with that with the hexanoic acid to CBGA pathway. Accordingly, in at least one embodiment, the present disclosure provides a recombinant host cell comprising a pathway capable of producing a cannabinoid and a nucleic acid derived from a Cannabis trichome mRNA, wherein the nucleic acid comprises a nucleotide sequence having at least 90% identity to any one of odd-numbered SEQ ID NOs: 15-539. In at least one embodiment, the nucleic acid comprises a nucleotide sequence having at least 95%, at least 96%, at least 97%, at least 98%, or at least 99% identity to any one of odd-numbered SEQ ID NOs: 15-539. In at least one embodiment, the nucleic acid comprises a nucleotide sequence of any one of odd-numbered SEQ ID NOs: 15-539.

In at least one embodiment, the present disclosure provides a recombinant host cell comprising a pathway capable of producing a cannabinoid and a nucleic acid derived from a Cannabis trichome mRNA, wherein the nucleic acid comprises a nucleotide sequence having at least 90%, at least 95%, at least 96%, at least 97%, at least 98%, or at least 99% identity to any one of SEQ ID NOs: 35, 73, 87, 93, 103, 105, 107, 109, 111, 113, 115, 119, 121, 123, 125, 127, 129, 131, 133, 135, 137, 139, 141, 143, 145, 147, 149, 167, 173, 183, 187, 205, 209, 231, 269, 273, and 395.

In at least one embodiment, the present disclosure provides an isolated nucleic acid derived from a Cannabis trichome mRNA, wherein the nucleic acid encodes a polypeptide comprising an amino acid sequence having at least 90% identity to any one of even-numbered SEQ ID NOs: 16-540. In at least one embodiment, the nucleotide sequence of the isolated nucleic acid is codon-optimized for expression in a recombinant host cell, wherein the host cell source is selected from Saccharomyces cerevisiae, Yarrowia lipolytica, Pichia pastoris, Escherichia coli, or an engineered cell derived from Saccharomyces cerevisiae, Yarrowia lipolytica, Pichia pastoris, Escherichia coli. In at least one embodiment, the isolated nucleic acid derived from Cannabis trichome mRNA comprises a nucleotide sequence having at least 90% identity to any one of odd-numbered SEQ ID NOs: 15-539.

In at least one embodiment, the present disclosure provides a vector comprising a nucleic acid derived from a Cannabis trichome mRNA, wherein the nucleic acid encodes a polypeptide comprising an amino acid sequence having at least 90% identity to any one of even-numbered SEQ ID NOs: 16-540. In at least one embodiment, the vector comprises nucleic acid that is codon-optimized for expression in a recombinant host cell, wherein the host cell source is selected from Saccharomyces cerevisiae, Yarrowia lipolytica, Pichia pastoris, Escherichia coli, or an engineered cell derived from Saccharomyces cerevisiae, Yarrowia lipolytica, Pichia pastoris, Escherichia coli. In at least one embodiment, the vector comprising the nucleic acid derived from Cannabis trichome mRNA comprises a nucleotide sequence having at least 90% identity to any one of odd-numbered SEQ ID NOs: 15-539.

In at least one embodiment, the present disclosure provides a vector comprising a nucleic acid encoding a pathway of enzymes capable of producing a cannabinoid and a nucleic acid derived from a Cannabis trichome mRNA, wherein the nucleic acid encodes a polypeptide comprising an amino acid sequence having at least 90% identity to any one of even-numbered SEQ ID NOs: 16-540. In at least one embodiment, the pathway capable of producing a cannabinoid comprises at least the exemplary enzymes AAE, OLS, OAC, and PT4, wherein the enzymes have the amino acid sequences of SEQ ID NO: 2 (AAE), SEQ ID NO: 4 (OLS), SEQ ID NO: 6 (OAC), and SEQ ID NO: 8 (PT4). In at least one embodiment, the vector comprises nucleic acid sequences encoding the pathway of enzymes that are codon-optimized for expression in a recombinant host cell, wherein the host cell source is selected from Saccharomyces cerevisiae, Yarrowia lipolytica, Pichia pastoris, Escherichia coli, or an engineered cell derived from Saccharomyces cerevisiae, Yarrowia lipolytica, Pichia pastoris, Escherichia coll.

In at least one embodiment, the present disclosure provides a recombinant host cell comprising a pathway capable of producing a cannabinoid and a nucleic acid derived from a Cannabis trichome mRNA, wherein the nucleic acid encodes a polypeptide comprising an amino acid sequence having at least 80% identity to any one of even-numbered SEQ ID NOs: 16-540. In at least one embodiment, the nucleic acid encodes a polypeptide comprising an amino acid sequence having at least 85%, at least 90%, at least 95%, at least 96%, at least 97%, at least 98%, or at least 99% identity to any one of even-numbered SEQ ID NOs: 16-540.

In at least one embodiment, the present disclosure provides a recombinant host cell comprising a pathway capable of producing a cannabinoid and a nucleic acid derived from a Cannabis trichome mRNA, wherein the nucleic acid encodes a polypeptide comprising an amino acid sequence having at least 80% identity, at least 85%, at least 90%, at least 95%, at least 96%, at least 97%, at least 98%, or at least 99% to any one of SEQ ID NOs: 36, 74, 88, 94, 104, 106, 108, 110, 112, 114, 116, 120, 122, 124, 126, 128, 130, 132, 134, 136, 138, 140, 142, 144, 146, 148, 150, 168, 174, 184, 188, 206, 210, 232, 270, 274, and 396.

The amino acid sequences of even-numbered SEQ ID NOs: 16-540 provided in the present disclosure begin with initiating methionine (M) residue at position 1, although it will be understood by the skilled artisan that this initiating methionine residue may be removed by biological processing machinery, such as in a host cell or in vitro translation system, to generate a mature protein lacking the initiating methionine residue. Accordingly, it is contemplated that any embodiment of the present disclosure comprising an enzyme of Table 2 can comprising an amino acid sequence of even-numbered SEQ ID NOs: 16-540 with the methionine residue at position 1 deleted.

As further described in the Examples, the off-pathway trichome mRNA derived genes of Table 2 are capable of providing at least 2-fold, and up to 5-fold or greater increased cannabinoid production when introduced into a recombinant host cell comprising a heterologous cannabinoid biosynthesis pathway. Accordingly, in at least one embodiment, the present disclosure provides a recombinant host cell comprising a pathway capable of producing a cannabinoid and a nucleic acid derived from a Cannabis trichome mRNA, wherein the recombinant host cell produces the cannabinoid with a titer that is increased at least 1.2-fold, at least 2-fold, at least 3-fold, at least 4-fold, at least 5-fold, or more relative to a control recombinant host cell comprising the pathway and not the nucleic acid derived from a Cannabis trichome mRNA. For example, a control recombinant yeast cell that can convert hexanoic acid to CBGA but that has not been transformed with a heterologous nucleic acid derived from a C. sativa trichome mRNA of Table 2.

The nucleic acids derived from C. sativa trichome mRNA that are identified in Table 2 encode a wide range of protein functions that generally would not be identified a priori as capable of enhancing cannabinoid production in a recombinant host cell. The exemplary protein functions include: amino acid biosynthesis, amino acid degradation, carbohydrate metabolism, carrier-mediated transport, cell cycle organization, cellular respiration, chromatin organization, circadian clock system regulation, coenzyme metabolism, EC_1 oxidoreductase, EC_2 transferase, EC_3 hydrolase, EC_4 lyase, effector-triggered immunity (ETI) network co-regulatory protein (RAR1), fatty acid biosynthesis, lipid degradation, lipid transfer type protein, messenger ribonucleoparticle (mRNP) export, mulatexin-like, organelle machinery ribonuclease activity, phytohormone action, protein folding, protein phosphorylation, protein S-glutathionylation, protein translocation, redox homeostasis, RNA biosynthesis, RNA processing, solute transport, terpenoid metabolism, and vesicle trafficking.

Without intending to be bound by theory, it is believed that these off-pathway proteins that are encoded by nucleic acids derived from Cannabis trichome mRNA can enhance cannabinoid production in a recombinant host via a range of mechanisms including but not limited to: (i) lipid transfer proteins can mediate cannabinoid transfer across the cytoplasm to cell membranes in the host cell; (ii) peroxidases can scavenge the peroxides that are formed as a result of THCA and CBDA synthesis, alleviating damage to the host cell; (iii) acyl-acyl carrier protein thioesterases can determine the chain length of fatty acids during their formation, thereby producing fatty acids with chain lengths favourable for cannabinoid synthesis; (iv) desaturases can introduce double-bonds in acyl lipids, eventually leading to the formation of hexanoyl-CoA, a key precursor in the cannabinoid pathway; (v) malonate-CoA ligase utilizes malonate and CoA to form malonyl-CoA, another key precursor in the cannabinoid pathway; (vi) major allergen Pru av 1-like proteins, which include major latex protein-like proteins, bind hydrophobic compounds, that can assist in intracellular trafficking of cannabinoid precursors and/or prevent degradation of these precursors; and (vii) patatin-like proteins, acting as lipases, break down phospholipids from into fatty acids which can serve as precursors for cannabinoid synthesis.

Accordingly, in at least one embodiment, the present disclosure provides a recombinant host cell comprising a pathway capable of producing a cannabinoid and a nucleic acid derived from a Cannabis trichome mRNA that does not encode an enzyme in the pathway, wherein the nucleic acid derived from a Cannabis trichome mRNA encodes a polypeptide having a protein function corresponding to a MapMan4 Bin Description selected from: amino acid biosynthesis, amino acid degradation, carbohydrate metabolism, carrier-mediated transport, cell cycle organization, cellular respiration, chromatin organization, circadian clock system regulation, coenzyme metabolism, EC_1 oxidoreductase, EC_2 transferase, EC_3 hydrolase, EC_4 lyase, effector-triggered immunity (ETI) network co-regulatory protein (RAR1), fatty acid biosynthesis, lipid degradation, lipid transfer type protein, messenger ribonucleoparticle (mRNP) export, mulatexin-like protein, organelle machinery ribonuclease activity, phytohormone action, protein folding, protein phosphorylation, protein S-glutathionylation, protein translocation, redox homeostasis, RNA biosynthesis, RNA processing, solute transport, terpenoid metabolism, and/or vesicle trafficking.

In at least one embodiment, the present disclosure provides a recombinant host cell comprising a pathway capable of producing a cannabinoid and a nucleic acid derived from a Cannabis trichome mRNA, wherein the nucleic acid derived from a Cannabis trichome mRNA encodes a polypeptide having a protein function selected from: carbohydrate metabolism, EC_1 oxidoreductase, EC_2 transferase, fatty acid biosynthesis, lipid degradation, lipid transfer type protein, and mulatexin-like protein.

For example, polypeptides having a protein function of carbohydrate metabolism can include pyruvate decarboxylase 1-like, and/or can include a polypeptide comprising an amino acid sequence having at 90% identity to an amino acid sequence of SEQ ID NO: 36; polypeptides having a protein function of EC_1 oxidoreductase can include 2-oxoglutarate-dependent dioxygenase, and/or can include a polypeptide comprising an amino acid sequence having at 90% identity to an amino acid sequence of SEQ ID NO: 74; polypeptides having a protein function of EC_2 transferase can include protein ECERIFERUM 26-like, or stemmadenine 0-acetyltransferase-like, and/or can include a polypeptide comprising an amino acid sequence having at 90% identity to an amino acid sequence of SEQ ID NO: 88, or 94; polypeptides having a protein function of fatty acid biosynthesis can include 3-ketoacyl-CoA synthase 6, acyl carrier protein 1 (chloroplastic-like), or malonate—CoA ligase, and/or can include a polypeptide comprising an amino acid sequence having at 90% identity to an amino acid sequence of SEQ ID NO: 104, 106, 108, 110, 112, or 114; polypeptides having a protein function of lipid degradation can include acyl-acyl carrier protein thioesterase ATL3, chloroplastic-like, or patatin-like protein 1, and/or can include a polypeptide comprising an amino acid sequence having at 90% identity to an amino acid sequence of SEQ ID NO: 116, or 120; polypeptides having a protein function of lipid transfer type protein can include a polypeptide comprising an amino acid sequence having at 90% identity to an amino acid sequence of SEQ ID NO: 122, 124, 126, 128, 130, 132, 134, 136, 138, or 140; and polypeptides having a protein function of mulatexin-like protein can include a polypeptide comprising an amino acid sequence having at 90% identity to an amino acid sequence of SEQ ID NO: 142, 144, 146, 148, or 150. Accordingly, in at least one embodiment, the nucleic acid derived from a Cannabis trichome mRNA encodes a polypeptide comprising an amino acid sequence having at least 80% identity, at least 85%, at least 90%, at least 95%, at least 96%, at least 97%, at least 98%, or at least 99% to any one of SEQ ID NOs: 36, 74, 88, 94, 104, 106, 108, 110, 112, 114, 116, 120, 122, 124, 126, 128, 130, 132, 134, 136, 138, 140, 142, 144, 146, 148, and 150.

In at least one embodiment, the present disclosure provides a recombinant host cell comprising a pathway capable of producing a cannabinoid and a nucleic acid derived from a Cannabis trichome mRNA, wherein the nucleic acid derived from a Cannabis trichome mRNA encodes a polypeptide having a gene annotation (e.g., based on cs10 assembly) selected from: ECERIFERUM 26-like, stemmadenine O-acetyltransferase-like, 3-ketoacyl-CoA synthase 6, acyl carrier protein 1 (chloroplastic-like), malonate-CoA ligase, acyl-acyl carrier protein thioesterase ATL3, chloroplastic-like, patatin-like protein 1, mulatexin-like, non-specific lipid-transfer protein 1-like, non-specific lipid-transfer protein 2-like, BURP domain protein RD22-like, cationic peroxidase 2-like, disease resistance protein RGA2-like, MLP-like protein 423, peroxidase 12-like, pyruvate decarboxylase 1-like, desiccation-related protein PCC13-62-like, or dormancy-associated protein 2-like. Accordingly, in at least one embodiment, the nucleic acid derived from a Cannabis trichome mRNA encodes a polypeptide comprising an amino acid sequence having at least 80% identity, at least 85%, at least 90%, at least 95%, at least 96%, at least 97%, at least 98%, or at least 99% to any one of SEQ ID NOs: 36, 74, 88, 94, 104, 106, 108, 110, 112, 114, 116, 120, 122, 124, 126, 128, 130, 132, 134, 136, 138, 140, 142, 144, 146, 148, 150, 168, 174, 184, 188, 206, 210, 232, 270, or 274.

Among the off-pathway nucleic acids derived from Cannabis trichome mRNAs that resulted in over 4-fold improvement of CBGA yield were nucleic acids encoding protein types including: cell cycle organization, cellular respiration, EC_1 oxidoreductase, EC_2 transferase, lipid degradation, lipid transfer type protein, circadian clock system regulation, protein folding, protein translocation, RNA processing, and vesicle trafficking.

More specifically, the off-pathway nucleic acids derived from Cannabis trichome mRNAs that resulted in over 3-fold improvement of CBGA yield encoded proteins having the following annotations: 1-acyl-sn-glycerol-3-phosphate acyltransferase 2; 1-aminocyclopropane-1-carboxylate oxidase homolog 1-like; acyl-acyl carrier protein thioesterase ATL3, chloroplastic-like; barwin-like; beta-adaptin-like protein B; BURP domain protein RD22-like; cationic peroxidase 2-like; cell division control protein 2 homolog 2; chloroplast stem-loop binding protein of 41 kDa b, chloroplastic; cysteine-rich receptor-like protein kinase 19; cytochrome B5-like protein; delta(12)-fatty-acid desaturase FAD2-like; desiccation-related protein PCC13-62-like; dormancy-associated protein 2-like; E3 ubiquitin-protein ligase SDIR1; gamma carbonic anhydrase-like 2, mitochondrial; germacrene-A synthase-like; glucan endo-1,3-beta-glucosidase 12; glucose-6-phosphate 1-dehydrogenase 6, cytoplasmic-like; glycine-rich RNA-binding protein-like; major allergen Pru av 1-like; malonate—CoA ligase; mannose-1-phosphate guanylyltransferase 1; mediator of RNA polymerase II transcription subunit 11; MLP-like protein 423; NADH dehydrogenase [ubiquinone] flavoprotein 2, mitochondrial; NADP-dependent glyceraldehyde-3-phosphate dehydrogenase; NDR1/HIN1-like protein 1; non-specific lipid-transfer protein 1-like; non-specific lipid-transfer protein 2-like; ornithine aminotransferase, mitochondrial; peptidyl-prolyl cis-trans isomerase CYP19-3; peroxidase 12-like; phosphoinositide phosphatase SAC1; probable gamma-secretase subunit PEN-2; probable protein phosphatase 2C 60; programmed cell death protein 2-like; protein CASP; protein ELF4-LIKE 4-like; protein REVEILLE 8; protein SRC1; protein TIC 56, chloroplastic; pyruvate decarboxylase 1-like; small acidic protein 1; sphingoid long-chain bases kinase 1; structural maintenance of chromosomes protein 1; translationally-controlled tumor protein homolog; tubulin beta-2 chain; ubiquitin-conjugating enzyme E2-17 kDa-like; U-box domain-containing protein 34; upstream activation factor subunit UAF30; uridine kinase-like protein 1, chloroplastic; V-type proton ATPase subunit B 1; and YTH domain-containing protein ECT4-like.

Accordingly, it is contemplated that recombinant host engineered with a pathway of heterologous enzymes capable of producing a cannabinoid can have the biosynthetic yield of the cannabinoid increased or otherwise enhance by introduction a heterologous off-pathway nucleic acid that encodes a recombinant polypeptide having a protein function or enzymatic activity selected from the above list. Thus, in at least one embodiment, the present disclosure provides a recombinant host cell comprising a pathway capable of producing a cannabinoid and a nucleic acid derived from a Cannabis trichome mRNA, wherein the nucleic acid encodes a recombinant polypeptide having a protein function or enzymatic activity selected from: 1-acyl-sn-glycerol-3-phosphate acyltransferase 2; 1-aminocyclopropane-1-carboxylate oxidase homolog 1-like; acyl-acyl carrier protein thioesterase ATL3, chloroplastic-like; barwin-like; beta-adaptin-like protein B; BURP domain protein RD22-like; cationic peroxidase 2-like; cell division control protein 2 homolog 2; chloroplast stem-loop binding protein of 41 kDa b, chloroplastic; cysteine-rich receptor-like protein kinase 19; cytochrome B5-like protein; delta(12)-fatty-acid desaturase FAD2-like; desiccation-related protein PCC13-62-like; dormancy-associated protein 2-like; E3 ubiquitin-protein ligase SDIR1; gamma-carbonic anhydrase-like 2, mitochondrial; germacrene-A synthase-like; glucan endo-1,3-beta-glucosidase 12; glucose-6-phosphate 1-dehydrogenase 6, cytoplasmic-like; glycine-rich RNA-binding protein-like; major allergen Pru av 1-like; malonate—CoA ligase; mannose-1-phosphate guanylyltransferase 1; mediator of RNA polymerase II transcription subunit 11; MLP-like protein 423; NADH dehydrogenase [ubiquinone] flavoprotein 2, mitochondrial; NADP-dependent glyceraldehyde-3-phosphate dehydrogenase; NDR1/HIN1-like protein 1; non-specific lipid-transfer protein 1-like; non-specific lipid-transfer protein 2-like; ornithine aminotransferase, mitochondrial; peptidyl-prolyl cis-trans isomerase CYP19-3; peroxidase 12-like; phosphoinositide phosphatase SAC1; probable gamma-secretase subunit PEN-2; probable protein phosphatase 2C 60; programmed cell death protein 2-like; protein CASP; protein ELF4-LIKE 4-like; protein REVEILLE 8; protein SRC1; protein TIC 56, chloroplastic; pyruvate decarboxylase 1-like; small acidic protein 1; sphingoid long-chain bases kinase 1; structural maintenance of chromosomes protein 1; translationally-controlled tumor protein homolog; tubulin beta-2 chain; ubiquitin-conjugating enzyme E2-17 kDa-like; U-box domain-containing protein 34; upstream activation factor subunit UAF30; uridine kinase-like protein 1, chloroplastic; V-type proton ATPase subunit B 1; and YTH domain-containing protein ECT4-like.

Additionally, the nucleic acids derived from C. sativa trichome mRNA of Table 2 include the following non-annotated (or “uncharacterized”) proteins that are identified by their location in the Cannabis sativa genome. The nucleic acids also resulted in 2-fold or greater improvement of CBGA yield. Accordingly, it is contemplated that a recombinant host engineered with a pathway of heterologous enzymes capable of producing a cannabinoid can have the biosynthetic yield of the cannabinoid increased or otherwise enhanced by introduction a nucleic acid that encodes any of the polypeptides listed as “uncharacterized” in Table 2. Accordingly, in at least one embodiment, the nucleic acid derived from a Cannabis trichome mRNA encodes a polypeptide comprising an amino acid sequence having at least 80% identity, at least 85%, at least 90%, at least 95%, at least 96%, at least 97%, at least 98%, or at least 99% to any one of SEQ ID NOs: 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, or 418.

Use of Recombinant Host Cells in Preparation of Cannabinoids

As described elsewhere herein, the recombinant host cells provided by the present disclosure comprise a pathway capable of producing a cannabinoid and a nucleic acid derived from a Cannabis trichome mRNA that does not encode an enzyme in the pathway. Furthermore, the recombinant host cells are capable of producing the cannabinoid with a titer that is increased (e.g., 2-fold or more) relative to a control recombinant host cell comprising the same pathway but without the off-pathway nucleic acid derived from a Cannabis trichome mRNA. As described elsewhere herein, the off-pathway nucleic acid can encode a protein described in Table 2 and the associate Sequence Listing.

Although the cannabinoid pathways of FIGS. 1-2 depict the production of the more common naturally occurring cannabinoids, CBGA, Δ⁹-THCA, CBDA, and CBCA, it is also contemplated that the recombinant host cells comprising cannabinoid pathways and a heterologous “off-pathway” nucleic acid derived from C. sativa trichome mRNA, and associated methods of the present disclosure can also be used to biosynthesize a range of naturally occurring rare and/or synthetic cannabinoid compounds. Table 3 (below) depicts the names and structures of a wide range of exemplary cannabinoid compounds that are contemplated for production using the recombinant host cells and methods of the present disclosure.

TABLE 3
Exemplary cannabinoid compounds

| | Abbrev. | |
|---|---|---|
| Compound Name | Name | Chemical Structure |
| cannabigerolic acid | CBGA | 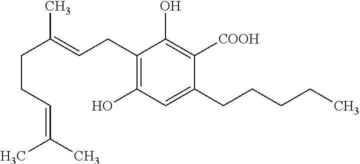 [SMILES: CCCCCc1cc(O)c(C/C=C(\C)CCC=C(C)C)c(O)c1C(=O)O] |
| cannabigerol | CBG | 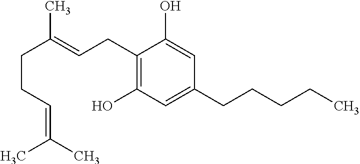 [SMILES: CCCCCc1cc(O)c(C/C=C(\C)CCC=C(C)C)c(O)c1] |
| Δ⁹-tetrahydrocannabinolic acid | Δ⁹- THCA | 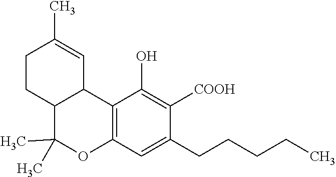 [SMILES: CCCCCc1cc2c(c(O)c1C(=O)O)C1C=C(C)CCC1C(C)(C)O2] |
| Δ⁹-tetrahydrocannabinol | Δ⁹-THC | 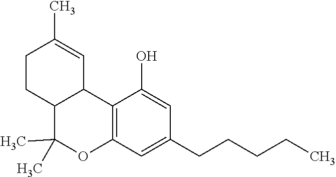 [SMILES: CCCCCc1cc(O)c2c(c1)OC(C)(C)C1CCC(C)=CC21] |
| Δ⁸-tetrahydrocannabinolic acid | Δ⁸- THCA | 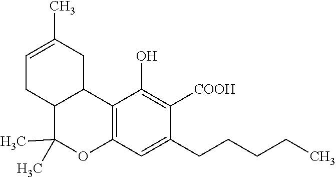 [SMILES: CCCCCc1cc2c(c(O)c1C(=O)O)C1CC(C)=CCC1C(C)(C)O2] |
| Δ⁸-tetrahydrocannabinol | Δ⁸-THC | 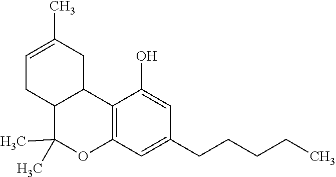 [SMILES: CCCCCc1cc(O)c2c(c1)OC(C)(C)C1CC=C(C)CC21] |
| cannabidiolic acid | CBDA | 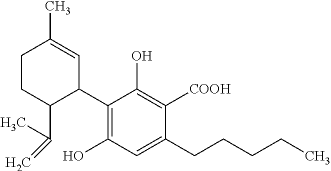 [SMILES: C=C(C)C1CCC(C)=CC1c1c(O)cc(CCCCC)c(C(=O)O)c1O] |
| cannabidiol | CBD | 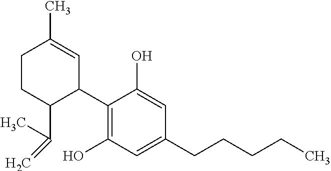 [SMILES: C=C(C)C1CCC(C)=CC1c1c(O)cc(CCCCC)cc1O] |
| cannabichromenic acid | CBCA | 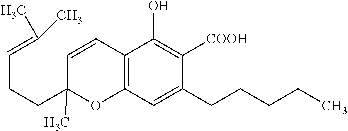 [SMILES: CCCCCc1cc2c(c(O)c1C(=O)O)C=CC(C)(CCC=C(C)C)O2] |
| cannabichromene | CBC | 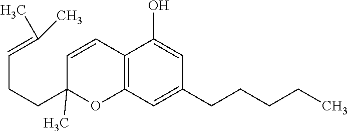 [SMILES: CCCCCc1cc(O)c2c(c1)OC(C)(CCC=C(C)C)C=C2] |
| cannabinolic acid | CBNA | 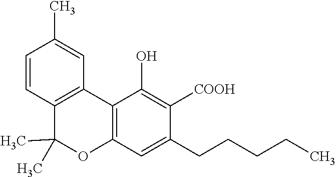 [SMILES: CCCCCc1cc2c(c(O)c1C(=O)O)-c1cc(C)ccc1C(C)(C)O2] |
| cannabinol | CBN | 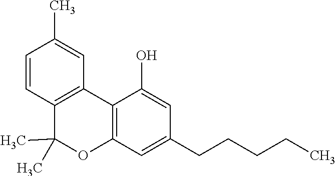 [SMILES: CCCCCc1cc(O)c2c(c1)OC(C)(C)c1ccc(C)cc1-2] |
| cannabidivarinic acid | CBDVA | 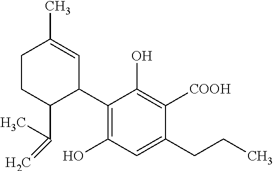 [SMILES: C=C(C)C1CCC(C)=CC1c1c(O)cc(CCC)c(C(=O)O)c1O] |
| cannabidivarin | CBDV | 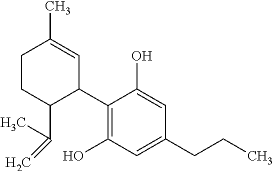 [SMILES: C=C(C)C1CCC(C)=CC1c1c(O)cc(CCC)cc1O] |
| Δ⁹- tetrahydrocannabivarinic acid | Δ⁹- THCVA | 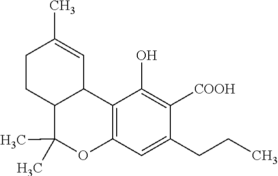 [SMILES: CCCc1cc2c(c(O)c1C(=O)O)C1C=C(C)CCC1C(C)(C)O2] |
| Δ⁹-tetrahydrocannabivarin | Δ⁹- THCV | 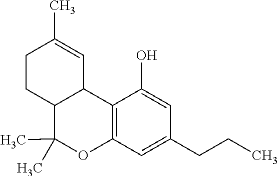 [SMILES: CCCc1cc(O)c2c(c1)OC(C)(C)C1CCC(C)=CC21] |
| Cannabidibutolic acid | CBDBA | 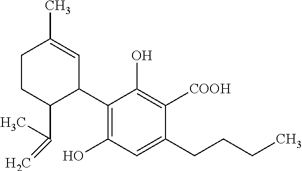 [SMILES: C=C(C)C1CCC(C)=CC1c1c(O)cc(CCCC)c(C(=O)O)c1O] |
| Cannabidibutol | CBDB | 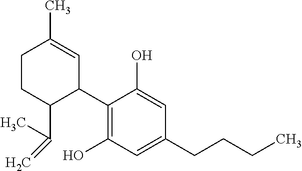 [SMILES: C=C(C)C1CCC(C)=CC1c1c(O)cc(CCCC)cc1O] |
| Δ⁹- tetrahydrocannabutolic acid | Δ⁹- THCBA | 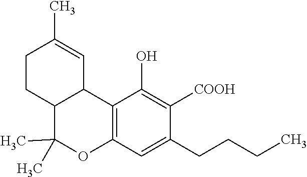 [SMILES: CCCCc1cc2c(c(O)c1C(=O)O)C1C=C(C)CCC1C(C)(C)O2] |
| Δ⁹-tetrahydrocannabutol | Δ⁹- THCB | 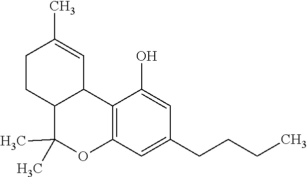 [SMILES: CCCCc1cc(O)c2c(c1)OC(C)(C)C1CCC(C)=CC21] |
| Cannabidiphorolic acid | CBDPA | 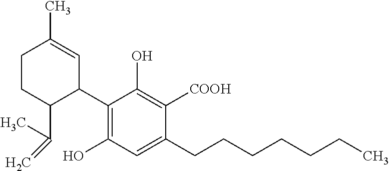 [SMILES: C=C(C)C1CCC(C)=CC1c1c(O)cc(CCCCCCC)c(C(=O)O)c1O] |
| Cannabidiphorol | CBDP | 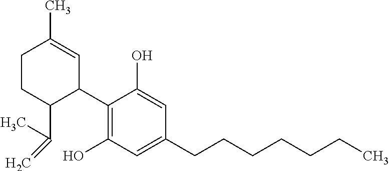 [SMILES: C=C(C)C1CCC(C)=CC1c1c(O)cc(CCCCCCC)cc1O] |
| Δ⁹- tetrahydrocannabiphorolic acid | Δ⁹- THCPA | 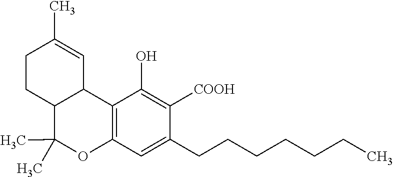 [SMILES: CCCCCCCc1cc2c(c(O)c1C(=O)O)C1C=C(C)CCC1C(C)(C)O2] |
| Δ⁹- tetrahydrocannabiphorol | Δ⁹- THCP | 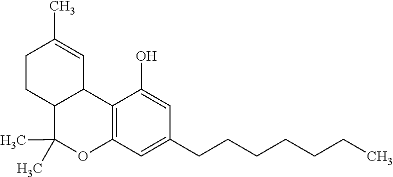 [SMILES: CCCCCCCc1cc(O)c2c(c1)OC(C)(C)C1CCC(C)=CC21] |
| cannabichromevarinic acid | CBCVA | 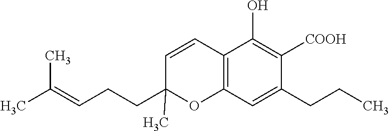 [SMILES: CCCc1cc2c(c(O)c1C(=O)O)C=CC(C)(CCC=C(C)C)O2] |
| cannabichromevarin | CBCV | 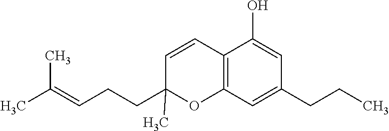 [SMILES: CCCc1cc(O)c2c(c1)OC(C)(CCC=C(C)C)C=C2] |
| cannabigerovarinic acid | CBGVA | 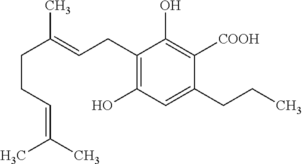 [SMILES: CCCc1cc(O)c(C/C=C(\C)CCC=C(C)C)c(O)c1C(=O)O] |
| cannabigerovarin | CBGV | 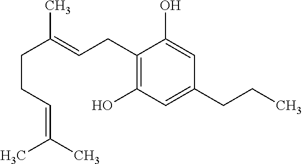 [SMILES: CCCc1cc(O)c(C/C=C(\C)CCC=C(C)C)c(O)c1] |
| cannabicyclolic acid | CBLA | 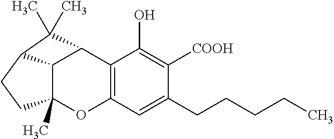 |
| cannabicyclol | CBL | 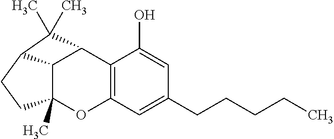 |
| cannabielsoinic acid | CBEA | 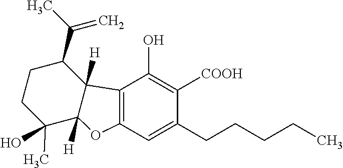 [SMILES: C=C(C)[C@@H]1CC[C@](C)(O)[C@H]2Oc3cc(CCCCC)c(C(=O)O)c(O)c3[C@@H]12] |
| cannabielsoin | CBE | 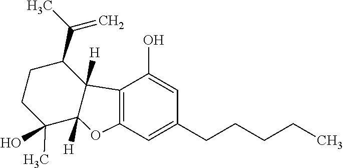 [SMILES: C=C(C)[C@@H]1CC[C@](C)(O)[C@H]2Oc3cc(CCCCC)cc(O)c3[C@@H]12] |
| cannabicitranic acid | CBTA | 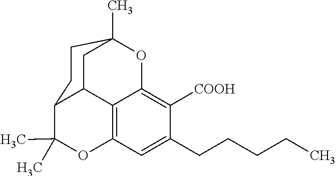 [SMILES: CCCCCc1cc2c3c(c1C(=O)O)OC1(C)CCC(C3C1)C(C)(C)O2] |
| cannabicitran | CBT | 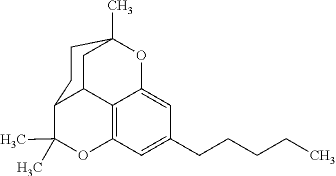 [SMILES: CCCCCc1cc2c3c(c1)OC(C)(C)C1CCC(C)(CC31)O2] |

In at least one embodiment, it is also contemplated that the nucleic acids derived from a Cannabis sativa trichome mRNA of the present disclosure (e.g., as in Table 2) can be introduced into a recombinant host cell to provide a method for the improved biosynthesis of cannabinoid precursor compounds or cannabinoid precursor derivatives in terms of titer, yield, and production rate. Cannabinoid precursors, or cannabinoid precursor derivatives can include, but are not limited to olivetolic acid, olivetol, divarin, PDAL, HTAL, GPP, polyketides, polyketide derivatives, and others known in the art (see e.g,, Elsohly and Slade, Life Sci. 2005 Dec. 22; 78(5):539-48, Epub 2005 Sep. 30; Bow, E. W. and Rimoidi, J. M., “The Structure-Function Relationships of Classical Cannabinoids: CB1/C62 Modulation,” Perspectives in Medicinal Chemistry 2016; 8 17-39 doi:10.4137/PMC.S32171.) Such precursor compounds can be useful products, and/or can be used to prepare other derivative compounds, either synthetically or biosynthetically. In at least one embodiment, a cannabinoid precursor compound, such as OA or divarinic acid (DA), can be produced, and then further modified or derivatized using an in vitro enzymatic biosynthesis, using e.g., a cannabinoid synthase.

In at least one embodiment, the present disclosure provides a method for producing a cannabinoid or cannabinoid precursor comprising: (a) culturing in a suitable medium a recombinant host cell of the present disclosure; and (b) recovering the produced cannabinoid or cannabinoid precursor.

In at least one embodiment of the method for producing a cannabinoid, a nucleic acid derived from a Cannabis sativa trichome mRNA of Table 2, can be introduced into a recombinant host cell comprising a pathway capable of producing a cannabinoid (e.g., CBGA) to provide an recombinant host cell that has improved biosynthesis of the cannabinoid in terms of titer, yield, and production rate.

In at least one embodiment, a recombinant host cell of the present disclosure can produce a cannabinoid compound, or a composition comprising a cannabinoid compound, wherein the cannabinoid is selected from cannabigerolic acid (CBGA), cannabigerol (CBG), cannabidiolic acid (CBDA), cannabidiol (CBD), Δ⁹-tetrahydrocannabinolic acid (Δ⁹-THCA), Δ⁹-tetrahydrocannabinol (Δ⁹-THC), tetrahydrocannabinolic acid (Δ⁸-THCA), Δ⁸-tetrahydrocannabinol (Δ⁸-THC), cannabichromenic acid (CBCA), cannabichromene (CBC), cannabinolic acid (CBNA), cannabinol (CBN), cannabidivarinic acid (CBDVA), cannabidivarin (CBDV), Δ⁹-tetrahydrocannabivarinic acid (Δ⁹-THCVA), Δ⁹-tetrahydrocannabivarin (Δ⁹-THCV), cannabidibutolic acid (CBDBA), cannabidibutol (CBDB), Δ⁹-tetrahydrocannabutolic acid (Δ⁹-THCBA), Δ⁹-tetrahydrocannabutol (Δ⁹-THCB), cannabidiphorolic acid (CBDPA), cannabidiphorol (CBDP), Δ⁹-tetrahydrocannabiphorolic acid (Δ⁹-THCPA), Δ⁹-tetrahydrocannabiphorol (Δ⁹-THCP), cannabichromevarinic acid (CBCVA), cannabichromevarin (CBCV), cannabigerovarinic acid (CBGVA), cannabigerovarin (CBGV), cannabicyclolic acid (CBLA), cannabicyclol (CBL), cannabielsoinic acid (CBEA), cannabielsoin (CBE), cannabicitranic acid (CBTA), cannabicitran (CBT), and any combination thereof. In at least one embodiment, a recombinant host cell of the present disclosure can be used to produce a cannabinoid selected from cannabigerolic acid (CBGA), cannabidiolic acid (CBDA), cannabichromenic acid (CBCA), and any combination thereof.

In at least one embodiment of the method for producing a cannabinoid, the method can further comprise contacting a cell-free extract of the culture containing the produced cannabinoid with a biocatalytic reagent or chemical reagent.

In at least one embodiment, the biocatalytic reagent is an enzyme capable of converting the produced cannabinoid to a different cannabinoid or a cannabinoid derivative compound. In at least one embodiment, the chemical reagent is capable of chemically modifying the produced cannabinoid to produce a different cannabinoid or a cannabinoid derivative compound.

Accordingly, in at least one embodiment of the method, the recombinant host cell with improved cannabinoid production in terms of titer, yield, and production rate can be used in the production of a cannabinoid (see e.g., compounds of Table 3), or a cannabinoid derivative compound. Cannabinoid derivative compounds can include a wide range of naturally-occurring and non-naturally occurring compounds.

Cannabinoid derivative compounds produced using the recombinant host cells of the present disclosure can include any compound structurally related to a cannabinoid compound (e.g., compounds of Table 3) but which lacks one or more of the chemical moieties present in the cannabinoid compound from which it derives. Exemplary chemical moieties that may be lacking in a cannabinoid derivative include, but are not limited to, methyl, alkyl, alkenyl, methoxy, alkoxy, acetyl, carboxyl, carbonyl, oxo, ester, hydroxyl, aryl, heteroaryl, cycloalkyl, cycloalkenyl, cycloalkylalkenyl, cycloalkenylalkyl, cycloalkenylalkenyl, heterocyclylalkenyl, heteroarylalkenyl, arylalkenyl, heterocyclyl, aralkyl, cycloalkylalkyl, heterocyclylalkyl, heteroarylalkyl, and the like.

Alternatively, cannabinoid derivative compounds using the recombinant host cells of the present disclosure can include one or more additional chemical moieties not present in the cannabinoid compound from which it derives. Exemplary chemical moieties that may be added in a cannabinoid derivative include, but are not limited to azido, halo (e.g., chloride, bromide, iodide, fluorine), methyl, alkyl, alkynyl, alkenyl, methoxy, alkoxy, acetyl, amino, carboxyl, carbonyl, oxo, ester, hydroxyl, thio, cyano, aryl, heteroaryl, cycloalkyl, cycloalkenyl, cycloalkylalkenyl, cycloalkylalkynyl, cycloalkenylalkyl, cycloalkenylalkenyl, cycloalkenylalkynyl, heterocyclylalkenyl, heterocyclylalkynyl, heteroarylalkenyl, heteroarylalkynyl, arylalkenyl, arylalkynyl, spirocyclyl, heterospirocyclyl, heterocyclyl, thioalkyl, sulfone, sulfonyl, sulfoxide, amino, alkylamino, dialkylamino, arylamino, alkylarylamino, diarylamino, N-oxide, imide, enamine, imine, oxime, hydrazone, nitrile, aralkyl, cycloalkylalkyl, haloalkyl, heterocyclylalkyl, heteroarylalkyl, nitro, thioxo, and the like.

Accordingly, in at least one embodiment, the present disclosure provides a method of producing a cannabinoid derivative, wherein the method comprises: (a) culturing in a suitable medium a recombinant host cell of the present disclosure; and (b) recovering the produced carinabirioid derivative. In at least one embodiment, the method of producing a cannabinoid derivative further contacting a cell-free extract of the culture containing the produced cannabinoid with a biocatalytic reagent or chemical reagent capable of converting the cannabinoid to a cannabinoid derivative. In at least one embodiment, the biocatalytic reagent is an enzyme capable of converting the produced cannabinoid to a different cannabinoid or a cannabinoid derivative compound. In at least one embodiment, the chemical reagent is capable of chemically modifying the produced cannabinoid to produce a different cannabinoid or a cannabinoid derivative compound.

Cannabinoid derivatives that can be produced with improved yield using a recombinant host cell of the present disclosure can include cannabinoid derivatives modified (e.g., biocatalytically or synthetically) to provide improved properties of pharmaceutical metabolism and/or pharmacokinetics (e.g. solubility, bioavailability, absorption, distribution, plasma half-life and metabolic clearance). Modifications typically providing such improved pharmaceutical properties can include, but are not limited to, halogenation, acetylation and methylation, It is also contemplated that the cannabinoids and cannabinoid derivatives produced by the methods disclosed herein can include pharmaceutically acceptable isotopically labeled cannabinoid and cannabinoid derivative compounds. For example, cannabinoid and cannabinoid derivative compounds wherein the hydrogen atoms are replaced or substituted by one or more deuterium or tritium atoms. Such isotopically labeled cannabinoids and derivatives can be useful in studies of in vivo pharmacokinetics and tissue distribution,

Upon production by the host cells or in the cell-free mixture of the cannabinoid precursors or cannabinoids in accordance with the recombinant host cells, and methods of the present disclosure, the desired compounds may be recovered from the host cell suspension or cell-free mixture and separated from other constituents, such as media constituents, cellular debris, etc. Techniques for separation and recovery of the desired compounds are known to those of skill in the art and can include, for example, solvent extraction (e.g. butane, chloroform, ethanol), column chromatography-based techniques, high-performance liquid chromatography (HPLC), for example, and/or countercurrent separation (CCS) based systems. The recovered cannabinoid compounds may be obtained in a more or less pure form, for example, the desired cannabinoid compound of purity of at least about 60% (w/v), about 70% (w/v), about 80% (w/v), about 90% (w/v), about 95% (w/v) or about 99% (w/v).

It is contemplated that the cannabinoid, cannabinoid precursor, cannabinoid precursor derivative, or cannabinoid derivative recovered using the methods of the present disclosure can be in the form of a salt. In at least one embodiment, the recovered salt of the cannabinoid, cannabinoid precursor, cannabinoid precursor derivative, or cannabinoid derivative is a pharmaceutically acceptable salt. Such pharmaceutically acceptable salts retain the biological effectiveness and properties of the free base compound,

As described elsewhere herein, the rare or synthetic derivatives of cannabinoid compounds that can be produced by the recombinant host cells and methods of the present disclosure are contemplated to exhibit biological and pharmacological properties like those of the more well-studied cannabinoids such as THC and CBD. Accordingly, in at least one embodiment, the present disclosure also provides a composition comprising a rare or synthetic cannabinoid, such as a varin cannabinoid, prepared using the recombinant host cells and methods disclosed herein. It is contemplated that the rare cannabinoid compositions provided by the recombinant host cells and methods of the present disclosure can include pharmaceutical compositions, food compositions, and beverage compositions, containing a rare cannabinoid. Generally, compositions comprising rare cannabinoid compounds can further comprise any of the well-known vehicles, excipients and auxiliary substances, such as wetting or emulsifying agents, pH buffering substances and the like, used in the art of formulating pharmaceutical, food, or beverage compositions. For example, pharmaceutical compositions can contain any of the typical pharmaceutically acceptable excipients including, but are not limited to, liquids such as water, saline, polyethylene glycol, hyaluronic acid, glycerol and ethanol. Pharmaceutically acceptable salts can also be included therein, for example, mineral acid salts such as hydrochlorides, phosphates, sulfates, and the like; and the salts of organic acids such as acetates, propionates, benzoates, and the like. In at least one embodiment, a pharmaceutical composition can comprise a pharmaceutically acceptable excipient that serves as a stabilizer of the rare cannabinoid composition. Examples of suitable excipients that also act as stabilizers include, without limitation, pharmaceutical grades of dextrose, sucrose, lactose, sorbitol, inositol, dextran, and the like. Other suitable pharmaceutical excipients can include, without limitation, starch, cellulose, sodium or calcium phosphates, citric acid, glycine, polyethylene glycols (PEGs), and combinations thereof.

### EXAMPLES

Various features and embodiments of the disclosure are illustrated in the following representative examples, which are intended to be illustrative, and not limiting. Those skilled in the art will readily appreciate that the specific examples are only illustrative of the invention as described more fully in the claims which follow thereafter. Every embodiment and feature described in the application should be understood to be interchangeable and combinable with every embodiment contained within.

### Example 1

### Generation and Screening of cDNA Library from Cannabis sativa Trichome mRNA

This example illustrates the generation of a cDNA library from mRNA extracted from isolated trichomes of Cannabis sativa flowers, transformation of the cDNA library into a strain of Saccharomyces cerevisiae already comprising an integrated cannabinoid biosynthesis pathway of genes encoding the enzymes AAE (SEQ ID NO: 2), OLS (SEQ ID NO: 4), OAC (SEQ ID NO: 6), and PT4 (SEQ ID NO: 10) capable of synthesizing cannabigerolic acid (CBGA), and screening the transformants for CBGA production. This cDNA library provides a useful tool for identifying genes and variations (alleles) of the cannabinoid biosynthesis genes that can be used to recreate the biosynthetic pathway in recombinant microorganisms such as yeast.

Materials and Methods

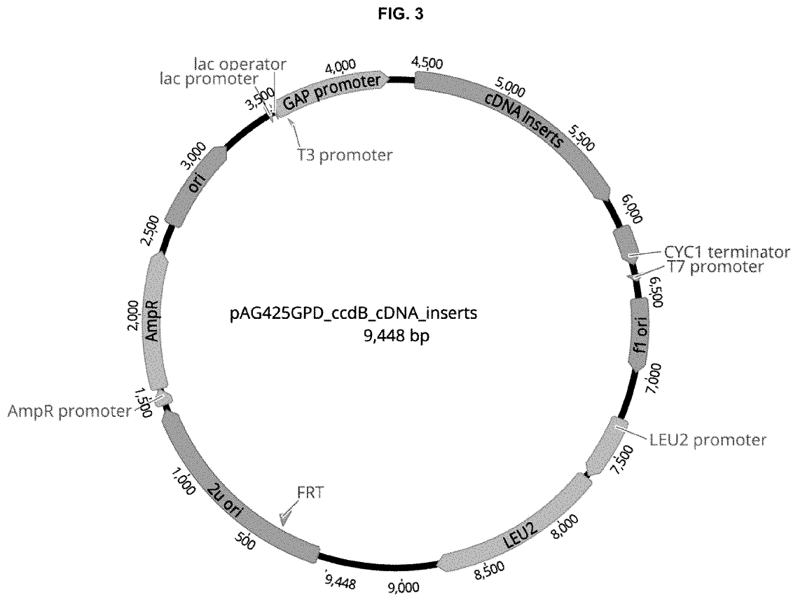

A. cDNA library creation: Total mRNA was extracted from isolated Cannabis sativa trichomes derived from flower tissues, and subsequently reverse-transcribed to cDNA using standard molecular biology techniques. Upon second strand cDNA synthesis and adapter ligation, the library was cloned into a plasmid cloning vector (pDONR-222) and subsequently sub-cloned into a yeast expression vector with auxotrophic marker (pAG425; LEU2 gene), as shown FIG. 3, to create the cDNA library. A constitutive promoter (GAP promoter) was used to express the individual cDNAs that were cloned into the yeast expression vector (pAG425).

B. cDNA library verification: In order to ascertain the quality (gene diversity and integrity) of the cDNA library that was cloned, we performed PacBio Single Molecule, Real-Time (SMRT) Sequencing of the pAG425 cDNA library. After trimming away vector sequences and sequence artifacts, a total of 142,367 cDNA sequences were identified in this library using the PacBio sequencing approach. A total of 129,263 cDNAs (90.8% of total) of these sequences were successfully mapped back to a cannabis genome assembly from which the trichomes and resulting cDNAs were isolated. This demonstrates that there is a large number of cannabis genes in the library. RNAseq analysis has identified hundreds of genes that are specifically expressed in the glandular trichomes where cannabinoid biosynthesis takes place. Moreover, the known cannabinoid pathway biosynthesis genes, AAE, OLS, OAC, PT4, were also found in the trichome specific RNAseq dataset, providing additional positive evidence that the unknown genes in the cDNA library contribute to promoting cannabinoid biosynthesis in cannabis.

In order confirm that the cDNA library was enriched in genes specifically expressed in cannabis glandular trichomes we performed conditional reciprocal best blast hit analysis between PacBio cDNA library and the trichome specific transcriptome assemblies that we had previously generated. A total of 105,871 cDNA sequences representing 74.4% of the total cDNA reads were successfully matched to a transcript in the Cannabis trichome specific RNAseq assembly. This data demonstrates that the cDNA library that was screened to identify nucleic acid sequences that aid a yeast host biosynthesize cannabinoids as described in the present disclosure, is derived from cDNAs that are expressed in the glandular trichomes of Cannabis sativa.

Finally, an EviGene gene prediction and analysis approach was used to evaluate the ‘completeness’ of the sequenced cDNA transcripts (see e.g., EvidentialGene described at web-site: arthropods.eugenes.org/EvidentialGene/plants/). A total of 99918, 37624, 4336, and 599 sequences were binned into the “complete” (complete reading frame, from amino acid conversion); “partial5” (partial reading frames including 5prime utr); “partial3” (partial reading frames including 3prime utr); and “partial” (partial reading frames), respectively. This analysis shows that the vast majority (70.2%) of sequences that were cloned are predicted to be full length protein coding sequences.

C. Express cDNA library in yeast: The pAG425 based cDNA library was transformed into a Saccharomyces cerevisiae strain that included a cannabinoid pathway for biosynthesis of CBGA from hexanoic acid (HA). The pathway included genes derived from C. sativa encoding the following enzymes expressed under constitutive promoters: AAE, OLS, OAC, and PT4. A constitutive promoter (GAP promoter) in the yeast expression vector (pAG425) was used to express the cDNA library.

D. Screen cDNA library for cannabinoid biosynthesis: Transformed yeast strains were plated on selective SC-LEU plates and 11,789 single colonies were using Molecular Devices Q-Pix 420 into 96-well mid well plates with 300 μL of SC-LEU+glucose media and incubated for 48-hr. Each plate is also inoculated with media blank, empty vector (negative) and parent strain (positive) controls. These pre-culture plates are then sub-cultured (1-10× dilution) into fresh SC-Leu +glucose media with 0.2 mM hexanoic acid and grown for another 48 hr at 30° C. for CBGA production. The whole cell broth production samples are extracted by adding equal volumes of acetonitrile and shaking for 30 min at 250 rpm. The plates are centrifuged and the supernatant is appropriately diluted for analysis by RapidFire 365 HTP-Mass Spectrometry instrument by Agilent. The CBGA titers in 96-well plates are quantified based on the calibration curve generated with pure standards prior to running the samples. The selection criteria for the top hits is 2x or higher fold improvement over the positive control.

Results

From a total of 11,789 independent yeast transformants a total of 619 yeast strains were identified as having a >2× fold improvement in CBGA titer. Furthermore, a total of 263 yeast strains were selected from these 619 strain hits and were sequenced to identify the causative cDNA that resulted in the increased CBGA titer. As summarized in Table 2, a total of 263 cDNAs capable of at least 2-fold improved CBGA titer production were identified and categorized based on plant protein function BIN Description and annotated.

Notwithstanding the appended claims, the disclosure set forth herein is also defined by the following clauses, which may be beneficial alone or in combination, with one or more other causes or embodiments. Without limiting the foregoing description, certain non-limiting clauses of the disclosure numbered as below are provided, wherein each of the individually numbered clauses may be used or combined with any of the preceding or following clauses. Thus, this is intended to provide support for all such combinations and is not necessarily limited to specific combinations explicitly provided below:
1. A recombinant host cell comprising a pathway capable of producing a cannabinoid and a nucleic acid derived from a Cannabis trichome mRNA that does not encode an enzyme in the pathway.
2. The cell of clause 1, wherein the nucleic acid derived from a Cannabis trichome mRNA encodes a polypeptide having a protein function selected from: lipid transfer type protein, mulatexin-like, amino acid biosynthesis, amino acid degradation, carbohydrate metabolism, carrier-mediated transport, cell cycle organization, cellular respiration, chromatin organization, circadian clock system regulation, coenzyme metabolism, oxidoreductase, transferase, hydrolase, lyase, effector-triggered immunity network co-regulatory protein, fatty acid biosynthesis, lipid degradation, messenger ribonucleoparticle (mRNP) export, organelle machinery ribonuclease activity, phytohormone action, protein folding, protein phosphorylation, protein S-glutathionylation, protein translocation, redox homeostasis, RNA biosynthesis, RNA processing, solute transport, terpenoid metabolism, and/or vesicle trafficking.
3. The cell of any one of clauses 1-2, wherein the nucleic acid derived from a Cannabis trichome mRNA encodes a polypeptide selected from: non-specific lipid-transfer protein 1-like; non-specific lipid-transfer protein 2-like; major allergen Pru av 1-like; mulatexin-like; 1-acyl-sn-glycerol-3-phosphate acyltransferase 2; 1-aminocyclopropane-1-carboxylate oxidase homolog 1-like; acyl-acyl carrier protein thioesterase ATL3, chloroplastic-like; barwin-like; beta-adaptin-like protein B; BURP domain protein RD22-like; cationic peroxidase 2-like; cell division control protein 2 homolog 2; chloroplast stem-loop binding protein of 41 kDa b, chloroplastic; cysteine-rich receptor-like protein kinase 19; cytochrome B5-like protein; delta(12)-fatty-acid desaturase FAD2-like; desiccation-related protein PCC13-62-like; dormancy-associated protein 2-like; E3 ubiquitin-protein ligase SDIR1; gamma-carbonic anhydrase-like 2, mitochondrial; germacrene-A synthase-like; glucan endo-1,3-beta-glucosidase 12; glucose-6-phosphate 1-dehydrogenase 6, cytoplasmic-like; glycine-rich RNA-binding protein-like; malonate--CoA ligase; mannose-1-phosphate guanylyltransferase 1; mediator of RNA polymerase II transcription subunit 11; MLP-like protein 423; NADH dehydrogenase [ubiquinone] flavoprotein 2, mitochondrial; NADP-dependent glyceraldehyde-3-phosphate dehydrogenase; NDR1/HIN1-like protein 1; ornithine aminotransferase, mitochondrial; peptidyl-prolyl cis-trans isomerase CYP19-3; peroxidase 12-like; phosphoinositide phosphatase SAC1; probable gamma-secretase subunit PEN-2; probable protein phosphatase 2C 60; programmed cell death protein 2-like; protein CASP; protein ELF4-LIKE 4-like; protein REVEILLE 8; protein SRC1; protein TIC 56, chloroplastic; pyruvate decarboxylase 1-like; small acidic protein 1; sphingoid long-chain bases kinase 1; structural maintenance of chromosomes protein 1; translationally-controlled tumor protein homolog; tubulin beta-2 chain; ubiquitin-conjugating enzyme E2-17 kDa-like; U-box domain-containing protein 34; upstream activation factor subunit UAF30; uridine kinase-like protein 1, chloroplastic; V-type proton ATPase subunit B 1; and YTH domain-containing protein ECT4-like.
4. The cell of any one of clauses 1-3, wherein the nucleic acid derived from Cannabis trichome mRNA encodes a polypeptide comprising (a) an amino acid sequence having at least 90% identity to any one of even-numbered SEQ ID NOs: 16-540; (b) an amino acid sequence sequence having at least 90%, at least 95%, at least 96%, at least 97%, at least 98%, or at least 99% identity to any one of SEQ ID NOs: 36, 74, 88, 94, 104, 106, 108, 110, 112, 114, 116, 120, 122, 124, 126, 128, 130, 132, 134, 136, 138, 140, 142, 144, 146, 14, 150, 168, 174, 184, 188, 206, 210, 232, 270, 274, and 396; or (c) an amino acid sequence having at least 90% identity to any one of even-numbered SEQ ID NOs: 122-150.
5. The cell of any one of clauses 1-4, wherein the nucleic acid derived from Cannabis trichome mRNA comprises: (a) a nucleotide sequence having at least 90% identity to any one of odd-numbered SEQ ID NOs: 15-539; (b) a nucleotide sequence having at least 90%, at least 95%, at least 96%, at least 97%, at least 98%, or at least 99% identity to any one of SEQ ID NOs: 35, 73, 87, 93, 103, 105, 107, 109, 111, 113, 115, 119, 121, 123, 125, 127, 129, 131, 133, 135, 137, 139, 141, 143, 145, 147, 149, 167, 173, 183, 187, 205, 209, 231, 269, 273, and 395; or (c) a nucleotide sequence having at least 90% identity to any one of odd-numbered SEQ ID NOs: 121-149.
6. The cell of any one of clauses 1-5, wherein the recombinant host cell produces the cannabinoid with a titer that is increased at least 1.2-fold, at least 2-fold, at least 3-fold, at least 4-fold, at least 5-fold, or more relative to a control recombinant host cell comprising the pathway and not the nucleic acid derived from a Cannabis trichome mRNA.
7. The cell of any one of clauses 1-6, wherein the pathway capable of producing a cannabinoid comprises enzymes capable of converting hexanoic acid to CBGA.
8. The cell of any one of clauses 1-7, wherein the pathway capable of producing a cannabinoid comprises enzymes capable of catalyzing reactions (i), (ii), (iii), and (iv).
9. The cell of any one of clauses 1-8, wherein the pathway capable of producing a cannabinoid comprises at least the following enzymes: AAE, OLS, OAC, and PT4; optionally, wherein the enzymes AAE, OLS, OAC, and PT4 have an amino acid sequence of at least 90% identity to SEQ ID NO: 2 (AAE), SEQ ID NO: 4 (OLS), SEQ ID NO: 6 (OAC), and SEQ ID NO: 8 or 10 (PT4), respectively. 10. The cell of clause 7, wherein the pathway further comprises an enzyme capable of catalyzing the conversion of CBGA to Δ⁹-THCA, CBDA, and/or CBCA
11. The cell of clause 9, wherein the pathway further comprises: THCA synthase, CBDA synthase, and/or CBCA synthase; optionally, wherein the pathway comprises a CBDA synthase having an amino acid sequence of at least 90% identity to SEQ ID NO: 12 or 14, or a THCA synthase having an amino acid sequence of at least 90% identity to SEQ ID NO: 542 or 544.
12. The cell of any one of clauses 1-11, wherein the cannabinoid produced by the host cell is selected from cannabigerolic acid (CBGA), cannabigerol (CBG), cannabidiolic acid (CBDA), cannabidiol (CBD), Δ⁹-tetrahydrocannabinolic acid (Δ⁹-THCA), Δ⁹-tetrahydrocannabinol (Δ⁹-THC), Δ⁸-tetrahydrocannabinolic acid (Δ⁸-THCA), Δ⁸-tetrahydrocannabinol (Δ⁸-THC), cannabichromenic acid (CBCA), cannabichromene (CBC), cannabinolic acid (CBNA), cannabinol (CBN), cannabidivarinic acid (CBDVA), cannabidivarin (CBDV), Δ⁹-tetrahydrocannabivarinic acid (Δ⁹-THCVA), Δ⁹-tetrahydrocannabivarin (Δ⁹-THCV), cannabidibutolic acid (CBDBA), cannabidibutol (CBDB), Δ⁹-tetrahydrocannabutolic acid (Δ⁹-THCBA), Δ⁹-tetrahydrocannabutol (Δ⁹-THCB), cannabidiphorolic acid (CBDPA), cannabidiphorol (CBDP), Δ⁹-tetrahydrocannabiphorolic acid (Δ⁹-THCPA), Δ⁹-tetrahydrocannabiphorol (Δ⁹-THCP), cannabichromevarinic acid (CBCVA), cannabichromevarin (CBCV), can nabigerovarinic acid (CBGVA), cannabigerovarin (CBGV), cannabicyclolic acid (CBLA), cannabicyclol (CBL), cannabielsoinic acid (CBEA), cannabielsoin (CBE), cannabicitranic acid (CBTA), cannabicitran (CBT), and any combination thereof.
13. The cell of any one of clauses 1-12, wherein recombinant host cell source is selected from Saccharomyces cerevisiae, Yarrowia lipolytica, Pichia pastoris, and Escherichia coli.
14. A method for producing a cannabinoid comprising: (a) culturing in a suitable medium a recombinant host cell of any one of clauses 1-13; and (b) recovering the produced cannabinoid.
15. The method of clause 14, wherein the method further comprises contacting a cell-free extract of the culture with a biocatalytic reagent or chemical reagent.
16. A method for producing a cannabinoid comprising: (a) culturing in a suitable medium a recombinant host cell comprising a pathway capable of producing a cannabinoid, wherein the host cell further comprises a nucleic acid derived from a Cannabis trichome mRNA that does not encode an enzyme in the pathway; and (b) recovering the produced cannabinoid.
17. The method of clause 16, wherein the method further comprises contacting a cell-free extract of the culture with a biocatalytic reagent or chemical reagent.
18. The method of any one of clauses 16-17, wherein the nucleic acid derived from a Cannabis trichome mRNA encodes a polypeptide having a protein function selected from: lipid transfer type protein, mulatexin-like, amino acid biosynthesis, amino acid degradation, carbohydrate metabolism, carrier-mediated transport, cell cycle organization, cellular respiration, chromatin organization, circadian clock system regulation, coenzyme metabolism, oxidoreductase, transferase, hydrolase, lyase, effector-triggered immunity network co-regulatory protein, fatty acid biosynthesis, lipid degradation, messenger ribonucleoparticle (mRNP) export, organelle machinery ribonuclease activity, phytohormone action, protein folding, protein phosphorylation, protein S-glutathionylation, protein translocation, redox homeostasis, RNA biosynthesis, RNA processing, solute transport, terpenoid metabolism, and/or vesicle trafficking.
19. The method of any one of clauses 16-18, wherein the nucleic acid derived from a Cannabis trichome mRNA encodes a polypeptide selected from: non-specific lipid-transfer protein 1-like; non-specific lipid-transfer protein 2-like; major allergen Pru av 1-like; mulatexin-like; 1-acyl-sn-glycerol-3-phosphate acyltransferase 2; 1-aminocyclopropane-1-carboxylate oxidase homolog 1-like; acyl-acyl carrier protein thioesterase ATL3, chloroplastic-like; barwin-like; beta-adaptin-like protein B; BURP domain protein RD22-like; cationic peroxidase 2-like; cell division control protein 2 homolog 2; chloroplast stem-loop binding protein of 41 kDa b, chloroplastic; cysteine-rich receptor-like protein kinase 19; cytochrome B5-like protein; delta(12)-fatty-acid desaturase FAD2-like; desiccation-related protein PCC13-62-like; dormancy-associated protein 2-like; E3 ubiquitin-protein ligase SDIR1; gamma-carbonic anhydrase-like 2, mitochondrial; germacrene-A synthase-like; glucan endo-1,3-beta-glucosidase 12; glucose-6-phosphate 1-dehydrogenase 6, cytoplasmic-like; glycine-rich RNA-binding protein-like; malonate--CoA ligase; mannose-1-phosphate guanylyltransferase 1; mediator of RNA polymerase II transcription subunit 11; MLP-like protein 423; NADH dehydrogenase [ubiquinone] flavoprotein 2, mitochondrial; NADP-dependent glyceraldehyde-3-phosphate dehydrogenase; NDR1/HIN1-like protein 1; ornithine aminotransferase, mitochondrial; peptidyl-prolyl cis-trans isomerase CYP19-3; peroxidase 12-like; phosphoinositide phosphatase SAC1; probable gamma-secretase subunit PEN-2; probable protein phosphatase 2C 60; programmed cell death protein 2-like; protein CASP; protein ELF4-LIKE 4-like; protein REVEILLE 8; protein SRC1; protein TIC 56, chloroplastic; pyruvate decarboxylase 1-like; small acidic protein 1; sphingoid long-chain bases kinase 1; structural maintenance of chromosomes protein 1; translationally-controlled tumor protein homolog; tubulin beta-2 chain; ubiquitin-conjugating enzyme E2-17 kDa-like; U-box domain-containing protein 34; upstream activation factor subunit UAF30; uridine kinase-like protein 1, chloroplastic; V-type proton ATPase subunit B 1; and YTH domain-containing protein ECT4-like.
20. The method of any one of clauses 16-19, wherein the nucleic acid derived from Cannabis trichome mRNA encodes a polypeptide comprising: (a) an amino acid sequence having at least 90% identity to any one of even-numbered SEQ ID NOs: 16-540; (b) an amino acid sequence sequence having at least 90%, at least 95%, at least 96%, at least 97%, at least 98%, or at least 99% identity to any one of SEQ ID NOs: 36, 74, 88, 94, 104, 106, 108, 110, 112, 114, 116, 120, 122, 124, 126, 128, 130, 132, 134, 136, 138, 140, 142, 144, 146, 14, 150, 168, 174, 184, 188, 206, 210, 232, 270, 274, and 396; or (c) an amino acid sequence having at least 90% identity to any one of even-numbered SEQ ID NOs: 122-150.
21. The method of any one of clauses 16-21, wherein the nucleic acid derived from Cannabis trichome mRNA comprises: (a) a nucleotide sequence having at least 90% identity to any one of odd-numbered SEQ ID NOs: 15-539; (b) a nucleotide sequence having at least 90%, at least 95%, at least 96%, at least 97%, at least 98%, or at least 99% identity to any one of SEQ ID NOs: 35, 73, 87, 93, 103, 105, 107, 109, 111, 113, 115, 119, 121, 123, 125, 127, 129, 131, 133, 135, 137, 139, 141, 143, 145, 147, 149, 167, 173, 183, 187, 205, 209, 231, 269, 273, and 395; or (c) a nucleotide sequence having at least 90% identity to any one of odd-numbered SEQ ID NOs: 121-149.
22. The method of any one of clauses 16-21, wherein the pathway capable of producing a cannabinoid comprises enzymes capable of converting hexanoic acid to CBGA; optionally wherein the pathway capable of producing a cannabinoid comprises enzymes capable of catalyzing reactions (i), (ii), (iii), and (iv).
23. The method of any one of clauses 16-22, wherein the pathway capable of producing a cannabinoid comprises at least the following enzymes: AAE, OLS, OAC, and PT4; optionally, wherein AAE has an amino acid sequence of at least 90% identity to SEQ ID NO: 2, OLS has an amino acid sequence of at least 90% identity to SEQ ID NO: 4, OAC has an amino acid sequence of at least 90% identity to SEQ ID NO: 6, and PT4 has an amino acid sequence of at least 90% identity to SEQ ID NO: 8 or 10.
24. The method of any one of clauses 16-23, wherein the pathway further comprises enzymes capable of converting CBGA to Δ⁹-THCA, CBDA, and/or CBCA.
25. The method of any one of clauses 16-23, wherein the pathway further comprises: THCA synthase, CBDA synthase, and/or CBCA synthase; optionally, wherein the pathway comprises (a) a CBDA synthase having an amino acid sequence of at least 90% identity to SEQ ID NO: 12 or 14, and/or (b) a THCA synthase having an amino acid sequence of at least 90% identity to SEQ ID NO: 542 or 544.
26. The method of any one of clauses 16-25, wherein the cannabinoid produced by the host cell is selected from cannabigerolic acid (CBGA), cannabigerol (CBG), cannabidiolic acid (CBDA), cannabidiol (CBD), Δ⁹-tetrahydrocannabinolic acid (Δ⁹-THCA), Δ⁹-tetrahydrocannabinol (Δ⁹-THC), Δ⁸-tetrahydrocannabinolic acid (Δ⁸-THCA), Δ⁸-tetrahydrocannabinol (Δ⁸-THC), cannabichromenic acid (CBCA), cannabichromene (CBC), cannabinolic acid (CBNA), cannabinol (CBN), cannabidivarinic acid (CBDVA), cannabidivarin (CBDV), Δ⁹-tetrahydrocannabivannic acid (Δ⁹-THCVA), ,Δ⁹-tetrahydrocannabivarin (Δ⁹-THCV), cannabidibutolic acid (CBDBA), cannabidibutol (CBDB), Δ⁹-tetrahydrocannabutolic acid (Δ⁹-THCBA), Δ⁹-tetrahydrocannabutol (Δ⁹-THCB), cannabidiphorolic acid (CBDPA), cannabidiphorol (CBDP), Δ⁹-tetrahydrocannabiphorolic acid (Δ⁹-THCPA), Δ⁹-tetrahydrocannabiphorol (Δ⁹-THCP), cannabichromevarinic acid (CBCVA), cannabichromevarin (CBCV), can nabigerovarinic acid (CBGVA), cannabigerovarin (CBGV), cannabicyclolic acid (CBLA), cannabicyclol (CBL), cannabielsoinic acid (CBEA), cannabielsoin (CBE), cannabicitranic acid (CBTA), cannabicitran (CBT), and any combination thereof.
27. The method of any one of clauses 16-26, wherein the recombinant host cell is a cell derived from a source selected from: Saccharomyces cerevisiae, Escherichia coli, Yarrowia lipolytica, and Pichia pastoris.
28. A method for making a recombinant host cell for producing a cannabinoid comprising introducing into a host cell: (a) a first set of nucleic acids that encode a pathway of enzymes capable of producing a cannabinoid; and (b) a nucleic acid derived from a Cannabis trichome mRNA that does not encode an enzyme in the pathway, wherein the nucleic acid encodes a polypeptide having a protein function selected from: lipid transfer type protein, mulatexin-like, amino acid biosynthesis, amino acid degradation, carbohydrate metabolism, carrier-mediated transport, cell cycle organization, cellular respiration, chromatin organization, circadian clock system regulation, coenzyme metabolism, oxidoreductase, transferase, hydrolase, lyase, effector-triggered immunity network co-regulatory protein, fatty acid biosynthesis, lipid degradation, messenger ribonucleoparticle (mRNP) export, organelle machinery ribonuclease activity, phytohormone action, protein folding, protein phosphorylation, protein S-glutathionylation, protein translocation, redox homeostasis, RNA biosynthesis, RNA processing, solute transport, terpenoid metabolism, and/or vesicle trafficking.
29. The method of clause 28, wherein the nucleic acid derived from Cannabis trichome mRNA encodes a polypeptide encodes a polypeptide selected from: non-specific lipid-transfer protein 1-like; non-specific lipid-transfer protein 2-like; major allergen Pru av 1-like; mulatexin-like; 1-acyl-sn-glycerol-3-phosphate acyltransferase 2; 1-aminocyclopropane-1-carboxylate oxidase homolog 1-like; acyl-acyl carrier protein thioesterase ATL3, chloroplastic-like; barwin-like; beta-adaptin-like protein B; BURP domain protein RD22-like; cationic peroxidase 2-like; cell division control protein 2 homolog 2; chloroplast stem-loop binding protein of 41 kDa b, chloroplastic; cysteine-rich receptor-like protein kinase 19; cytochrome B5-like protein; delta(12)-fatty-acid desaturase FAD2-like; desiccation-related protein PCC13-62-like; dormancy-associated protein 2-like; E3 ubiquitin-protein ligase SDIR1; gamma-carbonic anhydrase-like 2, mitochondrial; germacrene-A synthase-like; glucan endo-1,3-beta-glucosidase 12; glucose-6-phosphate 1-dehydrogenase 6, cytoplasmic-like; glycine-rich RNA-binding protein-like; malonate--CoA ligase; mannose-1-phosphate guanylyltransferase 1; mediator of RNA polymerase II transcription subunit 11; MLP-like protein 423; NADH dehydrogenase [ubiquinone] flavoprotein 2, mitochondrial; NADP-dependent glyceraldehyde-3-phosphate dehydrogenase; NDR1/HIN1-like protein 1; ornithine aminotransferase, mitochondrial; peptidyl-prolyl cis-trans isomerase CYP19-3; peroxidase 12-like; phosphoinositide phosphatase SAC1; probable gamma-secretase subunit PEN-2; probable protein phosphatase 2C 60; programmed cell death protein 2-like; protein CASP; protein ELF4-LIKE 4-like; protein REVEILLE 8; protein SRC1; protein TIC 56, chloroplastic; pyruvate decarboxylase 1-like; small acidic protein 1; sphingoid long-chain bases kinase 1; structural maintenance of chromosomes protein 1; translationally-controlled tumor protein homolog; tubulin beta-2 chain; ubiquitin-conjugating enzyme E2-17 kDa-like; U-box domain-containing protein 34; upstream activation factor subunit UAF30; uridine kinase-like protein 1, chloroplastic; V-type proton ATPase subunit B 1; and YTH domain-containing protein ECT4-like.
30. The method of any one of clauses 28-29, wherein the nucleic acid derived from Cannabis trichome mRNA encodes a polypeptide comprising: (a) an amino acid sequence having at least 90% identity to any one of even-numbered SEQ ID NOs: 16-540; (b) an amino acid sequence sequence having at least 90%, at least 95%, at least 96%, at least 97%, at least 98%, or at least 99% identity to any one of SEQ ID NOs: 36, 74, 88, 94, 104, 106, 108, 110, 112, 114, 116, 120, 122, 124, 126, 128, 130, 132, 134, 136, 138, 140, 142, 144, 146, 14, 150, 168, 174, 184, 188, 206, 210, 232, 270, 274, and 396; or (c) an amino acid sequence having at least 90% identity to any one of even-numbered SEQ ID NOs: 122-150.
31. The method of any one of clauses 28-30, wherein the nucleic acid derived from Cannabis trichome mRNA comprises: (a) a nucleotide sequence having at least 90% identity to any one of odd-numbered SEQ ID NOs: 15-539; (b) a nucleotide sequence having at least 90%, at least 95%, at least 96%, at least 97%, at least 98%, or at least 99% identity to any one of SEQ ID NOs: 35, 73, 87, 93, 103, 105, 107, 109, 111, 113, 115, 119, 121, 123, 125, 127, 129, 131, 133, 135, 137, 139, 141, 143, 145, 147, 149, 167, 173, 183, 187, 205, 209, 231, 269, 273, and 395; or (c) a nucleotide sequence having at least 90% identity to any one of odd-numbered SEQ ID NOs: 121-149.
32. The method of any one of clauses 28-31, wherein the pathway capable of producing a cannabinoid comprises enzymes capable of converting hexanoic acid to CBGA.
33. The method of any one of clauses 28-32, wherein the pathway capable of producing a cannabinoid comprises enzymes capable of catalyzing reactions (i), (ii), (iii), and (iv).
34. The method of any one of clauses 28-33, wherein the pathway capable of producing a cannabinoid comprises at least the following enzymes: AAE, OLS, OAC, and PT4; optionally, wherein AAE has an amino acid sequence of at least 90% identity to SEQ ID NO: 2, OLS has an amino acid sequence of at least 90% identity to SEQ ID NO: 4, OAC has an amino acid sequence of at least 90% identity to SEQ ID NO: 6, and PT4 has an amino acid sequence of at least 90% identity to SEQ ID NO: 8 or 10.
35. The method of any one of clauses 28-34, wherein the pathway further comprises enzymes capable of converting CBGA to Δ⁹-THCA, CBDA, and/or CBCA; optionally, wherein the pathway comprises (a) a CBDA synthase having an amino acid sequence of at least 90% identity to SEQ ID NO: 12 or 14, and/or (b) a THCA synthase having an amino acid sequence of at least 90% identity to SEQ ID NO: 542 or 544.
36. The method of any one of clauses 28-35, wherein the cannabinoid produced by the host cell is selected from cannabigerolic acid (CBGA), cannabigerol (CBG), cannabidiolic acid (CBDA), cannabidiol (CBD), Δ⁹-tetrahydrocannabinolic acid (Δ⁹-THCA), Δ⁹-tetrahydrocannabinol (Δ⁹-THC), Δ⁸-tetrahydrocannabinolic acid (Δ⁸-THCA), Δ⁸-tetrahydrocannabinol (Δ⁸-THC), cannabichromenic acid (CBCA), cannabichromene (CBC), cannabinolic acid (CBNA), cannabinol (CBN), cannabidivarinic acid (CBDVA), cannabidivarin (CBDV), Δ⁹-tetrahydrocannabivarinic acid (Δ⁹-THCVA), ,o,⁹-tetrahydrocannabivarin (Δ⁹-THCV), cannabidibutolic acid (CBDBA), cannabidibutol (CBDB), Δ⁹-tetrahydrocannabutolic acid (Δ⁹-THCBA), Δ⁹-tetrahydrocannabutol (Δ⁹-THCB), cannabidiphorolic acid (CBDPA), cannabidiphorol (CBDP), Δ⁹-tetrahydrocannabiphorolic acid (Δ⁹-THCPA), Δ⁹-tetrahydrocannabiphorol (Δ⁹-THCP), cannabichromevarinic acid (CBCVA), cannabichromevarin (CBCV), can nabigerovarinic acid (CBGVA), cannabigerovarin (CBGV), cannabicyclolic acid (CBLA), cannabicyclol (CBL), cannabielsoinic acid (CBEA), cannabielsoin (CBE), cannabicitranic acid (CBTA), cannabicitran (CBT), and any combination thereof.
37. The method of any one of clauses 28-36, wherein the recombinant host cell is a cell derived from a source selected from: Saccharomyces cerevisiae, Escherichia coli, Yarrowia lipolytica, and Pichia pastoris; optionally, wherein the source host cell is a cell from Saccharomyces cerevisiae.

While the foregoing disclosure of the present invention has been described in some detail by way of example and illustration for purposes of clarity and understanding, this disclosure including the examples, descriptions, and embodiments described herein are for illustrative purposes, are intended to be exemplary, and should not be construed as limiting the present disclosure. It will be clear to one skilled in the art that various modifications or changes to the examples, descriptions, and embodiments described herein can be made and are to be included within the spirit and purview of this disclosure and the appended claims. Further, one of skill in the art will recognize a number of equivalent methods and procedure to those described herein. All such equivalents are to be understood to be within the scope of the present disclosure and are covered by the appended claims.

Additional embodiments of the invention are set forth in the following claims.

The disclosures of all publications, patent applications, patents, or other documents mentioned herein are expressly incorporated by reference in their entirety for all purposes to the same extent as if each such individual publication, patent, patent application or other document were individually specifically indicated to be incorporated by reference herein in its entirety for all purposes and were set forth in its entirety herein. In case of conflict, the present specification, including specified terms, will control.

What is claimed is:

1. A recombinant host cell comprising a pathway capable of producing a cannabinoid and a nucleic acid derived from a Cannabis trichome mRNA that does not encode an enzyme in the pathway, wherein the nucleic acid derived from a Cannabis trichome mRNA encodes a polypeptide having a protein function selected from: lipid transfer type protein, mulatexin-like, amino acid biosynthesis, amino acid degradation, carbohydrate metabolism, carrier-mediated transport, cell cycle organization, cellular respiration, chromatin organization, circadian clock system regulation, coenzyme metabolism, oxidoreductase, transferase, hydrolase, lyase, effector-triggered immunity network co-regulatory protein, fatty acid biosynthesis, lipid degradation, messenger ribonucleoparticle (mRNP) export, organelle machinery ribonuclease activity, phytohormone action, protein folding, protein phosphorylation, protein S-glutathionylation, protein translocation, redox homeostasis, RNA biosynthesis, RNA processing, solute transport, terpenoid metabolism, and/or vesicle trafficking.

2. The cell of claim 1, wherein the nucleic acid derived from a Cannabis trichome mRNA encodes a polypeptide selected from: non-specific lipid-transfer protein 1-like; non-specific lipid-transfer protein 2-like; major allergen Pru av 1-like; mulatexin-like; 1-acyl-sn-glycerol-3-phosphate acyltransferase 2; 1-aminocyclopropane-1-carboxylate oxidase homolog 1-like; acyl-acyl carrier protein thioesterase ATL3, chloroplastic-like; barwin-like; beta-adaptin-like protein B; BURP domain protein RD22-like; cationic peroxidase 2-like; cell division control protein 2 homolog 2; chloroplast stem-loop binding protein of 41 kDa b, chloroplastic; cysteine-rich receptor-like protein kinase 19; cytochrome B5-like protein; delta(12)-fatty-acid desaturase FAD2-like; desiccation-related protein PCC13-62-like; dormancy-associated protein 2-like; E3 ubiquitin-protein ligase SDIR1; gamma-carbonic anhydrase-like 2, mitochondrial; germacrene-A synthase-like; glucan endo-1,3-beta-glucosidase 12; glucose-6-phosphate 1-dehydrogenase 6, cytoplasmic-like; glycine-rich RNA-binding protein-like; malonate--CoA ligase; mannose-1-phosphate guanylyltransferase 1; mediator of RNA polymerase II transcription subunit 11; MLP-like protein 423; NADH dehydrogenase [ubiquinone] flavoprotein 2, mitochondrial; NADP-dependent glyceraldehyde-3-phosphate dehydrogenase; NDR1/HIN1-like protein 1; ornithine aminotransferase, mitochondrial; peptidyl-prolyl cis-trans isomerase CYP19-3; peroxidase 12-like; phosphoinositide phosphatase SAC1; probable gamma-secretase subunit PEN-2; probable protein phosphatase 2C 60; programmed cell death protein 2-like; protein CASP; protein ELF4-LIKE 4-like; protein REVEILLE 8; protein SRC1; protein TIC 56, chloroplastic; pyruvate decarboxylase 1-like; small acidic protein 1; sphingoid long-chain bases kinase 1; structural maintenance of chromosomes protein 1; translationally-controlled tumor protein homolog; tubulin beta-2 chain; ubiquitin-conjugating enzyme E2-17 kDa-like; U-box domain-containing protein 34; upstream activation factor subunit UAF30; uridine kinase-like protein 1, chloroplastic; V-type proton ATPase subunit B 1; and YTH domain-containing protein ECT4-like.

3. The cell of claim 1, wherein the nucleic acid derived from Cannabis trichome mRNA encodes a polypeptide comprising (a) an amino acid sequence having at least 90% identity to any one of even-numbered SEQ ID NOs: 16-540; (b) an amino acid sequence having at least 90%, at least 95%, at least 96%, at least 97%, at least 98%, or at least 99% identity to any one of SEQ ID NOs: 36, 74, 88, 94, 104, 106, 108, 110, 112, 114, 116, 120, 122, 124, 126, 128, 130, 132, 134, 136, 138, 140, 142, 144, 146, 14, 150, 168, 174, 184, 188, 206, 210, 232, 270, 274, and 396; or (c) an amino acid sequence having at least 90% identity to any one of even-numbered SEQ ID NOs: 122-150.

4. The cell of claim 1, wherein the nucleic acid derived from Cannabis trichome mRNA comprises: (a) a nucleotide sequence having at least 90% identity to any one of odd-numbered SEQ ID NOs: 15-539; (b) a nucleotide sequence having at least 90%, at least 95%, at least 96%, at least 97%, at least 98%, or at least 99% identity to any one of SEQ ID NOs: 35, 73, 87, 93, 103, 105, 107, 109, 111, 113, 115, 119, 121, 123, 125, 127, 129, 131, 133, 135, 137, 139, 141, 143, 145, 147, 149, 167, 173, 183, 187, 205, 209, 231, 269, 273, and 395; or (c) a nucleotide sequence having at least 90% identity to any one of odd-numbered SEQ ID NOs: 121-149.

5. The cell of claim 1, wherein the recombinant host cell produces the cannabinoid with a titer that is increased at least 1.2-fold, at least 2-fold, at least 3-fold, at least 4-fold, at least 5-fold, or more relative to a control recombinant host cell comprising the pathway and not the nucleic acid derived from a Cannabis trichome mRNA.

6. The cell of claim 1, wherein the pathway capable of producing a cannabinoid comprises enzymes capable of converting hexanoic acid to CBGA.

7. The cell of claim 1, wherein the pathway capable of producing a cannabinoid comprises at least the following enzymes: AAE, OLS, OAC, and PT4; wherein AAE has an amino acid sequence of at least 90% identity to SEQ ID NO: 2, OLS has an amino acid sequence of at least 90% identity to SEQ ID NO: 4, OAC has an amino acid sequence of at least 90% identity to SEQ ID NO: 6, and PT4 has an amino acid sequence of at least 90% identity to SEQ ID NO: 8 or 10.

8. The cell of claim 1, wherein recombinant host cell source is selected from Saccharomyces cerevisiae, Yarrowia lipolytica, Pichia pastoris, and Escherichia coli.

9. A method for producing a cannabinoid comprising:
(a) culturing in a suitable medium a recombinant host cell of claim 1; and
(b) recovering the produced cannabinoid.

10. A method for making a recombinant host cell for producing a cannabinoid comprising introducing into a host cell:

11. (a) a first set of nucleic acids that encode a pathway of enzymes capable of producing a cannabinoid; and
12. (b) a nucleic acid derived from a Cannabis trichome mRNA that does not encode an enzyme in the pathway, wherein the nucleic acid encodes a polypeptide having a protein function selected from: lipid transfer type protein, mulatexin-like, amino acid biosynthesis, amino acid degradation, carbohydrate metabolism, carrier-mediated transport, cell cycle organization, cellular respiration, chromatin organization, circadian clock system regulation, coenzyme metabolism, oxidoreductase, transferase, hydrolase, lyase, effector-triggered immunity network co-regulatory protein, fatty acid biosynthesis, lipid degradation, messenger ribonucleoparticle (mRNP) export, organelle machinery ribonuclease activity, phytohormone action, protein folding, protein phosphorylation, protein S-glutathionylation, protein translocation, redox homeostasis, RNA biosynthesis, RNA processing, solute transport, terpenoid metabolism, and/or vesicle trafficking.

13. The method of claim 10, wherein the nucleic acid derived from a Cannabis trichome mRNA encodes a polypeptide selected from: non-specific lipid-transfer protein 1-like; non-specific lipid-transfer protein 2-like; major allergen Pru av 1-like; mulatexin-like; 1-acyl-sn-glycerol-3-phosphate acyltransferase 2; 1-aminocyclopropane-1-carboxylate oxidase homolog 1-like; acyl-acyl carrier protein thioesterase ATL3, chloroplastic-like; barwin-like; beta-adaptin-like protein B; BURP domain protein RD22-like; cationic peroxidase 2-like; cell division control protein 2 homolog 2; chloroplast stem-loop binding protein of 41 kDa b, chloroplastic; cysteine-rich receptor-like protein kinase 19; cytochrome B5-like protein; delta(12)-fatty-acid desaturase FAD2-like; desiccation-related protein PCC13-62-like; dormancy-associated protein 2-like; E3 ubiquitin-protein ligase SDIR1; gamma-carbonic anhydrase-like 2, mitochondrial; germacrene-A synthase-like; glucan endo-1,3-beta-glucosidase 12; glucose-6-phosphate 1-dehydrogenase 6, cytoplasmic-like; glycine-rich RNA-binding protein-like; malonate--CoA ligase; mannose-1-phosphate guanylyltransferase 1; mediator of RNA polymerase II transcription subunit 11; MLP-like protein 423; NADH dehydrogenase [ubiquinone] flavoprotein 2, mitochondrial; NADP-dependent glyceraldehyde-3-phosphate dehydrogenase; NDR1/HIN1-like protein 1; ornithine aminotransferase, mitochondrial; peptidyl-prolyl cis-trans isomerase CYP19-3; peroxidase 12-like; phosphoinositide phosphatase SAC1; probable gamma-secretase subunit PEN-2; probable protein phosphatase 2C 60; programmed cell death protein 2-like; protein CASP; protein ELF4-LIKE 4-like; protein REVEILLE 8; protein SRC1; protein TIC 56, chloroplastic; pyruvate decarboxylase 1-like; small acidic protein 1; sphingoid long-chain bases kinase 1; structural maintenance of chromosomes protein 1; translationally-controlled tumor protein homolog; tubulin beta-2 chain; ubiquitin-conjugating enzyme E2-17 kDa-like; U-box domain-containing protein 34; upstream activation factor subunit UAF30; uridine kinase-like protein 1, chloroplastic; V-type proton ATPase subunit B 1; and YTH domain-containing protein ECT4-like.

14. The method of claim 10, wherein the nucleic acid derived from Cannabis trichome mRNA encodes a polypeptide comprising: (a) an amino acid sequence having at least 90% identity to any one of even-numbered SEQ ID NOs: 16-540; (b) an amino acid sequence sequence having at least 90%, at least 95%, at least 96%, at least 97%, at least 98%, or at least 99% identity to any one of SEQ ID NOs: 36, 74, 88, 94, 104, 106, 108, 110, 112, 114, 116, 120, 122, 124, 126, 128, 130, 132, 134, 136, 138, 140, 142, 144, 146, 14, 150, 168, 174, 184, 188, 206, 210, 232, 270, 274, and 396; or (c) an amino acid sequence having at least 90% identity to any one of even-numbered SEQ ID NOs: 122-150.

15. The method of claim 10, wherein the nucleic acid derived from Cannabis trichome mRNA comprises: (a) a nucleotide sequence having at least 90% identity to any one of odd-numbered SEQ ID NOs: 15-539; (b) a nucleotide sequence having at least 90%, at least 95%, at least 96%, at least 97%, at least 98%, or at least 99% identity to any one of SEQ ID NOs: 35, 73, 87, 93, 103, 105, 107, 109, 111, 113, 115, 119, 121, 123, 125, 127, 129, 131, 133, 135, 137, 139, 141, 143, 145, 147, 149, 167, 173, 183, 187, 205, 209, 231, 269, 273, and 395; or (c) a nucleotide sequence having at least 90% identity to any one of odd-numbered SEQ ID NOs: 121-149.

16. The method of claim 10, wherein the pathway capable of producing a cannabinoid comprises enzymes capable of converting hexanoic acid to CBGA.

17. The method of claim 10, wherein the pathway capable of producing a cannabinoid comprises at least the following enzymes: AAE, OLS, OAC, and PT4; wherein AAE has an amino acid sequence of at least 90% identity to SEQ ID NO: 2, OLS has an amino acid sequence of at least 90% identity to SEQ ID NO: 4, OAC has an amino acid sequence of at least 90% identity to SEQ ID NO: 6, and PT4 has an amino acid sequence of at least 90% identity to SEQ ID NO: 8 or 10.

18. The method of any one of claims 30-37, wherein the recombinant host cell is a cell derived from a source selected from: Saccharomyces cerevisiae, Escherichia coli, Yarrowia lipolytica, and Pichia pastoris.


SEQUENCE LISTING (544 sequences)

SEQ ID NO: 1 - DNA, 2163 bp, Cannabis sativa
  source 1..2163: mol_type = other DNA
atgggaaaaaattataagtcacttgacagtgtggttgctagtgatttcatagccttgggtatcacatctgaggtagcggagactcttcatgggcgtttagctgagattgtgtgtaattacggggcggccacccctcagacttggatcaatatagcaaaccacatcttatctcctgatctacccttttctctacaccaaatgctgttctatgggtgttacaaagatttcgggcccgcaccgcccgcatggataccggatccagagaaagtcaaatccaccaacttaggcgcgttgcttgaaaaaagaggcaaggaattcctgggcgtcaagtacaaggaccccatctcatctttctcccacttccaagagttctctgtacgtaatccggaggtttactggcgtaccgtccttatggatgagatgaagatatcattctccaaggacccggaatgtatactaagaagggatgacattaacaacccagggggcagtgagtggcttccgggtggataccttaatagtgcaaaaaactgtcttaacgtgaactcaaacaaaaagttaaacgatacaatgatcgtgtggcgtgatgagggcaatgatgaccttccgctaaataagttgacgcttgatcagctaagaaagcgtgtctggcttgtcggatatgccctagaggagatgggccttgagaagggatgcgcgatcgccatagatatgcctatgcatgtcgatgctgtggttatatatctggccatcgttctagcgggctatgttgttgtgagtattgccgatagcttcagtgcacccgaaatatcaacgagactgcgtttgtccaaagccaaagcaatattcacacaggatcatatcattagaggaaagaagcgtattccgctatattcaagggtggtggaggccaaaagcccgatggctatagtgataccctgttcaggcagcaacatcggagctgaattaagagatggagatatatcctgggactattttttagagagggcgaaagaatttaagaactgcgagttcaccgcaagggaacagccagtggatgcgtatacaaacatactattcagttcaggtactacgggagaaccaaaggctataccctggacgcaggcaaccccgctaaaagctgccgctgacgggtggtctcatttggatatcaggaagggtgatgttatcgtatggccaacaaatcttggatggatgatgggaccgtggcttgtgtacgctagcttgttaaatggagcttcaatcgctctatataatggttcccctcttgtcagcggttttgcgaagttcgtacaggatgccaaggtaactatgctgggagtcgttcctagcatcgtcaggagttggaaatcaactaactgtgtgtctggttacgactggtccactatcagatgttttagctccagtggtgaggcctccaacgtagatgagtacctttggcttatgggacgtgccaactataaaccggtaatagagatgtgtgggggtactgaaataggaggagcgttttcagctggcagttttcttcaggctcaaagcttgtctagtttctcttcacagtgtatgggctgtacgttatacattcttgataagaacggttatccaatgcccaaaaacaaaccaggtataggagaattggcgctgggtccagtcatgtttggcgctagtaaaactctactgaacggaaatcatcacgacgtttactttaaaggtatgcctactcttaatggagaagtacttaggcgtcatggcgatatctttgagttgacatccaatggctactatcacgcccatggcagggcggatgacaccatgaacatcggggggatcaaaatctccagtatagagatcgagagagtgtgtaacgaggtagacgaccgtgtattcgagacaacagccattggggttccacccctaggtggtggccccgaacaacttgttatcttttttgtgctgaaggactcaaatgacactaccattgatttaaatcaactacgtttgtcattcaatctgggattacaaaagaagttgaatcctttattcaaggtcacaagagtagtaccccttagttccctgccaagaactgcgacaaacaagataatgcgtagagtgctaaggcagcagtttagtcattttgagtaa

SEQ ID NO: 2 - protein, 720 aa, Cannabis sativa
MGKNYKSLDSVVASDFIALGITSEVAETLHGRLAEIVCNYGAATPQTWINIANHILSPDLPFSLHQMLFYGCYKDFGPAPPAWIPDPEKVKSTNLGALLEKRGKEFLGVKYKDPISSFSHFQEFSVRNPEVYWRTVLMDEMKISFSKDPECILRRDDINNPGGSEWLPGGYLNSAKNCLNVNSNKKLNDTMIVWRDEGNDDLPLNKLTLDQLRKRVWLVGYALEEMGLEKGCAIAIDMPMHVDAVVIYLAIVLAGYVVVSIADSFSAPEISTRLRLSKAKAIFTQDHIIRGKKRIPLYSRVVEAKSPMAIVIPCSGSNIGAELRDGDISWDYFLERAKEFKNCEFTAREQPVDAYTNILFSSGTTGEPKAIPWTQATPLKAAADGWSHLDIRKGDVIVWPTNLGWMMGPWLVYASLLNGASIALYNGSPLVSGFAKFVQDAKVTMLGVVPSIVRSWKSTNCVSGYDWSTIRCFSSSGEASNVDEYLWLMGRANYKPVIEMCGGTEIGGAFSAGSFLQAQSLSSFSSQCMGCTLYILDKNGYPMPKNKPGIGELALGPVMFGASKTLLNGNHHDVYFKGMPTLNGEVLRRHGDIFELTSNGYYHAHGRADDTMNIGGIKISSIEIERVCNEVDDRVFETTAIGVPPLGGGPEQLVIFFVLKDSNDTTIDLNQLRLSFNLGLQKKLNPLFKVTRVVPLSSLPRTATNKIMRRVLRQQFSHFE

SEQ ID NO: 3 - DNA, 1158 bp, Cannabis sativa
  source 1..1158: mol_type = other DNA
atgaaccacttgagagcagaagggccggccagtgtactggctatagggacagccaaccccgaaaatatactgttgcaagatgagttcccagattattactttagagtgactaaatccgagcacatgacgcaacttaaggagaaattcaggaaaatatgcgacaaatctatgattagaaaaagaaattgtttcttaaatgaggagcatctaaagcagaatccccgtctggtagaacatgaaatgcaaactttggacgcgcgtcaagacatgctagttgtcgaagtgcctaaattaggcaaagacgcgtgtgcaaaggctataaaagagtggggccaaccgaagtccaaaattacacacctaatattcacttctgcgtccaccaccgacatgcccggagccgactaccactgtgcgaaacttctaggcctatccccttcagtcaagcgtgtaatgatgtatcaactggggtgctacggaggaggcaccgttttgaggattgcaaaggatatcgctgaaaataacaagggggctcgtgtacttgctgtgtgctgtgatatcatggcctgccttttcagaggcccctcagagtcagatcttgaactgttagtaggtcaggctatcttcggagatggcgctgcagccgtcatagttggggcggagcctgacgaatcagttggggagaggcccattttcgagctggtcagtacgggacagaccatcttgccaaatagcgagggcacgatcggaggccacataagggaggcgggtttgatatttgaccttcataaggatgtaccgatgttgatctccaataatattgagaagtgtcttattgaagcatttacccctattggtatttcagactggaacagtatcttctggattacgcatccgggaggtaaggcgattcttgataaagtcgaagaaaagctacacctgaagtcagacaagttcgttgactccagacacgttctttcagagcacggcaacatgagttcttccaccgtccttttcgtaatggacgagctgaggaaacgtagccttgaggaaggtaaaagtacgacaggagatgggtttgagtggggagtgttgtttggcttcggcccagggttaacagttgaacgtgtagtcgttagatctgtccctattaaatactaa

SEQ ID NO: 4 - protein, 385 aa, Cannabis sativa
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 5 - DNA, 306 bp, Cannabis sativa
  source 1..306: mol_type = other DNA
atggctgtcaagcaccttatcgtactgaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgagtcagtagagacgatccaagattacataatacacccggcccatgtgggattcggagacgtttaccgtagcttctgggaaaaacttttgattttcgactataccccgagaaaataa

SEQ ID NO: 6 - protein, 101 aa, Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 7 - DNA, 1517 bp, Cannabis sativa
  source 1..1517: mol_type = other DNA
atcaataataatcttcatgggactctcattagtttgtaccttttcatttcaaactaattatcatactttattaaaccctcataataagaatcccaaaaactcattattatcttatcaacaccccaaaacaccaataattaaatcctcttatgataattttccctctaaatattgcttaaccaagaactttcatttacttggactcaattcacacaacagaataagctcacaatcaaggtccattagggcaggtagcgatcaaattgaaggttctcctcatcatgaatctgataattcaatagcaactaaaattttaaattttggacatacttgttggaaacttcaaagaccatatgtagtaaaagggatgatttcaatcgcttgtggtttgtttgggagagagttgttcaataacagacatttattcagttggggtttgatgtggaaggcattctttgctttggtgcctatattgtccttcaatttctttgcagcaatcatgaatcaaatttacgatgtggacatcgacaggataaacaagcctgatctaccactagtttcaggggaaatgtcaattgaaacagcttggattttgagcataattgtggcactaactgggttgatagtaactataaaattgaaatctgcaccactttttgttttcatttacatttttggtatatttgctgggtttgcctattctgttccaccaattagatggaagcaatatccttttaccaattttctaattaccatatcgagtcatgtgggcttagctttcacatcatattctgcaaccacatcagctcttggtttaccatttgtgtggaggcctgcttttagtttcatcatagcattcatgacagttatgggtatgactattgcttttgccaaagatatttcagatattgaaggcgacgccaaatatggggtatcaactgttgcaaccaaattaggtgctaggaacatgacatttgttgtttctggagttcttcttctaaactacttggtttctatatctattgggataatttggcctcaggttttcaagagtaacataatgatactttctcatgcaatcttagcattttgcttaatcttccagactcgtgagcttgctctagcaaattacgcctcggcgccaagcagacaattcttcgagtttatctggttgctatattatgctgaatactttgtatatgtatttatataagaccataatataacatatatatgtttattacataaaattgggacacaaaaacgtcaattatttggacaaaagtactcagaaagacctctttcactacaaggggaggccatttagttatacttgggtttcaatcaacaaatttataaatttttaagattttatttacaaaacattttcatgtgtaattaaatcgatcgtcatttattttttggatacaacttggttcaacttattttaattagagtgcttcgtaatttaactacaattatagaagggcattttataaaaatactggatttggggt

SEQ ID NO: 8 - protein, 398 aa, Cannabis sativa
MGLSLVCTFSFQTNYHTLLNPHNKNPKNSLLSYQHPKTPIIKSSYDNFPSKYCLTKNFHLLGLNSHNRISSQSRSIRAGSDQIEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 9 - DNA, 954 bp, synthetic construct
  misc_feature 1..954: note = Synthetic polynucleotide
  source 1..954: mol_type = other DNA
atgatcgaaggttcacctcatcatgaaagtgataacagcatagccacgaagattttgaatttcggccatacttgttggaagctacagaggccgtacgtcgttaaggggatgatttccattgcgtgcggtctgtttggcagggaattatttaacaacagacacttattcagttggggcctgatgtggaaggccttcttcgctcttgtacccattctgtccttcaacttttttgcagcgatcatgaatcaaatatacgatgtagacatcgatagaataaacaagcccgatttacctctggtatcaggcgaaatgagcatcgaaactgcgtggattttatcaatcatcgttgcattgactgggctgatagtgaccataaagttaaagtcagccccgttgtttgtcttcatatacatcttcggcattttcgcgggctttgcgtatagtgtacctcccattagatggaagcagtacccgtttactaactttcttattacaattagcagccatgtcggtcttgcattcacgtcctactcagccaccacatccgcactggggctaccgtttgtgtggcgtccagccttcagcttcatcatcgcattcatgacagtaatgggtatgacgatagcttttgcaaaggatataagtgatatcgagggtgacgctaagtatggagtgtctactgtggccacgaagctgggggcccgtaatatgactttcgtggtatcaggtgtactattgcttaattaccttgtttctatatcaatcggaattatttggccacaagttttcaaatccaatataatgatcctatcacacgctattttagcgttttgtttgatatttcagactagagagcttgcactagcgaattacgcgagtgccccgagtaggcagtttttcgagttcatatggctattatactatgctgagtactttgtttacgtatttatttaa

SEQ ID NO: 10 - protein, 317 aa, synthetic construct
  REGION 1..317: note = Synthetic polypeptide
MIEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 11 - DNA, 1635 bp, Cannabis sativa
  source 1..1635: mol_type = other DNA
atgaagtgctcaacattctccttttggtttgtttgcaagataatatttttctttttctcattcaatatccaaacttccattgctaatcctcgagaaaacttccttaaatgcttctcgcaatatattcccaataatgcaacaaatctaaaactcgtatacactcaaaacaacccattgtatatgtctgtcctaaattcgacaatacacaatcttagattcacctctgacacaaccccaaaaccacttgttatcgtcactccttcacatgtctctcatatccaaggcactattctatgctccaagaaagttggcttgcagattcgaactcgaagtggtggtcatgattctgagggcatgtcctacatatctcaagtcccatttgttatagtagacttgagaaacatgcgttcaatcaaaatagatgttcatagccaaactgcatgggttgaagccggagctacccttggagaagtttattattgggttaatgagaaaaatgagaatcttagtttggcggctgggtattgccctactgtttgcgcaggtggacactttggtggaggaggctatggaccattgatgagaaactatggcctcgcggctgataatatcattgatgcacacttagtcaacgttcatggaaaagtgctagatcgaaaatctatgggggaagatctcttttgggctttacgtggtggtggagcagaaagcttcggaatcattgtagcatggaaaattagactggttgctgtcccaaagtctactatgtttagtgttaaaaagatcatggagatacatgagcttgtcaagttagttaacaaatggcaaaatattgcttacaagtatgacaaagatttattactcatgactcacttcataactaggaacattacagataatcaagggaagaataagacagcaatacacacttacttctcttcagttttccttggtggagtggatagtctagtcgacttgatgaacaagagttttcctgagttgggtattaaaaaaacggattgcagacaattgagctggattgatactatcatcttctatagtggtgttgtaaattacgacactgataattttaacaaggaaattttgcttgatagatccgctgggcagaacggtgctttcaagattaagttagactacgttaagaaaccaattccagaatctgtatttgtccaaattttggaaaaattatatgaagaagatataggagctgggatgtatgcgttgtacccttacggtggtataatggatgagatttcagaatcagcaattccattccctcatcgagctggaatcttgtatgagttatggtacatatgtagttgggagaagcaagaagataacgaaaagcatctaaactggattagaaatatttataacttcatgactccttatgtgtccaaaaatccaagattggcatatctcaattatagagaccttgatataggaataaatgatcccaagaatccaaataattacacacaagcacgtatttggggtgagaagtattttggtaaaaattttgacaggctagtaaaagtgaaaaccctggttgatcccaataacttttttagaaacgaacaaagcatcccacctcttccacggcatcgtcattaa

SEQ ID NO: 12 - protein, 544 aa, Cannabis sativa
MKCSTFSFWFVCKIIFFFFSFNIQTSIANPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 13 - DNA, 1554 bp, Cannabis sativa
  source 1..1554: mol_type = other DNA
atgaacccccgtgaaaattttttgaaatgtttctctcaatacatacccaacaatgcaaccaacttaaagctggtatatactcaaaacaaccccctatatatgtctgttctaaatagtactatccataacttacgtttcacctcagataccacccctaaaccgctggtcatcgtgactccgtctcatgtttcacacatacagggcacgatattgtgctcaaaaaaggtcgggttacagattcgtacccgttcaggaggtcatgatagtgagggaatgtcttacatctcccaggtcccttttgtaattgtcgaccttcgtaatatgagatccataaagatcgacgttcattcacagacggcgtgggtagaggctggtgcaaccctaggtgaagtctactactgggtcaacgaaaaaaacgagaacttatcattagctgcggggtattgccctacagtttgtgccggaggtcattttggaggtggaggctacgggccactgatgaggaactacggtctggcagcagacaacattatagatgcacacctagtgaacgtgcatggtaaagttttagatagaaagtccatgggagaagatttgttttgggcactacgtggaggaggggctgagtcattcgggattattgtagcgtggaagatccgtctggtcgcagtccctaaatctacgatgttttccgtgaaaaagattatggaaattcacgagctagtgaaacttgtcaataagtggcagaatatagcatacaaatatgacaaggatctattgttgatgacgcatttcatcacaagaaacattacggacaatcaaggtaagaataagacggctattcacacttacttcagctccgtttttctaggaggggtagattccctagttgacctgatgaataagagttttcccgagttgggtattaaaaaaactgattgtagacagctgtcttggatcgacacaatcatattctactctggtgtggtaaactatgacaccgataatttcaacaaagaaatcttactggatagatcagccggtcaaaacggcgcgtttaaaatcaagctggattacgtaaagaagcctatacccgaatccgtatttgtacagattctggaaaagttatacgaggaagacattggggcgggtatgtacgctctttacccttacggcgggatcatggatgagatttccgaaagtgctatcccgttccctcatcgtgctggcattctgtacgagttatggtatatttgcagttgggagaagcaggaggataatgaaaagcacctaaattggattcgtaatatttataatttcatgactccctatgttagtaagaaccccagactggcctaccttaattatagagacctggacatcgggataaatgatccgaagaacccaaataactatacgcaggccaggatttggggggaaaagtatttcggaaagaactttgacagactggtgaaagttaagaccctggtggatccaaataattttttcaggaacgagcagagtattcccccgcttccacgtcacaggcattaa

SEQ ID NO: 14 - protein, 517 aa, Cannabis sativa
MNPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 15 - DNA, 600 bp, Cannabis sativa
  source 1..600: mol_type = other DNA
atgattccaaaggtccaggatcctagagaggaagttcttcaagcatggttcatggatgacagtgatgaagaccagagacttcctcatcatcgggaaccaaaggaatttgtatctttggaccaacttgctgagcttggagtgctcagctggagacttgatgctgataactatgaaacagatgaggagctgaagaagattcgtacagaacgtggatactcctacacggatttctgtgaagtttgcccagagaagcttccaaattacgaagaaaagataaaaaatttctttgaggaacatcttcacactgatgaagagatccgctactgtgtggcaggaagtggttattttgatgtgagagaccgtgatgaacgctggatccgtgtatgggtgaagaaaggtggaatgattgttttgcctgctggaatttatcaccgttttactttggacacaaacaattatatcaaggcaatgaggctttttgttggtgacccagtttggactccattcaatcgtccccacgaccatcttccagctcggaaggagtatgtgaaatcttttgtggaaaaggatggtggtaatgctataaatgctgcagcttga

SEQ ID NO: 16 - protein, 199 aa, Cannabis sativa
MIPKVQDPREEVLQAWFMDDSDEDQRLPHHREPKEFVSLDQLAELGVLSWRLDADNYETDEELKKIRTERGYSYTDFCEVCPEKLPNYEEKIKNFFEEHLHTDEEIRYCVAGSGYFDVRDRDERWIRVWVKKGGMIVLPAGIYHRFTLDTNNYIKAMRLFVGDPVWTPFNRPHDHLPARKEYVKSFVEKDGGNAINAAA

SEQ ID NO: 17 - DNA, 1080 bp, Cannabis sativa
  source 1..1080: mol_type = other DNA
atgcttgctggtctagctttcaagagaaaatggcagcaaaagaggaaagcagagggaaagccctttgataagcctaatatagtcactggtgccaatgttcaggtgtgttgggagaagtttgcaaggtattttgaggttgagctgaaagaggttaaactaaaagagggatattacgtgatggatcctgagaaggcagttgagatggtggatgagaacaccatttgtgttgcagccattcttggctccacattgactggtgagtttgaggatgtgaaaaaagtcaatgaactccttactctcaagaacaaggaaacaggttgggacaccccgattcacgtagatgccgccagcggcggttttgttgcaccgttcctttatccggagattgaatgggatttcagattgcctctggtgaaaagcatcaatgtgagtggccataagtatgggcttgtctatgctggtattggatgggttgtgtggagggacaaagaggacttacctgaagaccttattttccatatcaattaccttggatctgaccaacctactttcaccctcaacttctccaaagggtctagtcaaatcattgctcagtattaccaattcatccgattgggatttgagggttacaagaacataatggaaaactgcatggcaactgcaaaaatactacaaaacagattagagaaaacagggcactttgacatagtctcaaaagacataggggtgcccgttgtggcattctctctcaaagacagtagcaaatacactgtgttcaacatagcagacagcttgagaaggtttgggtggattgtgccagcctacaccatgcctgccgacgccgaacacatcgctgttcttcgggttgtgattagggaggacttcagccgtagcttggtggagagactgatctcagacatattgaaagtgctgaaggatctggacgatcttccaaccaccagtgccacaccgatcaatactgacactgcaatcaagaagaagactgatagacagattgaagaagaagtgaccggacattggaagcgttttgtcaaggaaaggaaaactggagcatgttaa

SEQ ID NO: 18 - protein, 499 aa, Cannabis sativa
MVLTTASKGRGDSGEDDENADHYLNSTFASRYVREAVPKWRMPKKTIPKDAAFQFINDELMLDGTPRLNLASFVTTWMEPECDKLIMASINKNYVDMDEYPVTTEIQNRCVNMIAHLFNAPTGDTEAAIGVGTVGSSEAIMLAGLAFKRKWQQKRKAEGKPFDKPNIVTGANVQVCWEKFARYFEVELKEVKLKEGYYVMDPEKAVEMVDENTICVAAILGSTLTGEFEDVKKVNELLTLKNKETGWDTPIHVDAASGGFVAPFLYPEIEWDFRLPLVKSINVSGHKYGLVYAGIGWVVWRDKEDLPEDLIFHINYLGSDQPTFTLNFSKGSSQIIAQYYQFIRLGFEGYKNIMENCMATAKILQNRLEKTGHFDIVSKDIGVPVVAFSLKDSSKYTVFNIADSLRRFGWIVPAYTMPADAEHIAVLRVVIREDFSRSLVERLISDILKVLKDLDDLPTTSATPINTDTAIKKKTDRQIEEEVTGHWKRFVKERKTGAC

SEQ ID NO: 19 - DNA, 1446 bp, Cannabis sativa
  source 1..1446: mol_type = other DNA
atggcgtataggaggtgtttgcagctgaatcatgttctgggcagacttagttttactgcaaccaggaattttactggtctccctaaacccaacccctcttcttcttcttcctcccaagacctcatcaactccgaacaagagttcagtgctcacaactatcacccaataccagttgtgttctctcaggcaaagggatcatgtatatgggatcctgaaggcaaacggtatctagattttctctctgcttattctgcagttaatcagggacattgtcatccgaaaatcatgaaggcattacaagaacaggcggaaaggctcacccttagctctcgagcattctttaatgataaatttccgatatttgctgagcgtctaacagacatgttcggatatgacatggttctgcctatgaacactggtgctgaaggtgtagaaacagctctgaagttagccaggaaatggggttacgagaaaaagaaaattccaaatgataaggctatcattgtgtcatgctgtggatgcttccatggtcgtacattgggtgtcatctcaatgagctgcgacaatgaggcaactcgaggttttggacccttgttgcctggtcatctcaaagttgattttggtgatttagaagcccttgaaaaagcttttaaagaaaatggagatcatatagctggatttttgtttgaacccatccaaggagaggccggggttgttattcctcctgatggttatttaaaatctgtcagagaactttgctcaaaatacaacattttaatgattgctgacgaaatccaatctggcttagctagatcgggaaaattgctcgcttgtgattgggaagaagttcgcccagatgtggtgattttaggaaaagccttaggcggtggagtaatcccggtaagtgcagttcttgcagacaaagatgtgatgctctgtattagacctggagagcatggaagtacctttggcgggaacccattggccagtgcaattgccattgcgtcgcttgatgtaatcaaagatgagaaactttccgagagatctgcacaactaggacaggaacttatgaaaaaactacttaaaattcaacagaaattccccgactacattaagaaagttcgaggaagaggcttgttcagtgccatggagcttaacagtaagaacttgcaccctgtttcggcttatgatatctgtcttaagttgaaggaaagaggagttcttgcaaagcctacacatgacactattatcagattaactccaccattgacaataagtttggatgagctccaagaagggttaaatgcccttcatgatgttctaaaactggatttaccggacatgaaaaagacaaagccaaagccgaaagatacctcttcgaatgcttcttcgaacacatgcgaccgttgtggaagagacttgtatgcttctcaagacaaagaatga

SEQ ID NO: 20 - protein, 481 aa, Cannabis sativa
MAYRRCLQLNHVLGRLSFTATRNFTGLPKPNPSSSSSSQDLINSEQEFSAHNYHPIPVVFSQAKGSCIWDPEGKRYLDFLSAYSAVNQGHCHPKIMKALQEQAERLTLSSRAFFNDKFPIFAERLTDMFGYDMVLPMNTGAEGVETALKLARKWGYEKKKIPNDKAIIVSCCGCFHGRTLGVISMSCDNEATRGFGPLLPGHLKVDFGDLEALEKAFKENGDHIAGFLFEPIQGEAGVVIPPDGYLKSVRELCSKYNILMIADEIQSGLARSGKLLACDWEEVRPDVVILGKALGGGVIPVSAVLADKDVMLCIRPGEHGSTFGGNPLASAIAIASLDVIKDEKLSERSAQLGQELMKKLLKIQQKFPDYIKKVRGRGLFSAMELNSKNLHPVSAYDICLKLKERGVLAKPTHDTIIRLTPPLTISLDELQEGLNALHDVLKLDLPDMKKTKPKPKDTSSNASSNTCDRCGRDLYASQDKE

SEQ ID NO: 21 - DNA, 924 bp, Cannabis sativa
  source 1..924: mol_type = other DNA
atgattagagcctatacacaatctgctgcaactttgaatcttttgagggcatttgctactggaggttatgctgctatgcagagagtgacccactggaatctagatttcactgatcacagtgagcagggggatagataccgtgaactggctcatcgggttgatgaggcccttgggttcatggctgctgctggacttactcttgaccacccaataatgaccacaactgaattctggacatctcatgagtgcttgcttctgccttacgagcaagcacttactagggaagattctacttctgggctttactacgactgctctgctcacatggtgtgggttggagaacgcacccgccaactagacggtgcacatgttgaattcttgagaggagttgctaatccccttgggataaaggtgagtgataagatggatccaaacgagcttgttaagcttgtggacattttgaaccccaataacaagccaggaagaataacagtgattgtgagaatgggagcagaaaatttgagagtaaagtttcctcatcttatcaaggctgttcgtggagctggtcagattgtcacttgggtcagtgatcccatgcatgggaacaccatcaaagcaccttgtggactcaaaactcgctcttttgatgcaatcaggaatgaggtgagagccttcttcgacgtgcatgatcaagaaggaagtttccctggtggtgttcatctggagatgacagggcaaaatgtgacggagtgtgtcggaggctcaagaactataacttataatgatttaggctcacgctaccacacccattgtgacccaaggctcaatgcttcccagtcgctggagctcgccttcattattgcagagcggctgcgaaagagaaggctcaaatcactgaaccctgtggcctcagctggtttgtaa

SEQ ID NO: 22 - protein, 525 aa, Cannabis sativa
MALASTTPVTKSLLNNQNPKPLSPKFNLNLHSHLPRLPNRRKPISAVHAAEPSKPSKSTSQTASNPSKSWTIDSWKNKKAIQLPEYPDRAELDSVLKTLESFPPIVFAGEARSLEEKLGQAAMGKAFLLQGGDCAESFKEFNANNIRDTFRVLLQMGVVLMFGGQMPIVKVGRMAGQFAKPRSDSFEDRDGVKLPSYRGDNVNGDAFDEKSRIPDPNRMIRAYTQSAATLNLLRAFATGGYAAMQRVTHWNLDFTDHSEQGDRYRELAHRVDEALGFMAAAGLTLDHPIMTTTEFWTSHECLLLPYEQALTREDSTSGLYYDCSAHMVWVGERTRQLDGAHVEFLRGVANPLGIKVSDKMDPNELVKLVDILNPNNKPGRITVIVRMGAENLRVKFPHLIKAVRGAGQIVTWVSDPMHGNTIKAPCGLKTRSFDAIRNEVRAFFDVHDQEGSFPGGVHLEMTGQNVTECVGGSRTITYNDLGSRYHTHCDPRLNASQSLELAFIIAERLRKRRLKSLNPVASAGL

SEQ ID NO: 23 - DNA, 1263 bp, Cannabis sativa
  source 1..1263: mol_type = other DNA
atggctctgaagtccttcatcgatgttcttcccaattctcacttccccatacagaatcttccatttggagtattcaagcctgagccaagttcagctcctcgaccgggcgtagcaatcggtgactacgtcctcgacctctccgagatcgcctccgctggtctattcgatggtccgattctcaagaactccgattgttttcagcagcctaatctaaacaagttgttggcattgggacgacctgcatggaaggaatctcgaattactctgcaaaagctattatcatctactgaaccaaaattacgtgataatgcaaatttgaggcagaaagcactcgttccaatgaacaaggtggaattgcttcttcctattgccgtaggggattacacagatttcttttcctcaatgcatcatgcaaagaattgtggaaccatgtttcgtggaccggagaatgcaattccacccaattggtttcaccttcccattgcataccatgggagagcatcatctgttgtaatctctggaacagacattattcgaccgagaggtcaaggctatccaagtggaacctctccaccatattttggtccgacactgaagttagattatgaactggaaatggctgctgtagttggttcaggaaatgaacttggaaagcctgtgaatgtcaacgaggcagcagatcatatttttggacttgtgctgatgaatgactggagtgcaagagacattcaggcgtgggaatatgttcctcttgggccatttcttggtaaaagttttggcactactatatctccttggattgtgagccttgaagctctagaaccttttgcttgtgctgctcccaaacaggatccccttccattgccttatctggctgaaaaagtctctagaaattatgacattcagttagaggttgaaatcaagcctgctggcgtagaggattcaactgtggtcacaaagagtaatttcaaacatttatattggacattgacacaacagatagctcatcatactattaacggttgcaacttgagacctggcgatatcctgggaactggaaccattagtggacctgagccagattccttgggatgtttgttagagttgtcatggaatggacagaagacaatatcactgaaaggggcgacccgtaaattcctagaagatggtgatgaagttatcatcactggtttttgcaagggagatgaatacaccattgggtttggcacctgctcggggaagattgttcctccattgccttga

SEQ ID NO: 24 - protein, 420 aa, Cannabis sativa
MALKSFIDVLPNSHFPIQNLPFGVFKPEPSSAPRPGVAIGDYVLDLSEIASAGLFDGPILKNSDCFQQPNLNKLLALGRPAWKESRITLQKLLSSTEPKLRDNANLRQKALVPMNKVELLLPIAVGDYTDFFSSMHHAKNCGTMFRGPENAIPPNWFHLPIAYHGRASSVVISGTDIIRPRGQGYPSGTSPPYFGPTLKLDYELEMAAVVGSGNELGKPVNVNEAADHIFGLVLMNDWSARDIQAWEYVPLGPFLGKSFGTTISPWIVSLEALEPFACAAPKQDPLPLPYLAEKVSRNYDIQLEVEIKPAGVEDSTVVTKSNFKHLYWTLTQQIAHHTINGCNLRPGDILGTGTISGPEPDSLGCLLELSWNGQKTISLKGATRKFLEDGDEVIITGFCKGDEYTIGFGTCSGKIVPPLP

SEQ ID NO: 25 - DNA, 864 bp, Cannabis sativa
  source 1..864: mol_type = other DNA
atgaagaaccattgtatgatccaagtgaaacttcgttctatagcaccagtagaccataagcagcaattcgatatccgatcaatcattgcccgtattgttgatgggagcaaatttgacgaattcaaaaaattatatggcactacccttgtaacaggctttgctagaatttttggacagccagttggaataattggaaacaatggtatattgttcagtgagtctgctctgaaaggagctcattttattgaattatgcgctcaacgaaaaattcccttggtcttccttcaaaatatcaatggatttatggttggctcaagatctgaggcaggtggaatagcaaaggctggagcaaaactggtgatggcagtttcttgttgcaaagattccaaaagtaactataattgtgggtggaagctatggtgctggaattatgcaatgtgtggccgagcatacagtcccaatttcttgtttgtatggccaaatgcccgaatatcagtaatgggtggggctcaggctgctggtgtgctgtctcaaatagaaggtagcagcaagaaaaagcaaggaataaagtgggagaaagacgaggatgaggcattcaaggcgaaggtaatggaggcattcgataaagaaggaaatccttattactcgacagcaaggctctgggatgatggtgttattgatccagctgatacaagaaaggttattggactttccatctctgcttctttaaaccgtacccctgaagatactaagttggtgtctttaggatgtaaaaatagaatgactaactctcttactgtgcatttatatgtaattatgtatatattttactcttcaataatacaagacttgtgtaacaaaaaa

SEQ ID NO: 26 - protein, 288 aa, Cannabis sativa
MKNHCMIQVKLRSIAPVDHKQQFDIRSIIARIVDGSKFDEFKKLYGTTLVTGFARIFGQPVGIIGNNGILFSESALKGAHFIELCAQRKIPLVFLQNINGFMVGSRSEAGGIAKAGAKLVMAVSCCKDSKSNYNCGWKLWCWNYAMCGRAYSPNFLFVWPNARISVMGGAQAAGVLSQIEGSSKKKQGIKWEKDEDEAFKAKVMEAFDKEGNPYYSTARLWDDGVIDPADTRKVIGLSISASLNRTPEDTKLVSLGCKNRMTNSLTVHLYVIMYIFYSSIIQDLCNKK

SEQ ID NO: 27 - DNA, 1023 bp, Cannabis sativa
  source 1..1023: mol_type = other DNA
atggcttcctcagtagcaggggacagaactagtttctcgacaaaatatgtgtctgttaatgaccctgttgtttctgttgattggcttcgtgctaacatcaaggaccctaacttgaaggtgttggatgcatcttggtacatgcccgatgaacaaaggaatccacttcaagagtatcaggttgctcacattcccggtgcccttttctttgacgtagatggaatatcagaccgcacaacaaaattgccacacatgttgccatcagaggaagcctttgctgcagctgtttctgctcttggcatcaagaacaaagatgatctagttgtgtacgacgggaaggggatttttagtgcagctcgggtttggtggatgttccgagtttttggccatgatagaatttgggttttagatggcggcttgccaagatggcgagcatcaggatatgatgttgaatccagtgcttcaggagatgctattttgaaggctagtgctgccagtgaggcaatagagaaagtttatcaaggacaggcggttggcccaattacatttgagacaaagtttcagccacatcttgtgtggacacttgaccaggtaacaagtaacatcgagaacaagacacaccaacttatcgatgctcgatcaaaacccaggtttgatggagctgttccagaacctcgaaagggtataagaagtggccatgttcctgggagcaagtgtgttccttttgcacaggttttggatgcatctcagtcacttttaccggccgatgaactgaaaaaacggttcaatcaagaaggtatctctctggaaagccccgttgttacatcatgtggaaccggcgtactgcttgtattctcgctctgggtcttcatcggcttggcaagcctgatgttccggtgtacgatggatcatggaccgaatggggatcacattcagaaacgcccatcaacacatctgagtaaacttcacaatttgcagaggattggattttaccaaaccacaacccggacagcgaaaagattgtaa

SEQ ID NO: 28 - protein, 340 aa, Cannabis sativa
MASSVAGDRTSFSTKYVSVNDPVVSVDWLRANIKDPNLKVLDASWYMPDEQRNPLQEYQVAHIPGALFFDVDGISDRTTKLPHMLPSEEAFAAAVSALGIKNKDDLVVYDGKGIFSAARVWWMFRVFGHDRIWVLDGGLPRWRASGYDVESSASGDAILKASAASEAIEKVYQGQAVGPITFETKFQPHLVWTLDQVTSNIENKTHQLIDARSKPRFDGAVPEPRKGIRSGHVPGSKCVPFAQVLDASQSLLPADELKKRFNQEGISLESPVVTSCGTGVLLVFSLWVFIGLASLMFRCTMDHGPNGDHIQKRPSTHLSKLHNLQRIGFYQTTTRTAKRL

SEQ ID NO: 29 - DNA, 1554 bp, Cannabis sativa
  source 1..1554: mol_type = other DNA
atgggatcaggacagtggcatattgagaaaagaagcagttttaaaaacgattctgagtctatagaggccgaaaattttcctgaaaacgggtctctttcgattattgtcctcggagcttctggtgatcttgccaagaagaagacatttccggcactttttaaccttttcaaacagggacttcttccatccaatgaagttcacatttttggttatgcaagaaccaagatcacggatgatgagctaagagaccgcttgcgtggatatcttatccctaagaaaggtggctcatccaaggacttggaaactgtttcacagtttttgcaactgatcaagtacgtttgtggagcttatgatgctgaggaggggtttcggctattggataaggaaatttcaaagcatgaagtctcaaaacatagtgaaaatggattatctaggagactattttatcttgcacttcctccatcagtatatccgtctgtctgcaagatgatcaggcattactgtatgaataaaactgatcttggtggatggacacgagttgttgttgagaagccatttggcagggatttggcatctgccgaggaactcagttctcaaattggagagttgtttgaggaatctcaattgtatcgtattgatcattatttgggaaaggagttagtgcaaaatttgttggtccttcgttttgcaaaccgtttctttttgcccctttggaaccgtgataacattgacaacgtacagatcgtgttcagggaggatttcggaactgatggtcgtggtggttattttgatcaatacggaattatccgcgatattattcaaaatcacctgttgcaggttctctgtctggttgccatggagaagcccatttctctcaatcctgagaacattcgagatgagaaagtgaaggttcttcaatcaatcgatccaattaaggatgaggaagtcgttcttggacaatacgatgggtacaaagatgatccaacagttcccgatgactcaaatactccaacttttgcgactgtggttctgcgcattcataatgaaagatgggaaggtgtcccatttatagtaaaggcagggaaagcattaaattcaagaaaagcagaaattcgagttcagttcaaggatgtccctggtgatatattcaaatgtaaaaagcaagggagaaatgagtttgttatacgccttcaaccttcagaagccatgtacatgaagcttacggttaagcagcctggactggagatgcgaacagttcaaagtgaacttgacttatcgtatgggcaacgttatcaagatacgaaaatcccagaagcatatgaacgtcttatactcgacacaataagaggcgatcagcagcatttcgttcgcagagacgagttgaaggctgcatggaagatcttcactcctcttctgcataaaatcgatgacggtaagctcaagccgctcccatacaaaccgggaagtcgaggccccgaggcagcagatgagctgcaggcgaaagtgggttatgtacaaacccacggctatatatggattcctccaacattgtag

SEQ ID NO: 30 - protein, 517 aa, Cannabis sativa
MGSGQWHIEKRSSFKNDSESIEAENFPENGSLSIIVLGASGDLAKKKTFPALFNLFKQGLLPSNEVHIFGYARTKITDDELRDRLRGYLIPKKGGSSKDLETVSQFLQLIKYVCGAYDAEEGFRLLDKEISKHEVSKHSENGLSRRLFYLALPPSVYPSVCKMIRHYCMNKTDLGGWTRVVVEKPFGRDLASAEELSSQIGELFEESQLYRIDHYLGKELVQNLLVLRFANRFFLPLWNRDNIDNVQIVFREDFGTDGRGGYFDQYGIIRDIIQNHLLQVLCLVAMEKPISLNPENIRDEKVKVLQSIDPIKDEEVVLGQYDGYKDDPTVPDDSNTPTFATVVLRIHNERWEGVPFIVKAGKALNSRKAEIRVQFKDVPGDIFKCKKQGRNEFVIRLQPSEAMYMKLTVKQPGLEMRTVQSELDLSYGQRYQDTKIPEAYERLILDTIRGDQQHFVRRDELKAAWKIFTPLLHKIDDGKLKPLPYKPGSRGPEAADELQAKVGYVQTHGYIWIPPTL

SEQ ID NO: 31 - DNA, 1296 bp, Cannabis sativa
  source 1..1296: mol_type = other DNA
atggcgttctcttctctgctcagatctaccaccggagctgttctgattgaagcttctagacctgaaatctttgcgcctcttgcttcttccaatccttctaaggcatcgagcatttgtttcaatcgcaacccaaactctgcaagatttcaatcaagtttatttggcactgctgtcccaagtggatcatctgttttacaggtatgcaatgtcaagagtgtccaacccattaaagcaacggccactgaagtaccaccaactgttcagaaattgaagaccagtgggaagacaaaggttgggatcaatggttttggtcggattggaagattggttttacgagtagcaacattcagggatgatattgacgtagtggcggtgaatgatcctttcattgatgctaaatacatggcctacatgttcaaatatgattctactcatggattattcaaaggaagcattagggttgtagatgattcaaccttagaaatcaatgggaagcagataaaggttctgagcaaaagggaccctgctgagattccttggggtgattatggagctgagtatgtcgttgaatcttccggtgttttcacaacaattgacaaggcttcagcacataaaaagggtggagccaagaaagtagttatatcagctccatcagctgatgcacctatgttcgtggttggagtaaatgagcagacatacaagccaaacatggacattgtttccaatgcaagctgtactactaactgtcttgctcctctagctaaggtggttcatgaggaatttggtattcttgaaggtttaatgacaacggttcatgcaactacagcaacacagaagaccgttgatggcccatcaatgaaggattggagaggaggccgtggagctggacaaaatattattcctagttctactggtgcagcaaaggctgttggtaaggttctcccagaactgaatggaaagcttactggaatggccttccgtgttccaactcctaatgtctcagtggtggacttaacttgtcgacttgagaagagtgcttcttatgaagatgtcaaggcagccattaagtatgcatcagaaggaccacttaaaggcattcttgggtacacagatgaagatgttgtctccaatgatttcgttggtgacacaaggtcaagtattttcgatgccaaggctggaatagggcttagcacatccttcatgaagcttgtgtcatggtacgataacgaatggggatacagcaaccgtgttttggaccttattgagcacatggcgttggtagcggctacctactaa

SEQ ID NO: 32 - protein, 431 aa, Cannabis sativa
MAFSSLLRSTTGAVLIEASRPEIFAPLASSNPSKASSICFNRNPNSARFQSSLFGTAVPSGSSVLQVCNVKSVQPIKATATEVPPTVQKLKTSGKTKVGINGFGRIGRLVLRVATFRDDIDVVAVNDPFIDAKYMAYMFKYDSTHGLFKGSIRVVDDSTLEINGKQIKVLSKRDPAEIPWGDYGAEYVVESSGVFTTIDKASAHKKGGAKKVVISAPSADAPMFVVGVNEQTYKPNMDIVSNASCTTNCLAPLAKVVHEEFGILEGLMTTVHATTATQKTVDGPSMKDWRGGRGAGQNIIPSSTGAAKAVGKVLPELNGKLTGMAFRVPTPNVSVVDLTCRLEKSASYEDVKAAIKYASEGPLKGILGYTDEDVVSNDFVGDTRSSIFDAKAGIGLSTSFMKLVSWYDNEWGYSNRVLDLIEHMALVAATY

SEQ ID NO: 33 - DNA, 1089 bp, Cannabis sativa
  source 1..1089: mol_type = other DNA
atggctgacagcaaggtctctccgactccattgctgaaagatgaactcgatattgtgatacccacaatccggaacctcgatttcttggagatgtggaggcccttcttccagccataccatttgatcatcgtccaagatggtgacccctccaagactatcaaggtcccagagggcttcgattacgagctctacaacagaaacgacatcaatcgaattctgggtcctaaggcttcctgcatttcgttcaaggactctgcttgtcgttgcttcggctacatggtctccaagaagaagtacatctacaccattgacgatgactgctttgtcgcaaaagatccttccggcaaagaaatcaatgctcttgagcagcacattaagaacgttctgagtccttcaacaccatttttctttaacaccctctacgatccataccgtgagggtgcagattttgtccgtggttatccattcagtcttcgggaaggtgtcccaactgctgtttctcatggtctctggctcaacatcccagactacgatgctcccacccaacttgttaagcctcttgagagaaacagcaggtacgtggatgcagtcttgactatccctaagggaactctcttccccatgtgtggtatgaatcttgcattcaaccgtgaattgatcggccctgcaatgtactttggacttatgggtgatggccagccaatcggacgttacgatgatatgtgggctggttggtgtatcaaggttatctgtgaccacttgggatggggagtgaagacaggtttgccctacatttggcacagcaaggcaagcaacccatttgtgaaccttaagaaggagtacaagggaatctactggcaagaagagttgatccccttcttccaagctgtcacccttccgaaggaatgtaccaccgtccaaaagtgctacattgagctctccaagcaagtcaaggccaagcttggtaaggtggatgagtacttcatcaagctttcggatgccatggtcacttggattgaggcttgggatgagctgaacccggttgctggaagcactgctgctgaactcaccaatggacccaagaaatag

SEQ ID NO: 34 - protein, 362 aa, Cannabis sativa
MADSKVSPTPLLKDELDIVIPTIRNLDFLEMWRPFFQPYHLIIVQDGDPSKTIKVPEGFDYELYNRNDINRILGPKASCISFKDSACRCFGYMVSKKKYIYTIDDDCFVAKDPSGKEINALEQHIKNVLSPSTPFFFNTLYDPYREGADFVRGYPFSLREGVPTAVSHGLWLNIPDYDAPTQLVKPLERNSRYVDAVLTIPKGTLFPMCGMNLAFNRELIGPAMYFGLMGDGQPIGRYDDMWAGWCIKVICDHLGWGVKTGLPYIWHSKASNPFVNLKKEYKGIYWQEELIPFFQAVTLPKECTTVQKCYIELSKQVKAKLGKVDEYFIKLSDAMVTWIEAWDELNPVAGSTAAELTNGPKK

SEQ ID NO: 35 - DNA, 1887 bp, Cannabis sativa
  source 1..1887: mol_type = other DNA
atgaaattatcaaacgtactagtgacatgcaaccttaataatttgccagtcttgagatccaatcacaagatgagcactagtgcaaggtctcaccaaatccaacctaaaaatttggcattcaatataagatccaagcaccaaaattataactctcttggtggtcacctcgcccagcgactcgttcagattggcgtgtcagacgttttctctgtgcctggtgacttcaacctcgcgcttcttgaccatcttgtagccgagcctcaactcaacctaattggttgttgcaacgagttgaatgctgggtatgcagccgatggctatgctcggtcacgcggtgttggagcctgcgtggtgaccttcactgtcggcggtcttagcgttatgaacgctatagccggtgcgtacagtgaggatcttccggtcatttgtatagttgggggccctaactcaaatgattatggagctaataacagaattcttcaccattctattgggttagctgattttagtcaagagctcaaatgcttccaacaaatcacttgtcaacaggtggttgtgaataacttggataatgcacaagagcaaattgatggggccatagctgcagcattagagcatagcaaacctgtgtacattagcataagttgcaatctgcctggattatctcatccttcctttcttcgaaacccacttccattctccttaccagtcagactaagtaacactgtggagctagaactagcagtagaaacagcagcagagttcttgaacaaagcagtcaagccaatccttttggctggaccgaaactccgatcagcaaaagctaccgaggcattccacgagctttctaatgcgagtggttattcggtggctatcatgcctacagcaaaagggcttgtactagagaatcaccctcgcttcatcggtacatactggggcacaataagcactccctattgcaatgagactgttgaactggccgacgcttaccttatcgtcggcccagtattcaacgattatatcaatgtagggcactcgctcttacttaaagaagagcgagccataattgttcaaccccatagtgttagaatcggaaaaggtcctacctttcgattcgttgagatgagagacttcttagtggcattagcaaagagaataaaacacaatacaacatctcatgagaattatgagagaatgtatatggcagatgatggaggtcatcctctaataatgagatcttcttggtcaagtggtgatgagatcatgatgaagaaggaggcgttgagagttaatgtggtgtatgggcatttggagaaaatgttgtctagtaacacaactatgattgctgacactggtgtctcatggtttagttgccaaaagctcaaattgccacaaggatgtgggtttgaagttcaaatgcattatggatcaatcggttggtcagtaggagctagtcttgggtatgcacaggcagcttcaccaaacaaccgagtcattgcttgcattggagatggcagttttcagatgggtgcacaagaggtgtcaacaatgcttcgttgtggacaaaatagtataatatttctaattaacaatggaggctacaccatagaagaagtcatacatgaaggatcctacaatgttattaagaattggaactacactgctttagttgatgcaattaataatggtgaacacaaatgttggaccactaaggtttcatgtgaggaggaacttgttgaagcaatgaagatggcaacagaagaaaaaagagagtgtttgtgcttcattgaagtgttagtacacaaggatgacacaagcaaagagttggttgagttctgcttaagaacatcccttttcacttctcgagcacctacttatattttataa

SEQ ID NO: 36 - protein, 632 aa, Cannabis sativa
MLATMKLSNVLVTCNLNNLPVLRSNHKMSTSARSHQIQPKNLAFNIRSKHQNYNSLGGHLAQRLVQIGVSDVFSVPGDFNLALLDHLVAEPQLNLIGCCNELNAGYAADGYARSRGVGACVVTFTVGGLSVMNAIAGAYSEDLPVICIVGGPNSNDYGANNRILHHSIGLADFSQELKCFQQITCQQVVVNNLDNAQEQIDGAIAAALEHSKPVYISISCNLPGLSHPSFLRNPLPFSLPVRLSNTVELELAVETAAEFLNKAVKPILLAGPKLRSAKATEAFHELSNASGYSVAIMPTAKGLVLENHPRFIGTYWGTISTPYCNETVELADAYLIVGPVFNDYINVGHSLLLKEERAIIVQPHSVRIGKGPTFRFVEMRDFLVALAKRIKHNTTSHENYERMYMADDGGHPLIMRSSWSSGDEIMMKKEALRVNVVYGHLEKMLSSNTTMIADTGVSWFSCQKLKLPQGCGFEVQMHYGSIGWSVGASLGYAQAASPNNRVIACIGDGSFQMGAQEVSTMLRCGQNSIIFLINNGGYTIEEVIHEGSYNVIKNWNYTALVDAINNGEHKCWTTKVSCEEELVEAMKMATEEKRECLCFIEVLVHKDDTSKELVEFCLRTSLFTSRAPTYIL

SEQ ID NO: 37 - DNA, 1632 bp, Cannabis sativa
  source 1..1632: mol_type = other DNA
atgcttcttcagttagagcaagattgcttggatgtgtacaaaaggaaggttgagcaggctgcaaagtcaagggcactgctgcttcaggcattgtctgatgccaaacttgaactttctagtctcctatctgctcttggagataagagttctactggaataccagataaaagttcaggaacaatcaaggaacagcttgcagctattgcaccagcgcttgatcagctatggaaacagaaagaggagagagttaaggacttttctgacgttcaatcgcagatccaaaagatttgtggggaaattgctgggagcttgaacctgaatgaccaagtaggaacaccttctgttgatgagtctgacctaactctgaagaagttagacgaatatcatgcgcagcttcaagaacttcaaaaggagaagagtgataggttgcagaaggtgctggaatttgttagctctgtgcatgatttgtgtgctgtccttggaatggacttcttcagtaccgttactgaggttcatcctagcttaaatgactcaactggcgtgcagtccaaaagcattagtaatgataccctatctaggctggctaagacagtcatagcactaaaagaagataagaagcagaggcttcaaaaactccaagagctagcttctcaacttattgatttatggaatctgatggatactcctgaggaggaaagggaactatttgaccatgttacctgcaacatttcagcatcagttgatgaggtgactgttcccggggctctcgctttggatttgatcgagcaggctgaggtggaagtggataggcttgatcagctcaaatcgagcaggatgaaggaaattgcctttaaaaagcaatcagaacttgaagagatatttgctcgtgctcatatagaaatagatccagaagctgcaagggaaaaaataatggcaatgattgattctggggatgttgagcctgctgagttgctggctgacatggacaatcagatagcaaaagcaaaagacgaatctctcagtagaaaggacatattggacaaagttgaaaaatggatgtcagcctgtgaggaagaaagttggcttgaagactacaatcgggatgagaacaggtataatgcaagtagaggtgcacatctcaatctcaagcgtgcagagagagcccggattctggttaataaaattccagctcttgttgagactttggttgccaaaactcgtgcttgggaggaggatcatggcatgtcatttacttatgatggtgttcccctccttgctatgctagatgaatatgcaatgctcaggcaggaaagagaagaagagaagaggaggatgagagatcagaagaagtaccaagagctgcagcactcagaacaggaagccatatttggctccaagcctagtcctgctcgaccagttagtgcaaagaaggtggcaggtccacgtgcgaatggaggtgccaatggaacaccaaacagaaggctatcacttaatcaaaacggagctaggtccacaactaaagatgcaaagagagaaaataggcttagtgctcctgtgaactacgtgtcaatgtcaaaagaagatgctgcctcccatgtttcaggcaccgaccctattccggcttccccataa

SEQ ID NO: 38 - protein, 543 aa, Cannabis sativa
MLLQLEQDCLDVYKRKVEQAAKSRALLLQALSDAKLELSSLLSALGDKSSTGIPDKSSGTIKEQLAAIAPALDQLWKQKEERVKDFSDVQSQIQKICGEIAGSLNLNDQVGTPSVDESDLTLKKLDEYHAQLQELQKEKSDRLQKVLEFVSSVHDLCAVLGMDFFSTVTEVHPSLNDSTGVQSKSISNDTLSRLAKTVIALKEDKKQRLQKLQELASQLIDLWNLMDTPEEERELFDHVTCNISASVDEVTVPGALALDLIEQAEVEVDRLDQLKSSRMKEIAFKKQSELEEIFARAHIEIDPEAAREKIMAMIDSGDVEPAELLADMDNQIAKAKDESLSRKDILDKVEKWMSACEEESWLEDYNRDENRYNASRGAHLNLKRAERARILVNKIPALVETLVAKTRAWEEDHGMSFTYDGVPLLAMLDEYAMLRQEREEEKRRMRDQKKYQELQHSEQEAIFGSKPSPARPVSAKKVAGPRANGGANGTPNRRLSLNQNGARSTTKDAKRENRLSAPVNYVSMSKEDAASHVSGTDPIPASP

SEQ ID NO: 39 - DNA, 1698 bp, Cannabis sativa
  source 1..1698: mol_type = other DNA
atgagaaatttgttgcaagaactccttgaaaaacatggtgatgtgagaacaccaacaatactgggactaagggagcatatattcactggaagtgtctcatctctggcctggtttatgtcaaatcaggagaatagttttgttaccattgggcagagattgctggctaaccctctaaaagttcgcttccactacggtcatcctgatgtctttgataggctgttccacctgacaagaggaggtgttagcaaagcttcaaaaatcatcaacttaagcgaggacatatttgcaggatttaattctacactgcgtgaaggaaatgtaactcatcatgagtatattcaagttggtaaaggaagggatgttggactcaaccagatctcagtgtttgaggcaaagatagctaatgggaacggcgagcagactataagtcgtgatatatataggcttgggcatagatttgattttttccggatgttgtcatgttactttaccacagttggcttctacttcagcaccttgctgagtgtgctcacagtctatgtgtttctctatggccgtctttatcttgttcttagtggacttgaggaggggctgcgaactcagccagctattcgagataataagtctcttcaagtagctcttgcttctcagtcttttgtccaaattggacttttgatggctctgcctatggtgatggaaattggtttagaaagggggttccggaatgcactaactgatttcatattaatgcaattacaactagcacctgtatttttcacattttcgcttggaacaaagacgcactattatggaagaactctgctccatggaggggctgaatatagaggaacaggccgtggatttgtagtgttccatgcaaaatttgctgaaaactatagattctattcaaggagtcattttgtcaagggaacatggctttttgctccttttctcttcaacccatcaggatttgagtggcaaaagatagtcgatgactggaatgactggaataagtggataagcaatcaaggtggcattggcgtctctcctgataagagttgggaatcttggtggcaaaaagaacaacagcatcttcgacactctgggatgcgcggtgccattgctgagatactgttagcgctccgctttttcgtttatcaatatggtcttgtgtatcatttgagcatcacaaagaaaaccaaaagttttctggtttatggtatttcgctactcgtaattattgcagttttgcttttgacaaaggttgcatctattggaaggcgaagatttagtgcacagtttcagctactgtttcgactttttaagggatttgtttttctatcatttttaactcttttcatcaccttaattgcactcccccatatgacacttcaggacattgtagcaatcattcttgccttcatgccaactggatggggattgctcttgattgcgcaagcttgtaagccactaattgaaaatactgcattgtgggcatcagttagaacacttgcacgttattatgaagtaatcatgggaatgatcctctttattcctgttgcattcttggcttggttccctttcgtttcggaatttcagacacgaatgttgtttaaccaagcattcagcagaggtttgcagatttctcgtattcttggtggacaaaggaaggatcgctcatcacgaataaaaaaatag

SEQ ID NO: 40 - protein, 565 aa, Cannabis sativa
MRNLLQELLEKHGDVRTPTILGLREHIFTGSVSSLAWFMSNQENSFVTIGQRLLANPLKVRFHYGHPDVFDRLFHLTRGGVSKASKIINLSEDIFAGFNSTLREGNVTHHEYIQVGKGRDVGLNQISVFEAKIANGNGEQTISRDIYRLGHRFDFFRMLSCYFTTVGFYFSTLLSVLTVYVFLYGRLYLVLSGLEEGLRTQPAIRDNKSLQVALASQSFVQIGLLMALPMVMEIGLERGFRNALTDFILMQLQLAPVFFTFSLGTKTHYYGRTLLHGGAEYRGTGRGFVVFHAKFAENYRFYSRSHFVKGTWLFAPFLFNPSGFEWQKIVDDWNDWNKWISNQGGIGVSPDKSWESWWQKEQQHLRHSGMRGAIAEILLALRFFVYQYGLVYHLSITKKTKSFLVYGISLLVIIAVLLLTKVASIGRRRFSAQFQLLFRLFKGFVFLSFLTLFITLIALPHMTLQDIVAIILAFMPTGWGLLLIAQACKPLIENTALWASVRTLARYYEVIMGMILFIPVAFLAWFPFVSEFQTRMLFNQAFSRGLQISRILGGQRKDRSSRIKK

SEQ ID NO: 41 - DNA, 885 bp, Cannabis sativa
  source 1..885: mol_type = other DNA
atggaccagtatgaaaaagttgagaagattggggaaggaacttatggtgtggtgtacaaggctcgtgaccgcgtaactaataagactatagctttgaagaagatccgtttggagcaagaagatgagggagtcccaagcacagctatcagagaaatttctctcttgaaggaaatgcagcatggaaacattgtgcggttacaagatgtggtgcacagtgagaagcgtttgtatttggttttcgaatacttagacctggatctgaaaaagttcatggattcatctcccaatttttcaaaggatcttcagcagataagattgtttctctatcaaattctccggggcattgcttattgtcattctcatagagttcttcatcgagatctaaaaccccagaacttattaatagatcgtagcaataatgcacttaagcttgcagattttggactggcgagagcatttggtattccggtcaggacttttactcacgaggtggtgaccctctggtacagagcaccagaaatattgcttggatcccgtcattactccacccctgttgatatttggtcagtagggtgtatatttgctgagatgctgaatcaacgaccattattccctggggactctgagattgatgagttgttcaagatattcagaatattgggtactccgaccgaggatacatggccgggagtgggctctctacctgattataaatcttcatttcctaagtggccctcaaaggacatagcgagtcttgttccaaatctcgaatcagctggtgttgatcttctttcgaaaatgcttcgtctggatcccgtgaagagaattacggccagagctgctctcgagcacgagtacttcaaagatatttccctcgtgccttga

SEQ ID NO: 42 - protein, 294 aa, Cannabis sativa
MDQYEKVEKIGEGTYGVVYKARDRVTNKTIALKKIRLEQEDEGVPSTAIREISLLKEMQHGNIVRLQDVVHSEKRLYLVFEYLDLDLKKFMDSSPNFSKDLQQIRLFLYQILRGIAYCHSHRVLHRDLKPQNLLIDRSNNALKLADFGLARAFGIPVRTFTHEVVTLWYRAPEILLGSRHYSTPVDIWSVGCIFAEMLNQRPLFPGDSEIDELFKIFRILGTPTEDTWPGVGSLPDYKSSFPKWPSKDIASLVPNLESAGVDLLSKMLRLDPVKRITARAALEHEYFKDISLVP

SEQ ID NO: 43 - DNA, 795 bp, Cannabis sativa
  source 1..795: mol_type = other DNA
atggcaattactcaagcattttattccatggccttattgcctactcttctattaatcacactacacattcaagccaccgccgccgattacggcggctggcagaacggtcacgccaccttctacggcggaggggatgcctctggcacaatgggaggggcttgtgggtacggtaacttgtacagccaaggttacgggacccaaacagcagcactaagcacagctctattcaacaatggattgagctgtggtgcttgttatgaaatgaagtgtaacaatgaccctaggtggtgcctccccggaaccataaccgtcaccgccaccaacttctgtcccccgaatttcgctctctccaacgacaacggtggctggtgtaatcctcctcttcaacacttcgacttggccgagcctgctttccttaaaattgctcagtaccgcgctggaattgttcccgttgcatttagaagggtgccatgtgtgaagaaaggaggagtaagattcacgatcaacggtcattcttacttcaacttggttttgatcaccaacgtcggcggtgcaggggatgtccatgcggtctcaattaaaggctcgaaaactgggtggcaagccatgtccagaaactggggccagaattggcaaagcaataactacctcaacggtcaggctctctcctttcaagttaccaccagtgatggtagaacggtcacagcttataacgtagcaccctctggttggcagtttggtcagactttccaaggccctcagtttttaattatattagcctcattggcttatata

SEQ ID NO: 44 - protein, 265 aa, Cannabis sativa
MAITQAFYSMALLPTLLLITLHIQATAADYGGWQNGHATFYGGGDASGTMGGACGYGNLYSQGYGTQTAALSTALFNNGLSCGACYEMKCNNDPRWCLPGTITVTATNFCPPNFALSNDNGGWCNPPLQHFDLAEPAFLKIAQYRAGIVPVAFRRVPCVKKGGVRFTINGHSYFNLVLITNVGGAGDVHAVSIKGSKTGWQAMSRNWGQNWQSNNYLNGQALSFQVTTSDGRTVTAYNVAPSGWQFGQTFQGPQFLIILASLAYI

SEQ ID NO: 45 - DNA, 798 bp, Cannabis sativa
  source 1..798: mol_type = other DNA
atgactgtgatcgatatcttgactagggtagatgtgatctgcaaaaagtacgacaaatatgatattgataagcagaaggatctcaatgtatctggtgatgatgcctttgctcgtctcttctctgttgttgaagatgatatcgaggctgctcttcagaaagcggaggttgcagcgaaggagaagagtagggcgtctgcagtggccttgaatgcagaaattcgtcggactaaggctcggttgctcgaggaagtaccgaaattgcagagattagcttttaagaaggttaagggactatccactgaggagcttgccgctcgtaatgatttggtccttgcattgccagataggatccaagctataccggatgggactccagcaccccagaaacaaactggaggctgggcgtcatcagcctctcgtactgaaatcaagtttgattcagagggtcgatttgatgatgaatacttccagcagagtgaggagtcgagtcaattcagggaagagtatgaaatgcggaggacaagacaggaccaaggtttggatatgatctcagaaggtttagatacgttgaaaaacatggctcacgatatgaatgaggagcttgataagcaagttcctttgatggatgagatcgacgaaaaggcggacaaggctacatctgatcttaagcatacaaatgtgagacttaagcacactgtcaaccagctacggtccagccggaatttctgtatcgacattattttgctgtgtataatactgggaattgccgcatatttgtacaatgcactgaaaaagtga

SEQ ID NO: 46 - protein, 265 aa, Cannabis sativa
MTVIDILTRVDVICKKYDKYDIDKQKDLNVSGDDAFARLFSVVEDDIEAALQKAEVAAKEKSRASAVALNAEIRRTKARLLEEVPKLQRLAFKKVKGLSTEELAARNDLVLALPDRIQAIPDGTPAPQKQTGGWASSASRTEIKFDSEGRFDDEYFQQSEESSQFREEYEMRRTRQDQGLDMISEGLDTLKNMAHDMNEELDKQVPLMDEIDEKADKATSDLKHTNVRLKHTVNQLRSSRNFCIDIILLCIILGIAAYLYNALKK

SEQ ID NO: 47 - DNA, 738 bp, Cannabis sativa
  source 1..738: mol_type = other DNA
atggcggctctagctcgattctctaagaaggctttaaccactctctcgtctcagcagaatatctttcatcgtggactcgccactgaggctgggaaggccgtaaatccatcggccgatcgagtccaatgggactaccgaggtcagaggaagatcatcccgcttgggcagtggctcccaaaggtggccgtggacgcctatgttgcgccaaatgtagttttagccggacaggttaccgtctgcgatggtgcttcagtctgggccggttcggtcctacgaggcgatctcaacaagatcaccgttggattctgctccaatgttcaggaacgaagcgtcattcatgccgcttggtcttcgcctacaggccttccagcagagacatttattgagaggtttgtgacaatcggtgcatacagtctcttgcgttcttgcacaattgaaccggagtgcattattgggcagcactctattctcatggaaggttcgttggtggagactcactctatcttggaggctggatccgtggttcccccagggagacgaattccaactggcgaactttgggctggaaatcctgcaaggtttgtccgaactctttcccatgaagagaccttagagatcccgaaacttgctgtagcaatcaatgatctgagcaaagaacatttcatggagttccttccttactccacagcatatttggaggttgaaaagtttaagaagtcattgggcatcactatttga

SEQ ID NO: 48 - protein, 245 aa, Cannabis sativa
MAALARFSKKALTTLSSQQNIFHRGLATEAGKAVNPSADRVQWDYRGQRKIIPLGQWLPKVAVDAYVAPNVVLAGQVTVCDGASVWAGSVLRGDLNKITVGFCSNVQERSVIHAAWSSPTGLPAETFIERFVTIGAYSLLRSCTIEPECIIGQHSILMEGSLVETHSILEAGSVVPPGRRIPTGELWAGNPARFVRTLSHEETLEIPKLAVAINDLSKEHFMEFLPYSTAYLEVEKFKKSLGITI

SEQ ID NO: 49 - DNA, 1491 bp, Cannabis sativa
  source 1..1491: mol_type = other DNA
atggctgggactggagtttttgcagatgtaattgagggagatgtgtacaagtactactgtgatggagagtggaaaaaatcttcttctactaaaagtgttgctattatcaaccccacaactagaaagacccaatacaaggttcaagcttgtacacaagaggaagtgaacaaggttatggagatagcaaaatcagctcaaaaatcatgggcaaagactcctctttggaaacgagctgagcttcttcataaagcggctgctattctcaaggaacacaaagctccaattgcagagtgtcttgttaaggaaattgctaaaccagccaaagatgctgtcactgaggttgtgaggtctggagatctggtgtcatattgtgcagaagaaggggttaggattttgggagaagggaagtttttggtttctgacagttttccaggaaatgagaggaccaagtattgtctcacttccaagattcctcttggtgtggtattagctattccaccatttaactaccctgtcaaccttgctgtctcaaaaattgctcctgctcttatagctggaaactcccttgtgctcaagcccccaactcagggtgctgtgtcatgtctccacatggtgcactgttttcacttagctggttttcccaaaggccttattagctgtgtcactgggaaaggatctgagattggtgactttcttaccatgcaccctggagtaaactgtataagcttcacaggtggagacactggtattgcaatctcaaagaaggcagggatgatacctctccaaatggaattgggaggaaaagatgcttgcattgtgcttgaggatgctgatcttgatttggttgctgcaaacattataaaaggaggtttttcctacagtggtcaaaggtgtactgctgttaaagttgttttggtgatggaatcggttgcggatgaacttgttgagaaagtgaaggcaagggtagcaaaattgagtgttggaccacctgaggaagacagtgatatcactcctgttgtttcggaatcttcagctaattacattgaaggattggttaaggatgctaaagagaaaggagcaacattttgccaggaatacaagagagaaggcaatcttatttggcctttgttattagataatgtgaggcctgatatgagaattgcttgggaagagccatttggaccagttttaccagttattaggattagctctgttgaagaaggaatccaccactgcaatgccagcaattttggactccagggttgtgtcttcacaaaggatatcaacaaagcaatcctaattggagatgcaatggagactggaaccgttcagataaactctgcaccggctcgcggaccagatcatttcccatttcagggtataaaagacagtggtattgggtcacaagggatcacaaacagcattaacatgatgaccaaagtgaagaccactgtcataaacttgccaactccttcttacaccatgggttag

SEQ ID NO: 50 - protein, 496 aa, Cannabis sativa
MAGTGVFADVIEGDVYKYYCDGEWKKSSSTKSVAIINPTTRKTQYKVQACTQEEVNKVMEIAKSAQKSWAKTPLWKRAELLHKAAAILKEHKAPIAECLVKEIAKPAKDAVTEVVRSGDLVSYCAEEGVRILGEGKFLVSDSFPGNERTKYCLTSKIPLGVVLAIPPFNYPVNLAVSKIAPALIAGNSLVLKPPTQGAVSCLHMVHCFHLAGFPKGLISCVTGKGSEIGDFLTMHPGVNCISFTGGDTGIAISKKAGMIPLQMELGGKDACIVLEDADLDLVAANIIKGGFSYSGQRCTAVKVVLVMESVADELVEKVKARVAKLSVGPPEEDSDITPVVSESSANYIEGLVKDAKEKGATFCQEYKREGNLIWPLLLDNVRPDMRIAWEEPFGPVLPVIRISSVEEGIHHCNASNFGLQGCVFTKDINKAILIGDAMETGTVQINSAPARGPDHFPFQGIKDSGIGSQGITNSINMMTKVKTTVINLPTPSYTMG

SEQ ID NO: 51 - DNA, 399 bp, Cannabis sativa
  source 1..399: mol_type = other DNA
atgcgtgttgtaaaaagggttgataggccagagtacaagtactccattgccatgatggggtatgaagaagaggagaagacaactgttgtggagttgacttataattatggtgtgactgaatacacaaagggaaatgcatatgcacagatcgctattggtacagatgatgtatacaagagtgctgaggttgtgaacactgttatacaagagcttggaggaaagattacccgacaaccaggaccaattcctgggctcaacaccaagattacctcttttctggacccagatggctggaagactgttttggttgacaataaagatttttctcaaggaactccatgggtaactgccaagctcggtagcaataaggacgaggcatcttcacctttgaataagtga

SEQ ID NO: 52 - protein, 132 aa, Cannabis sativa
MRVVKRVDRPEYKYSIAMMGYEEEEKTTVVELTYNYGVTEYTKGNAYAQIAIGTDDVYKSAEVVNTVIQELGGKITRQPGPIPGLNTKITSFLDPDGWKTVLVDNKDFSQGTPWVTAKLGSNKDEASSPLNK

SEQ ID NO: 53 - DNA, 762 bp, Cannabis sativa
  source 1..762: mol_type = other DNA
atggtaattgtcgcttctggagctgttaagcatgaagattttgttggggcagtaaagaattctttcaccaagctgtcatctgattcaacaactgcttctcagttggttgaaaaggaacctgccatttttaccggttctgaggttagaatcattgatgatgatattccccttgcacaatttgccgttgctttcaatggagcatcttggacagatccagactctattccattgatggtcatgcaagctctgttgggctcgtggaataagaatgctggtggcggaaagcacatgggttctgagctggcacagaggattggcattaacgaaattgcagaaagcatgatggcttttaacaccaactacaaagacaccggcctgtttggtgtttatgctgttgctaagcctgattgcttagatgacttggcctgggcaattatgtatgagataacaaagttagcttatagagtttcagaagctgatgttatccgtgctcgtaatcagctgaaatcttctctgttgcttcacattgatgggactagtcctgtagcagaagatattgggcgccagttactaacttatggccgaagaattccgttcgctgaactgtttgcaaggattgatgctgttgacacaagcaccatcaaacgtgttgccaaccgatttatttacgacagggatattgctattgccgccatgggaccaatccagagtttgccagactacaactggttcagacgcaggacctactggaacagatactag

SEQ ID NO: 54 - protein, 480 aa, Cannabis sativa
MIYDRLAEAVKSKLQQLENPDPRFLKYGSPHPTVTDHTRILSAPETKITTLPNGLRVATESNLAAQSATVGVWIDAGSRFETEETNGTAHFLEHMIFKGTEKRTARELEEEIENMGGHLNAYTSREQTTYYAKVLDKDVPKALDILSDILQNSKFEEHRISRERDVILREMEEVEGQTEEVIFDHLHATAFQYTPLGRTILGPAQNIKTITKEHLQNYIKTHYTAPRMVIVASGAVKHEDFVGAVKNSFTKLSSDSTTASQLVEKEPAIFTGSEVRIIDDDIPLAQFAVAFNGASWTDPDSIPLMVMQALLGSWNKNAGGGKHMGSELAQRIGINEIAESMMAFNTNYKDTGLFGVYAVAKPDCLDDLAWAIMYEITKLAYRVSEADVIRARNQLKSSLLLHIDGTSPVAEDIGRQLLTYGRRIPFAELFARIDAVDTSTIKRVANRFIYDRDIAIAAMGPIQSLPDYNWFRRRTYWNRY

SEQ ID NO: 55 - DNA, 447 bp, Cannabis sativa
  source 1..447: mol_type = other DNA
atggcggaagattcatcatccaaatcgcccttcacgaagtactggtcaggttacaaagagttttggagcgagaggttttcgtttttggatcacttcaccaacttcgtcaaccgtgaaaaaccccttccccgttggactccttccgatgttgaagaatttattgcttccgaccctgttcatggccccactctgaagactacaagagaagcaatgaattatggcatcacaggaagtgtaattggagcagtatcaactgctggggttgcctggaagtattcaaggagtttgcatggtgctggactatctcttctagccgggggtgtttttggttggactttcggacaagaagctgcaaaccatgccctgcagctctacaagctggacaccatggttgctcaaaccaagttcttggagtggtgggaaaacaaaactcaaggacgattctga

SEQ ID NO: 56 - protein, 148 aa, Cannabis sativa
MAEDSSSKSPFTKYWSGYKEFWSERFSFLDHFTNFVNREKPLPRWTPSDVEEFIASDPVHGPTLKTTREAMNYGITGSVIGAVSTAGVAWKYSRSLHGAGLSLLAGGVFGWTFGQEAANHALQLYKLDTMVAQTKFLEWWENKTQGRF

SEQ ID NO: 57 - DNA, 561 bp, Cannabis sativa
  source 1..561: mol_type = other DNA
tggggtttgtgtggttggtgtagagagagagagagagagactaaagagagagagagagagaggaccagagaacggaagagaagagagagagaagcacgaatcccaaccctaatggcgttgcggagtctcgcactacctctggcttcagctccggcggtctccccaaggtcttcttcttcttcgtcatcaacttcatcatcttcttcctttttttcgtataccaactgtcgcaatagttctcttactccaacaccgttccatggccgccggcaattccctgtagtattctccaaccgtcgatttccaagtcatgtagttcgaatggctcccgatgaggaaaagatgactcgccgctctccgcttgatttccctattgaatgggaaagacccaagcccggaaggagaccagatatatttcctcaattcgcccccatgaagacgccgatgcctattccaatgccagcagatcctccggaagaagatgaggaagaggaggagaagagagaggaggaagaagaggaagatccaggggaaaatccagataacccggataagcaataa

SEQ ID NO: 58 - protein, 186 aa, Cannabis sativa
WGLCGWCRERERETKERERERTRERKRREREARIPTLMALRSLALPLASAPAVSPRSSSSSSSTSSSSSFFSYTNCRNSSLTPTPFHGRRQFPVVFSNRRFPSHVVRMAPDEEKMTRRSPLDFPIEWERPKPGRRPDIFPQFAPMKTPMPIPMPADPPEEDEEEEEKREEEEEEDPGENPDNPDKQ

SEQ ID NO: 59 - DNA, 1749 bp, Cannabis sativa
  source 1..1749: mol_type = other DNA
atggttgagaatacttctgatttagatggggagaactttgactgggatactgaagatgaacttgaaattggtaatatacctttatcttgttcaagtttgacagttcctggtggagttgctacaattggctctggggaggcgggtccttcatgctccaagacaattgatcattttgttgggatgggattctctgaaaagatggtggcaaaagtaattcaagaacatggagaggagaatacagatactattttggagacactcctcacatattcggctcttgaagaatctcctgaagaacaagtacaacataacgaacctgataacttttcttcagattgtgattatgaggggagctttttgagtgatttctcagatattgatagttctgataatgaggatcttgcaaatcttagttcagatgaggagaataaaattaggcccctgttggctatgggatattctaaagaagaggctgcaatcgcactagaaagatgtggccgtgactgttcacttgcagaattgactgattttatatgtgctgcacagatggcaagggcagcagatggggattatccccttgaagtgaagccaagacttaatggtgactattctaatcacaaaagaagaagacttcttgaatgttctaaacaaaggaggttggagaagaggattctgactgaagatgatgagacaattcgtcttccaaatccaatgattggatttggggttccaactgattcattccggcgggttcggagagatataccagaagctgctatagggcctccatatttctattatgagaatgtggcacttgcccccaaaggagtttggaccaccatctctcggttcttatatgatgtagaaccggaatttgttgattccaagtatttctgtgcagccgcaaggaaaagaggttatgttcacaatcttcccattgagaacagatttcctcttcttccccttcctgcacgcaccatacatgaagcaataccattaactaagaaatggtggccttcatgggactcaagaacaaagcttaactgcttgcagacatgcattggaagtgcaaaattaacagagaggattcggaaagctcttgaagagtatccagattctgaaccacctatgagtatccaaaagtacgtcatggatgaatgccggaagtggaatctggtctgggttggaaggaataaggttgcacctcttgagccagatgaaatagagatgcttctgggatttccaagaaaccatacaaggggaggtggaatcagcagaaccgatagatataaatctctcggtaattctttccaggttgacactgtagcataccatttatctactttgaaagacttgtacccgaatggcatcaatatgctttctctcttctctggaataggtggtgcggaagtagcactccaccggcttggtatccctctgaagaatgttgtctctgttgagatctcagaagtgaacaggaacatagtgagaagttggtgggagcaaacaagtcagaaagggtatttggagcaccttgatgacgtgcaacaactgaacagtgatcgtttggaagaatatatgaggaaatttggtgggtttgatcttgtagttggtgggagcccatgcaataatttagcaggcagtaacagacatcacagggatggacttgagggtaaggagtcttcacttttctatgactacttccgaattctagacttggttaagtgtattatgacaagaaatgaatag

SEQ ID NO: 60 - protein, 582 aa, Cannabis sativa
MVENTSDLDGENFDWDTEDELEIGNIPLSCSSLTVPGGVATIGSGEAGPSCSKTIDHFVGMGFSEKMVAKVIQEHGEENTDTILETLLTYSALEESPEEQVQHNEPDNFSSDCDYEGSFLSDFSDIDSSDNEDLANLSSDEENKIRPLLAMGYSKEEAAIALERCGRDCSLAELTDFICAAQMARAADGDYPLEVKPRLNGDYSNHKRRRLLECSKQRRLEKRILTEDDETIRLPNPMIGFGVPTDSFRRVRRDIPEAAIGPPYFYYENVALAPKGVWTTISRFLYDVEPEFVDSKYFCAAARKRGYVHNLPIENRFPLLPLPARTIHEAIPLTKKWWPSWDSRTKLNCLQTCIGSAKLTERIRKALEEYPDSEPPMSIQKYVMDECRKWNLVWVGRNKVAPLEPDEIEMLLGFPRNHTRGGGISRTDRYKSLGNSFQVDTVAYHLSTLKDLYPNGINMLSLFSGIGGAEVALHRLGIPLKNVVSVEISEVNRNIVRSWWEQTSQKGYLEHLDDVQQLNSDRLEEYMRKFGGFDLVVGGSPCNNLAGSNRHHRDGLEGKESSLFYDYFRILDLVKCIMTRNE

SEQ ID NO: 61 - DNA, 999 bp, Cannabis sativa
  source 1..999: mol_type = other DNA
ttggaaccatcttataagggactcatccatgaggtattcaccacagtattgcgtggtttatctggcgctaaagttacaaaaccaggaaagttccgtagttgtcaagatggatatgcagttaaatcatcactaaaagctgaagatggagtcctctatccccttgagaagagcttcttttttctacctaagcctcccacccttattcttcacgaggagattgactatgtggaatttgaaaggcatgctgccggtggctcaaatatgcattacttcgatcttcttattagacttaaaacggagcaagaacatctttttcgaaacattcagaggaatgagtaccataatctttttgacttcatgagtggaaaaggtttgaagatcatgaatttgggaaatacaagacctacagatggtgtggcatctgttcttcaagatgaggatgatgatgctgtcgatccacatcttgttcgtgttaagaatgaagctggcgatgatgaaagtgatgaagaggatgaagattttgttgccgaaaaggatgattctggttccccgactgatgattctggggaagatgattctgatgctagtgttagtggaggggagaaagagaagcctgtaaaaaaggatacaaaaaaagaaccttcatcttctaaggcatcttcttccaagaagaaatctaaagatggagatgaagatgggccgaagaagaaaaaacagaagaagaaaaaggatccaaatgcaccgaaaagagcattgtctggcttcatgttcttctcacaaatggaaagggagaatgtgaagaaaactaaccccggaatttccttcactgaagtgggaagagttttaggagataaatggaataagatgtcagtggaagagaaggaaccctatgaagccaaggctcgccaagataaaaaacgctacaaggacgaaattagtggatacaagaacccacaaccaatgaaggtagactcagggaatgaatcagactcagaatag

SEQ ID NO: 62 - protein, 448 aa, Cannabis sativa
MSMADVGAGGEDAVVTFEGIAILTPRGRYSVELHLSFLRLQGQANDFKIQYSSVVRLFLLPKSNQPFTFVVVTLDPPIRKGQTLYPHIVLQFETETVVQSDLSMSEDLLNTKYKDKLEPSYKGLIHEVFTTVLRGLSGAKVTKPGKFRSCQDGYAVKSSLKAEDGVLYPLEKSFFFLPKPPTLILHEEIDYVEFERHAAGGSNMHYFDLLIRLKTEQEHLFRNIQRNEYHNLFDFMSGKGLKIMNLGNTRPTDGVASVLQDEDDDAVDPHLVRVKNEAGDDESDEEDEDFVAEKDDSGSPTDDSGEDDSDASVSGGEKEKPVKKDTKKEPSSSKASSSKKKSKDGDEDGPKKKKQKKKKDPNAPKRALSGFMFFSQMERENVKKTNPGISFTEVGRVLGDKWNKMSVEEKEPYEAKARQDKKRYKDEISGYKNPQPMKVDSGNESDSE

SEQ ID NO: 63 - DNA, 414 bp, Cannabis sativa
  source 1..414: mol_type = other DNA
atgtctgggcgtggaaaaggaggaaagggtttgggcaagggaggtgcgaagaggcatcgcaaagtgcttcgtgataacatccagggtatcactaagccagcaattcggcgattggctcgacgaggtggcgtgaagcgtatcagtggtcttatctacgaagagacccgtggagttctcaagatcttcttggagaatgttattcgtgatgctgtcacttacactgagcacgccaggaggaagactgtgactgccatggaccgttgtttatgcactcaagagacaaggcaggactctctacggttttggaggttaaaagctttttctttgatgttgaaatttaaagtcttgctgtcaagaaagatttgggtcgttttttggggaagaatgggtggtgtttttgtatgggggtgttaa

SEQ ID NO: 64 - protein, 137 aa, Cannabis sativa
MSGRGKGGKGLGKGGAKRHRKVLRDNIQGITKPAIRRLARRGGVKRISGLIYEETRGVLKIFLENVIRDAVTYTEHARRKTVTAMDRCLCTQETRQDSLRFWRLKAFSLMLKFKVLLSRKIWVVFWGRMGGVFVWGC

SEQ ID NO: 65 - DNA, 570 bp, Cannabis sativa
  source 1..570: mol_type = other DNA
atgatcgatgaagatgaacctattctagagaaagcaatagggacggaaatcgaatggcttcctggaaagtgtttaacacagaaggttcttaaaaagaagcctaagaagggtgccaagaatgctaagccaatcacaaaaactgaaaattgtgatagtttcttcaatttcttaatccacctcaagttcccgaagaagatgaagacattgatgaggatgcagctgaggagcttcagaaccagatggaacaagattacgacattgggtcaaccattcgtgataagatcattcctcatgctgtttcttggtttacgggagaggccattcaaggagatgaatttggagacctggaagatgacgatgatgaagacattgatgaggatgaggatgatgaggaagatgaggatgaagatgaggaggacgacgatgaagatgaggacgatgatgaggacgaagaggaaaccaagaccaaaaagaagacatcagctgggtcaaagaaaaccggaagaacagcaggtacggacggtgcgcaaggcgagaggcctcctgaatgcaaacaacagtagaatttga

SEQ ID NO: 66 - protein, 189 aa, Cannabis sativa
MIDEDEPILEKAIGTEIEWLPGKCLTQKVLKKKPKKGAKNAKPITKTENCDSFFNFLIHLKFPKKMKTLMRMQLRSFRTRWNKITTLGQPFVIRSFLMLFLGLRERPFKEMNLETWKMTMMKTLMRMRMMRKMRMKMRRTTMKMRTMMRTKRKPRPKRRHQLGQRKPEEQQVRTVRKARGLLNANNSRI

SEQ ID NO: 67 - DNA, 594 bp, Cannabis sativa
  source 1..594: mol_type = other DNA
atggtaaggactcaaggggaattaacaggttccggtaacgtttccgagactgtacggagcttgagatcaataatgggggagattagggttttgaacaacatggatgaagatgaagtgtttgccttttcgaagaagatagctgcgccttatgatctggttgctcagaccaagcaaatgggtcgtcttcccgtagttcaatttgccgccggagggattgtgacaccggccgatgcggcactgatgatgcaattgggttgcgacggtgtttttgtgggttcggagattttcaattcttctgatccgtacaagcgtgtgaggggcataattcaggctgttaggcattacaatgatccccatgtgcttgtggaaacgagcactggtttggctgatgccatggctggtctgaacctcaatgaggataggattgaacagtttttaagtttaaaccatggttgtctctttgaggtttatatactttacttagtgaagagttgctttgcttgtttttgggtatcagagctagttttattgtatcagttgtacttcaaatctgatcttaaacttgctttttatataatgttgaagcatagatga

SEQ ID NO: 68 - protein, 197 aa, Cannabis sativa
MVRTQGELTGSGNVSETVRSLRSIMGEIRVLNNMDEDEVFAFSKKIAAPYDLVAQTKQMGRLPVVQFAAGGIVTPADAALMMQLGCDGVFVGSEIFNSSDPYKRVRGIIQAVRHYNDPHVLVETSTGLADAMAGLNLNEDRIEQFLSLNHGCLFEVYILYLVKSCFACFWVSELVLLYQLYFKSDLKLAFYIMLKHR

SEQ ID NO: 69 - DNA, 1098 bp, Cannabis sativa
  source 1..1098: mol_type = other DNA
atggatgagctcaactatgatagaaaaagtgagctaaaagcttttgatgacacaaaagctggtgtcaaagggctagttgattccggcatcacaaaaatcccaagaattttctatgagccaaaacaaaatgataacaacattattccatcacccgattcaaattcgggggaaacccaactcggtattccagttgttgatctcaaaaacgtcaaaaatgatttgactcgacgccaagagatcgtgcaagaagtcggggttgcagccgagtcatggggattttttcaagtggttaatcatggtattcctctaagtgttcttgaggaaatggagaccgcggtgaggaggttttttgagcaagaaaatgaggtgaagaaagagttttacactcgcgattcgtcgaggaagtttcggtataatagtaatttcgatctgtttaatgccccagcagctaattggagggacactgtgttttgtattatggctcctaatcctcctaagccagaggagttgccattggcattgagagatatactattggagtactcaaatgaggtgatgaaactaggaattgtgttgttggagttactttctgaagctcttggagtcaacaaaaattacttaaaagacatggattgcgctcaaggattggacgttttgtgccattattatccaccttgtccggagccagaactaactatgggaacaagcaagcacgccgatggtgatttctttacaattcttctacttgaccgcgtaattggtggtttgcaagttcttcatcagaatcgttggatcgatgttcctcctattcccggagctttggttattaacattggcgacttgctacagcttgtatcgaatgacatgttgaaaagtgttgagcatagagtagtggcaaatcacgacggtcctcgagtatctgttgcttgcttttttaggacagatatggaatcgtctttaaagtatggtccaattaaggaattgttaagcaaagataatcctcccaagtatagagagaccaccattaaagagtttgttgctcacttcaactcgaaaggccttggaacaacttctgctctatcccattttaggctctga

SEQ ID NO: 70 - protein, 365 aa, Cannabis sativa
MDELNYDRKSELKAFDDTKAGVKGLVDSGITKIPRIFYEPKQNDNNIIPSPDSNSGETQLGIPVVDLKNVKNDLTRRQEIVQEVGVAAESWGFFQVVNHGIPLSVLEEMETAVRRFFEQENEVKKEFYTRDSSRKFRYNSNFDLFNAPAANWRDTVFCIMAPNPPKPEELPLALRDILLEYSNEVMKLGIVLLELLSEALGVNKNYLKDMDCAQGLDVLCHYYPPCPEPELTMGTSKHADGDFFTILLLDRVIGGLQVLHQNRWIDVPPIPGALVINIGDLLQLVSNDMLKSVEHRVVANHDGPRVSVACFFRTDMESSLKYGPIKELLSKDNPPKYRETTIKEFVAHFNSKGLGTTSALSHFRL

SEQ ID NO: 71 - DNA, 672 bp, Cannabis sativa
  source 1..672: mol_type = other DNA
atgagaattctcattcttgtccggaacttcatacctggtcatcatcaggttatcaatggggattggaatgttgctggtattgcttacagagcttatgatctggaagggaaaacggtgggaactgttggtgctggacgtattggcaagcttttacttcaacggttgaagcctttcaactgtaatttgttgtatcatgacagactcaagatggaacccgaattggagaaggaaattggggcgaagtttgaggaggatcttgatgccatgcttcccaaatgtgacatagtcgtcatcaacatgcctcttactgacaaaactcggggaatgtttgacaaagaaaaaattgcaaagtgtaagaagggagtcctgtttgttaacaatgctcggggtgcaattatggatacacaagcagttgttgatgcttgttccagtggacacattgcaggctacagtggtgatgtttggtatccacaaccagctcctaaggaccatccatggcgttacatgccaaaccaagctatgactccccatatttctggcactacaattgacgcacagttgcgatatgcggatggagtgaaggatatgctcaataggtacttcaagggagaagatttcccagcacaaaactacattgtcaaggaaggtgaactggccagccagtaccgatga

SEQ ID NO: 72 - protein, 274 aa, Cannabis sativa
GERIKRAKNLKLVLTAGIGSDHVDLNAAAAAGLTVAEVTGSNTVSVAEDELMRILILVRNFIPGHHQVINGDWNVAGIAYRAYDLEGKTVGTVGAGRIGKLLLQRLKPFNCNLLYHDRLKMEPELEKEIGAKFEEDLDAMLPKCDIVVINMPLTDKTRGMFDKEKIAKCKKGVLFVNNARGAIMDTQAVVDACSSGHIAGYSGDVWYPQPAPKDHPWRYMPNQAMTPHISGTTIDAQLRYADGVKDMLNRYFKGEDFPAQNYIVKEGELASQYR

SEQ ID NO: 73 - DNA, 1041 bp, Cannabis sativa
  source 1..1041: mol_type = other DNA
atgggagaggttgatccggctttcatccaagatcccgagcacaggcctaggctggtggtaatcgaagcggagggcataccactcattgacctttcaccagcaataagcacctccgagaaactcgcgagagagatcggtggcgcgtgtagcgagtggggattctttcaggtgatcaatcacggggtaccactggagtgtcgacagaaactggagtcggcagcgaggaagttttttggtcaggatgtggaggagaagagaaaggtgaggaggaacgagacgagtgttttgggttactttgatgcagaaaacaccaagaacgtcagggactggaaggaggtgtttgacgtggcagtacaggagcccacggtggtccctgtttccactgatcccacggacgaccgagtcgttgagtggttcaatcgctggcctctctacccatcggagttaagggaagcatgtgaggaatatagtagagaagtggaaaaactagctcacaagatgctggaacttattgctctgagtctaggcttggcagcaaatagtctgaatgtgtacttcaaagaccatcaaactacattcatgagactcaatcactaccctccttgtccctctcctgccttggcacttggtgtgggtcgccataaggatggtggggccttaactgttctagctcaagatgaggttggaggattggaagttaggaagaaatcagatggagaatggattcgggtcaaacccacccccggtgcttttatcatcaatgttggggacattgtacaggtttggagtaatgacaaatacgagagcgtggaacacagggtgatggtaaactcagagaaggaaagattttcaattgcattttttctaaaccctgcacactacactgttgtcaagcccttggatgagctgattgatgagaacaatgcccctaaatttaagtcatacaattggggcaagtttatgacgcatagaaaagacagtgctttcaccaaacaaaatattgaaaatatccaaactcatcatttccgcatgcaggagtaa

SEQ ID NO: 74 - protein, 346 aa, Cannabis sativa
MGEVDPAFIQDPEHRPRLVVIEAEGIPLIDLSPAISTSEKLAREIGGACSEWGFFQVINHGVPLECRQKLESAARKFFGQDVEEKRKVRRNETSVLGYFDAENTKNVRDWKEVFDVAVQEPTVVPVSTDPTDDRVVEWFNRWPLYPSELREACEEYSREVEKLAHKMLELIALSLGLAANSLNVYFKDHQTTFMRLNHYPPCPSPALALGVGRHKDGGALTVLAQDEVGGLEVRKKSDGEWIRVKPTPGAFIINVGDIVQVWSNDKYESVEHRVMVNSEKERFSIAFFLNPAHYTVVKPLDELIDENNAPKFKSYNWGKFMTHRKDSAFTKQNIENIQTHHFRMQE

SEQ ID NO: 75 - DNA, 1170 bp, Cannabis sativa
  source 1..1170: mol_type = other DNA
atggtgtttacagtgtacggtgagcattggagaaagatgaggagaatcatgaccgttcctttcttcactaacaaggttgttcaacagtaccggtttggatgggaagccgaggcaggtagcgtggttgatgacgtgaagaagaatcctgaatctgccacaaacggcatcgttttgaggaagaggctccagcttatgatgtacaataacatgtacaggatcatgtttgacaggagattcgatagcgaggatgacccactcttcgttaaactcaaggctttgaatggtgagaggagtagactagcccagagcttcgagtacaactatggtgactttattcctgtcttgagaccctttttgagaggttacttgaagatatgtaaggaagtcaaacaaaagaggcttcaacttttcaaggactatttcgttgacgagaggaagaaattatctagcacaaagagtacagacaacgaaggattgaaatgcgccattgatcatattcttgaagctcaacaaaagggtgaaattaatgaggataacgttctttacatcgttgagaacattaatgttgctgctatcgaaacaacattgtggtcaattgaatggggtattgcggagcttgtgaaccacccagagatccagaacaagctaaggacagagctagacactattctaggaccaggtgtacaggtgaccgagccagacatgcacaagctaccataccttaacgccgtgatcaaggagacacttcggctacgtatggcaatcccactccttgtgccccacatgaacctcaaccaggccaagctcgcgggctacgacatcccagccgagagcaagatcctggtgaatgcatggtggttagccaatgaccccgcccactggaagaagccagaagagttcaggcctgagaggtttttcgaggaagagtctaaggtcgaggccaatggcaatgactttaggttccttcctttcggggttggaagaagggcatgccctggtatcattcttgctttgccaattcttgccatcactattgggcgtttggtgcagaactttgagctgttgccaccaccaggacagtccaagctcgacacagccgagaaaggtggacagtttagcttgcacatattgaagcattccaccattgttttgaagccaagatcagtttaa

SEQ ID NO: 76 - protein, 389 aa, Cannabis sativa
MVFTVYGEHWRKMRRIMTVPFFTNKVVQQYRFGWEAEAGSVVDDVKKNPESATNGIVLRKRLQLMMYNNMYRIMFDRRFDSEDDPLFVKLKALNGERSRLAQSFEYNYGDFIPVLRPFLRGYLKICKEVKQKRLQLFKDYFVDERKKLSSTKSTDNEGLKCAIDHILEAQQKGEINEDNVLYIVENINVAAIETTLWSIEWGIAELVNHPEIQNKLRTELDTILGPGVQVTEPDMHKLPYLNAVIKETLRLRMAIPLLVPHMNLNQAKLAGYDIPAESKILVNAWWLANDPAHWKKPEEFRPERFFEEESKVEANGNDFRFLPFGVGRRACPGIILALPILAITIGRLVQNFELLPPPGQSKLDTAEKGGQFSLHILKHSTIVLKPRSV

SEQ ID NO: 77 - DNA, 1395 bp, Cannabis sativa
  source 1..1395: mol_type = other DNA
atgcttaaatttgcaaagctacttcacaacaagggtttctacataacctttgtcaacactgagttcaaccacaatcgcttcctcaagaactcctccacaatttcctcctcaccgagcttccagttcgagacgattcccgatggccttccgccctcggatcaaggacaggattcgacacaagacatcccttctcttagtgattccactcgaaagaactttttgggcccattccttgagctcctcaaaaaacttgcataccaaaccaaaagtaatagtagcaaggatgatgttcctcctcgtgtgagttgcattattaatgatggagtcatgacttttgctattaaggcagctcaacagcttggacttcctgtggttcagtttttcacagttgctgcatgcgctgttatgggcatcgccaactaccgccctttacttgacaaaggaattgtaccactaaaagatgagaatgagttaaatgattttctagacacaagtatggaatggattccgggaataaaaggtctccgatttcgagatctaccatcattttgtaggatcactgattccaatgatattatgtttaattggatcatagagtcaattgagatggcttcagaagcttcagctgttgttattcagacatttgaagcattggaaccacatgttttggatgttctttcttcaactacttcacttccaaaaatatatccaattggaccactccaacctctactcgatcactatccacaacattctctacacactgtcacctacagtctttggaaggaagaaactgagtgtataaaatggcttagtactcaaaagcctaactcagtcatttatgtcaatttcggaagtataacggttctgagtgagcaacaagttattgaaattggttggggtctcgcaaatagcgatgttcctttcctgtgggtgattagaccagacttgataattggggaaggtaggctaccatctgagtttttggaccaaaccaaagaaagaagcctaatcataagttgggcaccacaggaggatgtgttaaatcacccctccataggaggatttttgacacattgtggttggaattcaatactggagagcttgactagtggggtgcctatgatttgttacccatactttgctgaccaacaaaccaattgttggtttgcatgcaatgaatggggtgtggcggtggagattgaaagtgatgtaaagagagatcaagtgacaaatttggttaaagagttattcgatggagaaaaaggtaagaaaataaagactacggcaatggaatttaagaaattggtacatgaagccactactccaagtgggagctcgtctacaaatttggatgatttgattaatgaagtgctcttgcatcaaactgattaa

SEQ ID NO: 78 - protein, 497 aa, Cannabis sativa
MDQISSDKKEGYSTESKPHAVCIPYPSQSHIKAMLKFAKLLHNKGFYITFVNTEFNHNRFLKNSSTISSSPSFQFETIPDGLPPSDQGQDSTQDIPSLSDSTRKNFLGPFLELLKKLAYQTKSNSSKDDVPPRVSCIINDGVMTFAIKAAQQLGLPVVQFFTVAACAVMGIANYRPLLDKGIVPLKDENELNDFLDTSMEWIPGIKGLRFRDLPSFCRITDSNDIMFNWIIESIEMASEASAVVIQTFEALEPHVLDVLSSTTSLPKIYPIGPLQPLLDHYPQHSLHTVTYSLWKEETECIKWLSTQKPNSVIYVNFGSITVLSEQQVIEIGWGLANSDVPFLWVIRPDLIIGEGRLPSEFLDQTKERSLIISWAPQEDVLNHPSIGGFLTHCGWNSILESLTSGVPMICYPYFADQQTNCWFACNEWGVAVEIESDVKRDQVTNLVKELFDGEKGKKIKTTAMEFKKLVHEATTPSGSSSTNLDDLINEVLLHQTD

SEQ ID NO: 79 - DNA, 672 bp, Cannabis sativa
  source 1..672: mol_type = other DNA
ttgggaaaacgtgtaaacatcattgaaggtgtcactcaagggcttctttatctccaagaatactcaagattgacaattatacatcgagacttgaaagctagcaatatattacttgatgatgagatgactcctaagatatcagattttggaatggcaagaattttcacaaaagatggacttgaagcaaacacaaggcgaatagttggaacatatggctacattccaccagaatatgccatatgttccaactattcgccttgtgtttgctttggagttctacttctacagatcataagtggcaaaaggacagctcttgtttatggattgaatgaagatctaaatctccttgaatatgcatatgaactgtggaaaacaggaaaaggaatggattttatggacccttcacttgaggacttacattcatcatgcaaattgatgaaaggcctagaaatagctctgttatgtgtacaagacaatgcaaatgacagaccatccatgttagaagtttcttcaatgctgaagaaccaaaatgcaactatggtgacaccccaaaagcctacttttgcaactagaaatagtaaaaatcaggagggatgtacagttaaatcttgcccagctgatacttcttcttcggtcaatgaagctacaatctctgaggtgctaccccgctag

SEQ ID NO: 80 - protein, 223 aa, Cannabis sativa
LGKRVNIIEGVTQGLLYLQEYSRLTIIHRDLKASNILLDDEMTPKISDFGMARIFTKDGLEANTRRIVGTYGYIPPEYAICSNYSPCVCFGVLLLQIISGKRTALVYGLNEDLNLLEYAYELWKTGKGMDFMDPSLEDLHSSCKLMKGLEIALLCVQDNANDRPSMLEVSSMLKNQNATMVTPQKPTFATRNSKNQEGCTVKSCPADTSSSVNEATISEVLPR

SEQ ID NO: 81 - DNA, 2412 bp, Cannabis sativa
  source 1..2412: mol_type = other DNA
atggaggttttatatgtagggtctttgaatgctgatgttgatgcaaatgagcaggatgatgtatcacagcttttgttggataagtttaagtgtgttccagcatttttacctgctgacattctgtcaaagttctatcatgggttctgtaagcagcatttgtggcccctttttcactacatgcttccttactcagctacacatggtggccggtttgaacgatctttgtgggaggcttatgttgcggcaaataagattttctcgcagaaggttattgaggttataaacccggaagatgactatgtttggattcatgattaccatttgatggtgttgcccacattcttgcggaggagatttaataggctcagaatggggttctttcttcatagcccatttccctcgtcggagatttacaggactttgcctgtgagagaagagattctaaaggcactgttaaattcagacctcattggtttccatacttttgattatgcccgtcatttcttgtcttgctgtagtcggatgcttggtctagagtaccaatcaaaaagaggttatattgggttggaatactatgggagaacggttggaattaaaatcatgcctgttgggatccacatggggcagatcgagtcagtcttgagaaatccggataatgagtggagagtaaaagagctcagacgaaagtttgcaggaaagactgtattgcttggagtcgatgatatggacatctttaaaggtgtcaacttgaagttgttggccatggagcagctgctgaggcagcatccctcctggcatggaaaggctgttatggtccaaattgcaaaccctgctagggcaagagggaaagaccttgaggaaattcaggatgaaatacatgctagctgcaagagaataaatgagacatttggtcgagccggttatgaaccaatcatctttattgacaggccagtctctctgagtgaaagggctgcttattatgcaattgctgagtgcgtggtagttgccgcagtgagggatggaatgaatcttacaccatatgagtacattgtatgtagacaaggggtgtctagttcagagtctagcacggactttgatgggcctaaaaagagcatgctagtagtatcagagttcattggatgctctccttctttgagtggtgcaatccgtattaatccatggaatattgagacaactgctgaggcaatgaatgaggcaatttctatggctgattctgagaagcaaatgcggcatgaaaagcactataggtatgtcagcacccacgatgtagcttactggtctagaagctttttccaggatatggagagaacatgtagagatcatttcagaagaaggtgctggggcatcggtcttagctttggtttcagggttgttgcccttgatcccaactttaaaaagttgtcaattgatcatattgtttcttcatattcaagatccaaaaacagggctatattgctggattatgatggtactgtaatgccacaaacatccatcaataaagtcccaagtccgacggtcatctccaccataaatatgctctgcgctgatgataaaaactccatatttgttgtcagcgggagaggaagagacagcttagggaagtggttttctccttgccataaacttggtattgctgcagaacatggttattatatgaggtggtctgctgacaaggattggcaaatttgtggcccgtgtaatgattttgggtggatacagatagttgaacctgttatgaaacaatacacggaatccactgatggttcttacatcgaaaagaaagagagtgccatagtttggcatcatcgtgatgcagatcctagttttggatcttgccaggccaaggagttactggaccatctagaaagtgttctagcaaatgaaccagttgcagtgaaaagtggtcagtttatcgtagaagtaaagccacagggagtaagtaaaggcttggttgctgaaaatatcttcacaacaatgaatgagaatggaagacaagccgattttgtactgtgtgttggtgatgacagatccgacgaggacatgtttgagattattggcagtgcaatgtcaagtggtgtactctcaaataattcatcggtgtttgcatgcacagttggacagaaaccgagtaaagccaagtactatttggatgacacggcggaagtcattaatatgcttgaagctttagcagaagaatcagattgtgctcccttggaattggaactgttagtcatgcttgaactctctccaccatggcctttcaaaggccctggaagaagtacatgtacttctgtttatttttcagccttgcggggaaaggtaaattttattggaagaaaacattggacgctacgtgttttgctgtggttgaacaaaacagggccatcttaa

SEQ ID NO: 82 - protein, 803 aa, Cannabis sativa
MEVLYVGSLNADVDANEQDDVSQLLLDKFKCVPAFLPADILSKFYHGFCKQHLWPLFHYMLPYSATHGGRFERSLWEAYVAANKIFSQKVIEVINPEDDYVWIHDYHLMVLPTFLRRRFNRLRMGFFLHSPFPSSEIYRTLPVREEILKALLNSDLIGFHTFDYARHFLSCCSRMLGLEYQSKRGYIGLEYYGRTVGIKIMPVGIHMGQIESVLRNPDNEWRVKELRRKFAGKTVLLGVDDMDIFKGVNLKLLAMEQLLRQHPSWHGKAVMVQIANPARARGKDLEEIQDEIHASCKRINETFGRAGYEPIIFIDRPVSLSERAAYYAIAECVVVAAVRDGMNLTPYEYIVCRQGVSSSESSTDFDGPKKSMLVVSEFIGCSPSLSGAIRINPWNIETTAEAMNEAISMADSEKQMRHEKHYRYVSTHDVAYWSRSFFQDMERTCRDHFRRRCWGIGLSFGFRVVALDPNFKKLSIDHIVSSYSRSKNRAILLDYDGTVMPQTSINKVPSPTVISTINMLCADDKNSIFVVSGRGRDSLGKWFSPCHKLGIAAEHGYYMRWSADKDWQICGPCNDFGWIQIVEPVMKQYTESTDGSYIEKKESAIVWHHRDADPSFGSCQAKELLDHLESVLANEPVAVKSGQFIVEVKPQGVSKGLVAENIFTTMNENGRQADFVLCVGDDRSDEDMFEIIGSAMSSGVLSNNSSVFACTVGQKPSKAKYYLDDTAEVINMLEALAEESDCAPLELELLVMLELSPPWPFKGPGRSTCTSVYFSALRGKVNFIGRKHWTLRVLLWLNKTGPS

SEQ ID NO: 83 - DNA, 1014 bp, Cannabis sativa
  source 1..1014: mol_type = other DNA
atggtttactctcatttggggtgtttttgtatttggggttggtctttgtttgtgcttacagttactcttgttaatgttgctggttcaactagtaataataacttgcccatcatagcttttgatgaagggtacacccaactttttggggatgataatctgattatccatagggatggaaaatctgttcatctttctcttaatgaaagaacagggtctggatttgtttctcatgacctttatcttcatgggtattttagtgcttctattaagttacctgctgattatactgctggagttgtggttgccttttatatgtctaatggtgacatgtttgagaagaaccatgatgaaattgattttgagtttttgggtaatataagaggcaaagaatggaggattcagactaatgtttatggaaatggaagcacaaatgttggcagagaagagagatataatctatggtttgatccttctgacgattttcatcagtacagcattctctggactgattctcatatcatattctatgtagacaatgttccaataagagaggtaaagagaacagagtcaatgggtggtgatttcccatctaagccaatgtcattatatgccacaatttgggatggttcagattgggcaactaatggtggaaaatacagagtaaactacaaatactctccctatgtagccaaattctccgaccttgtcctccacggttgctcggtcgatccaatggagcaattctccaagtgtgacaacatcccaaattccaaatccatcccaactggtatcacaccatcaaaacgaatcaaaatggagggcataaggaagaagcacatgacatactcatattgctatgacaaacttaggtacaagtctcctccttccgaatgcgtgatcaaccctcaggaggccgagagactacgcaaatttgatcccgttacctttggaaatggccgtcgccaccatgggaagcgccaccacaggaacaaagctagccaagcacagaaatag

SEQ ID NO: 84 - protein, 337 aa, Cannabis sativa
MVYSHLGCFCIWGWSLFVLTVTLVNVAGSTSNNNLPIIAFDEGYTQLFGDDNLIIHRDGKSVHLSLNERTGSGFVSHDLYLHGYFSASIKLPADYTAGVVVAFYMSNGDMFEKNHDEIDFEFLGNIRGKEWRIQTNVYGNGSTNVGREERYNLWFDPSDDFHQYSILWTDSHIIFYVDNVPIREVKRTESMGGDFPSKPMSLYATIWDGSDWATNGGKYRVNYKYSPYVAKFSDLVLHGCSVDPMEQFSKCDNIPNSKSIPTGITPSKRIKMEGIRKKHMTYSYCYDKLRYKSPPSECVINPQEAERLRKFDPVTFGNGRRHHGKRHHRNKASQAQK

SEQ ID NO: 85 - DNA, 264 bp, Cannabis sativa
  source 1..264: mol_type = other DNA
atgaaccacatgatttacgattactgtaccgataaatcccggaaccggtactccaccagagtgtgtcgccggctactagatcaactctcttccaatttctacattataacactcataatatatacttcaaaaaaaaaaaaatgttgttgttattgtttgaaaagaagaaatttgaaaagtccgtacgtatacgttacatggaactggcccccttttgtacactactaccctccttattgcatgttctttattaagggtatataa

SEQ ID NO: 86 - protein, 87 aa, Cannabis sativa
MNHMIYDYCTDKSRNRYSTRVCRRLLDQLSSNFYIITLIIYTSKKKKCCCYCLKRRNLKSPYVYVTWNWPPFVHYYPPYCMFFIKGI

SEQ ID NO: 87 - DNA, 1410 bp, Cannabis sativa
  source 1..1410: mol_type = other DNA
atggcggttagcaagagccgggtcagtgtccactctacgctaacagcggtatccagcaggccagtcgggtcgggtaataaaacccataagtttacgtctcttgatcacgccatgggtcttcatagccttcacatcgtattttactacagggataatgttctcaaggactttgatttggacccagccagggtatccctttcggacgctctttcgttgtacccacaagtaacgggtcggatagcccgaaacggagatggtaactgggaggttaagaacaacgatgcaggtgttcgggtcgtgaaggcccaagttggtgcctcgtttgatgagtggctaagatcagctgatgggatggaagaaaaagatctaatggtctgggatgagatgcctaaggaccccactaattggtcacctttccgaatccagataaatgaatttgaagggggaggagtagccgttgggctgagctgcactcacatgcatgcagatccaacatcggcaaccctactcttcaaatcatggattgacactcaccgtggtcgggccatagcccacccactcgtaaattcagggtccgatctctgtacccgacccgatacaaatccgaagaagaactctgtaacttactatcacgccaagtccatcgcacgtgcggcgcccttagtgaaaatggccacggcaaccttcaaattctcaaatgcaatactcaagcagtgcctctcaggtatctccgaaagttgccaagacgccactcccttcgattttctaggggcactcttttggacacgtgtcgcacaattgaaggtcctaaaaaataactccacatactccctctcaatatgtacggacttcaggaagttattgaagaaaactccgcttcctcttgggtattacggcaacgctttgcatttttcgatgctttctttacaagttcgagagctcagtagtggctctgatttaggacacgtggtgagagaactgcaccaccacgtggcgagcgtcgacgatgatgaggttaggtcagcgattgattggctggagtcgagaaggaagagagagggggaggaggaaaatagttacggcccaccttttaggatgtatgggcctgaattgacttgtgttaaggttgatcacttgggcagttcatgtgggccgaatggtagcgttgttgggccgttgatgtacgatactgcgtttgatgagggagcagagccggtccacgtgtcgtatcacattgggaatgtggaaggtgaagggttgatcatggtgatgccttcgtcggaaggtggtctgggtagggtggtgacggtgacgttgccggagaatgagttggccgcgttgtgtgaggaccaagcattattgggcctgcaacccataatgttgcttaatggtggacgtgggagatgctag

SEQ ID NO: 88 - protein, 469 aa, Cannabis sativa
MAVSKSRVSVHSTLTAVSSRPVGSGNKTHKFTSLDHAMGLHSLHIVFYYRDNVLKDFDLDPARVSLSDALSLYPQVTGRIARNGDGNWEVKNNDAGVRVVKAQVGASFDEWLRSADGMEEKDLMVWDEMPKDPTNWSPFRIQINEFEGGGVAVGLSCTHMHADPTSATLLFKSWIDTHRGRAIAHPLVNSGSDLCTRPDTNPKKNSVTYYHAKSIARAAPLVKMATATFKFSNAILKQCLSGISESCQDATPFDFLGALFWTRVAQLKVLKNNSTYSLSICTDFRKLLKKTPLPLGYYGNALHFSMLSLQVRELSSGSDLGHVVRELHHHVASVDDDEVRSAIDWLESRRKREGEEENSYGPPFRMYGPELTCVKVDHLGSSCGPNGSVVGPLMYDTAFDEGAEPVHVSYHIGNVEGEGLIMVMPSSEGGLGRVVTVTLPENELAALCEDQALLGLQPIMLLNGGRGRC

SEQ ID NO: 89 - DNA, 1035 bp, Cannabis sativa
  source 1..1035: mol_type = other DNA
atggcagttacttacttggataccaatcaacgaccacatcagccacctggtccgccactacacttatttggtgttggacatcaaggcccatttgtagatggtgcacatcggcctccaggcccaccaccacctccctatttaggtagggccggaccaagacaaagaggcccaccaaaacatggtagccaaccaccatgggttgatggattgaaagggtctattgctttgatgattaacacgtgtgattatcttgaacgtccattcatcgaatacttaaccaagcaaataggaaagcctgtgtggggtgtgggaccacttttgcccgaacaattttggaattctgttagttcgaattcgattcttcatgatcatgaaattcgtacaaatcgacaatcaaatgtttcagaggatgatgtcatccaatggttggattcgaagccacgtggctcggtgttgtatgtgtgttttggcactgaggttagtccatcaatggaagagtactcagaactagcagatgcattagaggcgtcgacccaaccatttatttgggtggtccattccgggacgggaaggtcaggtcctccaccaagtcgcggaccaactcaagaagattatttccctcatgggcttgctagccaagttggcccaaaaggtttgataataaatggttgggccccacaattactcattctaagccattcatcgattggtggatttttgacacactgtgggtggaactctacggttgaagcaattgggcttggagtcccactcttggcttggcccattagaggagaccagaattataatgcgaaactcgttgtggctcatctgaaattgggtttcatgatttcggataatttatcggaaaagattaagaaacatgaaattgttaagggaataaagacactaatgggggacgatgacataaaatcacgagcaagaaaccttgcagctatatttcaaaaaggatttccaataagttctaccactaatttggatgtttttagagacatgataaataactttacttaa

SEQ ID NO: 90 - protein, 493 aa, Cannabis sativa
MELCKQIASRNINTLLVIPSNLSFSIPSSLRQYPLLQIVEIQPTSAPSAQPGPDPIDQPPHGNPDQFEMGLENLLQAQVTGPDSVRPICAILDVMMDWTTEVFNKFDIPTIGFFTSGACSAAMEYGLWKAQPIDLKPGEVRLLPGLPEEMAVTYLDTNQRPHQPPGPPLHLFGVGHQGPFVDGAHRPPGPPPPPYLGRAGPRQRGPPKHGSQPPWVDGLKGSIALMINTCDYLERPFIEYLTKQIGKPVWGVGPLLPEQFWNSVSSNSILHDHEIRTNRQSNVSEDDVIQWLDSKPRGSVLYVCFGTEVSPSMEEYSELADALEASTQPFIWVVHSGTGRSGPPPSRGPTQEDYFPHGLASQVGPKGLIINGWAPQLLILSHSSIGGFLTHCGWNSTVEAIGLGVPLLAWPIRGDQNYNAKLVVAHLKLGFMISDNLSEKIKKHEIVKGIKTLMGDDDIKSRARNLAAIFQKGFPISSTTNLDVFRDMINNFT

SEQ ID NO: 91 - DNA, 522 bp, Cannabis sativa
  source 1..522: mol_type = other DNA
atgatgactgctgaaactgggacttatcggtggatggcaccagagctttacagcacggtcacattaaggcatggcgagaagaagcattacaatcacaaggtggatgcatacagctttgccattgttttatgggagctcgtccataataagttgccttttgaaggcatgtcgaatctacaagctgcatatgctgcagccttcaagaatgtgaggccaagtgctgagaacctccctgaggatttagcattgattgtaacttcatgttggaaagaagatccaaatgacagacccaacttcagccaaattatacaaatgctcctgcactatctctcgacgatatcacctgcagagccggtaattccattgcggattttcagatcagagaatgctgtactgccaccagagtcaccgggtaccagttcgttaatggcggttagagaagaagcaggagacacaccgaaaaccaacacagaagaagaaaagcctaaaggcctcttcttctgctttaaccagtgttactaa

SEQ ID NO: 92 - protein, 173 aa, Cannabis sativa
MMTAETGTYRWMAPELYSTVTLRHGEKKHYNHKVDAYSFAIVLWELVHNKLPFEGMSNLQAAYAAAFKNVRPSAENLPEDLALIVTSCWKEDPNDRPNFSQIIQMLLHYLSTISPAEPVIPLRIFRSENAVLPPESPGTSSLMAVREEAGDTPKTNTEEEKPKGLFFCFNQCY

SEQ ID NO: 93 - DNA, 1377 bp, Cannabis sativa
  source 1..1377: mol_type = other DNA
atggagctcaatattgtctcaaaagaaataatcaagccatcctcacaaactcacaatctgaaacgttacaacctctgccttttcgatcagctcatccctaatacttatctctccatcattctcttctaccctatccacaatgatcccaaagacctcaccacagaccattccaaagccataacccaactgaagaaatccttgtcagagacactcacattgtactacccactttctgggaaaaccaaaaataatctatacgtagatgaattctccactggtgtgattttcatagaagccaaagtcaaatgtcaaatgtctgagttgttccaaggagaaaatgagttacagaaccaactagtcccatttcaacccttttcccatgaaaaaacagaggactacgttcccataattgccttccaattgaacatattctcttgtggaggaattgcactcggcatttccttgggtcacaagaataacgacattgcaatgctaagcaattttctcaaatcttggtcggctatctccactggttcacctgagaaagtaataaagcctgatctttcttcccctgctatttactttcctccccgaacaaatgatgatgatgatgatgattttctcagcaagtacacatctttgatggatcgtttatggttcagcgaaggagattatgttacaaagaggtttgttttcccagccaagtccattgaaactcttaaacaaaaggcgaaaagcgaacgcgttccgaacccgtctcgcaacttggcaatctcgtgtttgatatggaaacacgcgatggcggcttcttgggctgtgtttggatcccctaaggcatccattgctgggcaagcggtgaacatgagaccccgaatgaagcctaaactggatggagctgttggaaactttttctggtggtcaatggcatcttatgatcccaacgattataataatgaagtggagttaagtgagttggtggtacagatgaaagaatcaataacgacaggactttttgatgaagattatctttctcaactgatgatgatggccaataacaaagaggggctggtgagtgaggttgagaatcagctagagattatgagttgcattgaattagaaaataatgctgatatatttgtgttcactaattggaaagatttctttggggaagttgattttgggtttgggaaaccgttgagcgtaggaagcataggaaaggttggccctgcttttaggaatttggtcatcttagttgatgctgatcaatggggtaaaggctcagtcgatgcatttattaccttaaaagccaaagaaataactctcctccaaagtgatgaagcctttttacattttacatttcagaatcctgtcaaataa

SEQ ID NO: 94 - protein, 458 aa, Cannabis sativa
MELNIVSKEIIKPSSQTHNLKRYNLCLFDQLIPNTYLSIILFYPIHNDPKDLTTDHSKAITQLKKSLSETLTLYYPLSGKTKNNLYVDEFSTGVIFIEAKVKCQMSELFQGENELQNQLVPFQPFSHEKTEDYVPIIAFQLNIFSCGGIALGISLGHKNNDIAMLSNFLKSWSAISTGSPEKVIKPDLSSPAIYFPPRTNDDDDDDFLSKYTSLMDRLWFSEGDYVTKRFVFPAKSIETLKQKAKSERVPNPSRNLAISCLIWKHAMAASWAVFGSPKASIAGQAVNMRPRMKPKLDGAVGNFFWWSMASYDPNDYNNEVELSELVVQMKESITTGLFDEDYLSQLMMMANNKEGLVSEVENQLEIMSCIELENNADIFVFTNWKDFFGEVDFGFGKPLSVGSIGKVGPAFRNLVILVDADQWGKGSVDAFITLKAKEITLLQSDEAFLHFTFQNPVK

SEQ ID NO: 95 - DNA, 1296 bp, Cannabis sativa
  source 1..1296: mol_type = other DNA
atgcaagggttgataggagccactacccaagccatagctttctccaaagcatcaattcgccatttcaactcacctactactcataaactcctcaacccttctcttcttcatctacccaacaaacccttattcttcaaccttcgaaccttcgccataatggcaaccctcgacaacttcgtcaacggaagcgttcacccgaacggcgtcgccgtcatcacccttgatcgttccaaagcgctcaacgccatgaatttagaaatggatgtcaaatacaaaagttatcttgatgactgggaatctgatccaagggtgaaatgtgttctggtcaatagcagctcgcctcgtgctttttgtgctggtatggatattaaaggagttgttgctgaaattcaaaaggacaagaacactcctcttgttcagaaggtttttcaagctgagtactctctgatatgcaaaatctctgattacaagaagccttatataagcttcatggatggtatcacaatgggctttgggattggtctttcaggtcatggtctctataggattataacagagagaacacttcttgctatgcctgaaaatggaattggtttgttcccagatgtggggttctcatatatagcagcacaaagtccgggaggaggagctgttggtgcttaccttggattgactggaaaaagaatctctacaccctcagatgcactatatgtggggcttgggtcacattttgtaccgtctggaaacttggcttcactaaaggaagcccttctttcaacaaacttttctgaggatccacatcaagatgtgaaagaacttttgggaaaattcggtagcaaccccgaggcggaggctcaattaagtttgcttttacctcaaataatatcgtctttcggttcaaataagtcagttactgagacaattgaatcactgaaaagttaccagcagagttcagatgaaactgtagtggagtgggcaaatgaagctcttcaagggatagctaaaggtgctcccttttcactttgcttaacacaaaagtacttctccagagttgcatctgcatatggaaaaagggagaatgaattatcttcgcttactggtgtaatgaaaactgaatatcgcattgctttgagatcttctttgaggaatgattttgctgaaggtgttcgagcagtgttggttgataaggatcagaaccccaaatggaacccgtcgacgttggaggaagttgatgagaaggaagtagaaggtgtgtttgaagcactgagcccgggagttgaggaactgcgtgtttga

SEQ ID NO: 96 - protein, 431 aa, Cannabis sativa
MQGLIGATTQAIAFSKASIRHFNSPTTHKLLNPSLLHLPNKPLFFNLRTFAIMATLDNFVNGSVHPNGVAVITLDRSKALNAMNLEMDVKYKSYLDDWESDPRVKCVLVNSSSPRAFCAGMDIKGVVAEIQKDKNTPLVQKVFQAEYSLICKISDYKKPYISFMDGITMGFGIGLSGHGLYRIITERTLLAMPENGIGLFPDVGFSYIAAQSPGGGAVGAYLGLTGKRISTPSDALYVGLGSHFVPSGNLASLKEALLSTNFSEDPHQDVKELLGKFGSNPEAEAQLSLLLPQIISSFGSNKSVTETIESLKSYQQSSDETVVEWANEALQGIAKGAPFSLCLTQKYFSRVASAYGKRENELSSLTGVMKTEYRIALRSSLRNDFAEGVRAVLVDKDQNPKWNPSTLEEVDEKEVEGVFEALSPGVEELRV

SEQ ID NO: 97 - DNA, 1428 bp, Cannabis sativa
  source 1..1428: mol_type = other DNA
atggagcgtttatcttttatttgttgttttctcttgctcatttctatctcagggttcgcagatgctggttcaataggagtgaactatgggcgaatagcgaataaccttccttctgccgtgaaagtggtacagcttctcaaatctcagggtcttcagaaagtcaaggtttacgacgccgacccttctgtcttgaaagccttagctggaactggtatcaaagtcaccgtagatctacccaacgagcttctttacgcggcagccaaacaacagtcctttgccaactcatgggttcagaaaaacgtcgctgcttattacccatctaccgagattgaagctcttgctgttggaaacgaagtcttcgtcgacccgcataacaccaccaagtttctcatcccagctatgagaaacattcactcggctctaattaagaacaagctggattctgcgatcaaaatctcctctcccattgctcttagtgccctacagaactcataccctgcatcggctggttcgttccgacctgagctcgtcgaaagcgtattcaaacccatgttggatttcattcgcgaaaccgggtcgttcctcatggtaaacgcttacccatttttcgcttacgagtcaaactctgatgttatatctttagactacgcactgttccgtgagaacccaggtgtcctggatgcgggttcaggtttacggtacttcaacctcttcgacgcacaaatcgacgccgtttttgctgcaatgtcagctttgaaatacgatgacattaagcttgtcgtttctgaaacgggttggccatcaaagggagataagaacgaagtgggtgcaagcttagacaatgcagccgcctacaacgggaatctgatccgtcgaattttaaccggtggtgggactccattaaggcccaaagatgatctcaccgtttatctcttcgctctcttcaacgaaaataaaaagaacggtcccacatcggaaagaaactatgggcttttctacccggacgaaaagaaagtttatgatatcccttttactgccgagggtttaaagagctaccacgatgaccggtcaccggctagtggaggagaaccatccggtcaacaggttactagccctgtcaatggtggaggtgtgtcgaagagtttaaccggtagcacttggtgtgttgcgaatgctcaactggggaaagaaaagttacaggcagctctggatttcgcttgcggtgagggcggtgctgattgccgtccgatccagccaggctccacgtgttacgatcctaataccattgaggcccatgcctcgttcgccttcaatagttattatcagaagcagagccgtcagatggggtcttgctattttggcggggcggcttacgtcgttactcaaccacccaggtatggaaagtgcgagtttcccacgggttactga

SEQ ID NO: 98 - protein, 475 aa, Cannabis sativa
MERLSFICCFLLLISISGFADAGSIGVNYGRIANNLPSAVKVVQLLKSQGLQKVKVYDADPSVLKALAGTGIKVTVDLPNELLYAAAKQQSFANSWVQKNVAAYYPSTEIEALAVGNEVFVDPHNTTKFLIPAMRNIHSALIKNKLDSAIKISSPIALSALQNSYPASAGSFRPELVESVFKPMLDFIRETGSFLMVNAYPFFAYESNSDVISLDYALFRENPGVLDAGSGLRYFNLFDAQIDAVFAAMSALKYDDIKLVVSETGWPSKGDKNEVGASLDNAAAYNGNLIRRILTGGGTPLRPKDDLTVYLFALFNENKKNGPTSERNYGLFYPDEKKVYDIPFTAEGLKSYHDDRSPASGGEPSGQQVTSPVNGGGVSKSLTGSTWCVANAQLGKEKLQAALDFACGEGGADCRPIQPGSTCYDPNTIEAHASFAFNSYYQKQSRQMGSCYFGGAAYVVTQPPRYGKCEFPTGY

SEQ ID NO: 99 - DNA, 615 bp, Cannabis sativa
  source 1..615: mol_type = other DNA
tggccaacttttttgtacaaagttgtccccccaacttttttgtacaaagttgtccccaaaaaaaaaaaaaaaaaatgcattgtatggctgttcaccaatttttctccatcaatagtatcatcactacctaccattagtactaataataatcacttttgtagatttttcacaccaaaaacatctattagtcctatttcaaaatcaaaatcaaaatcaaaatcaagtacttgctaccccatccaatgtactgtggtcaataatagtagccctagctctactactaaatcttctattgttcgaagatcggccaattatgaacctccgatttggtctttcgattacattcaatctctttcaacccaatataagggagaatcctatacaaggcaattaaataagttgaagaaagaagtgacaagaatgctccttggactggaaattaactctttagccctacttgagctaattgatacattacaaaggtttggaatatcttaccattttaagaatgaaataaacactattttgaagaaaaaatacactgataattatattaacaataatattattactaaccctaattataattttatatgccattgctctcgaatttag

SEQ ID NO: 100 - protein, 204 aa, Cannabis sativa
WPTFLYKVVPPTFLYKVVPKKKKKKCIVWLFTNFSPSIVSSLPTISTNNNHFCRFFTPKTSISPISKSKSKSKSSTCYPIQCTVVNNSSPSSTTKSSIVRRSANYEPPIWSFDYIQSLSTQYKGESYTRQLNKLKKEVTRMLLGLEINSLALLELIDTLQRFGISYHFKNEINTILKKKYTDNYINNNIITNPNYNFICHCSRI

SEQ ID NO: 101 - DNA, 753 bp, Cannabis sativa
  source 1..753: mol_type = other DNA
atggaagagggtaaaggtcctgttagggttcgctgccagcgaattggctgcgacgctactttctccgaggacgataatcccgaaggttcttgtcaataccatgattcgggacctttatttcatgacggaatgaaagagtggagttgctgcaagaagagaagtcatgatttttctttatttttagaaattcctggatgtaaaacaggtaaacacacaactgagaaacctgtgctaacaaagccagtgcgtaaaccaaatatttctatttcaccttctacttcgtcaccagcaatcaatgcatcagccaaagaatcctgctctagatgccggcaagggtttttctgctcagatcatggttcacaggctaaatacgttaaaccaaaacctacaggcacagattccgtggcaaaaatcaatacagatgctcagactattcctgctcctgcaaagaaggttattgatataaacgaaccccaaatctgcaaaaacaaaggatgtggtcaaactttcaaagagaaagataatcatgaaactgcatgcagttaccatccagggcctgctattttccacgaccgagtgagagggtggaagtgctgtgacattcatgtcaaggaattcgatgaagttcatcaccattcctccatgcacgaaggggtggcacaatgctcaagcgacatagttactatgataacacaatttttacgcaaagggccgctgaatgcatcttcaaccatttatcttttgctggactga

SEQ ID NO: 102 - protein, 250 aa, Cannabis sativa
MEEGKGPVRVRCQRIGCDATFSEDDNPEGSCQYHDSGPLFHDGMKEWSCCKKRSHDFSLFLEIPGCKTGKHTTEKPVLTKPVRKPNISISPSTSSPAINASAKESCSRCRQGFFCSDHGSQAKYVKPKPTGTDSVAKINTDAQTIPAPAKKVIDINEPQICKNKGCGQTFKEKDNHETACSYHPGPAIFHDRVRGWKCCDIHVKEFDEVHHHSSMHEGVAQCSSDIVTMITQFLRKGPLNASSTIYLLLD

SEQ ID NO: 103 - DNA, 1122 bp, Cannabis sativa
  source 1..1122: mol_type = other DNA
atggaagctgctagaggtgaagctgagcttgttatcttctccgccatggatgatttgttacaaaaaacagggcttaaacccaaagatatcgatattttaatcgttaattgcagtttattctcacctactccttctttatccgccatggttattaataaatacaagcttagaagtaatatcaagagctttaatctttccggtatgggatgtagcgccggagtaatctcgatcgacttagctcgtgatcttctacaggttcaccccaattcgaacgctgttgtgataagtactgaaattattaccccaaattactatcagggaatgagagagccatgcttttgcccaattgtctcttccgaatgggcggcgccgccattctcctctcgaatcggagatcggaacggcggagagccaaatataggttggtacatgttgtacggacacataaaggatctgacgacaaggcttacaggtgcgtttacgaagaggaagacaaggagggtaaagttggaatttcaactatcaaaggatctaatggctatagcaggagaagcattaaagtcaaacataacgacgataggtccgctagtactgcccgcatcagaacaattattatttttgatgagtttaatcggacggaagatattcaatccaaaatggaaaccttacattcctgatttcaaacaagcatttgaacatttttgtatccacgctggagggagggctgtgatcgatgagcttcagaaaaatctacagctgtcagctgagcactgtgaggcttctagaatgactttgcatcgttttggcaatacgtcgtcgtcttcactttggtatgaaatggcttatgttgaagccaaaggaagaatgaaaaaaggtgataggatttggcaaattgcttttggaagtggatttaagtgtaacagtgccgtttggaaatgtaatagactatcaaaacgacgtcggatggtccttggactgattgcattgaccgttatcccgttcatattcctgacgtcgtcaaactctaatttattattatatatatatataataataatgttttttttttttctttttttaagggtaatttgttgttatgtatgtgatgttatatatagatga

SEQ ID NO: 104 - protein, 373 aa, Cannabis sativa
MEAARGEAELVIFSAMDDLLQKTGLKPKDIDILIVNCSLFSPTPSLSAMVINKYKLRSNIKSFNLSGMGCSAGVISIDLARDLLQVHPNSNAVVISTEIITPNYYQGMREPCFCPIVSSEWAAPPFSSRIGDRNGGEPNIGWYMLYGHIKDLTTRLTGAFTKRKTRRVKLEFQLSKDLMAIAGEALKSNITTIGPLVLPASEQLLFLMSLIGRKIFNPKWKPYIPDFKQAFEHFCIHAGGRAVIDELQKNLQLSAEHCEASRMTLHRFGNTSSSSLWYEMAYVEAKGRMKKGDRIWQIAFGSGFKCNSAVWKCNRLSKRRRMVLGLIALTVIPFIFLTSSNSNLLLYIYIIIMFFFFLFLRVICCYVCDVIYR

SEQ ID NO: 105 - DNA, 663 bp, Cannabis sativa
  source 1..663: mol_type = other DNA
atgttgcataccatttcattgcctgcagcctataagtcagccacgtgtctcaatctacaccgttcatcttcaccgtcgccaccaatatggccaatatcaccatctccatcatcagttgcaactttaccggcaaataatcataataatattaacaatattcatcaacatcatcgtcgtctcaatatgattccacaacattctctcatccaacgtcggccaatattttcaacggcttgtagtaacagtagtagtagtaataatagtaataacgagtatttggaaattgtgctcaaagttcgcgattacgagctcgatcagtatggtgtcgtaaacaatgctatttactcaagttattgccaacatggtagtcatgagttcatggacagtattggtgtaagttgtgatgcaattgctcgtacaggaaatgcattagcaactgcagaagtgtctatcaaattcatttcacctttaagaagtggggataaatttgtggtgaaagtaagaatctctggattgacaccaactcgcatatactttgagcatcgaatcttcaagctttcaaataacgagcttattttggaaggaaagacgacggtggtttggttgaataagaagtatcgtccttctcgtataccatccgaaatatataaactccttaattaa

SEQ ID NO: 106 - protein, 220 aa, Cannabis sativa
MLHTISLPAAYKSATCLNLHRSSSPSPPIWPISPSPSSVATLPANNHNNINNIHQHHRRLNMIPQHSLIQRRPIFSTACSNSSSSNNSNNEYLEIVLKVRDYELDQYGVVNNAIYSSYCQHGSHEFMDSIGVSCDAIARTGNALATAEVSIKFISPLRSGDKFVVKVRISGLTPTRIYFEHRIFKLSNNELILEGKTTVVWLNKKYRPSRIPSEIYKLLN

SEQ ID NO: 107 - DNA, 1134 bp, Cannabis sativa
  source 1..1134: mol_type = other DNA
atgggagccggtggcaaaaatagtagacttgagcgagcaccacacaccacaccaccattcacactaagccaactcaagaaagccattccaccccattgcttcaaccgttctcttcttcgttccttctctcatgtccttcaagaccttttttttgcctttttgttctactacatagcaacctcttacttccaacttctcccacacccgctccaatacttagcttggccactttattggatcttccaaggcagcatttttgctggtatttgggtccttggtcatgattgtggtcaccaagctttcagtgaccaccaatgggtggatgacactgttggctttgtcctccactccgctcttctcttcccatacttctcttttaagtatagtcatcgtcgccatcattcaaacatcgcctcccttgaacatgatcaattgtttgttccagtccccgaatctcaaatcgcatggctctacaaacgttacttggacaatccactaggaagagccctaaagctttccactatagtgttccttggttttcctttgtacttaggtttcaatcttacaggcaaaccatatgatcgttctgcatgtcattatgatccttactctccactctactcaaaaagtgaaaggcttcatatattgatttcagatatcggtgttttcatcaccacattagtgttataccagcttggctcgactaaagggttgagttggcttgtgttcatgtatggggtgccattgtttacagggaatagcatccttgtgacaatcgcatacttgaatcatactcactcttcattgcctcattatgactcgtcggagtgggattggttgaaaggagcattgtcaacaactgatcgaaactatggatcaattctcaatagggttttccatcaccttacagatgctcatatggcacaccatttattcgcaacaatacctcactaccatgcaaatgaagccaccaaagttatcaaatccatattgggagaatactactcttttgatgatactccaataattaaagctctttggagagagactaaggagtgtgtctatattgagccaaatcatgaatcttctcctaataataacaaaggtgttttctggtacaacaacaagttctga

SEQ ID NO: 108 - protein, 377 aa, Cannabis sativa
MGAGGKNSRLERAPHTTPPFTLSQLKKAIPPHCFNRSLLRSFSHVLQDLFFAFLFYYIATSYFQLLPHPLQYLAWPLYWIFQGSIFAGIWVLGHDCGHQAFSDHQWVDDTVGFVLHSALLFPYFSFKYSHRRHHSNIASLEHDQLFVPVPESQIAWLYKRYLDNPLGRALKLSTIVFLGFPLYLGFNLTGKPYDRSACHYDPYSPLYSKSERLHILISDIGVFITTLVLYQLGSTKGLSWLVFMYGVPLFTGNSILVTIAYLNHTHSSLPHYDSSEWDWLKGALSTTDRNYGSILNRVFHHLTDAHMAHHLFATIPHYHANEATKVIKSILGEYYSFDDTPIIKALWRETKECVYIEPNHESSPNNNKGVFWYNNKF

SEQ ID NO: 109 - DNA, 945 bp, Cannabis sativa
  source 1..945: mol_type = other DNA
atgattgtggtcaccaagctttcagtgaccaccaatgggtggatgacaccgttggctttgtcctccactccgctcttctcttcccatacttctcttttaagtatagtcatcgtcgccatcattcaaacatcggctcccttgaacatgatcaattgtttgttccagtccccgaatctcaaatcgcatggctctacaaaacgttacttggacaatccactaggaagagccctaaagctttccactatagtgttccttggttttcctttgtacttaggtttcaatcttacaggcaaaccatatgatcgttatgcatgtcattatgatccttactctccactctactcaaaaagtgaaaggcttcatatattgatttcagatatcggtgttttcatcaccacattagtgttataccagcttggctcgactaaagggttgagttggcttgtgttcatgtatggggtgccattgtttacagggaatagcatccttgtgacaatcgcatacttgaatcatactcacccttcattgcctcattatgactcgtcagagtgggattggttgaaaggagcattgtcaacagttgatcgaaactatggatcaattctcaatagggtttttccatcaccttacagacgctcatgtagcacacatttattcgcaacaatacctcattaccatgcaaatgaagccaccaaagctatcaaacccatattgggagagtactactcttttgatgacactccaataattaaagctctttggagagagactaaggagtgtgtctatattgagccaaatcatgaatcttctcctaataataacaaaggtgtttttctggtacaacaacaaattctgatgatcatcaagttaaattataaatcaacatatgttttatgtgttttattaaaagtgtatgtgttattattgataagaataaaatattaa

SEQ ID NO: 110 - protein, 314 aa, Cannabis sativa
MIVVTKLSVTTNGWMTPLALSSTPLFSSHTSLLSIVIVAIIQTSAPLNMINCLFQSPNLKSHGSTKRYLDNPLGRALKLSTIVFLGFPLYLGFNLTGKPYDRYACHYDPYSPLYSKSERLHILISDIGVFITTLVLYQLGSTKGLSWLVFMYGVPLFTGNSILVTIAYLNHTHPSLPHYDSSEWDWLKGALSTVDRNYGSILNRVFPSPYRRSCSTHLFATIPHYHANEATKAIKPILGEYYSFDDTPIIKALWRETKECVYIEPNHESSPNNNKGVFLVQQQILMIIKLNYKSTYVLCVLLKVYVLLLIRIKY

SEQ ID NO: 111 - DNA, 471 bp, Cannabis sativa
  source 1..471: mol_type = other DNA
ggggggacaactttgtacaaaaaagttggcttgaatcatactcacccttcattgcctcattatgactcgtcagagtgggattggttgaaaggagcattgtcaacagttgatcgaaactatggatcaattctcaatagggttttccatcaccttacagacgctcatgtagcacaccatttattcgcaacaatacctcattaccatgcaaatgaagccaccaaagctatcaaacccatattgggagagtactactcttttgatgacactccaataattaaagctctttggagagagactaaggagtgtgtctatattgagccaaatcatgaatcttctcctaataataacaaaaggtgttttctggtacaacaacaaattctgatgatcatcaagttaaattataaatcaacatatgttttatgtgttttattaaaagtgtatgtgttattattgataagaataaaatattaa

SEQ ID NO: 112 - protein, 156 aa, Cannabis sativa
GGTTLYKKVGLNHTHPSLPHYDSSEWDWLKGALSTVDRNYGSILNRVFHHLTDAHVAHHLFATIPHYHANEATKAIKPILGEYYSFDDTPIIKALWRETKECVYIEPNHESSPNNNKRCFLVQQQILMIIKLNYKSTYVLCVLLKVYVLLLIRIKY

SEQ ID NO: 113 - DNA, 1695 bp, Cannabis sativa
  source 1..1695: mol_type = other DNA
atggaggtgttgaaggaagtagctaaggaaggatctgcagctcgtgagggagttgctattagagctgatcagaaaagttatagttataaacaactcatttcttctgcacagagtatatgcagcctgttatgtggcactgagttgaaggcaattcatgaagcaggcaaacaagctagaccttctgcgtcaatcaatggcgcgggtggtcatggacatcttggtggagctcgaataggaattgtagctaaaccttcagctgaatttgttgctggagtacttgggacttggctaagtggtggtgttgcagttccacttgcactcagctacccagaggctgaactcttgcatgtgatgaatgattcggacatctccatgatactgagtactgaagatcatcaagaacttatgcaaaaaattgctgcaaaaacatcagctcaattttctcttatcccgtccgttcccagtagttgttcacaggaagttgcagttgatcatcgacagactggagatataagcacagatagcattttgctcaatagggaaatctcaaatgaaaatcctgccttaattgtttacactagtggaacaacagggaaacctaaaggggttgttcatacgcatcaaagtattagcgcacaggtccaaacattggcaaaagcttgggagtatacacctgctgatcaatttttgcactgtttaccactacatcatgtccatgggctttttaatgctttatttgcacctctctatgctcgctccacggttgagttcttgccaaagtttagtgtacgaggtatctggcagagatggcgtgaatcatatccaacatctgaaactaaggccaatgattgtataaccgtatttacaggagtgccaacaatgtatactcgattgatacaaggttatgaagcaatggatccagagctgaaggaagcctctgcctcagctgcaaagcagttgcgtctaatgatgtgtggctcctctgcactccctgttcctgtcatgcaacaatggcaaaccattacaggacatcgtcttttggaacgatatggaatgacagaatttgtcatggccatatcaaatcccttaaaaggtgagaggaagtcaggcactgttggaaaaccattccctggtgtggaggtaaggattcttgcagaagatgagaatggagacgatgcaaccggggttggtgagctttgcgttagaagcccatccttatttaaagaatattggagacttcctgaggtaacaaaagcatcatttacagatgatgggttcttcaaaactggagatgccggtaaagtggatgaagatggatactatgtcattttgggacgtaccagtgctgatatcatgaaggttggtggatacaagttatctgccttagaaatcgaatctgtacttttagagcacccgactgtagaagagtgctgtgtgttgggcctacccgacaaagattacggtgaagctgtctctgcaataattgtgccagcagctgaagcaaagaagaaaagagaggaagaatcaagacctgctataagcttagaagaactcttcagttgggctcaacacaaacttgcaccatacaagctaccgactcgcctttttctgtgggactcgctacctcgtaacgctatgggaaaggtgaacaaaaaagagttgaagaaaaagttgaccgttgaacaaggcgtttga

SEQ ID NO: 114 - protein, 564 aa, Cannabis sativa
MEVLKEVAKEGSAAREGVAIRADQKSYSYKQLISSAQSICSLLCGTELKAIHEAGKQARPSASINGAGGHGHLGGARIGIVAKPSAEFVAGVLGTWLSGGVAVPLALSYPEAELLHVMNDSDISMILSTEDHQELMQKIAAKTSAQFSLIPSVPSSCSQEVAVDHRQTGDISTDSILLNREISNENPALIVYTSGTTGKPKGVVHTHQSISAQVQTLAKAWEYTPADQFLHCLPLHHVHGLFNALFAPLYARSTVEFLPKFSVRGIWQRWRESYPTSETKANDCITVFTGVPTMYTRLIQGYEAMDPELKEASASAAKQLRLMMCGSSALPVPVMQQWQTITGHRLLERYGMTEFVMAISNPLKGERKSGTVGKPFPGVEVRILAEDENGDDATGVGELCVRSPSLFKEYWRLPEVTKASFTDDGFFKTGDAGKVDEDGYYVILGRTSADIMKVGGYKLSALEIESVLLEHPTVEECCVLGLPDKDYGEAVSAIIVPAAEAKKKREEESRPAISLEELFSWAQHKLAPYKLPTRLFLWDSLPRNAMGKVNKKELKKKLTVEQGV

SEQ ID NO: 115 - DNA, 663 bp, Cannabis sativa; identical to SEQ ID NO: 105
  source 1..663: mol_type = other DNA

SEQ ID NO: 116 - protein, 220 aa, Cannabis sativa; identical to SEQ ID NO: 106

SEQ ID NO: 117 - DNA, 927 bp, Cannabis sativa
  source 1..927: mol_type = other DNA
atggccaaagatgctcccgctttcaatcctcatctcattgtctcacaaaaatttccagagaggacatatacctactccgagagagatgcaacgctctatgcgctaggtattggagcttgtgctagggatgccgtggatggtgacgaactgaaatatgtttatcatgaggacgggcagaagtttatacaggtattgccaacatttgctgctctattttccaatgggtctatatcattggggtctcttctaccaggattagaatatgatccacgccttctgctgcatggacaacaatacatagaattgtacaagccactgccctcaagtggtcgtatacaaaataaggttgctgtggcaggattgcatgacaaaggtaaagcagctatcgttgagattgaaacaaaaagttacaatgagtctggtgaattgttatgcatgaacaggttgactgcctttcttcgaggtgctggtggtttctcaagttcatctcaaccattttcttactcaaactatccagccaaccaggctccagcaattaaaattccagaacaacagccattcactgtgtttgaagattctacacaaccatctcaggcattactttataggctatccggtgactacaaccctctacattcagatcccgcgcttgcaaaagttgcaggattttcacagccgatactgcatggattgtgcacgcttggatttgcagttagagccatcattaaacgtatctgcaagggagatccaaacatggtgaagagcatatcaggacgattccttttgcatgtgtaccctggagaaacattgatcactgaaatgtggctacaaggcatgagggtcatatatcaaactaaggtgaaagaacgcaatcgggctgtgctatctggctttgttgatcttcgtaatttaacttcttccctgtaa

SEQ ID NO: 118 - protein, 308 aa, Cannabis sativa
MAKDAPAFNPHLIVSQKFPERTYTYSERDATLYALGIGACARDAVDGDELKYVYHEDGQKFIQVLPTFAALFSNGSISLGSLLPGLEYDPRLLLHGQQYIELYKPLPSSGRIQNKVAVAGLHDKGKAAIVEIETKSYNESGELLCMNRLTAFLRGAGGFSSSSQPFSYSNYPANQAPAIKIPEQQPFTVFEDSTQPSQALLYRLSGDYNPLHSDPALAKVAGFSQPILHGLCTLGFAVRAIIKRICKGDPNMVKSISGRFLLHVYPGETLITEMWLQGMRVIYQTKVKERNRAVLSGFVDLRNLTSSL

SEQ ID NO: 119 - DNA, 1215 bp, Cannabis sativa
  source 1..1215: mol_type = other DNA
atggctcagcaaatcttaaccccaaaaactctgcacccaaaatttggaaactatgtgacagtcctcagtatcgatggaggaggtattagaggaatgatccctggagttattcttgctcatcttgaagctgagcttcagagaatagatggagagcatgcaagaattgcggattactttgatgtaatttcaggaactagcacaggtggtcttatcacagccatgctaacagcaccaaacaaagagaataggcctatgtatgatgcaaaagaaattgtgcccttttaccatgaacactgccccaagattttcccacagccaagaactttacttggttgggctatggacacagtgaaagatttgacagggccaaaatatgatggaaaatatttgcatcaattgctgaaaaagttactaggaacaacaaggttgcatgacactttgacaaatgttgtcattcctactttcgatatcaagaagcttcagccagctatctttaccagttatgagacggtggatgatcataaagtgatggatgcacatctttcagacatttgcataggcacatctgcagccccaacttaccttcctgctcatcatttcacaaacaccgatgaaaatggaaaatctgaacgatttgatctcattgacggtggcgtctgcgctaataacccctctctggttgcgattagtgaagtgactaaaattatccttaagaaagatcccaactacacgacattcgcatcagaaaattttcgatttctgcttgtatcagtagggactggttcgaacagaggtgagaagaagtacgatgctaaatcatgtgccaagtggggacctctgtcgtggatagcacaccagggctctactcccatcactgatgttttcatggaggccagctctgacatggttgattaccataacactcttgttttcgaagcctttcgttctcaagatgaggtcctccgcatcgaggatgacacacttcatggagatttagcttcggtggataaggctaccccagaaaatttacgtaatctggaggaacttggtaagaaattactgaaaaagccagtttcacgtctcaatttgagaaccggaacccgtgaacctgttccaaatgctggtaccaacgaggaaggaatcagaaggtttgctgaggaactctcgaaggaaaagaagctgaggttgtcaaatgccaacgtggcagggagataa

SEQ ID NO: 120 - protein, 404 aa, Cannabis sativa
MAQQILTPKTLHPKFGNYVTVLSIDGGGIRGMIPGVILAHLEAELQRIDGEHARIADYFDVISGTSTGGLITAMLTAPNKENRPMYDAKEIVPFYHEHCPKIFPQPRTLLGWAMDTVKDLTGPKYDGKYLHQLLKKLLGTTRLHDTLTNVVIPTFDIKKLQPAIFTSYETVDDHKVMDAHLSDICIGTSAAPTYLPAHHFTNTDENGKSERFDLIDGGVCANNPSLVAISEVTKIILKKDPNYTTFASENFRFLLVSVGTGSNRGEKKYDAKSCAKWGPLSWIAHQGSTPITDVFMEASSDMVDYHNTLVFEAFRSQDEVLRIEDDTLHGDLASVDKATPENLRNLEELGKKLLKKPVSRLNLRTGTREPVPNAGTNEEGIRRFAEELSKEKKLRLSNANVAGR

SEQ ID NO: 121 - DNA, 486 bp, Cannabis sativa
  source 1..486: mol_type = other DNA
atgggtgttttcacttacgaatcagagttcacttcctcaattgctccagctaggcttttcaaggcctttgtccttgatggagacaatctcgtcccgaaaatctctcctcaagcagttgaaaaggttgaaattcttgaaggaaatggtggagttggaacaatcaagaagatcactttcggccaaggggttccattcaagtatgttaagcacaagatcgaggcgattgacaaggagagcctaacatactcttacagcataattgaaggagatgctttggaaggaaatcagcttgagaaaataacacatgaatcaaaattggttgcttcgggtgatggaggcaacgtaattaagactgttagcaagtactacagtgctggtgatgctcaagtgaatgaagagaaagttaaggaaggtgagaaacaagccactcaaatgctcaagactgttgaagcttacctcaaggaccaccctgaagcttacaactaa

SEQ ID NO: 122 - protein, 161 aa, Cannabis sativa
MGVFTYESEFTSSIAPARLFKAFVLDGDNLVPKISPQAVEKVEILEGNGGVGTIKKITFGQGVPFKYVKHKIEAIDKESLTYSYSIIEGDALEGNQLEKITHESKLVASGDGGNVIKTVSKYYSAGDAQVNEEKVKEGEKQATQMLKTVEAYLKDHPEAYN

SEQ ID NO: 123 - DNA, 600 bp, Cannabis sativa
  source 1..600: mol_type = other DNA
ggggggacaactttgtacaaaaaagttggggggacaactttgtacaaaaaagttggcaaacccatacattctctcaataaatttcccctttgttcattaatcaaacacaacaacatgggtgttttcacttacgaatcagagttcacttcctcaattgctccagctaggcttttcaaggcctttgtccttgatggagacaatctcgtcccgaaaatctctcctcaagcagttgaaaaggttgaaattcttgaaggaaatggtggagttggaacaatcaagaagatcactttcggccaaggggttccattcaagtatgttaagcacaagatcgaggcgattgacaaggagagcctaacatactcttacagcataattgaaggagatgctttggaaggaaatcagcttgagaaaataacacatgaatcaaaattggttgcttcgggtgatggaggcaacgtaattaagactgttagcaagtactacagtgctggtgatgctcaagtgaatgaagagaaagttaaggaaggtgagaaacaagccactcaaatgctcaagactgttgaagcttacctcaaggaccaccctgaagcttacaactaa

SEQ ID NO: 124 - protein, 199 aa, Cannabis sativa
GGTTLYKKVGGTTLYKKVGKPIHSLNKFPLCSLIKHNNMGVFTYESEFTSSIAPARLFKAFVLDGDNLVPKISPQAVEKVEILEGNGGVGTIKKITFGQGVPFKYVKHKIEAIDKESLTYSYSIIEGDALEGNQLEKITHESKLVASGDGGNVIKTVSKYYSAGDAQVNEEKVKEGEKQATQMLKTVEAYLKDHPEAYN

SEQ ID NO: 125 - DNA, 240 bp, Cannabis sativa
  source 1..240: mol_type = other DNA
tttttttttaataataatggtaagcaatattttcatattattcatatatattatatgataggaaaattaagatgtagagcaatatcatgtttcacaaaacattacagaatctcacaactctcacactcttattggcatataaagaaaaataaaaagccaagccaagccacacccacacaccttcataaccatttatataataataatagtagtattaaaatgtttattattatcatataa

SEQ ID NO: 126 - protein, 79 aa, Cannabis sativa
FFFNNNGKQYFHIIHIYYMIGKLRCRAISCFTKHYRISQLSHSYWHIKKNKKPSQATPTHLHNHLYNNNSSIKMFIIII

SEQ ID NO: 127 - DNA, 357 bp, Cannabis sativa
  source 1..357: mol_type = other DNA
atggcctcaaactcttcaagcatgaagctactctgcatggtggtcatggtcatggtggtagttgcaccaacgacacatgccctaacatgtggccaagtgtctagcagtcttagcccatgcatcaactacctcaagagcggcggtgctgtcccttcaccatgttgtaatgggatcaggtcgctgaacagtgcggccaagaccccagcggaccgcaagactgcatgcaagtgcttgcagtctgcggccggcagcatcaaaggactcaacttaaacctcgccgcgggacttcctggaaagtgtggtgtcaatgttccatacaagatcagtccctccaccaattgcaacagtgtgcagtga

SEQ ID NO: 128 - protein, 118 aa, Cannabis sativa
MASNSSSMKLLCMVVMVMVVVAPTTHALTCGQVSSSLSPCINYLKSGGAVPSPCCNGIRSLNSAAKTPADRKTACKCLQSAAGSIKGLNLNLAAGLPGKCGVNVPYKISPSTNCNSVQ

SEQ ID NO: 129 - DNA, 357 bp, Cannabis sativa
  source 1..357: mol_type = other DNA
atggcaagttcttcagttgtgaaagtactatgcttggtggtattggctatggtggtggcatcaccagtggtgcacgccaccataacctgtggccaggtgactagcagcctggcaccatgcttaagctacctaaaagttgggggagctgtcccggatgggtgttgtaatggtattaagtccttgagcggggcagctaaaaccccagccgatcgccaggctgcctgcaagtgcttgaagtcagctgctagcagtatcaaaggagtcaactttaaccttgcctctggacttccgggcaagtgtggcgttagcattccttacaaaattagcccatccaccgactgcagcagtgttcagtga

SEQ ID NO: 130 - protein, 118 aa, Cannabis sativa
MASSSVVKVLCLVVLAMVVASPVVHATITCGQVTSSLAPCLSYLKVGGAVPDGCCNGIKSLSGAAKTPADRQAACKCLKSAASSIKGVNFNLASGLPGKCGVSIPYKISPSTDCSSVQ

SEQ ID NO: 131 - DNA, 402 bp, Cannabis sativa
  source 1..402: mol_type = other DNA
atggcaatgtccttagctatgaagctcaccacttgtgtggtactcatgtgcatggttgttggtgcaccgctagcccaggccataacatgtggccaagtggctagctcggtagcaccatgcatcccctacttgagatctgggggtgctgtcccgacgggatgctgtaatggggttcgaagcctaaatagtcaggcctcgaacacagcaaaccgtcaagctgtgtgcaagtgcttggttagtgctgcaggaaattttcccggacttaaacctaatctcgtttctggcctcccttcggcttgtggggtttcccttccttttaaaatcagcacctcaaccaactgcaaacagcatcaagtgaatgaatgtgtagaaagaaagagagaggtattaataataaagtga

SEQ ID NO: 132 - protein, 133 aa, Cannabis sativa
MAMSLAMKLTTCVVLMCMVVGAPLAQAITCGQVASSVAPCIPYLRSGGAVPTGCCNGVRSLNSQASNTANRQAVCKCLVSAAGNFPGLKPNLVSGLPSACGVSLPFKISTSTNCKQHQVNECVERKREVLIIK

SEQ ID NO: 133 - DNA, 351 bp, Cannabis sativa
  source 1..351: mol_type = other DNA
atggcaagttcaattgtgaaggtactttgcttggtggtattggccatggtgatagtatcaccagtggcacatgccataacatgtggccaagtgtctagtagcattgcaccatgcttgggttacttaaggatgggcgggagtgtcccggctgggtgctgtagcggtattaggtcgttgagcaatgcagccaagacctctaccgaccgtcgagctgcctgcaactgcttgaaatcagctgcaaacggcatcaaaggaatcaactacaatcttgccagtggacttcccggcaagtgtggtgtcagcattccttacaagatcagcccctccaccaactgcaacagtgttcagtaa

SEQ ID NO: 134 - protein, 116 aa, Cannabis sativa
MASSIVKVLCLVVLAMVIVSPVAHAITCGQVSSSIAPCLGYLRMGGSVPAGCCSGIRSLSNAAKTSTDRRAACNCLKSAANGIKGINYNLASGLPGKCGVSIPYKISPSTNCNSVQ

SEQ ID NO: 135 - DNA, 408 bp, Cannabis sativa
  source 1..408: mol_type = other DNA
atgaagaacacgtcctttgtaacagtgtgtgtggtggcgatggtagccgcagcagtgctgttgtcggaagcgccgagtgcagaggcagtgacgtgcagcccactgcagatgagctcatgcctgtcggccatcactagtggatctccgccgtcgagtcaatgctgcgccaagctcaaggaacagaagccttgcctctgtgggtatctcaaggaccctaatcttcgccagtatgtcaactctcctaatgctagaatgttgctgccaagtgtggtgtttccttacccaaatgttgatccactactagtcgtgaacatcatcccttccaaattattattattaaataagacacttgttcactcctcttcaatactgcttacggaaaataaaatatatattatttgtgtttga

SEQ ID NO: 136 - protein, 135 aa, Cannabis sativa
MKNTSFVTVCVVAMVAAAVLLSEAPSAEAVTCSPLQMSSCLSAITSGSPPSSQCCAKLKEQKPCLCGYLKDPNLRQYVNSPNARMLLPSVVFPYPNVDPLLVVNIIPSKLLLLNKTLVHSSSILLTENKIYIICV

SEQ ID NO: 137 - DNA, 387 bp, Cannabis sativa
  source 1..387: mol_type = other DNA
atgaagaacacgtcctttgtaacagtgtgtgcggtggcgatggtagccgcagcagtgctgttgtcggaagcgccgagtgcagaggcagtgacgtgcagcccactgcagatgagctcatgcctgtcggccatcactagtggatctccgccgtcgagtcaatgctgcgccaagctcaaggaacagaagccttgcctctgtgggtatctcaaggacctaatcttcgccagtatgtcaactctcctaatgctagaaatgttgctgccaagtgtggtgtttccttacccaaatgttgatccactactagtcgtgaaacatcatcccttccatattattattataaataagacacttgttcacatcctcttcaatactgcttacggaaaataa

SEQ ID NO: 138 - protein, 128 aa, Cannabis sativa
MKNTSFVTVCAVAMVAAAVLLSEAPSAEAVTCSPLQMSSCLSAITSGSPPSSQCCAKLKEQKPCLCGYLKDLIFASMSTLLMLEMLLPSVVFPYPNVDPLLVVKHHPFHIIIINKTLVHILFNTAYGK

SEQ ID NO: 139 - DNA, 312 bp, Cannabis sativa
  source 1..312: mol_type = other DNA
atgcctgtcgggccatcactagtggatctccgccgtcgagtcaatgctgcgccaagctcaaggaacagaagccttgcctctgtgggtatctcaaggaccctaatcttcgccagtatgtcaactctcctaatgctagaaatgttgctgccaagtgtggtgtttccttacccaaatgttgatcccactactagtcgtgaacatcatcccttccatattattattataaataagacacttgtttcacgtcctcttcaatactgcttacggaaaataaaatatatattatttgtgtttgagtattttatatcataa

SEQ ID NO: 140 - protein, 103 aa, Cannabis sativa
MPVGPSLVDLRRRVNAAPSSRNRSLASVGISRTLIFASMSTLLMLEMLLPSVVFPYPNVDPTTSREHHPFHIIIINKTLVSRPLQYCLRKIKYILFVFEYFIS

SEQ ID NO: 141 - DNA, 837 bp, Cannabis sativa
  source 1..837: mol_type = other DNA
atgtttaaacatagaagtgattgtcaaagccagggcttctatagctacgaggctttcatcactgccactcaatcctttcctggctttgctactactggagatgtctcaactcgtaagagggagctcgctgctttctttgctcaaacctctcaactaaccactggctatggtcctagtgatccacatgcgtggggatattgtagtatcaatgggactgttcacacaacagctgggaatgattattgcacttcctctaagtggccttgtgctccaggcaaaaaatatattagcagaggacctatccaactcactcacaactacaactacggacttgccggtaaggctcttggagtagatttgataaaaaatccagatttggtggccatagaccctgttgtttcattcagaacagccttgtggttttggatgactaagcatgacagtaaaccagcttcttgccatgatattttgatcaatgctaattctaaaactaatgatcaagtctcctcaagctacgttgccatagataaaatagtaaacggcaattctcaaatagtccaaactgagctgggcgaaagtagcagagtttctaatagcattgggtactataaaagatattgtgatatgttagaagtgagttatgaaagctcatatgacaatatgtatgagaagaccctgtactacgtagctcaagaaagaaataaagtggaatggttctccccattttcatataggagattgagagatcatgtaatgtcaccttactatatgtatttgtgtcaagtgatcatatacatatgtaaagtgcttaaatatatatatatgctgataaaataa

SEQ ID NO: 142 - protein, 278 aa, Cannabis sativa
MFKHRSDCQSQGFYSYEAFITATQSFPGFATTGDVSTRKRELAAFFAQTSQLTTGYGPSDPHAWGYCSINGTVHTTAGNDYCTSSKWPCAPGKKYISRGPIQLTHNYNYGLAGKALGVDLIKNPDLVAIDPVVSFRTALWFWMTKHDSKPASCHDILINANSKTNDQVSSSYVAIDKIVNGNSQIVQTELGESSRVSNSIGYYKRYCDMLEVSYESSYDNMYEKTLYYVAQERNKVEWFSPFSYRRLRDHVMSPYYMYLCQVIIYICKVLKYIYMLIK

SEQ ID NO: 143 - DNA, 951 bp, Cannabis sativa
  source 1..951: mol_type = other DNA
atgaagtacttgagcagccttatcatcacactatgtttggctaatatattatgccttggattggtttatgcagatgaaagacccgatcaccaatgtgggcctaatctaggaaatcctccatgtggagacggaaggtgttgcagcatccataacttttgtggtagcggatctagttactgcagaggcggaaactgccgataccagtgctggttcgcagcagcaccgacgactggtactggtctccctcgtgataattctgtaaccaaaatagtcaccaaatcactctacaacgaaatgtttaaacatagaagtgattgtcaaagccagggcttctatagctacgaggctttcatcactgccactcaatcctttcctggctttgctactactggagatgtctcaactcgtaagagggagctcgctgctttctttgctcaaacctctcaactaaccactggctatggtcctagtgatccacatgcgtggggatattgtagtatcaatgggactgttcacacaacagctgggaatgattattgcacttcctctaagtggccttgtgctccaggcaaaaaatatattagcagaggacctatccaactcactcacaactacaactacggacttgccggtaaggctcttggagtagatttgataaaaaatccagatttggtggccatagaccctgttgtttcattcagaacagccttgtggttttggatgactaagcatgacagtaaaccagcttcttgccatgatattttgatcaatgctaattctaaaactaatgatcaagtctcctcaagctacgttgccatagataaaatagtaaacggcaattctcaaatagtccaaactgagctgggcgaaagtagcagagtttctaatagcattgggtactataaaagatattgtgatatgttagaagtgagttatggaaagctcatatga

SEQ ID NO: 144 - protein, 316 aa, Cannabis sativa
MKYLSSLIITLCLANILCLGLVYADERPDHQCGPNLGNPPCGDGRCCSIHNFCGSGSSYCRGGNCRYQCWFAAAPTTGTGLPRDNSVTKIVTKSLYNEMFKHRSDCQSQGFYSYEAFITATQSFPGFATTGDVSTRKRELAAFFAQTSQLTTGYGPSDPHAWGYCSINGTVHTTAGNDYCTSSKWPCAPGKKYISRGPIQLTHNYNYGLAGKALGVDLIKNPDLVAIDPVVSFRTALWFWMTKHDSKPASCHDILINANSKTNDQVSSSYVAIDKIVNGNSQIVQTELGESSRVSNSIGYYKRYCDMLEVSYGKLI

SEQ ID NO: 145 - DNA, 837 bp, Cannabis sativa; identical to SEQ ID NO: 141
  source 1..837: mol_type = other DNA

SEQ ID NO: 146 - protein, 278 aa, Cannabis sativa; identical to SEQ ID NO: 142

SEQ ID NO: 147 - DNA, 108 bp, Cannabis sativa
  source 1..108: mol_type = other DNA
atgtcaccttactatatgtatttgtgtcaagtgatcatatacatatgtaaagtgcttaaatatatatatgctgataaaataaatgagctacataaactttttacgtaa

SEQ ID NO: 148 - protein, 59 aa, Cannabis sativa
MSPYYMYLCQVIIYICKVLKYIYADKINELHKLFTYVPLNSFSYSFLIIIIKSIEVASG

SEQ ID NO: 149 - DNA, 201 bp, Cannabis sativa
  source 1..201: mol_type = other DNA
atggaaagctcatatgacaatatgtatgagaagaccctgtactacgtagctcaagaaagaaataaagtggaatggttctccccattttcatataggagattgagagatcatgtaatgtcaccttactatatgtatttgtgtcaagtgatcatatacatatgtaaagtgcttaaatatatatatgctgataaaataaatgag

SEQ ID NO: 150 - protein, 67 aa, Cannabis sativa
MESSYDNMYEKTLYYVAQERNKVEWFSPFSYRRLRDHVMSPYYMYLCQVIIYICKVLKYIYADKINE

SEQ ID NO: 151 - DNA, 345 bp, Cannabis sativa
  source 1..345: mol_type = other DNA
atggagggtggtatctatagaggcataggaaacgggacccaagtagatgggaaggttttgcagacatttcagaagaactttgttcaagttcaggacattttggaccagaaccggttgctaattagtgagattaaccagaaccatgagtcaaagataccagacaacttgaaccggaatgtgggactaatcagagaacttaacaacaacatcagaagggtggtagatctctatgctgatctctcaacttctttcaccaagtcggtggatgcttcatccgaaggcgaatcggctggtacttataaatcagatggaaaaactagtcagaagagaattagatccgggtag

SEQ ID NO: 152 - protein, 114 aa, Cannabis sativa
MEGGIYRGIGNGTQVDGKVLQTFQKNFVQVQDILDQNRLLISEINQNHESKIPDNLNRNVGLIRELNNNIRRVVDLYADLSTSFTKSVDASSEGESAGTYKSDGKTSQKRIRSG

SEQ ID NO: 153 - DNA, 285 bp, Cannabis sativa
  source 1..285: mol_type = other DNA
atgaaaattcaaaagatgcctcagcaggagctttcatcttcgatggtctctcagtcattcggtgttcctgatatgacattcaattcaattgactcgacaatcaacgatggcagcttcttgaacagggggccatgggcaccagcaccgccatttcaaagaatgcgaacatataccaaggtttacaaacgtggagctgtgggaaggtctatcgatattactcgctattcaggttatgatgagcttaaacaagatcttgctcgtagatttggtatcgaggacaattag

SEQ ID NO: 154 - protein, 94 aa, Cannabis sativa
MKIQKMPQQELSSSMVSQSFGVPDMTFNSIDSTINDGSFLNRGPWAPAPPFQRMRTYTKVYKRGAVGRSIDITRYSGYDELKQDLARRFGIEDN

SEQ ID NO: 155 - DNA, 621 bp, Cannabis sativa
  source 1..621: mol_type = other DNA
atggccatgtttgaaagaggagatgatgaactcaacctaaaccttgaggcaacagagcttaggctagggttaccaggctgtgatagtactactacagcaacttctactactcaagagtactctgagaacagcacaaatactacaaaaggcaacaacaagagatctttgccttctgatgaaacattcaaatctaactcttcatcagaaaccaaatcttttggtcaagaaaacccttcacccaccaagacacaagtagtgggatggcctccagtgagatcttaccgtaaaaactgcttacaatcaatgaaaaaagtagaggagagtggagtatacgtgaaagtcagcatggatggagctccttaccttagaaaaatggacttgaagctctacaaagggtacccagaactccttaaggccttggaagacatgtttaagtttaaagttggtgactttaacgagagagaaggctacaatggctctgaattcgtccctacttatgaagacaaagatggagattggatgttggttggagatgttccttgggaaatgtttacctcttcatgcaaaaggctgagaatcatgaaagggtcagaagctagaggcttgggttgttctgtttga

SEQ ID NO: 156 - protein, 206 aa, Cannabis sativa
MAMFERGDDELNLNLEATELRLGLPGCDSTTIATSTTQEYSENSTNTTKGNNKRSLPSDETFKSNSSSETKSFGQENPSPTKTQVVGWPPVRSYRKNCLQSMKKVEESGVYVKVSMDGAPYLRKMDLKLYKGYPELLKALEDMFKFKVGDFNEREGYNGSEFVPTYEDKDGDWMLVGDVPWEMFTSSCKRLRIMKGSEARGLGCSV

SEQ ID NO: 157 - DNA, 669 bp, Cannabis sativa
  source 1..669: mol_type = other DNA
cttagcatattggtattggctttggttttagtagtggtggccatcggtggagcaagggctgagcaatgcggacgccaagcgggcggcgttaaatgcccgaacgggctgtgctgcagccagcacgggtggtgtggcaacaccttcgattactgttcaccccaagctgggtgccagagcaattgttggggtggcccaacaccaccgagcggtgaaactgttgtaagagcaacataccatttctacaatccagcagagaacaactgggacttaatggctgttagtgctttttgctccacttgggatgccaataagcctttctcatggcgcagcaaatatggctggactgccttttgtggaccatccggacctaggggccgtgactcttgtggaaagtgtttgaggctaaggaatgtgggaactggggcaaccataacagcaagaattgtggatcagtgcagcaatggaggcttagacttggactttaacactatatttaagcctttagatactgatggaagaggatttcaacagggttctatgcaagtgggttatacatttgtcgattgtggtaatgagcttagtgaagatgagcaccagcaccctctgagctccattattgaggagaaaaactcaaggagtgtaattataaagctgttatggcttagttaa

SEQ ID NO: 158 - protein, 222 aa, Cannabis sativa
LSILVLALVLVVVAIGGARAEQCGRQAGGVKCPNGLCCSQHGWCGNTFDYCSPQAGCQSNCWGGPTPPSGETVVRATYHFYNPAENNWDLMAVSAFCSTWDANKPFSWRSKYGWTAFCGPSGPRGRDSCGKCLRLRNVGTGATITARIVDQCSNGGLDLDFNTIFKPLDTDGRGFQQGSMQVGYTFVDCGNELSEDEHQHPLSSIIEEKNSRSVIIKLLWLS

SEQ ID NO: 159 - DNA, 678 bp, Cannabis sativa
  source 1..678: mol_type = other DNA
ttgggtaagcttagcatattggtattggctttggttttagtagtggtggccatcggtggaacaagggctgagcaatgcggacgccaagcgggcggcgttaaatgcccgaacgggctgtgctgcagccagtacgggtggtgtggcaacaccttcgattactgttcaccccaagctgggtgccagagcaattgttggggtggcccaacaccaccgagcggtgaaactgttgtaagagcaacataccatttctacaatccagcagagaacaactgggacttaatggctgttagtgctttttgctccacttgggatgccaataagcctttctcatggcgcagcaaatatggctggactgccttttgtggaccatccggacctaggggccgtgactcttgtggaaagtgtttgaggctaaggaatgtgggaactggggcaaccataacagcaagaattgtggatcagtgcagcaatggaggcttagatttggactttaacactatattcaagcctttagatactgatggaagaggatttcaacagggttctatgcaagtgggttacacatttgtcgactgtggtaatgagcttagtgaagatgagcaccagcaccctctgagctccattattgaggagaaaaactcaaggagtgtaattataaagctgttatggcttagttaa

SEQ ID NO: 160 - protein, 225 aa, Cannabis sativa
LGKLSILVLALVLVVVAIGGTRAEQCGRQAGGVKCPNGLCCSQYGWCGNTFDYCSPQAGCQSNCWGGPTPPSGETVVRATYHFYNPAENNWDLMAVSAFCSTWDANKPFSWRSKYGWTAFCGPSGPRGRDSCGKCLRLRNVGTGATITARIVDQCSNGGLDLDFNTIFKPLDTDGRGFQQGSMQVGYTFVDCGNELSEDEHQHPLSSIIEEKNSRSVIIKLLWLS

SEQ ID NO: 161 - DNA, 717 bp, Cannabis sativa
  source 1..717: mol_type = other DNA
atgaggcctgcttctcctgctggccctccattacctgttgtactgcctgcatcaacaggtcaaggtttgcagatcagtgctcaactgacccgaagggatggtcaaatattttacaatttattgtttgagaacaatacacagactcctcttgatggcttcatgattcagttcaacaaaaatacttttggtcttgctgctgctggacccttgcaggttcccccattgcaacctgggtcatctgtgagaactcttttgccaatggttgttttccaaaatatgtctcaaggtcccccaagctcacttcttcaggttgctgtgaaaaacagtcaacaacctgtgtggtatttcaatgataaaatccaattgcatgtattcttcaccgaggaagggagaatggagcgtgcaaactttctcgagacatggagatcccttccggactcaaacgaggtctcaagggacttccctaacgcggtgattagctctgtcgaagcaactctagaccagctagctgcttcgaacatgttctttattgcaaaacgcaagcatgcaaaccaggatgttttctacttctctgctaaactccctcaaggagtacctttcttgatcgaactcaccacagtcgtcgggaaccccggagtgaagtctgcaatcaagactccaaaccctgaaatggcaccacttttctttgaatccattgaggctctcctgagggtttga

SEQ ID NO: 162 - protein, 312 aa, Cannabis sativa
AEGSEAGYSESPAHVTDSAAAASPPTSSGNVPYATARQSSPIPAAQTQPTPAAAPVPDLLGDLMGLDNSAIVPVDEPASPAGPPLPVVLPASTGQGLQISAQLTRRDGQIFYNLLFENNTQTPLDGFMIQFNKNTFGLAAAGPLQVPPLQPGSSVRTLLPMVVFQNMSQGPPSSLLQVAVKNSQQPVWYFNDKIQLHVFFTEEGRMERANFLETWRSLPDSNEVSRDFPNAVISSVEATLDQLAASNMFFIAKRKHANQDVFYFSAKLPQGVPFLIELTTVVGNPGVKSAIKTPNPEMAPLFFESIEALLRV

SEQ ID NO: 163 - DNA, 732 bp, Cannabis sativa
  source 1..732: mol_type = other DNA
gattacccactcgttactcaggatgatcttcttcacattttgtcttacttgccaaaaagtctcaactttgtgagtcattcaagctacattggctggagagagtctaagaagttaaaacccataattgtagatccagggctttatttatcagagaaatcatcaatgttttatgcttcacaaaagagggaactgcctaatgcttataaactatttacaggttcatcgtttgccatttacagccgtaatcttattgaattctgcattctaggtacggataacctccctagaactctgttgatgtatttttccaacacaccttcagcactttccagctacttccccaccattttatgcaattctcgacagttcaacaaaaccattgtaaaccacaacctacaatatgccaacttcgacaaacccccaaaggaggagccccgtaaggtcattcccgatgactttgatccaatgattcaaagtggggcagccttcgcttctaaattcaacctgaatgatccattacttgaccgtatcgaccaagaaattttaagccgaggcagaggtgatgtcgtgcctggtggctggtgcttaggtgagcctagaaacagcacgtgctcgatatggggagatgctgatgttctgaggcctggtctaggtgcgaaaagactcgagaaacttattgttaagctgctctctaacggtacctcaacaactaatagatgcatatttgaatga

SEQ ID NO: 164 - protein, 243 aa, Cannabis sativa
DYPLVTQDDLLHILSYLPKSLNFVSHSSYIGWRESKKLKPIIVDPGLYLSEKSSMFYASQKRELPNAYKLFTGSSFAIYSRNLIEFCILGTDNLPRTLLMYFSNTPSALSSYFPTILCNSRQFNKTIVNHNLQYANFDKPPKEEPRKVIPDDFDPMIQSGAAFASKFNLNDPLLDRIDQEILSRGRGDVVPGGWCLGEPRNSTCSIWGDADVLRPGLGAKRLEKLIVKLLSNGTSTTNRCIFE

SEQ ID NO: 165 - DNA, 975 bp, Cannabis sativa
  source 1..975: mol_type = other DNA
atgcatttcgactattgtacaaccgcgagcattgacttcatcttcttagaagaggcgaattttagcaatatcatacggcatccagatttgactgtaacatctgaagaaaaagttcttgatgcaatcttaatgtggggtatgaaagcgaagaacgcatatggatgggaggtggtggatgagctgatgatatattcgacgcctgaacttctctttggggacagactagagtcggttttcaatttcttatcttctgtgcgatttccaatcatgcctcttgtcctgctcaagaagttggagaagagcaacatcagcaggcacattcctagtttcgattttcttgtgaaagaggccatcagctatgttgaacatggattgtgtgagcctgaatttgatcaaaatgttagatttcaactaagaagatctagttataaggagcttcagtacttgtgtgatggagatagtaatggagttctttacttcgcgggcacatcatacgggcaacatcagtgggttaatcctgttctcgcgaaaacaatcacaattacagctagcagccccccttcaagattcaccgactccaaagttttggcgtctagaacttaccagggaacatcttttgctggaccaaggatagaagatggacatagtagcgcgtggtggatggttgacatcggccccaaccatcagcttatgtgcaactactacaccttaaggcaggatggatccagggcttacaacagatattggaatctacagggatctttggatgggaaaacatggacaaatttgagagtgcatgaaaatgatcaaacagtatgcaaaccagggcagtttgcatcgtggcccataataggaccaaatgctctgcgaccatttaggttctttagggttgttctaacgggtcctactactgatgtatccaaccccttcaacatttgcatttgctttttggagctctatggctttttccattag

SEQ ID NO: 166 - protein, 530 aa, Cannabis sativa
MILSLEFEVMTLVKQCEEIIQQFKINDKLFDSGKRVELSYPNTSPHCYRAFPFGLAVDKKRLKQLYSSGEYSDVDIYIEGGGLVSQPHKIILSLWSVPFAKMFTNGMSETNSSRVVLRDVSPEAFEGMLDFMYSGEVNLENTMESGAMLLQMLLLSDQFGVTLLHQECCKILLECLSEDTVCPILQAVSSIPSCKLIEETCERNFSMHFDYCTTASIDFIFLEEANFSNIIRHPDLTVTSEEKVLDAILMWGMKAKNAYGWEVVDELMIYSTPELLFGDRLESVFNFLSSVRFPIMPLVLLKKLEKSNISRHIPSFDFLVKEAISYVEHGLCEPEFDQNVRFQLRRSSYKELQYLCDGDSNGVLYFAGTSYGQHQWVNPVLAKTITITASSPPSRFTDSKVLASRTYQGTSFAGPRIEDGHSSAWWMVDIGPNHQLMCNYYTLRQDGSRAYNRYWNLQGSLDGKTWTNLRVHENDQTVCKPGQFASWPIIGPNALRPFRFFRVVLTGPTTDVSNPFNICICFLELYGFFH

SEQ ID NO: 167 - DNA, 1122 bp, Cannabis sativa
  source 1..1122: mol_type = other DNA
atggggttctacttcccatacaatatcctaccttttcttactttgttgtttgtagcaagccatgccgtgcaatctccagaactttattggaagtctgttctgccaaacacacctatgccaaaagctgttacaaatcttctacaatcagatatattaagggaagagaagagtacctctgtgaataatgttgataacaaaggaagtagcaacgttgatgttggtgttggtgttggtaacaaaggaggtaccaacgttggtgttggtgttggtgttggtaacaaaggaggtaccaacgttggtgttggtggtaaagaaggaggagtttctgtaaacacaacacataagggaaaacctgtttatgttggagtaggtgtgaaaggaaaagacccatttgtgtacaagtatgcagccaatgaaactcaacttcatgacaatcctagtgtagctctgtttttcttggacaaggacattcaacctgggactcaaatgaacttgaacttcgtcaagagtagtactagtatgagtagtacttcaactttcttgccccgtcaagtagctcaatcaatcccattctcatctaaaaaattgcctgaaattttgaacaaactttctgtgaaaccacactcagaggaggctgagatggtgaaacaaacaatcgaagagtgtgaagagccgagtatcaaaggtgaagtcaagtattgtgcaacttcattagagtcaatgatagacttcaccacttccaagcttggtaacaacaacaacgtacaagcaatttctacaaatgtagtagaggagaaagaatctgttgggatacaaaagtacacaataaaacaagtagaaaagaagagaaactcaaactccaaagtaaccgttgtgtgccacaagctaaactatcccttcgcagtcttctattgccacacttcacaagccactgaggcttatatggtgaccttacaaggtagtgatgatgggaacaaagttaaggcagtagctgcctgccataaagacacctcaggatggaacccaaaccacttggccttcaaagtccttaacgttaaacaaggaactgttccagtctgccatttccttcctcaggatcatattgtttggattcaaaactag

SEQ ID NO: 168 - protein, 373 aa, Cannabis sativa
MGFYFPYNILPFLTLLFVASHAVQSPELYWKSVLPNTPMPKAVTNLLQSDILREEKSTSVNNVDNKGSSNVDVGVGVGNKGGTNVGVGVGVGNKGGTNVGVGGKEGGVSVNTTHKGKPVYVGVGVKGKDPFVYKYAANETQLHDNPSVALFFLDKDIQPGTQMNLNFVKSSTSMSSTSTFLPRQVAQSIPFSSKKLPEILNKLSVKPHSEEAEMVKQTIEECEEPSIKGEVKYCATSLESMIDFTTSKLGNNNNVQAISTNVVEEKESVGIQKYTIKQVEKKRNSNSKVTVVCHKLNYPFAVFYCHTSQATEAYMVTLQGSDDGNKVKAVAACHKDTSGWNPNHLAFKVLNVKQGTVPVCHFLPQDHIVWIQN

SEQ ID NO: 169 - DNA, 882 bp, Cannabis sativa
  source 1..882: mol_type = other DNA
gggagcaacgaaaaaaaaatcacatctttactaaattccattaccaaaatccaatgtccggatacccacaaccgcccggaggatacggctacggcgggaggccgccgccgcaagccgcccaaccatacgccgcgccgccatacggcgctcctccgccaccacaagatcatcagcaccatcaccaacagccttcaccagcttacgcgagtccctacgctcagccatcggctccttacaatccagataagccaccaaaggacagctactctccgtacggcggaagctccggagggtagcgcgtatccaccaccgcaggcgtacggaaccccattcgcttcgcttgtgccttcgactttccctcccggtacggatcccaacgtggtggcttgctttcaggtggctgatcaagacggaagtggcttaatcgacgataaggagttgcagagagctctctcttcgtataatcagagttttagtctcagaacggttcatcttcttatgtatcttttcaccaattccaatagcaagaagatcggtccaaaggaattcactgatgttttctacagtcttcagagttggaggggaatatttgagcgatttgatagagacaggagtggcagaatcgacatgaatgagttgagagaagcacttttgagtctgggattttctgtttcaccagtggttttggatttactggtctccaaattcgacaaaactggtggcaaagtcaaagccattgaatatgataatttcattgagtgctgtctcactgttaagggattaactgagaaatttaaggagaaggacaccaagtactctggttctgcaactttcagttatgaagctttcatgctcactgtacttcctttcctcatagcttaa

SEQ ID NO: 170 - protein, 293 aa, Cannabis sativa
GSNEKKITSLLNSITKIQCPDTHNRPEDTATAGGRRRKPPNHTPRRHTALLRHHKIISTITNSLHQLTRVPTLSHRLLTIQISHQRTATLRTAEAPEGSAYPPPQAYGTPFASLVPSTFPPGTDPNVVACFQVADQDGSGLIDDKELQRALSSYNQSFSLRTVHLLMYLFTNSNSKKIGPKEFTDVFYSLQSWRGIFERFDRDRSGRIDMNELREALLSLGFSVSPVVLDLLVSKFDKTGGKVKAIEYDNFIECCLTVKGLTEKFKEKDTKYSGSATFSYEAFMLTVLPFLIA

SEQ ID NO: 171 - DNA, 456 bp, Cannabis sativa
  source 1..456: mol_type = other DNA
ggggggacaactttgtacaaaaaagttggtcacatttcgcttaagcatctcccccaaaaaaacaatatcaaaatggcaaccacaatggctggcatgagcctctcggccccaagaatcgtagcaggagtagactcagtgaaggtccagcccttcaagcccgccacgtggctgaaccagccatggaagaggagtactagcggccgcttggcttccgtacgtcccttgttcgcggcgccagatcagatatctgagaaggtagccgagagcatcaagagcgccgaaagcgcttgctctgacaacccggctagcggcgagtgcgtggcggcgtgggacgaagtggaggagctgagcgccgcggctagccacgccagggacaagaagaagacttctgaccccttggaggagtactgtaaggacaacccagaaactgatgagtgccgtacttatgaagattga

SEQ ID NO: 172 - protein, 151 aa, Cannabis sativa
GGTTLYKKVGHISLKHLPQKNNIKMATTMAGMSLSAPRIVAGVDSVKVQPFKPATWLNQPWKRSTSGRLASVRPLFAAPDQISEKVAESIKSAESACSDNPASGECVAAWDEVEELSAAASHARDKKKTSDPLEEYCKDNPETDECRTYED

SEQ ID NO: 173 - DNA, 972 bp, Cannabis sativa
  source 1..972: mol_type = other DNA
atggaagttaacaaattaatatttgttacgagaatatttttgttatttcttggtttgacatccacattggcacatggccaaggaaccaaagttgggtattactcaacaaagtgccctaaggccgagtctattgttagatcaacagttgagtctcatttcaaaacaaatcctcggattgccccgggcattctgaggatgcacttccacgattgttttgttcgtggttgcgacgcttctatcctcatcgaaggctccaacaccgagaagacagcagtacctaaccgtgctatacccggctataatgttatagacgacgccaagaagcagctcgaggctgcttgccctggcgtcgtttcttgcgctgatattattgctcttgctgctcgcgactctgtagttgtgaccaaagggattaattggaacgttcctactgggcgtagagatggacgagtttcaattgcttcggaggctggggatttgcctggtcctaatgaatcagtgtttaatcaaacaaggaagtttaagaacataaatctcagcgttcaagacctggtcactctagttggtggacatacaattggaacagcagcatgcggtttagtgacagggaggctatacaacgtgaattcaagcaatggtgctgatccatccatcgaccctacctttgtccctacactaagagctctctgcccacaaaatggcggcggttcgaaccgagttgatctcgacacgggaagcggctctcgttttgacaacagctacttcaacaacttgagcaagagacgaggcattctcacctcggatcagctgctgtggaccgatccctcaaccaagggctttgtcctccaatatttgaggagtgccacctctttcaactttgagtttgctaagtccatggttaagatgagtaaagttggtgtcttgactggtactcagggtgaaattcgaaaactttgtactgctattaattaa

SEQ ID NO: 174 - protein, 323 aa, Cannabis sativa
MEVNKLIFVTRIFLLFLGLTSTLAHGQGTKVGYYSTKCPKAESIVRSTVESHFKTNPRIAPGILRMHFHDCFVRGCDASILIEGSNTEKTAVPNRAIPGYNVIDDAKKQLEAACPGVVSCADIIALAARDSVVVTKGINWNVPTGRRDGRVSIASEAGDLPGPNESVFNQTRKFKNINLSVQDLVTLVGGHTIGTAACGLVTGRLYNVNSSNGADPSIDPTFVPTLRALCPQNGGGSNRVDLDTGSGSRFDNSYFNNLSKRRGILTSDQLLWTDPSTKGFVLQYLRSATSFNFEFAKSMVKMSKVGVLTGTQGEIRKLCTAIN

SEQ ID NO: 175 - DNA, 1014 bp, Cannabis sativa
  source 1..1014: mol_type = other DNA
atggaagttaacaaattaatatttgttaccaggatatttttgttatttcttggtttgacatccacattggcacatggccaaggaaccaaagttgggtattactcaacaaagtgccctaaggccgagtctattgttagatcaacagttgagtctcatttcaaaacaaatcctcggattgccccgggcattctgaggatgcacttccacgattgttttgttcgtggttgcgacgcttctatcctcatcgaaggctccaacaccgagaagaccgcagtacctaaccgtgctatacccggctataatgttatagacgacgccaagaagcagctcgaggctgcttgccctggcgtcgtttcttgtgctgatattattgctcttgctgctcgcgactctgtagttgtgaccaaagggattaattggaacgttcctactgggcgtagagatggacgagtttcaattgcttcggaggctggggatttgcctggtcctaatgaatcagtgtttaatcaaacaaggaagtttaagaacataaatctcagcgttcaagacctggtcactctagttggtggacatacaattggaacagcagcatgcggtttagtgacagggaggctatacaacgtgaattcaagcaatggtgctgatccatccatcgaccctacctttgtccctacactaagagctctctgcccacaaaatggcggcggttcgaaccgagttgatctcgacacgggaagcggctctcgttttgacaacagctacttcaacaacttgagcaagagacgaggcattctcacctcggatcagctgctgtggaccgatccctcaaccaagggctttgtcctccaatatttgaggagtgccacctctttcaactttgagtttgctaagtccatggttaagatgagtaaagttggtgtcttgactggtactcagggtgaaattcggaaagttgtactgctattaactaataacaatatgttttttaatatatatatatatatggtcaaataa

SEQ ID NO: 176 - protein, 337 aa, Cannabis sativa
MEVNKLIFVTRIFLLFLGLTSTLAHGQGTKVGYYSTKCPKAESIVRSTVESHFKTNPRIAPGILRMHFHDCFVRGCDASILIEGSNTEKTAVPNRAIPGYNVIDDAKKQLEAACPGVVSCADIIALAARDSVVVTKGINWNVPTGRRDGRVSIASEAGDLPGPNESVFNQTRKFKNINLSVQDLVTLVGGHTIGTAACGLVTGRLYNVNSSNGADPSIDPTFVPTLRALCPQNGGGSNRVDLDTGSGSRFDNSYFNNLSKRRGILTSDQLLWTDPSTKGFVLQYLRSATSFNFEFAKSMVKMSKVGVLTGTQGEIRKVVLLLTNNNMFFNIYIYMVK

SEQ ID NO: 177 - DNA, 1035 bp, Cannabis sativa
  source 1..1035: mol_type = other DNA
atggaatcaaagtcctcagcctcactaaacaagaaagggttgatgagggaatggtggaatcccttgttgttcacagctactattgcctcagccaaatgtttctttacccatgttttaaggaaaaatactctaacacaagctcgcagaaacatctctcgtcattacgacctgagtaatgaactctttgctctattcttggatgagacaatgcaatactcttgtgctatatttaagtcaattgatgaagacttgaagaatgcacaaatgcgaaaaatgttaattctaattgaaaaagcaagaattgataaaagtcatgaagttcttgatattgggtgtggttggggaagtttgggtattgaaattgtcaaaagaactggatgcaattacactggcatcactttatctgaggagcaattgaaactagcacaaaagaaagctagagatgctggccttcaagaaaacattaaatttcttctttgtgactatcgacaattgcctgctacctacaaatgtgacagaatcatatcttgtgggatgttagaacatgttggccacaaatttattgaagagttttttagttgttgtgagtctgttttagcagagaatggccttgttgttctacagtttgtatcaatgcccgatgatcgatactatgagtacataagaagtccagggtttataaacgaatacattttccctggtggaagtctaccttcattgagtagagtaacatcagccatggcttcttcatctacactctgtgtggagcacgttgaaaatattggaattcattactaccaaacactgaggatttggagaaagaattttctagaaaaccaaagtaaaattaaggcccttggattcaatgacaagtttattaggacttgggagtactattttgattattgtgcatcgggtttcaaatcacgcactattggtgattatcagattgtgttttctcgtcctggcaatgtaacaacttttaccaatccatataggggctttccttctgcctatggggatgactttaattaa

SEQ ID NO: 178 - protein, 380 aa, Cannabis sativa
MMEADLGLADAYINGDFTLVDKDEGLLNLFLIFISNMESKSSASLNKKGLMREWWNPLLFTATIASAKCFFTHVLRKNTLTQARRNISRHYDLSNELFALFLDETMQYSCAIFKSIDEDLKNAQMRKMLILIEKARIDKSHEVLDIGCGWGSLGIEIVKRTGCNYTGITLSEEQLKLAQKKARDAGLQENIKFLLCDYRQLPATYKCDRIISCGMLEHVGHKFIEEFFSCCESVLAENGLVVLQFVSMPDDRYYEYIRSPGFINEYIFPGGSLPSLSRVTSAMASSSTLCVEHVENIGIHYYQTLRIWRKNFLENQSKIKALGFNDKFIRTWEYYFDYCASGFKSRTIGDYQIVFSRPGNVTTFTNPYRGFPSAYGDDFN

SEQ ID NO: 179 - DNA, 480 bp, Cannabis sativa
  source 1..480: mol_type = other DNA
tggaaaaacctatcttgtcttcgtttcattcttgtaagcgaaaagattcaaactttccatcagtttggccggaaaatcaacaacccttttactcaaatccacagaatttgggtttgtaagatgatagcagttgtagtagctgtggttctggctttacttttgggagcttttgttttgattcccagacccccaaaatctggtcatacaaagaatgatcagtcaaatgttaaactcaataagggaacaaaactatacaccaaatcagaagtctctttgcacaacaagggaactgattgttggatcatcattaaagacaaggtatatgatgttacatcgtatgttgaggaacaccccggtggtgatgccattcttgcacatgccggggatgattcaaccgaaggcttttacgggccacaacatgcaactcgagttttcgatatgatcgaagacttttacataggcgatttggagaagtcataa

SEQ ID NO: 180 - protein, 159 aa, Cannabis sativa
WKNLSCLRFILVSEKIQTFHQFGRKINNPFTQIHRIWVCKMIAVVVAVVLALLLGAFVLIPRPPKSGHTKNDQSNVKLNKGTKLYTKSEVSLHNKGTDCWIIIKDKVYDVTSYVEEHPGGDAILAHAGDDSTEGFYGPQHATRVFDMIEDFYIGDLEKS

SEQ ID NO: 181 - DNA, 780 bp, Cannabis sativa
  source 1..780: mol_type = other DNA
ttggccaacttttttgtacaaagttgtccccgaattattgagggtgcaaaggatggaaagggtagaggaaggcaggttataagcactgctaggacttgcaattgcattttaattgttcttgatgcaataaagccaataactcataagcgcctgatagagaaagagcttgagggatttggcataaggttgaacaaagaaccacctaatctaacgttcagaaagaaagacaaaggtggaattaattttacctcaacagttactaacacacatctggaccttgatacagtgaaggcaatttgtagtgaatacaggattcacaatgctgatatcactcttaggtttgacgcaactgctgatgatctcatagacgtaattgagggaagtaggatatacatgccttgcatctatgttgtgaacaagatcgatcagattactcttgaagagttggaaattttggataaacttccacattattgtccaattagtgctcacttggagtggaacctcgatggtttgcttgataaagtatgggagtatcttaatttaactcgaatttacacaaaacctaaagggatgaatcctgattacgaagaccctgtaattctatcatccaagaggagaacagttgaggatttctgtgagcgaattcacaaggacatgcttaaacagtttaaatatgctctggtttgggggtctagtgcgaaacacaagccccagagagttggcaaggagcacgagttggaagatgaagatgttgtccagatcataaaaaaggtgtga

SEQ ID NO: 182 - protein, 259 aa, Cannabis sativa
LANFFVQSCPRIIEGAKDGKGRGRQVISTARTCNCILIVLDAIKPITHKRLIEKELEGFGIRLNKEPPNLTFRKKDKGGINFTSTVTNTHLDLDTVKAICSEYRIHNADITLRFDATADDLIDVIEGSRIYMPCIYVVNKIDQITLEELEILDKLPHYCPISAHLEWNLDGLLDKVWEYLNLTRIYTKPKGMNPDYEDPVILSSKRRTVEDFCERIHKDMLKQFKYALVWGSSAKHKPQRVGKEHELEDEDVVQIIKKV

SEQ ID NO: 183 - DNA, 2076 bp, Cannabis sativa
  source 1..2076: mol_type = other DNA
atgttgctaaagtgtggaggaaagggtttagtaaccactcgtagtattaaggttgctaggataactcagagtacagttgagccaattgaagtaaagggtttgagcatagacatgtcttggttattatttaaaagaatggcatttaaaaaagctcaagagccggtggagggttcaaaagaaatggagctagggatggaggttgttgaaaaatgtaagggagttcctttggccataagaactataggaagattattgcaggaaaggttatggcagtctggagacccacaaaaggagttgtcaaggtttttaaatactgagtttgctaaaatagatcaatatgaaaacgatatcatgcccaccctcaagctaagttataatcatcttccgtcacatttgaagcattgttttgcttactgtagattgttcccaaaagatcatgaaattgatgtgatagagttgatacaactatggatggcacaaggatttataaaatactctgatgacaaaggccaatgtctagaagaaattggttatgagtattttataaatctactttggagatcctttttccaagagccaaggactgataaattaggttatataaggacatgcaaaatgcatgaccttatgcatgatcttgcaattaaggtggcaggatcaagtagtgtcataatcaatcagaattctaaaatttctgatgaaaaatgtctccatgtgtcattagtaaattttaatcttgaaaccaaaacctcactcgagattttcgaattcttttctcaacaaaaaagacttcgaactttacttttacataattgtgaagggttgagcttctcaaaacattttttgtccaagtataagtgtttacgagctttgagtgtacctaccttggaaattaaggtgttgccaaggaatctgtgtgaattggaacatttgagatatcttaatctttcttgtaatgacaaattaaaaacacttcctagctctttggtaagattacagaatttgcagactctgaatctagcatggtgtagaaatcttgtacgattacctcaaaacatgaagaaattagtcaacttaaggcatcttatgattaaacattgttatagtttgagtgagatgccccctgaatttgggtatatgagttcccttcacacattagatacatttgttgcatctgagttgagtgggtttcatgaattgagcaaccttattaataacttagaaggaaacttaacaattaaaggactcagtcacacacaaaaaacaaaccattctgcatcaaaaataacttcaaagcgagtcatttgtgaaggaaagcatagtcttcaatttttaaagttaaaatggacatatatgaatagagacgaaatgtcacttgaaggtttgcagccaccttccagcctaaaagagttgaaggtgtcatgtttcaatggtgtgacatttggaggttgggattcctcatggaataagcttgttaaattgaagttgtgggtttgtaacaggtgccaatatctcccaaaattagataaattatgttctcttaaggagcttgagattcgttgggttaaagcagagtacatgtgctcctctgtagaagatgatgattttgatgccaactcgttctttccatccctggtaactcttcaaatttatatgtgtccaaatctcaagtggtggtggtggaaaaaggaagtggatgggaataatattagatcatttcctcgtctttctaatctaacaatcatcaactgtcctaacctcacttcaatgcctttgtttccatctattgaaaaagttaaacttcgtagaagtagctcaatggcattagaagatacgtttaagatgatgagcaaaaggaaggcacacaacactcacccaaacatttcattgctcaagtctatcaagatcatttactgtcaagatttgacatcaatactggtggaagggattgagagccttccctccctagagtatatgcgtataaccaactgccccaatttgacatcaattcccaacaacttcagaaaagtgaagaattctactatttcagattgtcctcatcttgaacagagataa

SEQ ID NO: 184 - protein, 691 aa, Cannabis sativa
MLLKCGGKGLVTTRSIKVARITQSTVEPIEVKGLSIDMSWLLFKRMAFKKAQEPVEGSKEMELGMEVVEKCKGVPLAIRTIGRLLQERLWQSGDPQKELSRFLNTEFAKIDQYENDIMPTLKLSYNHLPSHLKHCFAYCRLFPKDHEIDVIELIQLWMAQGFIKYSDDKGQCLEEIGYEYFINLLWRSFFQEPRTDKLGYIRTCKMHDLMHDLAIKVAGSSSVIINQNSKISDEKCLHVSLVNFNLETKTSLEIFEFFSQQKRLRTLLLHNCEGLSFSKHFLSKYKCLRALSVPTLEIKVLPRNLCELEHLRYLNLSCNDKLKTLPSSLVRLQNLQTLNLAWCRNLVRLPQNMKKLVNLRHLMIKHCYSLSEMPPEFGYMSSLHTLDTFVASELSGFHELSNLINNLEGNLTIKGLSHTQKTNHSASKITSKRVICEGKHSLQFLKLKWTYMNRDEMSLEGLQPPSSLKELKVSCFNGVTFGGWDSSWNKLVKLKLWVCNRCQYLPKLDKLCSLKELEIRWVKAEYMCSSVEDDDFDANSFFPSLVTLQIYMCPNLKWWWWKKEVDGNNIRSFPRLSNLTIINCPNLTSMPLFPSIEKVKLRRSSSMALEDTFKMMSKRKAHNTHPNISLLKSIKIIYCQDLTSILVEGIESLPSLEYMRITNCPNLTSIPNNFRKVKNSTISDCPHLEQR

SEQ ID NO: 185 - DNA, 702 bp, Cannabis sativa
  source 1..702: mol_type = other DNA
atgccatggactgtggaaggaatcacccccgatattattgtgaacccacatgctattccttctcgaatgacaatcggtcagcttattgagtgtatcatgggaaaagttgctgctcatatggggaaggaaggagatgcgactccgtttacagacgtaactgtggataatattagtaaagcacttcataaatgtggttatcaaatgcgtggattcgaaaccatgtacaacggtcacactggccggcgtcttcctgctatgatcttcctcggaccaacgtattaccaaagattgaagcatatggtggatgataagatccattcaaggggacgaggccccgtgcagattcttacaaggcagcctgctgaaggacgatctcgtgatggaggtcttcgctttggagaaatggaacgggattgtatgattgctcacggggcagctcactttctcaaagagagattgttcgatcagagtgatgcctatagagttcatgtctgtgagcgttgtgggttgatagcaattgcaaatcttaagaagaattctttcgagtgcagaggttgcaaaaacaaaaccgacattgtacaggttcatattccttatgcctgtaagctgctattccaagagcttatggccatggccatagctccgagaatgctgacaaaggaagtaaaaccattgaaagacctaaagaagaaaggaggctga

SEQ ID NO: 186 - protein, 233 aa, Cannabis sativa
MPWTVEGITPDIIVNPHAIPSRMTIGQLIECIMGKVAAHMGKEGDATPFTDVTVDNISKALHKCGYQMRGFETMYNGHTGRRLPAMIFLGPTYYQRLKHMVDDKIHSRGRGPVQILTRQPAEGRSRDGGLRFGEMERDCMIAHGAAHFLKERLFDQSDAYRVHVCERCGLIAIANLKKNSFECRGCKNKTDIVQVHIPYACKLLFQELMAMAIAPRMLTKEVKPLKDLKKKGG

SEQ ID NO: 187 - DNA, 489 bp, Cannabis sativa
  source 1..489: mol_type = other DNA
atgccaactttgtacaaaaaagttggacacagagctcgagatctctttcgggatattagccgcttttttcggtctctcagtctcctcttaaaaacttacgttaaccatatgggcctacttgaccagctttgggacgacaccgtcgccggacctcgtccggagaacggtttaggaaagcttcgaaagcattccaccttcagtttccggtctaattcgggcaagggatctgacggtggaaacgtaagatcgtacggtgaggatcagccggaggagccgacgagagttacgcggagtattatgatagtaagaccaccagcgagcctgagcggttcgcctccggtttcgccagctggatcaactcctccagtgtcgccgttttctggaggcagagagtcgtttaggtttcgaagaaagtcagcatcggatgcgtacgagaaaacaggcgaagttggacccagaagcagccctcctcctccttacgacgtgtga

SEQ ID NO: 188 - protein, 162 aa, Cannabis sativa
MPTLYKKVGHRARDLFRDISRFFRSLSLLLKTYVNHMGLLDQLWDDTVAGPRPENGLGKLRKHSTFSFRSNSGKGSDGGNVRSYGEDQPEEPTRVTRSIMIVRPPASLSGSPPVSPAGSTPPVSPFSGGRESFRFRRKSASDAYEKTGEVGPRSSPPPPYDV

SEQ ID NO: 189 - DNA, 690 bp, Cannabis sativa
  source 1..690: mol_type = other DNA
atgggttctgtttgttgctgcttgaatgtggacgattttgaagattacgtgaatccaaacagttctatatacaggaactgtatgtgtcttagttgctttattcagaatttcataaatgtgtatacatcactatttcgaagaggggaagcacattctgtcccatcatcaattcagggcgcagcatctattacttcttcagcatcgcttgacaactctctagctgacatgtatcgttctcctccaagacccttgccttatgatgctgacccaagatacttccgtttgcagcgggatggactgatttcaaggcgtgataaaggctcaagtcattcacatgaagaggcggaacccctcagaagtgatactgatgcagattcagaatctctgagtacagcagacaaatggagtgaatctgtttgtgaagatgggtcaaaagagcaccgttccaagtcttccaactccttaaagctctcagtgactaaacctacaactggagttgggaatatctatacatcttcagaagaagaggatgtctgtcctacttgtctcgaagaatatactccagaaaaccccaaaataatgacaaaatgctctcatcattttcaccttggttgtatctatgagtggatggagagaagtgaaagctgtccggtttgtggcaaggtgatggtattcaatgaaacgccttaa

SEQ ID NO: 190 - protein, 229 aa, Cannabis sativa
MGSVCCCLNVDDFEDYVNPNSSIYRNCMCLSCFIQNFINVYTSLFRRGEAHSVPSSIQGAASITSSASLDNSLADMYRSPPRPLPYDADPRYFRLQRDGLISRRDKGSSHSHEEAEPLRSDTDADSESLSTADKWSESVCEDGSKEHRSKSSNSLKLSVTKPTTGVGNIYTSSEEEDVCPTCLEEYTPENPKIMTKCSHHFHLGCIYEWMERSESCPVCGKVMVFNETP

SEQ ID NO: 191 - DNA, 1167 bp, Cannabis sativa
  source 1..1167: mol_type = other DNA
atggcttcgtccgctaccacaacgacgtcgtatttgtgttataggtgcaaccggttcgtacggatttgcgtaaacgaatcggtcgcttgcccccactgccaaactgggtttttagaagagatcgagtcaacaccttcgccattgccggaaccggttgcggagactactcaccgccatccttttgtagctacttacatgtttggtgaagaatctgctcaaatcccgagttcacgtagggttcgccgtggatcttctcgccgatccgctttgaaccctgtaatcgtcttacgtggcaccgtcgaggaaggaggtggtggtggtttggttgagggcgagaggaatagctttcagttgttctacgacgacggtgccggttcgggtctccggcctctcccgacgagtatgtccgagtttttaatgggttccggtatggataggttgttggaacagcttgcacagattgaagttaccggttttggaagatccgaaaatccaccggcctccaaagctgcgattgagtcgatgccgacgattgagatcgtcgatagccatgtaggttcagaatcgcactgtgcagtgtgtaaagaagctttcgagctgggtacggaggcccgagaaatgccctgtaagcatatataccactctgactgtatccttccatggctttctctccgaaactcttgcccagtttgccggctcgaacttccgacgaaccaaaccacagctgatccaaagctgtcgccggcggccgtagacgaagagactgttggtttgactatttggaggcttccagggggtggttttgcggttgggagatttttctccggcggaagaggcggcgagagggaagtgcccgtcgtttatacagaaattgatggtggactccattcgggtggggctccgagaagggtttcatgggtatcgagaagaaatagaggaggaggagagagtcatggaatcggcagtgtttttcgtaacatgttctcgtttttggggagaataagacgcaattctaattcgtcttcttcatcgagtcggactcatactgaatctggttctcttaatcgtagtcgtagttactcaattggttcagtgttcagtaggcatatgcggaggcgtaacagagcttgggttttggatgatcagactggaaatgaacggtggtga

SEQ ID NO: 192 - protein, 388 aa, Cannabis sativa
MASSATTTTSYLCYRCNRFVRICVNESVACPHCQTGFLEEIESTPSPLPEPVAETTHRHPFVATYMFGEESAQIPSSRRVRRGSSRRSALNPVIVLRGTVEEGGGGGLVEGERNSFQLFYDDGAGSGLRPLPTSMSEFLMGSGMDRLLEQLAQIEVTGFGRSENPPASKAAIESMPTIEIVDSHVGSESHCAVCKEAFELGTEAREMPCKHIYHSDCILPWLSLRNSCPVCRLELPTNQTTADPKLSPAAVDEETVGLTIWRLPGGGFAVGRFFSGGRGGEREVPVVYTEIDGGLHSGGAPRRVSWVSRRNRGGGESHGIGSVFRNMFSFLGRIRRNSNSSSSSSRTHTESGSLNRSRSYSIGSVFSRHMRRRNRAWVLDDQTGNERW

SEQ ID NO: 193 - DNA, 432 bp, Cannabis sativa
  source 1..432: mol_type = other DNA
atgccggtattattaagatggttcccaccaagccccatggttgttgagactttctccgagtacccaccactcggtcgtttcgccgtgagggacatgcgtcagaccgtggctgttggagtcatcaaggctgtggagaagaaggacccatcaggtgccaaggtgaccaagtcccgccgccaagaagaagtgaagtgttactgtttaggaagggagctttttatagttaggctagttttgtttctggtgtctttgcctttttcgatttcgttttgctttaatatgcggacaattatatgggttagcattgcaagttttactagcttgtttttcaaggttggtttagcttttgatgtcgacttttgttttcctattattgtttttttttgtttaaacttttgggttttaatatcattgagaattgagttgtcaaaa

SEQ ID NO: 194 - protein, 144 aa, Cannabis sativa
MPVLLRWFPPSPMVVETFSEYPPLGRFAVRDMRQTVAVGVIKAVEKKDPSGAKVTKSRRQEEVKCYCLGRELFIVRLVLFLVSLPFSISFCFNMRTIIWVSIASFTSLFFKVGLAFDVDFCFPIIVFFCLNFWVLISLRIELSK

SEQ ID NO: 195 - DNA, 663 bp, Cannabis sativa
  source 1..663: mol_type = other DNA
atgttgttcttcacgctgatgctattcacagaggtggtggacaggtcatcccaactgccaggagggttatctatgcttcccaaatcactgccaagcctagacttcttgagccagtgtacttggtcgagatccaagctccagagggtgctctcggtggtatctacagtgttcttaaccagaagcgtggacacgtcttcgaggagatgcagaggcagggtaccccactttacaacatcaaggcataccttccagtcattgagtctttcggtttctcagcctacattgagagctgctacctcaggacaggctttcccacaatgtgtgttcgaccattgggacatgatgtcttctgatccattggaggccggaactcaagcttccgtacttgttgctgatatcaggaagagaaagggtttgaaggagcaaatgacccccactttccgagttcgaagacaagctgtaagtttctactacaacttgtgcttgttgagaacatacagcttaggtttgaggaggaaagatgaggagaagggaaagaagttatcaggaatcgacattttttatgcttttctccattgccttatctattttgcttattttctagtctgttgttttttaagttttgttacaggggatatttggttgtatcctttggcattttag

SEQ ID NO: 196 - protein, 220 aa, Cannabis sativa
MLFFTLMLFTEVVDRSSQLPGGLSMLPKSLPSLDFLSQCTWSRSKLQRVLSVVSTVFLTRSVDTSSRRCRGRVPHFTTSRHTFQSLSLSVSQPTLRAATSGQAFPQCVFDHWDMMSSDPLEAGTQASVLVADIRKRKGLKEQMTPTFRVRRQAVSFYYNLCLLRTYSLGLRRKDEEKGKKLSGIDIFYAFLHCLIYFAYFLVCCFLSFVTGDIWLYPLAF

SEQ ID NO: 197 - DNA, 1014 bp, Cannabis sativa
  source 1..1014: mol_type = other DNA
atggaagacgacgacgagttcggagatctctacaccgatgttcttcagccctttgcttcctcgtcgtcttcctctgttcagcagcctcaccacccttccgttgagccgcctcaaccttttcgccctccgatcgatctcaatctcaatctcaagagcgatgtcgaagacactatgcttcgagctcccagcctcaattccgtcctacccgttaatgagacggacgaaaccctagttccaggtcctcctcccgccgtctcggttgattctgttactaataattcgatcattcccggtgaggtggtggttgtggatgatgcagtggctcggacgagggtttctttgtccggggatttgaaattgcgtgatggggttgtggaggattcaaattcgaatttgaacttgaactatggtgttggtgagtctgggaacgatgttgttatgaagcaatcgtgtggtgaagttgagaatttgatggagaaagatgtaacttttgatattgaagaaggtaatatggggatggaagatgttggttcggaaccgatgattccaggtctcgagagtagcgtacctattggtgggacgatgggggatgttgataacgtggatgcttcaatgggagttagaagtttgggtggtgatggagttgatgaagatgatgccgctggcggtgatggtgatgattgggatagtgatagtgaggacgatttgcaaatagtccttaatgataacaatcatggacctttggctatggagagaggtggaatgggaggtggcgacgacgatgaagatgaagacgggctcgttattgtgactaatgacgatcctaatcaagtgatggaagaacaagaatggggagaggattctgcgcaggtggcggatggcgagaggaaggaggtgggtgaagctgggaaagttggtgccggcattgtggcgccacagaagattggttataacactcatggttatcatccatttcattctcagtttaaggtcagtgtcttcatatgctgctga

SEQ ID NO: 198 - protein, 337 aa, Cannabis sativa
MEDDDEFGDLYTDVLQPFASSSSSSVQQPHHPSVEPPQPFRPPIDLNLNLKSDVEDTMLRAPSLNSVLPVNETDETLVPGPPPAVSVDSVTNNSIIPGEVVVVDDAVARTRVSLSGDLKLRDGVVEDSNSNLNLNYGVGESGNDVVMKQSCGEVENLMEKDVTFDIEEGNMGMEDVGSEPMIPGLESSVPIGGTMGDVDNVDASMGVRSLGGDGVDEDDAAGGDGDDWDSDSEDDLQIVLNDNNHGPLAMERGGMGGGDDDEDEDGLVIVTNDDPNQVMEEQEWGEDSAQVADGERKEVGEAGKVGAGIVAPQKIGYNTHGYHPFHSQFKVSVFICC

SEQ ID NO: 199 - DNA, 1017 bp, Cannabis sativa
  source 1..1017: mol_type = other DNA
atgattttgcttcgattaccaaaagctcccatggccatgaccttcttcttagctcaaccttattcttcttcttcttcactctactccttctcccattcccatttcagtctcatcccaaaaccccattcactcaccattaatccaactcttaaaccctttcaccttccctcatctccaaaatcaactctttcaccttcccaactacagaccttatccacctccgacactacacacccccaaatcgaaacccctcaagaagatttcgatacccaaatccaagtatccctcgaaaacctcttcgtgcctccggaaacagaaattggtgatggtacttcagccctgacctcgagaatcttgaggggctctaatattgttctgagcaagtacgcaagggatgctcaggttgcccaagctgagttcgttaagagcagtgttgccactgaggattgtccctctgatggtcttcctgagttcgctctcgttggtcgctccaatgtgggaaagtcttcgcttcttaattcccttgttcgcgagaaagaaacttgctttaacctcaaagaagcctgggaagacacaatgcatcaaccatttcgaatcaatgatagctggtaccttgttgatttgcctggatatggatatgcttctgcaccgaaggatcttcgaacagattgggcaaaatttactaaagattacttcctaaataggtcgaaactagtttcagtattccttctcattgatgctagcattcctgccaaaagcattgaccttgagtatgctagttggctggcacagaatcagatccccatgacattaatcttcacgaaatgcgacaagcgaaagaagaagaagaatggaggcaaaagaccggaagaaaatgtgaaagatttccaggagttagtacgtggtttcttcaagacggtacccccatggattatgaccagcagtgttacaaatcagggtcgtgacgagatattactgcatatggcccagctacgcaactactggctcaagcactag

SEQ ID NO: 200 - protein, 338 aa, Cannabis sativa
MILLRLPKAPMAMTFFLAQPYSSSSSLYSFSHSHFSLIPKPHSLTINPTLKPFHLPSSPKSTLSPSQLQTLSTSDTTHPQIETPQEDFDTQIQVSLENLFVPPETEIGDGTSALTSRILRGSNIVLSKYARDAQVAQAEFVKSSVATEDCPSDGLPEFALVGRSNVGKSSLLNSLVREKETCFNLKEAWEDTMHQPFRINDSWYLVDLPGYGYASAPKDLRTDWAKFTKDYFLNRSKLVSVFLLIDASIPAKSIDLEYASWLAQNQIPMTLIFTKCDKRKKKKNGGKRPEENVKDFQELVRGFFKTVPPWIMTSSVTNQGRDEILLHMAQLRNYWLKH

SEQ ID NO: 201 - DNA, 1422 bp, Cannabis sativa
  source 1..1422: mol_type = other DNA
ttggggaagattgtcgagaagataaggcaggagagtggtgctcaggttagagttttgcctaaagatcagattccggcttgtgcttcacctggagatgagttaattcaaataacagggaacatttcagctgtaaagaaagcacttctatctgtttcaggttgtcttcaggataagcctagggagaatgcaaccaactatggtggcagtaaagtttcagggagaatgcatcatggaaatgggcctgaaaatatggggtcgggtcataatagaatgtttatagaagaggaggtggtgtttaagctgttatgccaagttgataaggttggtagtttgatagggaaaggtggctctgtaattcgacatttgcaaaatgatactggcgcatacatcaagatagctgatgctccacctgattcagatgagcgagttgttatgatatctgcacgggagaattcagagcaaaaacattccccttcacaggacgctgttattcgtgtacattctcgaattgcagagattggatttgaacccggtgctgcagtagttgctaggcttcttgtacattcgcaacagataggttgtttattgggtaaaggaggtttcattatttctgaaatgagaagagctactggtgctagcattcgcatatttacaaaggaccaagtcccgaaatgtgcctcacacaatgatgaagttgtgcaggttattggaagctttcagtgtgtacaggatgccttatttcacattacaagtagactacgggaaacaattattcctttgaagccaccgcatccaaattttgatggcccaccatacatgcctccataccctgaaatgccacctccaatgttcaggcctagacataacccagcttccccaggtccatgtccttctcctgttgggcctcctcatggaattgatcggtcttctgttccatcccaaccacatgatcaccagcatggatttttacatggcatggaccacaatgctccatctccaatggacagagctccctatcattatggaagtgaacgccatggacaaggtcgaccatttgatagaccatcttctccaagaatgtggaattctcagggtgttaactgtggaaatcctaggggaattgataatagtccaagcttccatgcaagaaatggttccttgggaagtgaaaatcaagcacagcatttagctcccgcaactgttgagatcacaatccctcaaaagttgttaacccatgtttatggagataacaacagtaatataacacagatccgccagatctcaggtgctcaggtggtggtaaacgatatgaaacctggagccacggaagggctggtttcagtttcgggtactcctaatcaaatatctagtgctcaatgccttattcatgctttcatcctttgtgggcagacaccaggttga

SEQ ID NO: 202 - protein, 473 aa, Cannabis sativa
LGKIVEKIRQESGAQVRVLPKDQIPACASPGDELIQITGNISAVKKALLSVSGCLQDKPRENATNYGGSKVSGRMHHGNGPENMGSGHNRMFIEEEVVFKLLCQVDKVGSLIGKGGSVIRHLQNDTGAYIKIADAPPDSDERVVMISARENSEQKHSPSQDAVIRVHSRIAEIGFEPGAAVVARLLVHSQQIGCLLGKGGFIISEMRRATGASIRIFTKDQVPKCASHNDEVVQVIGSFQCVQDALFHITSRLRETIIPLKPPHPNFDGPPYMPPYPEMPPPMFRPRHNPASPGPCPSPVGPPHGIDRSSVPSQPHDHQHGFLHGMDHNAPSPMDRAPYHYGSERHGQGRPFDRPSSPRMWNSQGVNCGNPRGIDNSPSFHARNGSLGSENQAQHLAPATVEITIPQKLLTHVYGDNNSNITQIRQISGAQVVVNDMKPGATEGLVSVSGTPNQISSAQCLIHAFILCGQTPG

SEQ ID NO: 203 - DNA, 1545 bp, Cannabis sativa
  source 1..1545: mol_type = other DNA
atgagtggcggtggagaaggtgagtccttggagtatactcccacatggatcgtcgccgtcgtctgctctgtcatcgtcgccatttctctcgccgcggagcgtcttctccactatctcggtcaggttttgaataataagaaccagaagcctctatacgaggccttgcagaagatcaaggaggagttgatgcttttgggtttcatttcgcttctcctcactgtttcgcagaacacaatttcaaagttttgcgtacctgcttctttggtcgacaatctcctcccttgtaagcgccctcatcaggaagctaatgattcgtcttcttcaacctctcatcacatctccggctctttcagacgccttcttgaagaaacttccactgctcatttgggttattgcgctcataagaaaaaagtaccccttttatccgttgaagcattgcaccatttgcatatctttatcttcgtcctagccattgtccatgtcactttctgtgttctcactgttgtgtttggaggtgtaaagatacgtcaatggaaacgttgggaggattctattgcaaatgagagttatgacactgaacaagctgttcttaagtctaagaaaaaggtaacaagcgtccatcaacacgcgttcattaaggaccactttttgggttttggtaaagactcagctctactaggctggctgcatgcttttgttaagcaattttatgcatctgtgaccaaatcagattatgcgaccatgcggttaggattcatcatgactcactgcaggggaaatcttaagtttaattttcacaaatatatgatacgtgctctcgaagatgatttcaagaaagttgttgggataagttggtacctttgggtttttgtggtcatcttcttgttgctgaatgttaatggctggcacacatatttttggatagcattcattcctttctttcttctacttgccgtgggcactaaactggagcatgtcattactcaattggctcatgaagttgctgagaaacatgtagcaatagaaggagatctaatagttcagccatcagatgatcacttttggtttcatcgtccccagattgtcctcttcctgattcatttcatccttttccaaaattcatttgagattgcgtttttcttctggatattggttcaatattcatttaactcctgtataatgggacacgttcgttacattgtcccaagacttgtcattggggtgttcattcaggttctctgtagttacagcactctcccgctctatgccattgtcacacagatgggaacttcttacaagaaagccatatttgaggagcgcatgcaagtaggaattgttaattgggctcagaaggtcaagaaaaagaagggatttaaagcagccctcattggttccggtgctggctcaggctcaggctcaggctcgggatcaggctctactcctgctcaaccaatttctgatactggttctgcatctgtgggaattcagctgggaagatttgtgcccaaagcatcaaagccagagcggagtgatgactctaagtga

SEQ ID NO: 204 - protein, 514 aa, Cannabis sativa
MSGGGEGESLEYTPTWIVAVVCSVIVAISLAAERLLHYLGQVLNNKNQKPLYEALQKIKEELMLLGFISLLLTVSQNTISKFCVPASLVDNLLPCKRPHQEANDSSSSTSHHISGSFRRLLEETSTAHLGYCAHKKKVPLLSVEALHHLHIFIFVLAIVHVTFCVLTVVFGGVKIRQWKRWEDSIANESYDTEQAVLKSKKKVTSVHQHAFIKDHFLGFGKDSALLGWLHAFVKQFYASVTKSDYATMRLGFIMTHCRGNLKFNFHKYMIRALEDDFKKVVGISWYLWVFVVIFLLLNVNGWHTYFWIAFIPFFLLLAVGTKLEHVITQLAHEVAEKHVAIEGDLIVQPSDDHFWFHRPQIVLFLIHFILFQNSFEIAFFFWILVQYSFNSCIMGHVRYIVPRLVIGVFIQVLCSYSTLPLYAIVTQMGTSYKKAIFEERMQVGIVNWAQKVKKKKGFKAALIGSGAGSGSGSGSGSGSTPAQPISDTGSASVGIQLGRFVPKASKPERSDDSK

SEQ ID NO: 205 - DNA, 468 bp, Cannabis sativa
  source 1..468: mol_type = other DNA
atggcttgtactggaaagcttcatgttgaagttgaggtgaaatctcatgcagataaggtttgggaatcccttagggatttcatctacatttacccaaaggcatttcctcatgattacaaaagtgttgaggttttggaaggtgatggcatcaagcctggatctgtcagattcattacatatggagaaggttctccacttgtgaaagtgtcaaaagaaaggatcgaagtagttgatgtgccaaacaagatattttgttacaatgtgatagaaggagatctgttaaagtactacaagagtttcaaggccaatgttcaagtggttcctaaagaagaaggaagtgtccttaaatggagttgtgagttcgaaaaggctactgatgagacccctgatccagatgccatcaaggattttgctatcaagaattttaaagagctcgatgactacattattgccaacaacaaggcataa

SEQ ID NO: 206 - protein, 155 aa, Cannabis sativa
MACTGKLHVEVEVKSHADKVWESLRDFIYIYPKAFPHDYKSVEVLEGDGIKPGSVRFITYGEGSPLVKVSKERIEVVDVPNKIFCYNVIEGDLLKYYKSFKANVQVVPKEEGSVLKWSCEFEKATDETPDPDAIKDFAIKNFKELDDYIIANNKA

SEQ ID NO: 207 - DNA, 630 bp, Cannabis sativa
  source 1..630: mol_type = other DNA
atgtcgtccaaggactgcggccaccacgggaagaaacgggaaaagcttatccggcgaatctgcgccggtttcctgatcttcctcttcatcgttttggtgttggttctgttaatttgggctattcttcaaccttcaaaacccagatttattctccaagacgtgaccgtttacgccttcaacaccaccgtccccaatttcttaacttccaattttcaggtaactatttcctctcgaaacccaaacgataagatcggtgtctactacgacagactcgacgtttacggagtctatcataaccaacaaattacttaccgtactcgaataccgcccacctaccaaggtcacaaagacgttaacgtttggtcaccgtttgtttacggtaaccttgttcctgttgcgccgtttaattccatggcgttgactcaagaccaagctaacggcgccgttatgatcatgttcaaggctgacggaagggtgcgttggaaagtggggacgtttatctctggccgttatcatttctacgtcaagtgtttggcgtttatcaatttgggaaataagaactcaggaattattgtgggtgaaaacgccgttaagtatcagattgtccagagatgcagcgtcaacgtatga

SEQ ID NO: 208 - protein, 209 aa, Cannabis sativa
MSSKDCGHHGKKREKLIRRICAGFLIFLFIVLVLVLLIWAILQPSKPRFILQDVTVYAFNTTVPNFLTSNFQVTISSRNPNDKIGVYYDRLDVYGVYHNQQITYRTRIPPTYQGHKDVNVWSPFVYGNLVPVAPFNSMALTQDQANGAVMIMFKADGRVRWKVGTFISGRYHFYVKCLAFINLGNKNSGIIVGENAVKYQIVQRCSVNV

SEQ ID NO: 209 - DNA, 861 bp, Cannabis sativa
  source 1..861: mol_type = other DNA
atggttatggctagttcatcatcttcttcttcttctactagtaataaaaatctggttttgataatctgttgtgctgtactaatttgtagtagtattattagtgtgtcaagtgcaaattttgaaggcccagctgagaaaacagttcctttaaccaaaggcctctcatggcaattctatgcaaaatcatgtccttctgctgagtccattataagaaaagaactcaagaaaatcttcaaaaatgatattgcccaagctgctggcttgcttcgtctccatttccatgattgctttgttcagggatgtgatggttcagtgttgttagatgggtcagcaagtggaccaagtgagaaagatgcaccacctaatctcaccttgagagccaccgcattcaagatcattaacgatcttcgagccctaattcacaagaagtgtggaagagttgtttcttgcgctgatattaccgctcttgctgctcgtgattctgttttcttgtccggtggcccagactacgctgtacccattgggaagaagagacggcttagcattcgcaacccaaaacgaaaccaatgccaaccttccacccaccctcaagtggcgcccaagccattctcaaacaactcgccgacaaccaaaacctcgaccccacagacgtcgtcgccctctcaggcggtcacaccatcggaatcgcccactgcggatccttcgacaaccgtctctacccaacccaagatccagtcatggaccaaaccttcgccaagaatctcaaatccatatgcccaactaaagacttcgacgccttcaccttccaagatctccgaacacccaacaccttcgacaacaaatactacgtcgatctcatga

SEQ ID NO: 210 - protein, 286 aa, Cannabis sativa
MVMASSSSSSSSTSNKNLVLIICCAVLICSSIISVSSANFEGPAEKTVPLTKGLSWQFYAKSCPSAESIIRKELKKIFKNDIAQAAGLLRLHFHDCFVQGCDGSVLLDGSASGPSEKDAPPNLTLRATAFKIINDLRALIHKKCGRVVSCADITALAARDSVFLSGGPDYAVPIGKKRRLSIRNPKRNQCQPSTHPQVAPKPFSNNSPTTKTSTPQTSSPSQAVTPSESPTADPSTTVSTQPKIQSWTKPSPRISNPYAQLKTSTPSPSKISEHPTPSTTNTTSIS

SEQ ID NO: 211 - DNA, 1050 bp, Cannabis sativa
  source 1..1050: mol_type = other DNA
atgggttccaaagctctcttcttcttcgctttactatcttttcaggcactttcatgtctcatcaaaacctcttctgcctctgataatcaaaatgagcaagacccaggtcttgtcatgaacttttacaaggattcatgtcctcaagctgaagatatcatcaaagaacaagtcagactcttatacaagcgccacaagaacactgctttctcttggctcagaaacatttttcatgactgtgctgttcagtcgtgtgatgcttcattgctgctggactcaacaaggagatccttgtctgagaaagaaatggatagaagctttgggatgagaaatttcaggtacattgagaccattaaagaagctgtggagagagaatgccctggcgtagtttcatgctctgatattcttgtgctctctgctagagatggcatagcttcgctaggaggaccttacattcctctgaaaactggaagaagagatggtcgtagaagcagagcagagatcctggagcagtaccttcctgaccacaacgaaagcatgtccgttgttcttgacaggttcgcctccatgggcattgacacccctggcctcgttgctttactagggtctcacagtgtggggcgaacccactgcgtgaaattggtgcaccgtttgtaccctgaagtggacccagcactgaacccagaccacgtagagcacatgctatacaagtgcccagactccattccagacccaaaagcggtgcagtacgtgaggaatgacaggggtacgcccatgattcttgacaacaattactacaggaacatattggacaacaaggggcttcttcaagtggatcatcaattggccactgataagaggaccaagccatatgtgaaaaagatggccaagagccaagcctacttcttcaaagagttctctagagccatcaaaattctctccgagaataaccctctaactggtaccaagggtgagattagaaagcagtgtaatcttgtcaacaagctccatttgagaattgagatatcaatattcaaacatttctgggagtga

SEQ ID NO: 212 - protein, 349 aa, Cannabis sativa
MGSKALFFFALLSFQALSCLIKTSSASDNQNEQDPGLVMNFYKDSCPQAEDIIKEQVRLLYKRHKNTAFSWLRNIFHDCAVQSCDASLLLDSTRRSLSEKEMDRSFGMRNFRYIETIKEAVERECPGVVSCSDILVLSARDGIASLGGPYIPLKTGRRDGRRSRAEILEQYLPDHNESMSVVLDRFASMGIDTPGLVALLGSHSVGRTHCVKLVHRLYPEVDPALNPDHVEHMLYKCPDSIPDPKAVQYVRNDRGTPMILDNNYYRNILDNKGLLQVDHQLATDKRTKPYVKKMAKSQAYFFKEFSRAIKILSENNPLTGTKGEIRKQCNLVNKLHLRIEISIFKHFWE

SEQ ID NO: 213 - DNA, 879 bp, Cannabis sativa
  source 1..879: mol_type = other DNA
atgaatctatttctgggttacttcaaaccacaggaagggaaacctgctctttgggaacttgattctgattattatcttcatgtctctgggattggagacgacctttttccagatcggtgtctagaaaccaacaacaagcatgtgggagctggatttcctctatcccctattccagcttgcagagaggatttctcacaaatgaagttgacgtcatttgataaattgattgaaaaaacttgcagttcaataaagaatgtaaggctttgtcgtgagccaaatcagagaccagggggtggtgcaggaagttatggtgtggctcctgatgcagctgaaatacagctcaaaagcccaaactggttgtttggtcagaggaaatatgaagaaaatgtttcttccccaaaagttatttcaggagaaactaatggcgaatccactgacgagaaagaagttgattgttttcgtaaccaatattgggtttcttctggtgataatggcaatgatgaggatatcattcacaggtatctggcaatgtcatccgtggatgaagccaatggttggtatggtggaactctagttggcgatcaagatgaaagcagtgaaatatataagcactatgctcaactatgcgagggacctgccgtggaacccttccaaaatgatactgaacaggagcagcaatatgctgatgttcttcgaatggaaacaatggacattgttgaggatgctgctacagaagaagagatgggggcagctctcaaggagtacgatcaaattggtgctgatcttgggatcagccctttatcacgccgatcctttgccggagatcctagttggttgaccaggtggattgttggggaagatcagctggaaagagcttaa

SEQ ID NO: 214 - protein, 292 aa, Cannabis sativa
MNLFLGYFKPQEGKPALWELDSDYYLHVSGIGDDLFPDRCLETNNKHVGAGFPLSPIPACREDFSQMKLTSFDKLIEKTCSSIKNVRLCREPNQRPGGGAGSYGVAPDAAEIQLKSPNWLFGQRKYEENVSSPKVISGETNGESTDEKEVDCFRNQYWVSSGDNGNDEDIIHRYLAMSSVDEANGWYGGTLVGDQDESSEIYKHYAQLCEGPAVEPFQNDTEQEQQYADVLRMETMDIVEDAATEEEMGAALKEYDQIGADLGISPLSRRSFAGDPSWLTRWIVGEDQLERA

SEQ ID NO: 215 - DNA, 459 bp, Cannabis sativa
  source 1..459: mol_type = other DNA
aaagtggcgagccgtagagcaagtggtagagctttcggagcggctcttgcggcggagacggttacttttccgatcacggtagataaggtgatgagtacagttgtgagaagacctaagaagagtaagaggagcaagaaggagaaggaagaggaagaggaggtgttggtgattgaaggtattgagtatgaccagaatttagtgttgaagttggatgtttacgtgaacgacgaggatgataagtcgccgagtggacctgagatatctgagttcgctggaagcttcgttaacgtgccacacaaacatgtgggtgtgaagaaggctaagacttctctgaggttgggcttaactgatttgcttgaggatttggaggctgaagatgatgagactgtggtcgtgactttggttcccaaggtcggaattgccactgtttctggaattaagattgagtttttgtcttga

SEQ ID NO: 216 - protein, 158 aa, Cannabis sativa
RRAKSTKVASRRASGRAFGAALAAETVTFPITVDKVMSTVVRRPKKSKRSKKEKEEEEEVLVIEGIEYDQNLVLKLDVYVNDEDDKSPSGPEISEFAGSFVNVPHKHVGVKKAKTSLRLGLTDLLEDLEAEDDETVVVTLVPKVGIATVSGIKIEFLS

SEQ ID NO: 217 - DNA, 759 bp, Cannabis sativa
  source 1..759: mol_type = other DNA
atggaaaaagtgaggatcgaagttgatgaaaggaggaaaaaacaagctcggagaataatggaggcaatagagataggaatgatgaagagactaaaagccaaagaagaagagattgagaagattgggaagttaaattgggcattagaagaaagagtaaagacattatgcgtagagaatcaaatatggagagacttagcacaaaccaatcaagaaaccgccaatgctctcagaacaaatctcgaacatgtattagcacaagtcaaagacaacaacaatcacaacaaccgcaatactcgctgcgtcgccggagccggtgccggattattaccagaagacgtaaacatcgataacgaaaacgacgaagacgaagacgatgaccttcaacccaacacattggcagccgcactagctgacgatgcccaatcttgctgtggcgaaaacgacgacgtaatagaaacaggagcagtattaggagagtggaggagtacattaaacgacgtcgtcaatcataataacaacaaaattgatgtcgttgatttggaaacgacaagtaacgtgaggtgtacgacaaccagtaataatactacgagtacaatcaatcacaataggctttgcaggaattgtgggaaggaagagtcatgcgtgttgcttttaccatgtagacatctctgtctttgtactgtgtgtggttcttctcttcacacttgccctatctgtaaatcaaccaagaatgctagtgtccatgttaacatgtcctaa

SEQ ID NO: 218 - protein, 354 aa, Cannabis sativa
MYNDSSVPPPKTAKKSDSGLTYNIPTTTTTSFSRKRSRDSSAAINNVNPSFLSFPPCTSTTTTQPIHYYKNSSTSPFSFLGEDFSYQIQQQQFDLDRLISQHMEKVRIEVDERRKKQARRIMEAIEIGMMKRLKAKEEEIEKIGKLNWALEERVKTLCVENQIWRDLAQTNQETANALRTNLEHVLAQVKDNNNHNNRNTRCVAGAGAGLLPEDVNIDNENDEDEDDDLQPNTLAAALADDAQSCCGENDDVIETGAVLGEWRSTLNDVVNHNNNKIDVVDLETTSNVRCTTTSNNTTSTINHNRLCRNCGKEESCVLLLPCRHLCLCTVCGSSLHTCPICKSTKNASVHVNMS

SEQ ID NO: 219 - DNA, 1173 bp, Cannabis sativa
  source 1..1173: mol_type = other DNA
atgttccctagaggtgtaagtgactatgttgattcaatgcaaaaacttatccctgaaatgacagatgggaccgttcggactgccattgatactggctgtggggttgcaagttggggaggtgatttgttggatcggggaatactaactatatcccttgctccaagagataatcatgaagctcaagtccagtttgccttggaacgtggaattccagcgatcctcggcattatttctactcagaggctccctttcccgtcaaactcatttgatatggctcattgttctagatgtctcattccgtggacagaattcggtggggtttaccttcaggaaattcaccgcatactccgtcctggaggtttttgggtgttgtctggtccaccagtaaactatgagcgccggtggcgtggatggaacacaaccattgaggatcaaagaagcgattataacaatctgcagaaactgcttactgccatgtgcttcaaattgtacaacaagaaggatgacattgctgtctggcggaaggcttcagacaatagttgctacaataaactttctaataatgctgacgcctatccaccaaaatgtgatgacagccttgaacctgatgctgcttggtatactcctctccggccttgcattgttgttccaaaccaaaaatataagagaacagatttaaagtcggtctctaaatggcctgcgcggttgcatgctgctccagaacgtgtctcggatgttcacggagggagtgctagcacattcaagcatgatgatgacaagtggagggtgcgtgcgaagcactacaaaaagttgctttcatcaattgggtccaataagattagaaatgtaatggacatgaatacagtttacggaggcttcgctgcagctgtgattgatgatcctttgtgggtcatgaatgttgtctcttcttatgctaccaacacattagctgtggtctttgatcggggccttattggaacttaccatgattggtgtgaagcattctctacttatcctcgtacttatgatcttcttcatcttgatggcctctttactgccgaaagtcacagatgtgacatgaagtatgtgctgttagagatggaccggatcctgcgaccaaatggctatgcaataattcgagaatctagttactttgtggatgctgtggctgcg

SEQ ID NO: 220 - protein, 391 aa, Cannabis sativa
MFPRGVSDYVDSMQKLIPEMTDGTVRTAIDTGCGVASWGGDLLDRGILTISLAPRDNHEAQVQFALERGIPAILGIISTQRLPFPSNSFDMAHCSRCLIPWTEFGGVYLQEIHRILRPGGFWVLSGPPVNYERRWRGWNTTIEDQRSDYNNLQKLLTAMCFKLYNKKDDIAVWRKASDNSCYNKLSNNADAYPPKCDDSLEPDAAWYTPLRPCIVVPNQKYKRTDLKSVSKWPARLHAAPERVSDVHGGSASTFKHDDDKWRVRAKHYKKLLSSIGSNKIRNVMDMNTVYGGFAAAVIDDPLWVMNVVSSYATNTLAVVFDRGLIGTYHDWCEAFSTYPRTYDLLHLDGLFTAESHRCDMKYVLLEMDRILRPNGYAIIRESSYFVDAVAA

SEQ ID NO: 221 - DNA, 291 bp, Cannabis sativa
  source 1..291: mol_type = other DNA
ttggggcggccgcactactttgtacaagaaagttgggttttttcaacctttaatctacattacagattaacagccgtgactcaattggcacatcatggtatgatatatgtccctattgggtacacattcggaagtggaatgtttgagatgaatgaggtaaagggtggctctccttatggtgctgggacatatgctgcagatgggtctcgtgagcctacggagcttgaacttaagcaagccttttaccagggtaaatatgttgcagaaatggccaaaaagctcaagcattaa

SEQ ID NO: 222 - protein, 96 aa, Cannabis sativa
LGRPHYFVQESWVFSTFNLHYRLTAVTQLAHHGMIYVPIGYTFGSGMFEMNEVKGGSPYGAGTYAADGSREPTELELKQAFYQGKYVAEMAKKLKH

SEQ ID NO: 223 - DNA, 918 bp, Cannabis sativa
  source 1..918: mol_type = other DNA
atggcgataatctccgagttcgaggaagagcacaaagagaccaaacctaagcccaaccccaacctcaagccatccgcatccccttcttcatcatcatcagccacggcgtcctttactgccattttgaatccctccaatccgctagggttcttggacaaagttttcgaatttgtcgctcaacatagtgattttctcgagaaagttaccgcggagaaggagattttgtctgttgctcgcgtcgctaaggagaaggagaaggagagtaagaggagaaaggtggaagaggtcgaggagaagaaggtgagtgtggtacctgagaatgtgattaaggtgcctaagaaagaagaagaagaggaggaaaagtctcccgtgaatgtagaaaacaaggaggaaagtggtctcagagctcccaatcttgggaatgggcttgatcttgaaaaatactcatggacgcagagcctgcaagaagttactgcagttatcccagtgcccgctggaaccaagtcaaggtcggttgtctgtgatataaagaaaaaccatctaaaggttggactcaagggtcagccacctatcattgatggggagctctttcggcctgttaagcctgatgattgttactggagtatagaggatcaaagttctatctctattctcttgaccaagcatgatcaaatggattggtggaaatctttggtgaaaggtgatcctgagattgacacccaaaaagttgaacctgaatcaagcaagttatctgacctggacccagaaactcgacagacagttgaaaagatgatgtttgatcagagacagaagtctatggggctcccaagcagtgaagaaatgcagaaagagaaccttctgaagaaattcatggccgagcacccggagatggatttttcaagggctaagatttcataa

SEQ ID NO: 224 - protein, 305 aa, Cannabis sativa
MAIISEFEEEHKETKPKPNPNLKPSASPSSSSSATASFTAILNPSNPLGFLDKVFEFVAQHSDFLEKVTAEKEILSVARVAKEKEKESKRRKVEEVEEKKVSVVPENVIKVPKKEEEEEEKSPVNVENKEESGLRAPNLGNGLDLEKYSWTQSLQEVTAVIPVPAGTKSRSVVCDIKKNHLKVGLKGQPPIIDGELFRPVKPDDCYWSIEDQSSISILLTKHDQMDWWKSLVKGDPEIDTQKVEPESSKLSDLDPETRQTVEKMMFDQRQKSMGLPSSEEMQKENLLKKFMAEHPEMDFSRAKIS

SEQ ID NO: 225 - DNA, 663 bp, Cannabis sativa
  source 1..663: mol_type = other DNA
atgacaaagatacatcctagctctgcctccgcttccatcaccgccgctccaaccccatctttggtctcagaatctccaaatgaggcggtcgtcttaactgtgtggacaaaatcccttctattcaattgtaatggctttacggtatatgactccaagggcaatctagtctaccgtgttgataattacatcgccggtagcaagggagagatcgtcctcatggacgccgccggcaagtctattctcaccattcgacgcaagaggttaagtcttggtgataattggatggtttacgacggagacacgaaggtgaatccgcgattcagcgtaaaaaaacacgtgaaaatcctaaacacgaggtgtttggcgcgcgtgatgagctctggcagaggtggaagtgaatcgccatcgccgaagagcagtagcccatcgtcaacattgtacgaaattgagggatcgtatgcccagcggtgctgtgtggtttacgaccacaagagacagagagtagcggagataaggcggaaggaggctgggatcggcggagtttctttgggaggggacgtcttccgcctcgtggtggagcctcatttcgatacctcagtcgccatggcccttgttattctgctagatcagatgtttggtcggtcatcttcttttaagcgttga

SEQ ID NO: 226 - protein, 220 aa, Cannabis sativa
MTKIHPSSASASITAAPTPSLVSESPNEAVVLTVWTKSLLFNCNGFTVYDSKGNLVYRVDNYIAGSKGEIVLMDAAGKSILTIRRKRLSLGDNWMVYDGDTKVNPRFSVKKHVKILNTRCLARVMSSGRGGSESPSPKSSSPSSTLYEIEGSYAQRCCVVYDHKRQRVAEIRRKEAGIGGVSLGGDVFRLVVEPHFDTSVAMALVILLDQMFGRSSSFKR

SEQ ID NO: 227 - DNA, 1083 bp, Cannabis sativa
  source 1..1083: mol_type = other DNA
atgaatttgagatgcagaaatcttgcttgtcgtagtccactgcctgtggatgaatgtgattgcaaggtttgttctcagaagaagggtttttgcagttcatgcatgtgtcttgtctgctcgaaatttgacatggcatccaatacgtgtagttggcttgggtgtgatgtttgtcttcattggtgtcatgcagattgtgcgttacgtgactcttatattagaaatgggcgcagtgcaatgggggcacaaggaacatctgagatgcaattccattgtgttgcctgtgatcacccgtctgagatgtttggctttgtgaaagaagtttttcaaaattttgccaaggactggtcagctgaaactctatccaaggaacttgaatatgtcaagagaatatttgctgcaagcaaggacttgagagggaaacgactctatgagattgctgttcagatgttggcaagattgaaaaataagaacgaccttcctgaagtttacagttacataatgagcttcctcaacgatgttgattctttcaagttaaataatatgcctcttccatctgggaaggaacaaggtaaaggaagcatcagtgggaatgggattgctgggccaagccaagaacccacatggctaaaatcggtatattcagaaaaggttcctcagttggatatacctgctagtttacttcctagttacagctatgaccgaaatgacaagcgcaacatggacttggagttgcggacaaatactcccaaggagcctcttttcgatgagctggagagtattgtaagaattaaacaagcagaggcaaagatgttccaaacaaaggcggatgatgcaagaagggatgcagaaggcctgcatcagatagccattgccaaaaatggaaagattgaggaggagtatgctagtagaattgccaacttgcacttggatgaagcagagaggttgcgtaaacaaaaacttgaagaactgcaggctcaggaacgagaaaatcgtgaatactttagtatgaaaatgagaatggatgcagaaattaaagacctgttattgaaaatggaagctaccaagcggaacctagcagcatga

SEQ ID NO: 228 - protein, 360 aa, Cannabis sativa
MNLRCRNLACRSPLPVDECDCKVCSQKKGFCSSCMCLVCSKFDMASNTCSWLGCDVCLHWCHADCALRDSYIRNGRSAMGAQGTSEMQFHCVACDHPSEMFGFVKEVFQNFAKDWSAETLSKELEYVKRIFAASKDLRGKRLYEIAVQMLARLKNKNDLPEVYSYIMSFLNDVDSFKLNNMPLPSGKEQGKGSISGNGIAGPSQEPTWLKSVYSEKVPQLDIPASLLPSYSYDRNDKRNMDLELRTNTPKEPLFDELESIVRIKQAEAKMFQTKADDARRDAEGLHQIAIAKNGKIEEEYASRIANLHLDEAERLRKQKLEELQAQERENREYFSMKMRMDAEIKDLLLKMEATKRNLAA

SEQ ID NO: 229 - DNA, 957 bp, Cannabis sativa
  source 1..957: mol_type = other DNA
atgccttatcttttggatcttcatctatttctctcgaaaccaatttggcctgcaccagctgaggaggaagttcttgatgataaagatggtatattgtccaaaattgatgatgccattgcaaatggagaaacagtctatggacgctttccccagacagtaattgatcttgtttcagatagacttagggaagtcattgattcctctggggaattgagttctttgatgaaaacctgtgtaaatgcatttcgtttgtattcaaagacaaaaccatcacctgcaaaggaatccattagaagatcaaaggaattacctcctgaaggtttgcatccaatgttcaaaaatgttcttggtgggggagagttaatggcactggctttttctgagcgtttgaagacatttagaccgaagcagaccatacttgaagctgaaggggaagctgctaaatccaagcatttacagggttcttctagtcaatggactgatgtgatgaagaaaaaaagagctattcatgagcagataattaacttggttcaacagcagcgttctaatactattgcggagaaggaaatagaagatgacagagcttctgtgataggaaaggataataaagaagctggttctaaaaggaaagaaagaaacttcaaggatgaagagttctttataagttctataccgacaaatcatatttcagagtcaggactctctgttggagctaaccaggaatttggttcaaataggtttgaagcggcggttttagatttagttgccgatgatactgcaggaatgcagagacaaaaatctgtgtatcactgggacaggaggagtaaaaagtacatcaaattaaatagtggtgaccgtgttacagctagtgggaaggtaaagacagaaagtggtgcaagagtaaaagctaacaaaactgggatatacaagaaatggaaagaaacattcacacaataa

SEQ ID NO: 230 - protein, 318 aa, Cannabis sativa
MPYLLDLHLFLSKPIWPAPAEEEVLDDKDGILSKIDDAIANGETVYGRFPQTVIDLVSDRLREVIDSSGELSSLMKTCVNAFRLYSKTKPSPAKESIRRSKELPPEGLHPMFKNVLGGGELMALAFSERLKTFRPKQTILEAEGEAAKSKHLQGSSSQWTDVMKKKRAIHEQIINLVQQQRSNTIAEKEIEDDRASVIGKDNKEAGSKRKERNFKDEEFFISSIPTNHISESGLSVGANQEFGSNRFEAAVLDLVADDTAGMQRQKSVYHWDRRSKKYIKLNSGDRVTASGKVKTESGARVKANKTGIYKKWKETFTQ

SEQ ID NO: 231 - DNA, 1011 bp, Cannabis sativa
  source 1..1011: mol_type = other DNA
atgcctacagcaaaagggcttgtactagagaatcaccctcgcttcatcggtacatactggggcacaataagcactccctattgcaatgagactgttgaactggccgacgcttaccttatcgtcggcccagtattcaacgattatatcaatgtagggcactcgctcttacttaaagaagagcgagccataattgttcaaccccatagtgttagaatcggaaaaggtcctacctttcgattcgttgagatgagagacttcttagtggcattagcaaagagaataaaacacaatacaacatctcatgagaattatgagagaatgtatatggcagatgatggaggtcatcctctaataatgagatcttcttggtcaagtggtgatgagatcatgatgaagaaggaggcgttgagagttaatgtggtgtatgggcatttggagaaaatgttgtctagtaacacaactatgattgctgacactggtgtctcatggtttagttgccaaaagctcaaattgccacaaggatgtgggtttgaagttcaaatgcattatggatcaatcggttggtcagtaggagctagtcttgggtatgcacaggcagcttcaccaaacaaccgagtcattgcttgcattggagatggcagttttcagatgggtgcacaagaggtgtcaacaatgcttcgttgtggacaaaatagtataatatttctaattaacaatggaggctacaccatagaagaagtcatacatgaaggatcctacaatgttattaagaattggaactacactgctttagttgatgcaattaataatggtgaacacaaatgttggaccactaaggtttcatgtgaggaggaacttgttgaagcaatgaagatggcaacagaagaaaaaagagagtgtttgtgcttcattgaagtgttagtacacaaggatgacacaagcaaagagttggttgagttctgcttaagaacatcccttttcacttctcgagcacctacttatattttataa

SEQ ID NO: 232 - protein, 336 aa, Cannabis sativa
MPTAKGLVLENHPRFIGTYWGTISTPYCNETVELADAYLIVGPVFNDYINVGHSLLLKEERAIIVQPHSVRIGKGPTFRFVEMRDFLVALAKRIKHNTTSHENYERMYMADDGGHPLIMRSSWSSGDEIMMKKEALRVNVVYGHLEKMLSSNTTMIADTGVSWFSCQKLKLPQGCGFEVQMHYGSIGWSVGASLGYAQAASPNNRVIACIGDGSFQMGAQEVSTMLRCGQNSIIFLINNGGYTIEEVIHEGSYNVIKNWNYTALVDAINNGEHKCWTTKVSCEEELVEAMKMATEEKRECLCFIEVLVHKDDTSKELVEFCLRTSLFTSRAPTYIL

SEQ ID NO: 233 - DNA, 897 bp, Cannabis sativa
  source 1..897: mol_type = other DNA
atgctgaccaacaatatcgatcaaacagccagcacaagcactaataataaccacaatcgaagcgatgatcatgatgatcaagaagaagagtttcgagatatccacgcgctgactccacctcacgcgccgttacctctaccgccgggaattcgcgcttccggtcgccggagagatcagacctccgcctgggagacgagcagtcaccgatcgtcgtcgacttcgttgggaagcgaaatcggagctttgagcgagaatttcacgagtatgagccgagaattcagcgcgctggtgttagccggctccaccatcgacgggagccaaaacgacatcgatttggggcaaacgacaggaaacaatacgaataacaacaacaatttggggaggattggagaagatgaggaggtggaaaccaatcccttggcgatcgtaccagataattataataacaataatccggtttcatctccgaatcgacggcgggatttggcggcgccgcctagctcgggcgaaattagcggcggagacgatcgggttgttacgtcggttgaggtagtgaagaaggaggaagttgagtcgaaaatttcggcttggcaaaacgctaaagtggctaagattaacaatcggtttaagagggaagatgctgttattaatgggtgggagaatgaacaagttcaaatggctacttcttggatgaaaaaagttgagaggaaactagaggagaaaagagtaagagccctagaaaagatgcaaaatgatgtagcgaaagcacgtagaaaagcagaagaaagaagggcttcggccgaggccaaaagaggtaccaaagtggctagggttcttgaaatatcaaacttgatgagagccgttggaagagcaccagtcaaacgctccttcttttag

SEQ ID NO: 234 - protein, 298 aa, Cannabis sativa
MLTNNIDQTASTSTNNNHNRSDDHDDQEEEFRDIHALTPPHAPLPLPPGIRASGRRRDQTSAWETSSHRSSSTSLGSEIGALSENFTSMSREFSALVLAGSTIDGSQNDIDLGQTTGNNTNNNNNLGRIGEDEEVETNPLAIVPDNYNNNNPVSSPNRRRDLAAPPSSGEISGGDDRVVTSVEVVKKEEVESKISAWQNAKVAKINNRFKREDAVINGWENEQVQMATSWMKKVERKLEEKRVRALEKMQNDVAKARRKAEERRASAEAKRGTKVARVLEISNLMRAVGRAPVKRSFF

SEQ ID NO: 235 - DNA, 237 bp, Cannabis sativa
  source 1..237: mol_type = other DNA
ggtgtgaatgatgaattctttaaaacaagttctaattttggggcgcaaattgtacaggagaaaaagagacgacttccttcagcaggtaagaactcagccaagacagttctgactgctgctgctactgatactgctggctgtcatcgtgattccattctctcattggcttctgtcaaactcaatcaaagactcttaatatcaagcagtagagatggagctatcaaggtctggaagtga

SEQ ID NO: 236 - protein, 78 aa, Cannabis sativa
GVNDEFFKTSSNFGAQIVQEKKRRLPSAGKNSAKTVLTAAATDTAGCHRDSILSLASVKLNQRLLISSSRDGAIKVWK

SEQ ID NO: 237 - DNA, 1140 bp, Cannabis sativa
  source 1..1140: mol_type = other DNA
atggagctatcatctttgactcagacaaacattagtcttcccttcattactgccactgctgatggccccaaacacattgagaccacccttacaagggccaagtttgaggaattgtgctcagatttacttgacaggcttaaaacaccagttgaaaattccttgagggatgctaaactctcctataaagacttggatgaggtaatcctagttggtgggtcaacccgtattccagctgtccaggagcttgtgaggaaattgagtgggaaggaccccaatgtcacagtcaatcctgatgaagttgtagctctaggtgctgcagttcaggctggtgtgttggctggagatgtcagtgacattgttctgttggatgtaacaccattgtcactgggtttggaaaccctgggtggtgtcatgacaaagatcatcccaagaaacactactctgcccacatcaaaatcggaggtgttctccacagctgcagatgggcaaacaagtgtggagattaatgtccttcagggtgagagagaatttgtcagggacaacaagtctcttggaagcttccgattggatggtatcccacctgcgcctcgtggggttcctcagattgaggtcaagtttgacattgatgctaatggtatcctctcagtcgctgctgttgacaagggtactgggaagaagcaagacattaccattactggtgctagcactttacccagcgatgaggtggagagaatggtcagtgaagcagagaaattctcgaaggaggataaggaaaagagagaagcaattgacacgaagaaccaggctgattccgttgtctaccagacagagaaacagctaaaggagctgggagacaaggttcctggtgaagtgaaggagaaggttgaggccaaattaggagaactcaaggaagccatttcaggaggatctacccaaaccattaaagatgccattgctgccctcaaccaggaagtgatgcagctgggtcagtccctatacaaccaacctggtgctggtgcaggacctggtgctgcccctggtgcagggcctacccctggcgctgaagctggcccatcagattcatcgtcccagggaccagacggagatgtcattgatgcagatttcaccgacagcaagtga

SEQ ID NO: 238 - protein, 379 aa, Cannabis sativa
MELSSLTQTNISLPFITATADGPKHIETTLTRAKFEELCSDLLDRLKTPVENSLRDAKLSYKDLDEVILVGGSTRIPAVQELVRKLSGKDPNVTVNPDEVVALGAAVQAGVLAGDVSDIVLLDVTPLSLGLETLGGVMTKIIPRNTTLPTSKSEVFSTAADGQTSVEINVLQGEREFVRDNKSLGSFRLDGIPPAPRGVPQIEVKFDIDANGILSVAAVDKGTGKKQDITITGASTLPSDEVERMVSEAEKFSKEDKEKREAIDTKNQADSVVYQTEKQLKELGDKVPGEVKEKVEAKLGELKEAISGGSTQTIKDAIAALNQEVMQLGQSLYNQPGAGAGPGAAPGAGPTPGAEAGPSDSSSQGPDGDVIDADFTDSK

SEQ ID NO: 239 - DNA, 378 bp, Cannabis sativa
  source 1..378: mol_type = other DNA
gccctcgaccaatacgaagctctaaaagagaaagaaagagctgtaaatgaagagtttgaagcagccaggaaagaggaaaaggaaatagcagataaatttaacgctgtcaagttggagaggaatgagaagttcatggctgctttcagacatattgagggtattattgataagatatacaaacagcttacaaaaagcaacacacatccattgggcggaacggcttatttgaacttagacaacgaagatgatccgttcttaagcggcaccaagtatacagttatgccgccaacaaagcgttttcgtgatatggaacaactgtctggtggagagaaaacagtttgcagctcttgctttgctcttttccatccacagttttag

SEQ ID NO: 240 - protein, 125 aa, Cannabis sativa
ALDQYEALKEKERAVNEEFEAARKEEKEIADKFNAVKLERNEKFMAAFRHIEGIIDKIYKQLTKSNTHPLGGTAYLNLDNEDDPFLSGTKYTVMPPTKRFRDMEQLSGGEKTVCSSCFALFHPQF

SEQ ID NO: 241 - DNA, 597 bp, Cannabis sativa
  source 1..597: mol_type = other DNA
atgtcaaccccgccaatgagaagttttgttcagccccaacaagatgaaattaatgatgatgaagaagatgatatgttgttgcgaaggccctcggctcctttaatgctttcacccccagcaatgagaagtttcaatcagctccaacaagatgaaatcgatgatggtgaaggagatgataatgatgtgttgttgcgaaggccctcggctccaactaagtatcgttcggctccttcaatgctttcaaccccagcaatgagaagtttagatagagcagaatctgtaacaaagccagtgaatcccaattcggtaaccaccaccccaataccaaagaaaccgaaacctatggaagctgttaaaagagatatgacctatgaagagaaacaaatgcttagtacaaaccttcagaatttaccttcagagaagctagacaatgttgttcagatcattaagaagagaactcctggactttctcatcaagaagatgaaattgaagtggacattgctagtcttgaagctgaaacactttgggagttggataggtttgtgaacagttacaagaggagtttgatgagcacaaataagagaaaagaaatgtag

SEQ ID NO: 242 - protein, 198 aa, Cannabis sativa
MSTPPMRSFVQPQQDEINDDEEDDMLLRRPSAPLMLSPPAMRSFNQLQQDEIDDGEGDDNDVLLRRPSAPTKYRSAPSMLSTPAMRSLDRAESVTKPVNPNSVTTTPIPKKPKPMEAVKRDMTYEEKQMLSTNLQNLPSEKLDNVVQIIKKRTPGLSHQEDEIEVDIASLEAETLWELDRFVNSYKRSLMSTNKRKEM

SEQ ID NO: 243 - DNA, 600 bp, Cannabis sativa
  source 1..600: mol_type = other DNA
ttgggcagcgtctctctcgttcgtagtctctctctctcctctttatcgcaaaaaccctttgttggagagatccgagagatttttctgcaaacatgttggtgtaccaggaccttctcagtggtgacgagcttctctctgaactcctttccctacaaggagattgagaatggtgttctttgggaggttgaaggaaagtgggttgttcaaggtgccgttgatgtagatattggtgcaaacccatctgctgagggtgctgatgccgatgagggtgttgatgaccagactgttaaggttgtggacatcgttgacacattcaggcttcaggaacaacctccttttgacaagaagcagtttgtgacatggatcaagaggtacattaaacttttgacccccaagttggaaggagagaaacaagaggaatttaagaagggcattgagggtgcaaccaagtacctgctccccaaaattaaagacctccagttctttgtcggtgagagcatgcacgatgatggatgcttggtattcgcttactacaaggatggatgcgccgacccaactttcctctactttgcccatggattgaaggaggtgaagtgctaa

SEQ ID NO: 244 - protein, 199 aa, Cannabis sativa
LGSVSLVRSLSLSSLSQKPFVGEIREIFLQTCWCTRTFSVVTSFSLNSFPYKEIENGVLWEVEGKWVVQGAVDVDIGANPSAEGADADEGVDDQTVKVVDIVDTFRLQEQPPFDKKQFVTWIKRYIKLLTPKLEGEKQEEFKKGIEGATKYLLPKIKDLQFFVGESMHDDGCLVFAYYKDGCADPTFLYFAHGLKEVKC

SEQ ID NO: 245 - DNA, 564 bp, Cannabis sativa
  source 1..564: mol_type = other DNA
atgttggtgtaccaggaccttctcagtggtgacgagcttctctctgactcctttccctacaaggagattgagaatggtgttctttgggaggttgaaggaaagtgggttgttcaaggtgccgttgatgtagatattggtgcaaacccatctgctgagggtgctgatgccgatgagggtgttgatgaccagactgttaaggttgtggacatcgttgacacattcaggcttcaggaacaacctccttttgacaagaagcagtttgtgacatggatcaagaggtacattaaacttttgacccccaagttggaaggagagaaacaagaggaatttaagaagggcattgagggtgcaaccaagtacctgctccccaaaattaaagacctccagttctttgtcggtgagagcatgcacgatgatggatgcttggtattcgcttactacaaggatggatgcgccgaccccaactttcctctactttgcccatggattgaaggaggtgaaagtgctaagaaattgagttttagtggcttcaaagtagaagcggtttgccacccgccctattag

SEQ ID NO: 246 - protein, 187 aa, Cannabis sativa
MLVYQDLLSGDELLSDSFPYKEIENGVLWEVEGKWVVQGAVDVDIGANPSAEGADADEGVDDQTVKVVDIVDTFRLQEQPPFDKKQFVTWIKRYIKLLTPKLEGEKQEEFKKGIEGATKYLLPKIKDLQFFVGESMHDDGCLVFAYYKDGCADPNFPLLCPWIEGGESAKKLSFSGFKVEAVCHPPY

SEQ ID NO: 247 - DNA, 387 bp, Cannabis sativa
  source 1..387: mol_type = other DNA
atgatgaatgtccaaaacaagaactcttcttactttgtggaatggattccaaacaatgtcaagtccactgtttgtgacattcctcctaccgggttgaagatggcttcgactttcatcggcaactccacatcaatccaagagatgttccgtcgagttagcgagcagtttactgccatgttcagaaggaaggctttcttgcattggtacactggagaaggaatggatgagatggagttcactgaggctgaaagcaatatgaatgacttggttcggagtaccagcaataccaggatgcaactgcagacgaggaagaagagtatgaagaggaagacgggggttatgaagaagatgtttaaacaagcttcttttggttttcttatagtataa

SEQ ID NO: 248 - protein, 128 aa, Cannabis sativa
MMNVQNKNSSYFVEWIPNNVKSTVCDIPPTGLKMASTFIGNSTSIQEMFRRVSEQFTAMFRRKAFLHWYTGEGMDEMEFTEAESNMNDLVRSTSNTRMQLQTRKKSMKRKTGVMKKMFKQASFGFLIV

SEQ ID NO: 249 - DNA, 879 bp, Cannabis sativa
  source 1..879: mol_type = other DNA
ggcagaaatctaaattgttctgtccggtttcatccttggtgtgctcatcagaagggtctgttacaaagtgaagttgaaggtgttgataatgagaaggttgggttttatggaagatgtgtgcttcatgctacacagtcaatgtgtgaatctgattgtaacccaggtgatggggtaactggtagttcagggtcagaggaagtaacgtgtgcgaggactgagggttataagggccgtaaacgagatggtttctggtataaaaaattttatagtcaaattaaaggcaggggtggatgccatgttcctcaagagcaattaaatgcttggattcacattaatgggcagaaatcttgcgcacagggacattcaaaattgacaatatcagacattgaacatgattgtcggaaggaatacgctcgttataaacaatcaatgggctggaaacatctggtggtttacaagtctggcatacatgctcttggtctttacacatctcgcttcatatctcgggctgaaatggttgttgagtacattggtgagatcgtagggttgcgtgttgctgataagagagaaaatgagtatcagtctggaagaaaacttcagtacaaaagtgcttgctactttttcaggatagataaagagcatataattgatgctacttgcagaggcggaattgctcgctttgttaatcactcatgcctgccaaattgcgttgccaaggtgatttctgtaaggaatgaaaagaaggttgtctttttcgcggagagagacatattccctggagaggagatcacgtacgattaccattttaaccacgaggatgaaggtaagaagattccatgtttctgcaattcaaaaaattgcaggcgctacctgaactaa

SEQ ID NO: 250 - protein, 292 aa, Cannabis sativa
GRNLNCSVRFHPWCAHQKGLLQSEVEGVDNEKVGFYGRCVLHATQSMCESDCNPGDGVTGSSGSEEVTCARTEGYKGRKRDGFWYKKFYSQIKGRGGCHVPQEQLNAWIHINGQKSCAQGHSKLTISDIEHDCRKEYARYKQSMGWKHLVVYKSGIHALGLYTSRFISRAEMVVEYIGEIVGLRVADKRENEYQSGRKLQYKSACYFFRIDKEHIIDATCRGGIARFVNHSCLPNCVAKVISVRNEKKVVFFAERDIFPGEEITYDYHFNHEDEGKKIPCFCNSKNCRRYLN

SEQ ID NO: 251 - DNA, 879 bp, Cannabis sativa; identical to SEQ ID NO: 249
  source 1..879: mol_type = other DNA

SEQ ID NO: 252 - protein, 292 aa, Cannabis sativa; identical to SEQ ID NO: 250

SEQ ID NO: 253 - DNA, 639 bp, Cannabis sativa
  source 1..639: mol_type = other DNA
ggcttctggttattatctgactctctttctctctcttttaatcaacacattttctcattttcttacccatttccctctttgaatctttcgtttagcaagaacctggctaactctcaggaaattctggaggagagtggttctagtaatcaaagggtggttcgggccctgtatgaagccttgagctcccgagacgtggacaaggttcacagccttctctcgccggatctagattggtggttccatggtccgccgtcacacccagttcttgatgcgattgctcaccggtgtttcatcaccagacgacgtcgttttccaattccagccccaatccttcgcctcattcggacccaccgtccttgtcgaaggctgcgatcagatctgtcaatagcttgggttcacgcttggaccgtgacggatgggataattacccaggttagggagtacttcaacacttcccttaccgtcacccgcctcggcgattcttcctcctcttacggcaaatactctccgctaggattcgcttcgtcgtcttcctcatcaacgtccgagattgcttcgtaccactgcccctccgtttgggagagtagcctctccagtcgggtcggaaagtcggtgccgggtcttgtcctggcgatttaa

SEQ ID NO: 254 - protein, 212 aa, Cannabis sativa
GFWLLSDSLSLSFNQHIFSFSYPFPSLNLSFSKNLANSQEILEESGSSNQRVVRALYEALSSRDVDKVHSLLSPDLDWWFHGPPSHPVLDAIAHRCFITRRRRFPIPAPILRLIRTHRPCRRLRSDLSIAWVHAWTVTDGIITQVREYFNTSLTVTRLGDSSSSYGKYSPLGFASSSSSSTSEIASYHCPSVWESSLSSRVGKSVPGLVLAI

SEQ ID NO: 255 - DNA, 435 bp, Cannabis sativa
  source 1..435: mol_type = other DNA
atgaagctgttgcacaatggtgcaaagacatattcattgccaaggattctttattggataaacataaagctgaggacacttttgctggtcaagaattgcaagacactgtcgaccaaaaaaatctcttgtggtggttgtatcttggcatgtcttcttgcattcggggctttgaagttcctccaatggtcttcattcttatcctcatggaaagggcattgcactctcagccttccttttggggttagttaccatccttatgcaattcttgattctcttctctcagtccgaacgttcaacccctgccaaggtggctccaacaaagaacaagaaacgacggagagccttctgaatctgcaaaacaagataaacagaactaaatcaccgctccgatcccctcccaacattccttctgtaacagaaaggttcaaattttaa

SEQ ID NO: 256 - protein, 144 aa, Cannabis sativa
MKLLHNGAKTYSLPRILYWINIKLRTLLLVKNCKTLSTKKISCGGCILACLLAFGALKFLQWSSFLSSWKGHCTLSLPFGVSYHPYAILDSLLSVRTFNPCQGGSNKEQETTESLLNLQNKINRTKSPLRSPPNIPSVTERFKF

SEQ ID NO: 257 - DNA, 492 bp, Cannabis sativa
  source 1..492: mol_type = other DNA
atgtggcggatcctgaaacgaaaaacgacaactatttacacatcgcattgttggtggggccttcttctcaacttattgatgtttgctcctcgtctcactactgtagagccagtcgtagacagttggagagtttggttcagcttgtcaaagagggtgccgcctaggcctttggtagcaaggttgctgcctgggttaaacacgctcctccacacgttgtctttgctacgcgccaacgacgccgaaagaggagtcgttggtgtgactggtgtcactggactaaccggcatcgacagtgatctctggtacaccatattactcgtttccccttcgccctctttgataatcatcaagggcgatggcctggtggtgatcttcctgagtttaccaagcccacgctcaggtgaaggcccggcaacgacgtcgtcccacagtttatctagcagaaccatatttcccggtgatatttcaatgagtctctcacctaactctccc

SEQ ID NO: 258 - protein, 164 aa, Cannabis sativa
MWRILKRKTTTIYTSHCWWGLLLNLLMFAPRLTTVEPVVDSWRVWFSLSKRVPPRPLVARLLPGLNTLLHTLSLLRANDAERGVVGVTGVTGLTGIDSDLWYTILLVSPSPSLIIIKGDGLVVIFLSLPSPRSGEGPATTSSHSLSSRTIFPGDISMSLSPNSP

SEQ ID NO: 259 - DNA, 201 bp, Cannabis sativa
  source 1..201: mol_type = other DNA
atgaagattagttcacacccgaaaatcgaaaaagagaaaaaaaaaactgaaaaaaacagaaagaagaagaacaaaaaaaaagatgggaactttagacagaaggaagaggaggagaaaattggaaagcacaagtgtatcattaaaagggtttttctttttgtttttttttttaactggttgacagtagcattatgtttttag

SEQ ID NO: 260 - protein, 66 aa, Cannabis sativa
MKISSHPKIEKEKKKTEKNRKKKNKKKDGNFRQKEEEEKIGKHKCIIKRVFLFVFFFNWLTVALCF

SEQ ID NO: 261 - DNA, 216 bp, Cannabis sativa
  source 1..216: mol_type = other DNA
atgcatgcatgcatgcatgcatccaccttatatgtgaataataatcctgtattagtgtgtatgaagtcttatatgtattcagttatgtactatatatatcattatttaattatatatatatcgtattgtggctccgttaagagacttcttgttgaaaggtctagagatttgtattttccctttatttacaaattatatagaagaataaatttgaga

SEQ ID NO: 262 - protein, 72 aa, Cannabis sativa
MHACMHASTLYVNNNPVLVCMKSYMYSVMYYIYHYLIIYISYCGSVKRLLVERSRDLYFPFIYKLYRRINLR

SEQ ID NO: 263 - DNA, 306 bp, Cannabis sativa
  source 1..306: mol_type = other DNA
atgtatatattatccaaggagaaggaacgatctgagccggtcaatcggatcaagatcattatcaaccgtccgttaccggagaaggaaagtccgttactattaataaccgttaaaaagaatagaaaatcgcgcgaaaaaaagtcaaaaggcttaaattatgcaaaaagaaaaaaaaaagttatatgtttattatctgtttctttaatattaaaaattagatgtattatttgtataatttgtactttttggctttgttttttcttttttataattaacaatatagcaattgggggctctgtattttga

SEQ ID NO: 264 - protein, 72 aa, Cannabis sativa
MYILSKEKERSEPVNRIKIIINRPLPEKESPLLLITVKKNRKSREKKSKGLNYAKRKKKVICLLSVSLILKN

SEQ ID NO: 265 - DNA, 435 bp, Cannabis sativa
  source 1..435: mol_type = other DNA
atgactactgaaaatgatgattccgtcgacaaagctgtggccgaacgtcgagagaggctgaaagccctcaaagcttcacaagagctctcaaataccccagaagaagatgacaatcaaaccaataaagaaacgaatcttaacatgaaatttcgaaattatgttcctcatgataagaagcttcaagaggccaaggttgcccctcccgttcttccgaaatttgaagaccctgttgcagtagagcctccaccatctgagaagaaagaggacccatttgtcaatatagctcctaaaaaacctaattgggatctcagaagggatgtgcaaaaaaaacttgataagcttgaaaaacgaacccagaaggcaatttatactctcatggaggaacaagtaaagcaaaaagaattggctgaagatggtacaaatgaggtggcttaa

SEQ ID NO: 266 - protein, 144 aa, Cannabis sativa
MTTENDDSVDKAVAERRERLKALKASQELSNTPEEDDNQTNKETNLNMKFRNYVPHDKKLQEAKVAPPVLPKFEDPVAVEPPPSEKKEDPFVNIAPKKPNWDLRRDVQKKLDKLEKRTQKAIYTLMEEQVKQKELAEDGTNEVA

SEQ ID NO: 267 - DNA, 132 bp, Cannabis sativa
  source 1..132: mol_type = other DNA
caatttgagattacttctaacatttgcataaagattggacataattttaaaaaaccaaaagaagaaaaaaaaaaaaaaaagacaaagcatataatacaaatacagagcactgcagagagcaagaaaatataa

SEQ ID NO: 268 - protein, 43 aa, Cannabis sativa
QFEITSNICIKIGHNFKKPKEEKKKKKTKHIIQIQSTAESKKI

SEQ ID NO: 269 - DNA, 918 bp, Cannabis sativa
  source 1..918: mol_type = other DNA
atgagggccactactccaatttttgctcttatgctattcttcctcctccgcctcccaaaatctctatcaaagaataattatgggacctctgtcccaaaatctgatgttgatctcttggaatttcctttaaatcttgagttttttgaagctgaattcttcttgtatggagcattgggttatggtttggacaaagttgctccacaattaaccaaagggggcccaccacccattggtgctacaaaggccaatttagatactctcactaaagatgtcatcttgcaatttggattgcaagaagtaggacacttgagggctattcaaaaaacagtgaaagggttcccaagaccactattgaatttaagttcatcatcatttgcaaatgtaatagatagtgcatttgggagaaagctaaggcctccctttaatccttacaaaaatagcattaattttcttatagcatcatatgtgattccctatgtgggactcactggctatgttggaacaaacccaaatctcaatggaactacttccaaaagacttgtagcaggtcttttgggtgtggaatcaggccaagatgctgtgattagagcaatgctatatgagcgtaggaagatgatagtgaagccatataacatgaatgtggcagagttcacaaatcaaatttctaagctaaggaacaagttgggaaaagcaggtttgaaagacgaagggcttatggtaccaacatctctaggcgctgagggaagagtttctggaaatgtactctctgcaaaccaagactcgctttcttatggaaggacaccgatggagatacttagaattgtttatggaagtggtaatgaacatgtgcctggtggtttctatcccaagggtggtaatggtcgcattgctaagtcttatttggatcatcatgcctaa

SEQ ID NO: 270 - protein, 305 aa, Cannabis sativa
MRATTPIFALMLFFLLRLPKSLSKNNYGTSVPKSDVDLLEFPLNLEFFEAEFFLYGALGYGLDKVAPQLTKGGPPPIGATKANLDTLTKDVILQFGLQEVGHLRAIQKTVKGFPRPLLNLSSSSFANVIDSAFGRKLRPPFNPYKNSINFLIASYVIPYVGLTGYVGTNPNLNGTTSKRLVAGLLGVESGQDAVIRAMLYERRKMIVKPYNMNVAEFTNQISKLRNKLGKAGLKDEGLMVPTSLGAEGRVSGNVLSANQDSLSYGRTPMEILRIVYGSGNEHVPGGFYPKGGNGRIAKSYLDHHA

SEQ ID NO: 271 - DNA, 750 bp, Cannabis sativa
  source 1..750: mol_type = other DNA
atggctaatggggaagagaaaagcagtgacttttatgcagttttgggtttgaaaaaggaatgcacaactttggaactgaggaatgcttacaagaaattagcaatgagatggcatccggatcggtgttcggcttccgggaattcaaagttcgtggaggaagccaaaaagaaattccaggccattcaacaagcctattctgttttatcagacgagaataagaggttcatgtacgacgtcggagtctacgatagcgatgatgacgaaagtggtatgggtgattttttgagcgagatggctgtaatgatgagtcaaacaaagcctaatgaaagtggggaagaaacctttgaagaattgcagcaactatttgaagatatgtttcaagggaataatgatgcatttggatcaagcaatagtacttcttactgtgaaacctcaacgacatcttctacgtcctatgcatcttataattgtgaaagttcaagttccagtaataaacgtagctccaccgatatgaacaatggaattaggacaaatgggaatgattcttcttcaggctttgctgctcatcttcagagcttttgtgtaggggtggatcagcggcggggtttgagggaggggagggtagtaggaggaggggcagttcgaggaggagcaagcgataaccaccgacgacatggaaggaaacaaaaggtttcgtctggtcacgatgtttcttcgaacgactgctctagtggtatatcagcagcttga

SEQ ID NO: 272 - protein, 249 aa, Cannabis sativa
MANGEEKSSDFYAVLGLKKECTTLELRNAYKKLAMRWHPDRCSASGNSKFVEEAKKKFQAIQQAYSVLSDENKRFMYDVGVYDSDDDESGMGDFLSEMAVMMSQTKPNESGEETFEELQQLFEDMFQGNNDAFGSSNSTSYCETSTTSSTSYASYNCESSSSSNKRSSTDMNNGIRTNGNDSSSGFAAHLQSFCVGVDQRRGLREGRVVGGGAVRGGASDNHRRHGRKQKVSSGHDVSSNDCSSGISAA

SEQ ID NO: 273 - DNA, 333 bp, Cannabis sativa
  source 1..333: mol_type = other DNA
atgggctctaaaactttggtttaccttggtcttttacttgctgttgttcttctcatatgctcagaggtagcagccaaggagcaagcagtgaatactgtagaaaacaaaaacgacgatgcaaaatacggtaactataatggatatccagggaatgggggcaacggtggcggccataatggcggttattaccaaggcggcggcggcggcggtggtagtggtggggctatcacaatggtggtggtggtggtggtggtggtggtggaggtagctggggcagtcatcattgctaccatagttgctgccagtggagctacggacacaaacagtgtctaa

SEQ ID NO: 274 - protein, 110 aa, Cannabis sativa
MGSKTLVYLGLLLAVVLLICSEVAAKEQAVNTVENKNDDAKYGNYNGYPGNGGNGGGHNGGYYQGGGGGGGSGGAITMVVVVVVVVVEVAGAVIIATIVAASGATDTNSV

SEQ ID NO: 275 - DNA, 804 bp, Cannabis sativa
  source 1..804: mol_type = other DNA
atgccaaagttgggttctttgacaaccaaacaagataatcatgaagaacaagcaaatgtgactgaagttctctcaagttttagacccaaagatgatggaaaacaagttaggaggagagatccaaacccaccacctaagaaaatctttagaggtcatcatagagacaacaatattggctcttcatcatcattaccatcaagctcaaatatttcttcccaagatgaaaaattgaagctaatggagatgataatggagttgagtcaagaagattcaatactcaaatataacacctttacttctcttcttcctccacccccaccaagaacagtttctaagatcttacctccatcaattaggccaatcccagttcaagtaagaagcagattatcaccacatttgattcaaacttcattggaagaccaccataagaaagatgaggaacaatggcttggtaaaaaaccaagtgttattgttaaaccatgtcattatcctctgtcatcaaatctgaatgcttatactacaacaacatcaccatcatcatcaggtattaagtccagtcatctatcaagaaccatgttcagtgatcaatctcatccacacaaaatagccccagcagttcaaataaggtcagtgataccagtctgtgcagcagcaccagtaacacactcagctactactaaaccaagagcagaagaggtctcatcatcatcatcagttactactactactactactactacttctaagttcaataaaccacagtttagtatgcttggttctgatttcaaaaagttacaattatga

SEQ ID NO: 276 - protein, 267 aa, Cannabis sativa
MPKLGSLTTKQDNHEEQANVTEVLSSFRPKDDGKQVRRRDPNPPPKKIFRGHHRDNNIGSSSSLPSSSNISSQDEKLKLMEMIMELSQEDSILKYNTFTSLLPPPPPRTVSKILPPSIRPIPVQVRSRLSPHLIQTSLEDHHKKDEEQWLGKKPSVIVKPCHYPLSSNLNAYTTTTSPSSSGIKSSHLSRTMFSDQSHPHKIAPAVQIRSVIPVCAAAPVTHSATTKPRAEEVSSSSSVTTTTTTTTSKFNKPQFSMLGSDFKKLQL

SEQ ID NO: 277 - DNA, 231 bp, Cannabis sativa
  source 1..231: mol_type = other DNA
atgcctaggcatctctgtagaataaaagaaagatgtttcatggtgaaacgtgaatatgtatataaatatattaaaaaaaaaggaaaaaaaggtagtagaaagttatattcttaccattcttttctgttcatatttgatatgcctctctcaatcttaaagattatcttgaatgtattaaataagagacgagcgcaaattcccaaattccattgttataatatagtttcttag

SEQ ID NO: 278 - protein, 76 aa, Cannabis sativa
MPRHLCRIKERCFMVKREYVYKYIKKKGKKGSRKLYSYHSFLFIFDMPLSILKIILNVLNKRRAQIPKFHCYNIVS

SEQ ID NO: 279 - DNA, 1092 bp, Cannabis sativa
  source 1..1092: mol_type = other DNA
atgcttgccaaactatggctcactcttcttttccttggattggtggagctcaccgcaaccaccaccggtgttgttgctgcaagcactcacttgcctcgtaatttgaatgaattacctcatcgtgagaaaaagccacctcgaaaagatcaaccgaaacttaatccaggtgataagccacctttgaatccaggtgataagccgccacctgaagacaaaccacctttaaatccaggagataaacctccattgaatcctggtgataagccaccaccagaagacaagccaccattgaatccacgagataaactcccgttgaatccaggtgataagcctccaccagaagacaagccaccactgaatccaggagataagcccccgttgaatccaggtgataggcctccaccagaagacaaaccaccactgaatccaggagataagcccccgttgaatccaggtgataagcctccaccagaagacaaaccaccactgaatccaggagataagccccccttgaatccaggtgataagcctccaccagaagacaagccaccactgaatccaagagataagcccccgttgaatccaggtgataagccaccattgaatccaggtgataagcctccaccagaagataagccaccactgaattcaggtaatgataagcctccaccaaaagacaagtcaccactcaattcaggtcataagcccctgttgaatcctcgtgataagcctccaccaaaagacaagcaacctttgaatccaggtgataagccaccactgaatccaggagataaaccgccattgaatcccggtgataagccactgccaaaagataaaccaccactaaatccaggtgataagccaccaccagaagataaaccaccactaaatcctggtgataagccaccaccagaggataaaccaccattaaatccaggtgataaaccaccaccggaggataacccgcctttgaatccaggtgacgaaccgctaccaccaaaaaataatccaccagaaaaaggaccatcaagaaagactgagattgtgagagacaacctatcaaggaaggtgatgaagaacttgcaaccctaa

SEQ ID NO: 280 - protein, 112 aa, Cannabis sativa
MVLFLVDYFLVVAVRHLDSKAGYPPVVVYHLDLMVVYPLVVAYHQDLVVVYLLVVAYHLDLVVVYLLAVAYHRDSMAVYLLDSVVAYHLDSKVACLLVEAYHEDSTGAYDLN

SEQ ID NO: 281 - DNA, 297 bp, Cannabis sativa
  source 1..297: mol_type = other DNA
atgaaacaagtgaagaagcctgtaagtaaggggcttggaaaaaagatgtttgaaaaaacaacctctccaacaaagagacaaggttgtcatggcagacatatatataacctgctaccatggccaagacccccaaaaaacactaggcaccaaaaacttgtacaactgccaaacgcaactctaatatatgcgaatgaactcacaaaactagggacggacctacccttttatggaaatattaaaaagacccaaccatcacgcaaagaaccccaaagttcaaaaacgtggaagactcggtaa

SEQ ID NO: 282 - protein, 98 aa, Cannabis sativa
MKQVKKPVSKGLGKKMFEKTTSPTKRQGCHGRHIYNLLPWPRPPKNTRHQKLVQLPNATLIYANELTKLGTDLPFYGNIKKTQPSRKEPQSSKTWKTR

SEQ ID NO: 283 - DNA, 966 bp, Cannabis sativa
  source 1..966: mol_type = other DNA
ggaaagactgctcctcaacccgatagatggtacaacctgtctctaggctcttcattcaaggatgattcctccaagtattgcactttacgatatgagtttaagccggcttctattgataagagcaagcctgggtccttacacaaaacaaaggaaaatagggttagtgtagaattccagaacaatcaattgggaaaacctaaagtggcattcgagggaaaaagtgaggattacaaggagagcgatgctgtattgttctttgatggggagacttttcgcttggagagacttcatcggggcgtcaaacaactccggcacgtacgacagcctggtgaatcgaacgctgctgctgctgctgctgttgctgcggtgatagagcctcgattatctccagttgcaaaggctcagaagactattcatgttggtagaagctcataccctgcagtgccggttgaggtggagcgaattgatattggtggaccggagaatgctgtgagtgcaaaatctgccagtaaggggactgttgctgattatacctctgaaccgccaaacgtatctgaaccaccaaacatatctgctgtatcacccgaacccaagaacgtggaagtagatgaacatcatgagatagacatggaagacatttttggttctacatcacctgtagccgaagatggagatgatactgctgttgcagaagataaatctaacgcagcagggtttgatatgaatgtaccacgacaaaatgacacggatgatgagattgctgatgttgacgatagtggtgatgaagtcgacaaaggtccaaatgctgcagaagcactaaaagctcaagttactgcagagggtagagacgaacagacttcctcttcgagtagcagcagcggaagtggaagtagtagcagtagtgcaagcagtagcagcagtagcgacaaggaaggcagcgatgaagattccatcaattccatttga

SEQ ID NO: 284 - protein, 321 aa, Cannabis sativa
GKTAPQPDRWYNLSLGSSFKDDSSKYCTLRYEFKPASIDKSKPGSLHKTKENRVSVEFQNNQLGKPKVAFEGKSEDYKESDAVLFFDGETFRLERLHRGVKQLRHVRQPGESNAAAAAAVAAVIEPRLSPVAKAQKTIHVGRSSYPAVPVEVERIDIGGPENAVSAKSASKGTVADYTSEPPNVSEPPNISAVSPEPKNVEVDEHHEIDMEDIFGSTSPVAEDGDDTAVAEDKSNAAGFDMNVPRQNDTDDEIADVDDSGDEVDKGPNAAEALKAQVTAEGRDEQTSSSSSSSGSGSSSSSASSSSSSDKEGSDEDSINSI

SEQ ID NO: 285 - DNA, 288 bp, Cannabis sativa
  source 1..288: mol_type = other DNA
atgatgtgctcaatgatatttgggtgttcaatggggacgagtgtcacaagtgggtccaaatactatatgaactacaaaacattccggaggagggtcactccctcgagttggtcactcagctactcatatcctaggtggtcgagtgctaatatatggaggggagattcttctagacaccggaaagacgaatttctgggtgttagacttgagtgcagttccatccattggagtgcagccagatacagtgacctccaaacgaattttggcaaaaatgtggaaatgttttaa

SEQ ID NO: 286 - protein, 95 aa, Cannabis sativa
MMCSMIFGCSMGTSVTSGSKYYMNYKTFRRRVTPSSWSLSYSYPRWSSANIWRGDSSRHRKDEFLGVRLECSSIHWSAARYSDLQTNFGKNVEMF

SEQ ID NO: 287 - DNA, 1365 bp, Cannabis sativa
  source 1..1365: mol_type = other DNA
atggcccaatacggcccaacagttatggtccccatcgacgtgaagaagaagccccgggaacagaaggttccacttcacaaccggtggcaccctgacatacctccggtggcggaggcggcggtcggagatgtatttagagttgaaatggtggattttagcggcggcggtattaccaaggagtactcggctaatgacatcaaatttgctgatccttttattgttcattatcttagtgggccaatcagaatagtggacaaagatgggataccggccaagccaggggatcttctagtggttgaaatatgcaacttgggtccactcccaggagatgaatgggggttcacagctacctttgatagagaaaatggaggtggttttcttactgatcattttcctgctgcaaccaaagctatttggtattttgaaggaatttatgcttactctcctcacataccaggagtaagatttcctggtttgactcacccgggaatagttggaacagctccatcaatggaacttcttaatatttggaatgaaagggaaagacagcttgttgagaatggagttgagtctttgaaactttgtgaagttgtgcatcaacggccaatggctagcttaccaacaccaaaaggttgtgttctaggaaagattcaagaaggtaccccagagtgggagaaaatagctagagaggctgcaaggacaataccaggaagagaaaatggtgggaactgtgacataaaaaacctaagtagaggatcaaagatctacctacctgtattcgtagaaggagccaatctaagtactggtgacatgcatttttctcagggagatggtgaagtctcattttgtggggcaattgagatgagtggctttcttgatcttaagtgtgaaattataaggggtggaatgaaagagtatttgacaccaatggggccaactcctcttcatgtaaacccaatatttgagataggccctgttgaaccaagattctcagaatggcttgtctttgagggtataagtgttgatgagagtgggagacagcattatctcgacgcaactgtggcttacaagcgtgcagttctcaatgctattgactacctctccaaatttggatacagcaaagaacaggcctaccttctcttgtcttgttgcccatgtgaagggagaatttctggaatagtagatgccccaaatgctatggctaccttggcaattccaacagctatatttgatcaggatattcgtccaaaagcaagtaaagtacccatagggccccgggtagtgaggaaaccagatgtactgaaatgtagttatgatggaaatttgcccacaacaaccaatctttcttctagctctacctcttaa

SEQ ID NO: 288 - protein, 454 aa, Cannabis sativa
MAQYGPTVMVPIDVKKKPREQKVPLHNRWHPDIPPVAEAAVGDVFRVEMVDFSGGGITKEYSANDIKFADPFIVHYLSGPIRIVDKDGIPAKPGDLLVVEICNLGPLPGDEWGFTATFDRENGGGFLTDHFPAATKAIWYFEGIYAYSPHIPGVRFPGLTHPGIVGTAPSMELLNIWNERERQLVENGVESLKLCEVVHQRPMASLPTPKGCVLGKIQEGTPEWEKIAREAARTIPGRENGGNCDIKNLSRGSKIYLPVFVEGANLSTGDMHFSQGDGEVSFCGAIEMSGFLDLKCEIIRGGMKEYLTPMGPTPLHVNPIFEIGPVEPRFSEWLVFEGISVDESGRQHYLDATVAYKRAVLNAIDYLSKFGYSKEQAYLLLSCCPCEGRISGIVDAPNAMATLAIPTAIFDQDIRPKASKVPIGPRVVRKPDVLKCSYDGNLPTTTNLSSSSTS

SEQ ID NO: 289 - DNA, 522 bp, Cannabis sativa
  source 1..522: mol_type = other DNA
ggtaaaattctcttcaccatctttcttctcactgctgcatcttcactctgttactcgacccgacccgcttccgacaaacccttctctgctgaactcaagcactcctccaagtccaagggaaccccttcaaaccagaacaacaacaaaggtggtagtggcaaccccggtattccaggcgggtttttcggacccggacccgctttcaacatacccggattcggaaacggtttcggcaacgggatcgtaggtggcgggtacggatccgggtatggtggtccgagtgggggccattccagaaatggagtagtgaggcccactgtggtgtgcaaggaaaagggaccctgttaccaaaagaagctgacgtgtccagctaagtgtttcacctcctatagccggtcagggaaggggtacggtggaggtggcggtggtggcgggtgcaccatggactgcaagaaaaaagtgtattgcttattgttagaaggatattggctgaggcttcggtgtgacagtgtcagccggtag

SEQ ID NO: 290 - protein, 173 aa, Cannabis sativa
GKILFTIFLLTAASSLCYSTRPASDKPFSAELKHSSKSKGTPSNQNNNKGGSGNPGIPGGFFGPGPAFNIPGFGNGFGNGIVGGGYGSGYGGPSGGHSRNGVVRPTVVCKEKGPCYQKKLTCPAKCFTSYSRSGKGYGGGGGGGGCTMDCKKKVYCLLLEGYWLRLRCDSVSR

SEQ ID NO: 291 - DNA, 96 bp, Cannabis sativa
  source 1..96: mol_type = other DNA
atgatgatgatgatggtgatgttgatgatgatgctttgctttagttgtggagcaaaattttccagattttcatacacacaatatgtgtttggctaa

SEQ ID NO: 292 - protein, 31 aa, Cannabis sativa
MMMMMVMLMMMLCFSCGAKFSRFSYTQYVFG

SEQ ID NO: 293 - DNA, 177 bp, Cannabis sativa
  source 1..177: mol_type = other DNA
atgctatggatgacgattatgacttcacagtggattataagaaagggtcttctatggatgtcagtgatgatgaggaataaatcatttttttcttacctttttttcggctattttgttagctgcagttttccttttaccttaaattttttgtttttatttctgggggaaacaaattga

SEQ ID NO: 294 - protein, 58 aa, Cannabis sativa
MLWMTIMTSQWIIRKGLLWMSVMMRNKSFFSYLFFGYFVSCSFPFTLNFLFLFLGETN

SEQ ID NO: 295 - DNA, 591 bp, Cannabis sativa
  source 1..591: mol_type = other DNA
atgatcgtcattcattataatattttttggggctgggttctaagcaatgctacacaacttcatcttgtgttatggctcaaaactcagtaccaagtctcctccaaatctctactcgtcatcatcatccatcatctccactctcgtcctcctcatcctcatcatcattcacttctgactttgacttgtcagactcttcttcgtctgctccagtatctccatcagccagcttcttgttgtaagctaccatgtttttgttgtactcaactttcctcttctcagctcttgaaacaaatggagctttctcagcttcagtcaatgatttccatttctcaccaccagctttaccgacagcggaaacggatttgtttgttaggatgttccttcttgtattttttctctaaattcctccatgaaaacgaagaaagcactggcaggcctcttcggcttgttaggatcctttccagcctttccagccttcttaccagctccagcgctcttgcttttcagcttggtatcagttctcctgggagcagccttagattttccacctttcatggcgaacctacactaaaaaaagtcgtaacgcggtga

SEQ ID NO: 296 - protein, 196 aa, Cannabis sativa
MIVIHYNIFWGWVLSNATQLHLVLWLKTQYQVSSKSLLVIIIHHLHSRPPHPHHHSLLTLTCQTLLRLLQYLHQPASCCKLPCFCCTQLSSSQLLKQMELSQLQSMISISHHQLYRQRKRICLLGCSFLYFFSKFLHENEESTGRPLRLVRILSSLSSLLTSSSALAFQLGISSPGSSLRFSTFHGEPTLKKVVTR

SEQ ID NO: 297 - DNA, 381 bp, Cannabis sativa
  source 1..381: mol_type = other DNA
ttgggtttcaacacggacgatatcactaaaagagcagaggaagacataaaaaagaccaaagagacagcacaagatctttcagacagtgcgaaagggtatgctgatgagactaaaaagaaggctgagggagcagcggcttcactgggtgacaaggccaaagaagtatccgctagagctggagagaatgcggaggctgcaaaggagaacatcatgggagcagccgagtcagcgaaggaaaaggcgaaagaaggaggtgagaaactggccgagaacgtcgagagtgctaaggaaagtctcaaagacgcagctgaatccgccgtagacaaggccaaggagactggccagaagctcaaggatgcaattactggagatggcaaatag

SEQ ID NO: 298 - protein, 126 aa, Cannabis sativa
LGFNTDDITKRAEEDIKKTKETAQDLSDSAKGYADETKKKAEGAAASLGDKAKEVSARAGENAEAAKENIMGAAESAKEKAKEGGEKLAENVESAKESLKDAAESAVDKAKETGQKLKDAITGDGK

SEQ ID NO: 299 - DNA, 213 bp, Cannabis sativa
  source 1..213: mol_type = other DNA
atgtgtaatcttatcttcagtgtatatataaataagtcaagtcagtttatctgcaaaaaaaaaaaaaaaaaaaaaaaaaaaaaaaaaaaaaaaaaaaaaaaaaaaaaaaaaaaaaaaacccaactttgtacaagaaagttgggtttttttttttttttttttttttttttttttttttttttttttttttttttttttttgcagataaactga

SEQ ID NO: 300 - protein, 70 aa, Cannabis sativa
MCNLIFSVYINKSSQFICKKKKKKKKKKKKKKKKKKKKKTQLCTRKLGFFFFFFFFFFFFFFFFFFLQIN

SEQ ID NO: 301 - DNA, 138 bp, Cannabis sativa
  source 1..138: mol_type = other DNA
ggggggacaactttgtacaaaaaagttggcttctctcttcaccatctaagccaaaagtattttattttattttatatagaaaaatgtcttgcaactgcggctcaggctgtaagtgctaaaatgatgggcgaatcatag

SEQ ID NO: 302 - protein, 45 aa, Cannabis sativa
GGTTLYKKVGFSLHHLSQKYFILFYIEKCLATAAQAVSAKMMGES

SEQ ID NO: 303 - DNA, 843 bp, Cannabis sativa
  source 1..843: mol_type = other DNA
ggggttactgctatatctgatgacaacatcacagaaccaaaagctgagaatcaggaaactactaacaagactattgatgaagagaaagaacaggttgcaaatgacccatctacagcagaagaattgaaggaagaaactgaaaaggtcccatctacagcagaagaattgaaggaagaaactgaaaaggacccatctacagcagaagcactggaggaagaagctgaaaaggaaaaagagcctgaagctccatcagatctctcccatcaggttgaggcggtcccagtgcctacaccagtagcagatgtgctagtatcagaagacgtggaggtagctgatgagcctgcggttgaggttgaagaaggtaatgagacacaaccacttgagaaagctgagcaagatgtaatggaagcagtttttgaggccgctgatgaaaatgaagcgctagtattagaggacgtggaagaggtagcttatgagcaggcacttgaggttggagaacgtaatgagacagaaccacttgagaaagctgagcaagatgtaatggaagcagtatttgaggctgctgatgaacacgaagcgctagtatcagaggacgtggaagtggtagctggtgagcaggcacttgaggttgaagaacataatgagacagaagcacttgagaaagctgagcaagatgtaatggaagcagtttttgaggctgctgatgaaaatgaagaaaagaaaccagaggaggagaaagctggggtcttcaccccaaaactgactagcgatgaaaactctaattattcggtaaaccttgaggagaagatgtcacaggagcccaaggcttcacaagttagttga

SEQ ID NO: 304 - protein, 280 aa, Cannabis sativa
GVTAISDDNITEPKAENQETTNKTIDEEKEQVANDPSTAEELKEETEKVPSTAEELKEETEKDPSTAEALEEEAEKEKEPEAPSDLSHQVEAVPVPTPVADVLVSEDVEVADEPAVEVEEGNETQPLEKAEQDVMEAVFEAADENEALVLEDVEEVAYEQALEVGERNETEPLEKAEQDVMEAVFEAADEHEALVSEDVEVVAGEQALEVEEHNETEALEKAEQDVMEAVFEAADENEEKKPEEEKAGVFTPKLTSDENSNYSVNLEEKMSQEPKASQVS

SEQ ID NO: 305 - DNA, 315 bp, Cannabis sativa
  source 1..315: mol_type = other DNA
atgtttattgatgacaccatgtgctataaatttaacaggcatcaaggtctcggcatggaggaggtttgggctcacttaacaaagttgtgttttcctccttctggtccacatttagttcgtcctggatttttgagtgccatgcggtggcttctcgccctttctcaacttttcaactatctctacaactttctttggagtaacatcttcaaaatagttataagtatatccttcagatccattggagtagtctgccacagtgatcatgggagcatttacacagcaacccatgcattccatttctccaacagaaaataa

SEQ ID NO: 306 - protein, 104 aa, Cannabis sativa
MFIDDTMCYKFNRHQGLGMEEVWAHLTKLCFPPSGPHLVRPGFLSAMRWLLALSQLFNYLYNFLWSNIFKIVISISFRSIGVVCHSDHGSIYTATHAFHFSNRK

SEQ ID NO: 307 - DNA, 237 bp, Cannabis sativa
  source 1..237: mol_type = other DNA
atgataattgcaaatttcttttataatatatataatttattctacaatttccggttcacatataatattgcaattttgctatacctgtttttatcatctctcatactcagatattttttattctatagcaaaatgtacaattcaaggacttatacgtgtatcatatacggtccaatattcaaacagatgaaactaattaataaacacagaaagaaaaaaagaaaaaaaaaacactaa

SEQ ID NO: 308 - protein, 78 aa, Cannabis sativa
MIIANFFYNIYNLFYNFRFTYNIAILLYLFLSSLILRYFLFYSKMYNSRTYTCIIYGPIFKQMKLINKHRKKKRKKKH

SEQ ID NO: 309 - DNA, 537 bp, Cannabis sativa
  source 1..537: mol_type = other DNA
atggcaagaacgggagttttaacattaactgggtctcactggaccaaattaaaaagtaagaactttaaaactgaaaataaagaacactttgtcagcaaaaggattaataacaagcccatcaaaagacaccacaccgaaaagcagagaccgaactccactacttcagaaatccccaccacgaagacgaagaacaaggtggagggtagactccttctgaatgttgtaatcagcaagagtacgaccatcctccaattgcttcccgcaaagatgagacgctgctggtctggagggatgccttccttgtcttggattttagccttgacattatcaattgtgtcagagctctccacctccaaagtgatggttttgcccgtaagggtcttcacgaagatttgcataccacccctcaaacgcagcaccaagtgcagagtcgactccttcctgaatgttgtagtcagcaagggtgcgaccgtcctccaactgctttccagcgaaaatcagacgttgctggtccggggaattccttccttgtcctga

SEQ ID NO: 310 - protein, 178 aa, Cannabis sativa
MARTGVLTLTGSHWTKLKSKNFKTENKEHFVSKRINNKPIKRHHTEKQRPNSTTSEIPTTKTKNKVEGRLLLNVVISKSTTILQLLPAKMRRCWSGGMPSLSWILALTLSIVSELSTSKVMVLPVRVFTKICIPPLKRSTKCRVDSFLNVVVSKGATVLQLLSSENQTLLVRGIPSLS

SEQ ID NO: 311 - DNA, 174 bp, Cannabis sativa
  source 1..174: mol_type = other DNA
atttgctgccaatgcagaatttcctctccttcaacagaattacattggaaagattcttcacttcgtagtcatgtatgctatgtacagcaaataaacaacacaattacaatatacaacaaaaattataactttaagaaatacactacaagtacaacaatcttagtaagaaagtga

SEQ ID NO: 312 - protein, 57 aa, Cannabis sativa
ICCQCRISSPSTELHWKDSSLRSHVCYVQQINNTITIYNKNYNFKKYTTSTTILVRK

SEQ ID NO: 313 - DNA, 144 bp, Cannabis sativa
  source 1..144: mol_type = other DNA
atgatgaatcctttacaactttacaatggtggaacgaaattacatcaatggctccaatcacaaatcaacaaagagacaactcttctaagtacacaatacaattctcaactctcttggttctatttcctcaattcatacctttga

SEQ ID NO: 314 - protein, 47 aa, Cannabis sativa
MMNPLQLYNGGTKLHQWLQSQINKETTLLSTQYNSQLSWFYFLNSYL

SEQ ID NO: 315 - DNA, 543 bp, Cannabis sativa
  source 1..543: mol_type = other DNA
ccaagctttgtagcagaaaaaagttgggtaaattggcttcattctcatcagtggagaaggctatattggtccatagatggttggccctggctcaacgagtttgaagaaatcagtggcatttgtgtcttcaggagtggctttagcttctgggatctttggaatttggaccaaagagttccatgggggagtagagggattgttggcaatttctatagtctcctattcttggatgggcttagctttgcatgtcgcttgtttggtgttcagtggatgatgggttttatgagcttttggcacagtaggagtggtgcggagagtgaggaggaaggtattggcgtgggacgtgtttgttggtctgtacaacgtatgggctggcggtggcgacaggccgaaacaggggcttttggagaaggtgacattgttacaaacgaagaggaacttgtgtatagcgatgtatggagtccattgttgtcaatggcttgggacttggcttggttctggctaaggcggcattgtaatattggcttgctgtatctcgcctaa

SEQ ID NO: 316 - protein, 180 aa, Cannabis sativa
PSFVAEKSWVNWLHSHQWRRLYWSIDGWPWLNEFEEISGICVFRSGFSFWDLWNLDQRVPWGSRGIVGNFYSLLFLDGLSFACRLFGVQWMMGFMSFWHSRSGAESEEEGIGVGRVCWSVQRMGWRWRQAETGAFGEGDIVTNEEELVYSDVWSPLLSMAWDLAWFWLRRHCNIGLLYLA

SEQ ID NO: 317 - DNA, 633 bp, Cannabis sativa
  source 1..633: mol_type = other DNA
atggctgaaagtatgaggggtgattcggtggaagagttcaggggtcttcgtattacctccttggatgacgaagacgaagaagatcaagaaattgatattgatgaagaagatgatgaagatgaagaaactgattatgcggttttaggttttgtagaaaagccgaagaacccccattcccttcatagccattttttcccatccaaggctggaggtgttcctgcttggttggatcctgtgaatctaccatcaggcagatcttttctttgcgacatatgtggagaacctttgcagttcatgctgcaggtctatgcaccagaggatgtggaatatgcatttcatcggacgttatttgtgtttatttgtacgtcaatggcttgtcttcgcatagatcaacatgaacaatggaagcgtcatcccgataaaccttgtagaagtgtgaagtttttcgttgccaattaccacgtggtaatcctttctactctagcgagcctccgaaagaagatgacaaacctcctggaattaaagttccatcttgtacttggtgcggtacatggaaagggattaaaccgtgctcaagttgtgaaaaggcaatttattgttcagagaaacatagggtcaagcattctcagttag

SEQ ID NO: 318 - protein, 210 aa, Cannabis sativa
MAESMRGDSVEEFRGLRITSLDDEDEEDQEIDIDEEDDEDEETDYAVLGFVEKPKNPHSLHSHFFPSKAGGVPAWLDPVNLPSGRSFLCDICGEPLQFMLQVYAPEDVEYAFHRTLFVFICTSMACLRIDQHEQWKRHPDKPCRSVKFFVANYHVVILSTLASLRKKMTNLLELKFHLVLGAVHGKGLNRAQVVKRQFIVQRNIGSSILS

SEQ ID NO: 319 - DNA, 876 bp, Cannabis sativa
  source 1..876: mol_type = other DNA
atggagatcgtaagaaggccatcgcatgctggttcttggtacaccgataatcctactaagttagcggaagaacttgaaggttggctgagagaatctggcctaactaaatctcctgaagtacggggtgttattgctccgcatgctggctattcctattctggtcgtgctgctgcctatgcatttggaaatattgatccgacgagcattactcgggtgttccttcttggtccgtctcatcactattacaccccaaaatgtgctctttcaatggcttcagtttacaagacgccaattggggacctacctattgatttggaagttattaaggagctaaaggctactgggaaatttgaacaaatgactattcgtgttgatgaggctgaacatagcatggaaatgcatttgccatatcttgccaaagtttttgaggggcaccctgtcaaagtagtaccaattttagttggtgctgttaatgcagaaaatgaagccatgtatggacagttgctttcaaaatatgtggatgatccccaaaatttcttctctgtatcttcagatttttgtcactggggctctcgatttagttacacacactatgacaagaaacatgggccaatatacaaatctattgaggctttggacaagatgggaatggatataattgaaacaggagatccagatgcatttaaaaattatctgatggagtatgataataccatctgtggacgacatccgattagtgtttttcttcatatgctgagaggttgctctacaaagataaagataaattttctccgatatgagcagtcgagtaagtgcaagagtatgagagacagcagcgtaagttatgcatcagcagctgcaaaggtagttccttga

SEQ ID NO: 320 - protein, 291 aa, Cannabis sativa
MEIVRRPSHAGSWYTDNPTKLAEELEGWLRESGLTKSPEVRGVIAPHAGYSYSGRAAAYAFGNIDPTSITRVFLLGPSHHYYTPKCALSMASVYKTPIGDLPIDLEVIKELKATGKFEQMTIRVDEAEHSMEMHLPYLAKVFEGHPVKVVPILVGAVNAENEAMYGQLLSKYVDDPQNFFSVSSDFCHWGSRFSYTHYDKKHGPIYKSIEALDKMGMDIIETGDPDAFKNYLMEYDNTICGRHPISVFLHMLRGCSTKIKINFLRYEQSSKCKSMRDSSVSYASAAAKVVP

SEQ ID NO: 321 - DNA, 258 bp, Cannabis sativa
  source 1..258: mol_type = other DNA
atgacaaacataaacgcgccagaggatttaaacaagatccttgtgtgtagctttttccaacaaaccaaccccaaaaagaagagaaagaaaacaaagcatgaaaacaaaaacctatctgtaaaaagtgaaaactatttaatgggggcttcctcacaacaaaccataagccccctcccctatatgcggcacttacccaaaaaaactcaatgccttcaagaactgttacccttgcatggcagcaatagtgctgactcttaa

SEQ ID NO: 322 - protein, 85 aa, Cannabis sativa
MTNINAPEDLNKILVCSFFQQTNPKKKRKKTKHENKNLSVKSENYLMGASSQQTISPLPYMRHLPKKTQCLQELLPLHGSNSADS

SEQ ID NO: 323 - DNA, 429 bp, Cannabis sativa
  source 1..429: mol_type = other DNA
atgggtcgtggaaatagcagcggtggggccaaagttccttgggttatctttttggtagtggagaggctcccagtcccaacaaaaccagccacaaacaaagctccagctgcaccaagtgaagcacctgctgtgactactgtggctgctgccaaacctgctcctgctcctgctcctcaacctctgtggatattactaagcagatcccagctggtattcacagcacatctaccaacaactacatgcgggctgatggccagaacaccggcaactttatcacggatcgaccctcaactaaggtccatgctgccccctggtggtggatcttctatgggttacctctttggtgacggtgtggtaactaaggatactgcatcttggacaatgggaaactgttacattgatttttgtggaatcatctactttagatga

SEQ ID NO: 324 - protein, 142 aa, Cannabis sativa
MGRGNSSGGAKVPWVIFLVVERLPVPTKPATNKAPAAPSEAPAVTTVAAAKPAPAPAPQPLWILLSRSQLVFTAHLPTTTCGLMARTPATLSRIDPQLRSMLPPGGGSSMGYLFGDGVVTKDTASWTMGNCYIDFCGIIYFR

SEQ ID NO: 325 - DNA, 315 bp, Cannabis sativa
  source 1..315: mol_type = other DNA
gccacaagcctgaagatcaacacaaatccggcgaacaccacaaggaagagcaccacaagggagaacaccacaaggtcgacgaacacaagggagaacacaaggaaggtatcatggagaagatcaaggacaagatccatggcgaaggaggtcacgaagataagggagagaagaagaagaagaagaaggagaagaagaaggacggtcacgatcatcatggacacgatagtagcagcagcgacagcgattagattcactgtatatatatcaaaacatcatgatccgtcgatcgatttaggtatttcggttgaatgctaa

SEQ ID NO: 326 - protein, 106 aa, Cannabis sativa
LEATSLKINTNPANTTRKSTTRENTTRSTNTRENTRKVSWRRSRTRSMAKEVTKIRERRRRRRRRRRRTVTIIMDTIVAAATAIRFTVYISKHHDPSIDLGISVEC

SEQ ID NO: 327 - DNA, 765 bp, Cannabis sativa
  source 1..765: mol_type = other DNA
ggagcagatgtatatgcggatgctcggcctacttacccaattgaatggtattcaaagttggctgctcttacttctcaccacaccttggcctgggacgttgccactggaagtggccaagctgctatcgatgtagcaaagcactacgaacaagtaatagcaacagatgtgagcgagtctcagctagagcgagcaaagccccacagtcgagtccgatacattcacactccactgaccacaaacgacaacgaaatagtgtcgttaattggaggcggttgtgaaaactcagtggacttgataacagtggctcaggccatccactggttcgatctaccgagcttctactctctggtcagtcgccttctgcgcaagccaggtggagttattgccgtatggggctacaacgacatagttgtgagctcctcatttgacgctgtattgaagaaattccacgacacgacgctgccatattgggacagtaacattaagcacatcttcgatgggtacaaaacgatgccgtttccatttgaagaggttgggttgggacgtgagggtaatccattggatctggatattcctaaggagctttcgtttgatggagttttgaatatgttgaaatcttggtctgctgtgattactgctaagagtctaggcgtggatctgctatgtgaagaggttgttgaggagcttaagagtgcttggggtggccctaatttggtcaggtctgtttgttacaaggcttttatgattgctggaaagttaccttaa

SEQ ID NO: 328 - protein, 254 aa, Cannabis sativa
GADVYADARPTYPIEWYSKLAALTSHHTLAWDVATGSGQAAIDVAKHYEQVIATDVSESQLERAKPHSRVRYIHTPLTTNDNEIVSLIGGGCENSVDLITVAQAIHWFDLPSFYSLVSRLLRKPGGVIAVWGYNDIVVSSSFDAVLKKFHDTTLPYWDSNIKHIFDGYKTMPFPFEEVGLGREGNPLDLDIPKELSFDGVLNMLKSWSAVITAKSLGVDLLCEEVVEELKSAWGGPNLVRSVCYKAFMIAGKLP

SEQ ID NO: 329 - DNA, 477 bp, Cannabis sativa
  source 1..477: mol_type = other DNA
atgttaaaaagaaaaaatggtgagaggggagaggaaatggtgtggaatagagaggcagaactcgaagattgttgttgttgtcgtctctttacacctattctttcaaatcacaatatggagcaagccttttgtgcttgctttctttttcttctgtttcggcggttgaaggacaactcttatggctgcatcaaagactcctttcacattctgctgtgttttcgagctgcattcgatgtaagcaggtgcaccgataagctttcgaagttcctctccctgagctgtagtaattggaacagcgccaggatggtcgataaagaactgcttgtcatctcgaaggtcaagctttgttccaacaagaatgatcgggacaccaggtgcataatgcttcaattcaggaatccactttttggaaacattttcatagctagctttgctaatgagagagaatgcagtataaacacatcggccccgcgataa

SEQ ID NO: 330 - protein, 158 aa, Cannabis sativa
MLKRKNGERGEEMVWNREAELEDCCCCRLFTPILSNHNMEQAFCACFLFLLFRRLKDNSYGCIKDSFHILLCFRAAFDVSRCTDKLSKFLSLSCSNWNSARMVDKELLVISKVKLCSNKNDRDTRCIMLQFRNPLFGNIFIASFANERECSINTSAPR

SEQ ID NO: 331 - DNA, 204 bp, Cannabis sativa
  source 1..204: mol_type = other DNA
ttttttcatttcttagttcaactagtattagtagaaaaagtcttaacattacttgaggctagttctatacttttaagttttaacaccaactctaactacattacaaagatgttggacaaaaggggggaggtaaggaaaaaagaaagtacaactgatatcagaatacatgttactagtaaagctctacctcaaaaatttacctaa

SEQ ID NO: 332 - protein, 67 aa, Cannabis sativa
FFHFLVQLVLVEKVLTLLEASSILLSFNTNSNYITKMLDKRGEVRKKESTTDIRIHVTSKALPQKFT

SEQ ID NO: 333 - DNA, 189 bp, Cannabis sativa
  source 1..189: mol_type = other DNA
atgattccgatatcaattgatgaatttagctttctagggttggtttcctttgctctaaaggagaagactttattatggttagtttttatttcagattcctttcttgttaaattcaaatcacacaattcaaacttctttttgttgtattactatttggtatcaatttcaaatggtaaaaaatgggtttga

SEQ ID NO: 334 - protein, 62 aa, Cannabis sativa
MIPISIDEFSFLGLVSFALKEKTLLWLVFISDSFLVKFKSHNSNFFLLYYYLVSISNGKKWV

SEQ ID NO: 335 - DNA, 510 bp, Cannabis sativa
  source 1..510: mol_type = other DNA
atggagaattccaccgttctcagagaatggttcgaccgagttgactccgacaagtccggctccattacagcttctcagctcaagaaagctctcgccgtcggaaacctcgaattctctatctccgttgttcaacaaatgatcaggatgtatgattttgacaggaatggaactatgagctttgatgagttcgttgcccttaacaagtttcttctcaaggttcaacaagccttttcagacctggaaagggatcgtggttatcttgttactgatgaagtgtttgaggcattggtgaaaatcggattctcactggactctcctgctttttacactgtctgtgagagttttgataaaaagaagaatggaagattcagattagatgattttatatctctttgtatattcttgcaatcagctcgtaatatgtttgattcatttgatactgctaagcagggtcgggttactcttgatcttaaccaatttgtatattgcactgcgaattgtaggatatga

SEQ ID NO: 336 - protein, 169 aa, Cannabis sativa
MENSTVLREWFDRVDSDKSGSITASQLKKALAVGNLEFSISVVQQMIRMYDFDRNGTMSFDEFVALNKFLLKVQQAFSDLERDRGYLVTDEVFEALVKIGFSLDSPAFYTVCESFDKKKNGRFRLDDFISLCIFLQSARNMFDSFDTAKQGRVTLDLNQFVYCTANCRI

SEQ ID NO: 337 - DNA, 306 bp, Cannabis sativa
  source 1..306: mol_type = other DNA
atgatcttaatattcgctacaaacagagatttgttttgtaaagtaattggttatcaatcagtcccggtttatttaaacaaaaaaaaaaaaaaaaaaaaaaaaaaacccaactttgtacaagaaagttgggtttttattttttttttttttttttttttttgttttttcataaataaaccgggacccctgattgataaccaatactttacaaaaacaaatctctgttgtagcaatatgaagatcatcacaaacaaagagtaccagaattccaattacacaaaacagctcgcactcgaacctggataa

SEQ ID NO: 338 - protein, 101 aa, Cannabis sativa
MILIFATNRDLFCKVIGYQSVPVYLNKKKKKKKKKPNFVQESWVFIFFFFFFFCFFINKPGPLIDNQYFTKTNLCCSNMKIITNKEYQNSNYTKQLALEPG

SEQ ID NO: 339 - DNA, 426 bp, Cannabis sativa
  source 1..426: mol_type = other DNA
ggccaacttttttgtacaaagttgtccccctcgaatcgcctgcggctctcttcttctccaaccaggatctattttattgcggctgtaaccatgactacatcaaggcgtctatcagataggaaggtggagaagttcgataagaacataacaagaagaggatctgtgcctgagaccaccgccaagaagggaaaggactaccctgttggccctattcttcttgggttcttcattttcgttgttattggatcatctctctttcagataatcaggactgccacaacggaggcatggcataattgcaacgcggaccgtactctgaagtgttacttatcgtcttctacatttttttttcttttttggtttaatttaatttgttttaggaagttaatatacttttacgagaccagagggttacacatgttatga

SEQ ID NO: 340 - protein, 141 aa, Cannabis sativa
GQLFCTKLSPSNRLRLSSSPTRIYFIAAVTMTTSRRLSDRKVEKFDKNITRRGSVPETTAKKGKDYPVGPILLGFFIFVVIGSSLFQIIRTATTEAWHNCNADRTLKCYLSSSTFFFLFWFNLICFRKLIYFYETRGLHML

SEQ ID NO: 341 - DNA, 231 bp, Cannabis sativa
  source 1..231: mol_type = other DNA
atgttaccttggtgggatcaagttcaacaccagaaaaatcataactatgtccaatctctaactgaaaaaaagctctgcatttcctcaaatgaatttatattcacaaagacattttgggagaatctttctttagctgaaggtgaaccagcttctgcatatgctgtttccggggtttctgatctccaaagagaagtggctgtgtgtaacaatcacctgggattggccatctag

SEQ ID NO: 342 - protein, 76 aa, Cannabis sativa
MLPWWDQVQHQKNHNYVQSLTEKKLCISSNEFIFTKTFWENLSLAEGEPASAYAVSGVSDLQREVAVCNNHLGLAI

SEQ ID NO: 343 - DNA, 354 bp, Cannabis sativa
  source 1..354: mol_type = other DNA
atgagtatggcgcggccagtaattctaccaatcgctacagaaaactcgggagagagtacactggcattcgaatcggggagccaagggaataaaacagcgatctcaatgccaatccggccgattccgcttcacccggccgtacttcccccgccatcggcaaaaatggctaatctaaatttgaacaagtcgttagagccctcaaatctatcgctgaagctctctggcccttcatcgtcatcgacggatgaaaaatcgcagtccccgccgcctcaacgaccgccgcaacatccgccactaatttccaggctatgccgaatggtgacagcatcattagtgttgcttgaagagtattaa

SEQ ID NO: 344 - protein, 117 aa, Cannabis sativa
MSMARPVILPIATENSGESTLAFESGSQGNKTAISMPIRPIPLHPAVLPPPSAKMANLNLNKSLEPSNLSLKLSGPSSSSTDEKSQSPPPQRPPQHPPLISRLCRMVTASLVLLEEY

SEQ ID NO: 345 - DNA, 564 bp, Cannabis sativa
  source 1..564: mol_type = other DNA
atgtccggtggaacgccggttgccggcggacttatgagacataggcatagccagggttatgcgtctagtggtgatgatcttgaggatgatgcgtgctctaggatgcgatattcaatggagccaactccaagaatttggtcacgaatggagattttggagaacatcctttggcttatttcagcagcctttattgtctactatggtgataggcattccaacttgatataccttctctggcatgatggtcgaatcagaagaatgcctttatacattgggatggtgggggtggttttgaattttatcatcttttgctatacaactatactagcatggagtgctaagagatttgatgaaaagtgggaaataacgagtacatcggctctgccatttgtcactcttcttggaatgatctcgtatttcttgttcacctttgctttgtggccaatatggggtttcttaacacttcctcttttgttcacgttgtttatggcatacatgattactttaccttacttcataattgggacatttaggccacaagacgacgagttccgtacagattaa

SEQ ID NO: 346 - protein, 187 aa, Cannabis sativa
MSGGTPVAGGLMRHRHSQGYASSGDDLEDDACSRMRYSMEPTPRIWSRMEILENILWLISAAFIVYYGDRHSNLIYLLWHDGRIRRMPLYIGMVGVVLNFIIFCYTTILAWSAKRFDEKWEITSTSALPFVTLLGMISYFLFTFALWPIWGFLTLPLLFTLFMAYMITLPYFIIGTFRPQDDEFRTD

SEQ ID NO: 347 - DNA, 489 bp, Cannabis sativa
  source 1..489: mol_type = other DNA
atgtcagctggtcttctttttggctctttaataggcacaatcattgtctctatcagcggaacagtagcggcaagtgttgcctttctcattgctagatattttgctcgcgagagaattctcaaactagttgaaggaaacaaaaagtttcttgctattgacaaggctattggagagaatggcttcagagttgttaccctgcttcgcttaagccctttgcttcctttctctttgggcaactatctttatggtttgacatcggtgaagttcgtgccttatgttttgggaagttggttggggatgcttccaggaacatgggcttatgtgagcgctggtgcatttgggcgagcaataatacaagaagagtcagaggttggtttggtcagcgggaacggtcaattattgacgttaggccttggattgttagccactgctttggctgcagcttacgtgacaaaacttgcaaaggatgctgtaaaggatattgagtag

SEQ ID NO: 348 - protein, 162 aa, Cannabis sativa
MSAGLLFGSLIGTIIVSISGTVAASVAFLIARYFARERILKLVEGNKKFLAIDKAIGENGFRVVTLLRLSPLLPFSLGNYLYGLTSVKFVPYVLGSWLGMLPGTWAYVSAGAFGRAIIQEESEVGLVSGNGQLLTLGLGLLATALAAAYVTKLAKDAVKDIE

SEQ ID NO: 349 - DNA, 555 bp, Cannabis sativa
  source 1..555: mol_type = other DNA
atgaaaggacacagctcattgttcttaatatcacattacaaacaattggaaaataacaggagttccttttcattttttcaattaacaggctatggacctctttctttagtacatttcaggtttcaagaaacagaagaaacattcatctttaagctaaatgatcgaacacttacaataatacggaactcccgatgcacatatcatatttccttaacccatggcatacttctgggtccagctcctagcagtcgtctcgtacttgctcctgtctgtcttgtacatgtgagcaatctcaggcaccagtggaatcatcaggattcgggtctgttagcagcgagcagatcgaaagcagcaccttcgagatggttagagctgggcctccactgctccttcaagatgtcaaggcaaatgctaccattgctgtttatattcgggtggaatacctttgtttctaaatgccaccttgggaggcttaaagggataatctggaggaaaatgaatagtaaccaagaagaaaacacccccagcataaggactatctggaaggtcccatga

SEQ ID NO: 350 - protein, 184 aa, Cannabis sativa
MKGHSSLFLISHYKQLENNRSSFSFFQLTGYGPLSLVHFRFQETEETFIFKLNDRTLTIIRNSRCTYHISLTHGILLGPAPSSRLVLAPVCLVHVSNLRHQWNHQDSGLLAASRSKAAPSRWLELGLHCSFKMSRQMLPLLFIFGWNTFVSKCHLGRLKGIIWRKMNSNQEENTPSIRTIWKVP

SEQ ID NO: 351 - DNA, 150 bp, Cannabis sativa
  source 1..150: mol_type = other DNA
atgccaaatcgttatttatacatttcttttggaggagacccacatacacaaacaggctatgttttggctttccctgtcttgattttgctcaaagtctctacaaggtgccattgtcccaccatgagtgtaacaatgaaaatgctctcttaa

SEQ ID NO: 352 - protein, 49 aa, Cannabis sativa
MPNRYLYISFGGDPHTQTGYVLAFPVLILLKVSTRCHCPTMSVTMKMLS

SEQ ID NO: 353 - DNA, 288 bp, Cannabis sativa
  source 1..288: mol_type = other DNA
ggccaacttttttgtacaaagttgtccccgataagtaaagcaatttcagatcaagaaagaaaaatgacgatgaagaaaacagtgcttgtggcggctatgattatgttgttggctttgtcatacacagaaatagtagcagccgacgcctttgattgcctcgatgcttgccaaactggctgcgttcaacgcgactctagattaacggcacgctgtgagcagaaatgcgtgatcagatgtggtccagattctacaactaagaagaatgttaaagcagtgataaattggtaa

SEQ ID NO: 354 - protein, 95 aa, Cannabis sativa
GQLFCTKLSPISKAISDQERKMTMKKTVLVAAMIMLLALSYTEIVAADAFDCLDACQTGCVQRDSRLTARCEQKCVIRCGPDSTTKKNVKAVINW

SEQ ID NO: 355 - DNA, 570 bp, Cannabis sativa
  source 1..570: mol_type = other DNA
atggcttccaatccaaaatcccaccaagctctgatctttcttctctttcaactctttttcttcttggtctctacaaccaccggtacctcatcttcttcttcacctccggcaccgacggtcttcgacatcctcccccaattcgggcttccgagtgggctcttacccgactcagtaatcaactacacgctctccgaagacggtcgattcgtcgtcgttttgaacaacccatgctacatcgagttcgattatttggtctattacgataaaaccatcaagggtaagctcagctatggatcgattaccgagcttgaaggtattcaggtcagaagattcttcttgtggcttagtgtggatgagatcagagtcgatttacctccttcggatagtatttacttccaggttggatttattaacaagaagcttgatttggatcagtttaagattgttcgttcttgctattttgctgctactgcctctggttgtggtggtggtggtggtctccgttcgtggaagcaatttcttgagattccagccccgaaggaagagctcgagatgcttattaccgagtag

SEQ ID NO: 356 - protein, 189 aa, Cannabis sativa
MASNPKSHQALIFLLFQLFFFLVSTTTGTSSSSSPPAPTVFDILPQFGLPSGLLPDSVINYTLSEDGRFVVVLNNPCYIEFDYLVYYDKTIKGKLSYGSITELEGIQVRRFFLWLSVDEIRVDLPPSDSIYFQVGFINKKLDLDQFKIVRSCYFAATASGCGGGGGLRSWKQFLEIPAPKEELEMLITE

SEQ ID NO: 357 - DNA, 753 bp, Cannabis sativa
  source 1..753: mol_type = other DNA
ttggatcagcttcaaggctctatcaagcagccaatctttagttgcaaatttagccgagacagggtgggccagacagaccctctgagcaactactggtcaggttctagtgacaataataccgatcctgagtgcgagagaagagagaggaagggatggaaggtgaagatacatgatctttctggctcagctgttgccgcagcgttcatgacaactccgtttgtgccttccacaggttgtgattgggttgccaagtccaacccaggagcttggttgattgttcggcctgatgcttgcaggccagagagctggcagccatggggtaaactcgaagcttggcgtgaacgtggtatgagggactctgtttgctgtagatttcgtctcatgtctgaaggtcatcaggaggcaggggagcttctcatgtctgagatttatatcaatgctgaaaggggaggggagtttttcatagacacggataggcaggcgcttgcaacggcagcaactccaatcccaagcccgcaaagcagcggagactttgcaggactcagcccagttgttggaggctttgtcatgagatgtaaggtgcaaggagagggaaagactagcaaaccattggtacagcttgccatgcgtcatgtaacatgtgttgaggatgctgcaatttttatggctcttgcagctgcagttgatctcagtatcgaggcatgccgccccttccgtcggaagataaagagagggtcatgttactctttgtga

SEQ ID NO: 358 - protein, 250 aa, Cannabis sativa
LDQLQGSIKQPIFSCKFSRDRVGQTDPLSNYWSGSSDNNTDPECERRERKGWKVKIHDLSGSAVAAAFMTTPFVPSTGCDWVAKSNPGAWLIVRPDACRPESWQPWGKLEAWRERGMRDSVCCRFRLMSEGHQEAGELLMSEIYINAERGGEFFIDTDRQALATAATPIPSPQSSGDFAGLSPVVGGFVMRCKVQGEGKTSKPLVQLAMRHVTCVEDAAIFMALAAAVDLSIEACRPFRRKIKRGSCYSL

SEQ ID NO: 359 - DNA, 456 bp, Cannabis sativa
  source 1..456: mol_type = other DNA
atggcgaaatctatgagatcgaagaaactgaaacgtctcagggccattcgtagagagatggtggagcccctatacgaaaagaaagaacttgcgaagtacgccgccatggaagccgccttggctgcccccaagctccctgttaaacctatcccgaatgctgaatcttcaatggagctcgtccccaccaccagtgctgctacaattacaactaacactatggatgtggatatggatgataatagtaaaaccaaggactcgcttaaagcaattgggggtgtagggaagatgtccaaaaggcagttcaaggtgggtaaagaaagcgtcctggaaaggggaaaggcaagggaaagtcaagagacgtcacatttaacttttttaagcttagcttttcataaatttcttttgagggtatcctgtttttgtttgcacactattaagatccaattcggactatag

SEQ ID NO: 360 - protein, 151 aa, Cannabis sativa
MAKSMRSKKLKRLRAIRREMVEPLYEKKELAKYAAMEAALAAPKLPVKPIPNAESSMELVPTTSAATITTNTMDVDMDDNSKTKDSLKAIGGVGKMSKRQFKVGKESVLERGKARESQETSHLTFLSLAFHKFLLRVSCFCLHTIKIQFGL

SEQ ID NO: 361 - DNA, 213 bp, Cannabis sativa
  source 1..213: mol_type = other DNA
atgctacaattaaggggaagtaacgatatcacgccgcctacctctacctctctacccgacgacgatcgacgtcgaaggccgaggcacgcaggtgctagtaaaattagaaaaccagagaatttacgtcatacgctattatatgatcgatttctccctatagggtttccatttttctgtccccaaatccaaagcatatgtagcccatgtacgtga

SEQ ID NO: 362 - protein, 70 aa, Cannabis sativa
MLQLRGSNDITPPTSTSLPDDDRRRRPRHAGASKIRKPENLRHTLLYDRFLPIGFPFFCPQIQSICSPCT

SEQ ID NO: 363 - DNA, 318 bp, Cannabis sativa
  source 1..318: mol_type = other DNA
ggggggacaactttgtacaaaaaagttgggaaaaaaaaaaaaaaaaaaaaaagtagaagagaatacgaaaggatgccgcttagtacgaccgcgatcggagcccttttgggtttgggcacccagatgtactccaatgccctccgcaagctcccttacatgcgccatccttgggaacacgttttgggcatgggaatcggtgtcgtgtttgtaaatcagcttgtgaaatgggacgcccagctccaggtagacctggacaagatgctcgagaaggccaaggctgccaacgaacgccgttactttgatgaagatgatgagtaa

SEQ ID NO: 364 - protein, 105 aa, Cannabis sativa
GGTTLYKKVGKKKKKKKSRREYERMPLSTTAIGALLGLGTQMYSNALRKLPYMRHPWEHVLGMGIGVVFVNQLVKWDAQLQVDLDKMLEKAKAANERRYFDEDDE

SEQ ID NO: 365 - DNA, 684 bp, Cannabis sativa
  source 1..684: mol_type = other DNA
atgagcggccttagcaagctcggagctacgcttattgtagttttcatactctgttttctagttctcatcgcagacctcgtttacgtcatctggcgacggcgccgttttcaacgacggaccttctcacccggagaatcagttgccaccaccggtgatgactcaacctacgctctttcctctaaagaacttctttacttcttctgctggaacaacaaccgggctcggatcgaacccaatattaccaacaatcgacccgacccgaacaataacacaactactcagacggcgcttgaggtggacgaattgttgaagcttcaagagctttacagacagacgaggttgcttttcactataaaggaggaagatttcgaagctgggaaaacagatcaggagaacaaagaacagagtagtattagtattacttcttcagctgatcgcgatgatgatgacgacgaccagggttttgtaaggtcaacgttgaccgacgatggtgattcggtgatgagtatgaaggtggatgaagcgacgccgttttcgactccttgtgcttcgcctctgtactttactccttctccttctccaagtcgtgagcagttaaaacagttctactcaaatgaaaatattggccgagtggaagaagagggtggtaccttccattgtagcttagatatgattggagtttga

SEQ ID NO: 366 - protein, 227 aa, Cannabis sativa
MSGLSKLGATLIVVFILCFLVLIADLVYVIWRRRRFQRRTFSPGESVATTGDDSTYALSSKELLYFFCWNNNRARIEPNITNNRPDPNNNTTTQTALEVDELLKLQELYRQTRLLFTIKEEDFEAGKTDQENKEQSSISITSSADRDDDDDDQGFVRSTLTDDGDSVMSMKVDEATPFSTPCASPLYFTPSPSPSREQLKQFYSNENIGRVEEEGGTFHCSLDMIGV

SEQ ID NO: 367 - DNA, 216 bp, Cannabis sativa
  source 1..216: mol_type = other DNA
atggatgagaagatccactaccggtccatcaaaacaaattgtgttgaaagtccacagggaactttgattgatagatccttggagaatttctcattaatcacaacagcttctgaaccatccatctcaacttggaagccagaccgtctcaaaaacaatgttgtcctctcccagttctcagaagaatccaccagcaaaagaacactaaacgaggcttct

SEQ ID NO: 368 - protein, 72 aa, Cannabis sativa
MDEKIHYRSIKTNCVESPQGTLIDRSLENFSLITTASEPSISTWKPDRLKNNVVLSQFSEESTSKRTLNEAS

SEQ ID NO: 369 - DNA, 165 bp, Cannabis sativa
  source 1..165: mol_type = other DNA
atggtacctcttatctgtaatttttttttttctttttccaaatcaaatttactctcagcctcttctccaaattacatgtacttgtccttttttgtctttgagatttggtattttgtagccatagtttgtttaaaaaatgccttcctcaaaagttgtaagaagtaa

SEQ ID NO: 370 - protein, 54 aa, Cannabis sativa
MVPLICNFFFSFSKSNLLSASSPNYMYLSFFVFEIWYFVAIVCLKNAFLKSCKK

SEQ ID NO: 371 - DNA, 1530 bp, Cannabis sativa
  source 1..1530: mol_type = other DNA
atgggagcgtgtgtttcaacgcctcatgggtgtgttggagggagattaaggtcctctaagaaggccaacggtcgtcggagaaggagaagaggtctgaagggaagagtctcgtctcgcttgtctgaggcctcatccgacagagcagataggtctgctcaacttgattccatctccaccttcaccaacccaactttccaagcaggaagcatcgatgaagcatggtttgactcaattcccgtatttgattctgattgtgatgaagattaccaaagtgttcaagatgatgtgctctctcttaatgaatatgaaggtgtgtcagtgtcaggaaattcgggcaactctgtcactgaaggtgcccagaactctagttttcaggttatgagttcagatgatattgactcccaacctaaatgtgatggaccgtcaacaaatgaagcaaaccatcctgtgttcctagatgaaatttcatcatctgtggatgaaaattcttgcaagggagaaggaatattggataactgcggaattcttccaagtaactgtttgccttgtcttgcgtctacagtaccttcagttgataaaagaagatcattaagttctagtccaccgagtgcaaggaagaaggcacccctgaagcttccttttaaatggaaagaaggccatgctatttctgctttactgtcatcaaagatgtttctgcaacgaccaattgccggttctcaagtacctttttgtcccatagaaaagaaaatcctagacagttggtcccccatcgagccaagtacatttaaagtgcgggcagtgaattactttcgtgacaaaaagaaagaatttgcttccaactatgctgcttattatccatttggagtggatgtattcttgtgccagcggaagatagatcatattgctagatttgtgaagctccctgctattaattctgcaggggatctcccttctatccttgttgtaaatgtacagataccattgtatccagctgcacttttccagggtgaaactgatggtgaaggaatgagttttgttctgtactttaaactttctgaaacctactctaaggaacttccacctcttttccaagagaacttcaggaggttaatcgatgatgaagttgaaaaggtaaaaggttttacggtagattctctcgtaccctttagggagaggctaaagatattaggacgtgttgcaaacgtggaagaacttcatctgggcggtgcagaaaggaagctcatgcaggcttacaatgaaaagcctgtgttgtcgcgtcctcaacatgagttttactttggggaaaattacatggagattgatttggatatgcatagattcagttacatttctaggaaaggatttgaagctttcttagacagacttaagagctgcgttttggatgttggcctcactattcaggggaacaaagctgaagagttgccagaacagatcttatgttgtatacgattaaatggaattgattatattaattatcagcaactggggttgaatcaagatcccctctga

SEQ ID NO: 372 - protein, 509 aa, Cannabis sativa
MGACVSTPHGCVGGRLRSSKKANGRRRRRRGLKGRVSSRLSEASSDRADRSAQLDSISTFTNPTFQAGSIDEAWFDSIPVFDSDCDEDYQSVQDDVLSLNEYEGVSVSGNSGNSVTEGAQNSSFQVMSSDDIDSQPKCDGPSTNEANHPVFLDEISSSVDENSCKGEGILDNCGILPSNCLPCLASTVPSVDKRRSLSSSPPSARKKAPLKLPFKWKEGHAISALLSSKMFLQRPIAGSQVPFCPIEKKILDSWSPIEPSTFKVRAVNYFRDKKKEFASNYAAYYPFGVDVFLCQRKIDHIARFVKLPAINSAGDLPSILVVNVQIPLYPAALFQGETDGEGMSFVLYFKLSETYSKELPPLFQENFRRLIDDEVEKVKGFTVDSLVPFRERLKILGRVANVEELHLGGAERKLMQAYNEKPVLSRPQHEFYFGENYMEIDLDMHRFSYISRKGFEAFLDRLKSCVLDVGLTIQGNKAEELPEQILCCIRLNGIDYINYQQLGLNQDPL

SEQ ID NO: 373 - DNA, 495 bp, Cannabis sativa
  source 1..495: mol_type = other DNA
atgcaagagcagtctgttaaaattcttataggctcagttggatctaagagcaataaggtaccctacgtcaaagagcttctaggattcttatctgagcattcaaacttttcaaaatcagttttatggactccagcaactacacgagttgctgcactttattctgctgcagatgtctatgtcattaattcccagggagttggagaaacatttgggagggtgacagttgaagccatggcatttggtcttccggtgcttggaacagatgctggagggacaaaagagattgttgagaacaatgtaacaggtcttcttcatcctacaggaaggtctggtgcgcctgtccttgccgagaaccttcggttcttgcttaagaaccctataacaaggaagcaaatggggatgaaaggtagagaaaaagtggagaggatgtacttaaagcagcacatgtacaagaaatttgtagacgttgtaaccaagtgcatgaaaaccaaataa

SEQ ID NO: 374 - protein, 164 aa, Cannabis sativa
MQEQSVKILIGSVGSKSNKVPYVKELLGFLSEHSNFSKSVLWTPATTRVAALYSAADVYVINSQGVGETFGRVTVEAMAFGLPVLGTDAGGTKEIVENNVTGLLHPTGRSGAPVLAENLRFLLKNPITRKQMGMKGREKVERMYLKQHMYKKFVDVVTKCMKTK

SEQ ID NO: 375 - DNA, 639 bp, Cannabis sativa
  source 1..639: mol_type = other DNA
tccagcgtcggtagggacccagagagctgccttcgcttttggcgttccttcgtagatctccggatttcacccctacacacgaaattccactctcctctgtctcactcaagtgaattggtttcgagagcattccaccagtttttggcgactttcactttcaacccgattcaccgcctacgtgccctttacgcccagtcattccgaagaacacttgcccccccgtcttaccgcggctgctggcacggagttagccggggcttcttcctcgagtcctgtcatgatcgcgcactcgacgaaagagctttacaagcggcattgcccttcttcactcacgcgatattgctggattcgggctttcgcccattgtccaagattccccactgctgccccccgtgggagtccggccgtgtctcagtcccagtgtggctgatcatccgaaaagaccagctaagcatcattggcttggtcagcctttacctgaccaactacctaatactacgcaggctcatcaaacagcgctttttagctttcttcaggattttggcccgaactgttcggcagattcccacgcgttacgcacccgttcgccactttgttctcaactgttcccgcctcctgggcgagacaagctacctttag

SEQ ID NO: 376 - protein, 212 aa, Cannabis sativa
SSVGRDPESCLRFWRSFVDLRISPLHTKFHSPLSHSSELVSRAFHQFLATFTFNPIHRLRALYAQSFRRTLAPPSYRGCWHGVSRGFFLESCHDRALDERALQAALPFFTHAILLDSGFRPLSKIPHCCPPWESGRVSVPVWLIIRKDQLSIIGLVSLYLTNYLILRRLIKQRFLAFFRILARTVRQIPTRYAPVRHFVLNCSRLLGETSYL

SEQ ID NO: 377 - DNA, 222 bp, Cannabis sativa
  source 1..222: mol_type = other DNA
ttggctttacacggatatagagatgaaaaagagtattgcagaagaagccaaagcaaagggtttagacctttgctaactttgtttatgacttcaattggattagaaaattgggtaatgagtattgcatttgcatttgcttcaattggatgtttcattgataaacatccctggaaagcacaccctcggcaagggatgttttgttttggttactatttttgctga

SEQ ID NO: 378 - protein, 73 aa, Cannabis sativa
LALHGYRDEKEYCRRSQSKGFRPLLTLFMTSIGLENWVMSIAFAFASIGCFIDKHPWKAHPRQGMFCFGYYFC

SEQ ID NO: 379 - DNA, 834 bp, Cannabis sativa
  source 1..834: mol_type = other DNA
ggccaacttttttgtacaaagttgtcccctctttacaagcaatgttctgatagaaatgtgtcatctactggggttaagaatatacctatacctggggtaaaagatgtctcacttgattgggatggacgtagtgctccattttgcaggccaaattcatacatctccgtgagagacaatgaagtcaagagagccttggcaaataagaacgcaaattatgacatggatttagaggatgaggcatggctaaagaaattgaacagtgataatggaatacctgagcttgtttcagaggatatttttgaatcattgtttgataccttagagatagcctacttttataacgaagttgatatcactacggacattaatttagcaattcgtttatgtcaaaatttgggtgggagagaagtagttgtagccgtttatgaatattggttggaaaagcggaagcagaataaggcatcatcactgcttcgggtttttcagggtaatgaaatgaaaagagcctcgacaaccataaagacttcatatcgcaggaagagatcctttaaacgacaaccaagccaactaggaagaggcaaacaatccagttttttgtttcacgccatggtgtctgaaataggcagtgttgatgaacagaatgcctcacttactaaagctcaaatctcagcaaaaaggtgtatggaatcagcagtttgcaaacgtcgtagggctcagatgcttatgaacaatgcagatcttgctacatacaaggcaacaatggcgaggagaattgctgaagccatggctttatatcccaattgtgcttctgcttctgcttattttcttgattga

SEQ ID NO: 380 - protein, 277 aa, Cannabis sativa
GQLFCTKLSPLYKQCSDRNVSSTGVKNIPIPGVKDVSLDWDGRSAPFCRPNSYISVRDNEVKRALANKNANYDMDLEDEAWLKKLNSDNGIPELVSEDIFESLFDTLEIAYFYNEVDITTDINLAIRLCQNLGGREVVVAVYEYWLEKRKQNKASSLLRVFQGNEMKRASTTIKTSYRRKRSFKRQPSQLGRGKQSSFLFHAMVSEIGSVDEQNASLTKAQISAKRCMESAVCKRRRAQMLMNNADLATYKATMARRIAEAMALYPNCASASAYFLD

SEQ ID NO: 381 - DNA, 183 bp, Cannabis sativa
  source 1..183: mol_type = other DNA
ggccaacttttttgtacaaagttgtcccccagaaatatacctagttttgtcataacaactttcgatcggattattattaaattttattttgacaaaaaattagttttggatactctattttgtatcattttagaaataaatacagagtctaatgtgttattttacaaaacagaagcccaataa

SEQ ID NO: 382 - protein, 60 aa, Cannabis sativa
GQLFCTKLSPRNIPSFVITTFDRIIIKFYFDKKLVLDTLFCIILEINTESNVLFYKTEAQ

SEQ ID NO: 383 - DNA, 345 bp, Cannabis sativa
  source 1..345: mol_type = other DNA
atgcgtatagaggtgaccccggtgtcccgcatgctgatccagaccgcttctggaacatatggattggatcagctaccttttcagcactcacctggttcaatccttacatgtggcaactctctaacctgttcaactggcacgacaaagccatgttgttttgaacaatatcactggaagaaagcaatgaagaaagggcagccttacaagtttaagtggaacgagtacatggataaggaccaaccagagattcttattatgccaactggcctgtttatttcccatagttttctttgtctctatatatatataggcatgaacttccatcttttggtcatgaacttttag

SEQ ID NO: 384 - protein, 114 aa, Cannabis sativa
MRIEVTPVSRMLIQTASGTYGLDQLPFQHSPGSILTCGNSLTCSTGTTKPCCFEQYHWKKAMKKGQPYKFKWNEYMDKDQPEILIMPTGLFISHSFLCLYIYIGMNFHLLVMNF

SEQ ID NO: 385 - DNA, 234 bp, Cannabis sativa
  source 1..234: mol_type = other DNA
tggggggacaactttgtacaaaaaagttggggggacaactttgtacaaaaaagttggggggacaactttgtacaaaaaagttggaaaaaacaaaatgaagtaaattttaaaaacagatttttgatgattttaaaaatttttaaaaacacttttttcattttgcttctctctctctcgatcacttcctcaactcacagtcttccatggctagcttcaccagatcttgagacttga

SEQ ID NO: 386 - protein, 77 aa, Cannabis sativa
WGDNFVQKSWGDNFVQKSWGDNFVQKSWKKQNEVNFKNRFLMILKIFKNTFFILLLSLSITSSTHSLPWLASPDLET

SEQ ID NO: 387 - DNA, 465 bp, Cannabis sativa
  source 1..465: mol_type = other DNA
atgagaggctcttctatggattctgttgtttgccctaaacctcgtcgtcctaccctcctaaattcaccaacgccggtcagatctttccgatggcatctcaattaccaatcagagctttgtgattctaaagctgggactgatctctttgatgccatcctcacaaagggtggttatggtgttgaaccatctagttatacggcccaagtagcctcgtcgcccccatttttcagcgggtcgccgccgagcagagtagctaacccattaattcaggatgctcgattcggagatgagaagttgtccccattggctgcgtcgcagccaattcagccaccgtccggcctttcttccccgaggaaaggaggctgcgttcggggcaattttgggaacaaaccaactgtgagaattgagggtttcgattgtttggacagggatagacgcaattgcagcatccctgcccttgcatag

SEQ ID NO: 388 - protein, 154 aa, Cannabis sativa
MRGSSMDSVVCPKPRRPTLLNSPTPVRSFRWHLNYQSELCDSKAGTDLFDAILTKGGYGVEPSSYTAQVASSPPFFSGSPPSRVANPLIQDARFGDEKLSPLAASQPIQPPSGLSSPRKGGCVRGNFGNKPTVRIEGFDCLDRDRRNCSIPALA

SEQ ID NO: 389 - DNA, 1257 bp, Cannabis sativa
  source 1..1257: mol_type = other DNA
atggcctccgtcttctccttccttcttctcttcttccacttctcttcatttcaagtgttcgccgccgaaaaagtcgtaatcgaagaaggttacacagtaaagacggtgatcgacggccacaagctcgggatcagtccttactctgtcatccttcgtcctggatcgtccgatttggttgtcctcgattctactggtagttctttctacaccgtaccgtttccgatctctaatgataatgttgtcaaacggttttctggaaccggtgaagctgggtatgaagatggtgaaccgggcatggcccaatttagtaagcctcggagttttgcgatagatttgaaagggaatgtttatgttgctgataagagcaacaatgtgattagaaagatcactgattcaggtgtgactacaattgctggtctgaataagaaagtgggccataaggatggaccagtatcaaatgcaactttctcaagtgattttgagcttgtttttgtgccagagatttgtgctttactgatttctgatcatgggaatcaactagtgcggcagattgatctcaaggccgaggattgccggagggattccgggtctggttccggttctggtgtgggagctatttcgatttggagtgtggcagtgggagttgcatgcatactcggttttgctggtgggtttgcactcaggccttatatccttcctcatggaggcatcaggatgcgcaatttcagcgggacatggaagctttgcctaacccatctggagaaacaagttcagatactctgcttcgccatcagaagcgcaactgctaactcctctttagtttgctcgctcttggaaaggctcttttggctaagcctatctcatatatctcttttatttagaatagattatctagtcccccaagtttccccaaaggaacacgtatctttgcttggtagcgacagtgtcgataatagtagcagcagcagcagcagcagcagcagtagtagtgagacaatgaagtcatttaagtatgacaatcaacttaaggatttgataagttttgacgaaagctcggtgtcatcgaaccttgcatccacaggaatggttgagcaaagagaagagtatcaggagaggcgtgatgttgttctgtctgaaaatgggaagatagagaatatgatagagactaacattttgcattttgtggacaaggctaaagaaactactctgatagacgcagctgtgtatggcaactctggtctcatcagaagaagatga

SEQ ID NO: 390 - protein, 418 aa, Cannabis sativa
MASVFSFLLLFFHFSSFQVFAAEKVVIEEGYTVKTVIDGHKLGISPYSVILRPGSSDLVVLDSTGSSFYTVPFPISNDNVVKRFSGTGEAGYEDGEPGMAQFSKPRSFAIDLKGNVYVADKSNNVIRKITDSGVTTIAGLNKKVGHKDGPVSNATFSSDFELVFVPEICALLISDHGNQLVRQIDLKAEDCRRDSGSGSGSGVGAISIWSVAVGVACILGFAGGFALRPYILPHGGIRMRNFSGTWKLCLTHLEKQVQILCFAIRSATANSSLVCSLLERLFWLSLSHISLLFRIDYLVPQVSPKEHVSLLGSDSVDNSSSSSSSSSSSSETMKSFKYDNQLKDLISFDESSVSSNLASTGMVEQREEYQERRDVVLSENGKIENMIETNILHFVDKAKETTLIDAAVYGNSGLIRRR

SEQ ID NO: 391 - DNA, 954 bp, Cannabis sativa
  source 1..954: mol_type = other DNA
atgtccgaaagagacgaaacgataacaatgtcacaaacccaggagagttccatcttcctaactcccaagattttctttcaatccatccaaattttcctccaaaacaaacaaattttcttttcaatttttaccctcataaccctcccactctcagccctactcttctccctttccctctcttcccacccatcaaaatctcgaatctaccacctagaatacctagcaagcctggcctcaactcagttcgaagctcgacacgtgtggaaagattcccgcgaagacgccgcatcgatcttccgcgccaaaatcctctttttcccaccttgctacgctctctccttactcgccgccatctccaccgtcagctccgtcaacctcgccgttaacggaaaacgccccactctcaactccgccgttacggcgtttaagttaacgtggaagagacccttcgttacctccatcgtcgtctacgctctcctcctcgcctacgtccaaattccccgaacactttccgccgtgttttattcacctgcgccgagactgttcatcctcaccgtcggatcaatcgtcgaggtttatctaatggcggtgatgagtctaggtatggtggtctcgatcgcagaggataatttcggatgggatgctattcgggtcgggtcggttatcatgagtgggagacgggttaccgggtgggttttaagtgctgtgtttgtttttgtaacggggcttatagcgagggatgtggagaagttgatgaacgatcaagatccgtttgggaaaccaacatcgacggttgtgattgtggcgagaggtgtggtggacaaggtgggtctgattggtttgtatggggtagttatgctatggggttatgttgttaccactgttttttactgcgactgtagaagacaatacagtaataagagtgaaattgaaaatgtaacacttacagtgtag

SEQ ID NO: 392 - protein, 317 aa, Cannabis sativa
MSERDETITMSQTQESSIFLTPKIFFQSIQIFLQNKQIFFSIFTLITLPLSALLFSLSLSSHPSKSRIYHLEYLASLASTQFEARHVWKDSREDAASIFRAKILFFPPCYALSLLAAISTVSSVNLAVNGKRPTLNSAVTAFKLTWKRPFVTSIVVYALLLAYVQIPRTLSAVFYSPAPRLFILTVGSIVEVYLMAVMSLGMVVSIAEDNFGWDAIRVGSVIMSGRRVTGWVLSAVFVFVTGLIARDVEKLMNDQDPFGKPTSTVVIVARGVVDKVGLIGLYGVVMLWGYVVTTVFYCDCRRQYSNKSEIENVTLTV

SEQ ID NO: 393 - DNA, 774 bp, Cannabis sativa
  source 1..774: mol_type = other DNA
atggctacagcacctgtcaagtctccgttgcacaacttccctctcacattcttgaaatggggcggcgcaaagaatcacactagcggtggtcatcatcgttaccgtcgaccagtctcggctgagaactctcccgtcgccgatcatggtgaggttgacccggataggaacgattcggagcctgaagctggtcggactagtaaggtcggatcgcgtactgttcgcaacagatttgggtttgcgtcttgctcttcagcggagaaggtgcagaaacagcaagggagcaacgagagtgatgaggttgtcagagatgggaagcagcagcatcgtgatggagaagaagttgaggctgaggagacggttcagaagccgtggaaattgaggccgaggaaggttttgttctcgaaacctgcttcgattggtgttaagaatggagaatttcaggaaccaggtgggactgttactggcagcggtgtaggaagtggtcagaatcagagtgagaatcagaaccagcaacaaccgaaatcgctgaggctaaggggattctcggagcagcagagtacggagaagaaagagaagaggaagttttggatcgcgctgtctagggaggagattgaagaggatatctttttgatgactgggtcaaggcctgctcggaggccgaggaagcgtcccaagaatgttcagaaacagcttgacgctgttttccctggattgtggttggtgggtgttactgctgatgcttaccggatcgctgaggctcctcctaagaggtag

SEQ ID NO: 394 - protein, 257 aa, Cannabis sativa
MATAPVKSPLHNFPLTFLKWGGAKNHTSGGHHRYRRPVSAENSPVADHGEVDPDRNDSEPEAGRTSKVGSRTVRNRFGFASCSSAEKVQKQQGSNESDEVVRDGKQQHRDGEEVEAEETVQKPWKLRPRKVLFSKPASIGVKNGEFQEPGGTVTGSGVGSGQNQSENQNQQQPKSLRLRGFSEQQSTEKKEKRKFWIALSREEIEEDIFLMTGSRPARRPRKRPKNVQKQLDAVFPGLWLVGVTADAYRIAEAPPKR

SEQ ID NO: 395 - DNA, 573 bp, Cannabis sativa
  source 1..573: mol_type = other DNA
atggctgccaaatttttcttgaaagccctctcaatcatcgctcttacttttgctctttgccttcaaggcaccttaggaggagtaacatgcgaggagctagaacaagatacatgcgcattcgcagtgtcatcagtggggaaacggtgcgttttggagaagcacgtgaaaaggagtggtgaggaagcgtacacgtgtcgtacatcagatatagaagcagacaaactgaaggacataatcgaaaccgaccactgcatcaaagcttgtggacttgaccgtaaaacccttggaatctcatccgactcactccttgaatctcgcttcactaaccaactttgctctccttcttgtcacaactcttgtcccaacgttgttgacctttacttcaatctcgccgctggggaaggtgtgtttcttcccaaactgtgtgaagcacaagctggaaatgacgctcgtcgaggaatggctgagatcaaaagctcaggttttgtggttgcccaaggaccgggcgtccaacctgccaggctaattgcagctactgcaccaatcattagcccagctctcccaccttactga

SEQ ID NO: 396 - protein, 190 aa, Cannabis sativa
MAAKFFLKALSIIALTFALCLQGTLGGVTCEELEQDTCAFAVSSVGKRCVLEKHVKRSGEEAYTCRTSDIEADKLKDIIETDHCIKACGLDRKTLGISSDSLLESRFTNQLCSPSCHNSCPNVVDLYFNLAAGEGVFLPKLCEAQAGNDARRGMAEIKSSGFVVAQGPGVQPARLIAATAPIISPALPPY

SEQ ID NO: 397 - DNA, 216 bp, Cannabis sativa
  source 1..216: mol_type = other DNA
atgagcaatgctttttataatgccaactttgtacaaaaaagttggccaacttttttgtacaaagttgtccccccaactttcttgtacaaagttggcattataagaaagcattgcttatcaatttgttgcaacgaacaggtcactatcagtcaaaataaaatcattatttgccatccagctgatatccctatagtgagtcgtattacatggtcatag

SEQ ID NO: 398 - protein, 71 aa, Cannabis sativa
MSNAFYNANFVQKSWPTFLYKVVPPTFLYKVGIIRKHCLSICCNEQVTISQNKIIICHPADIPIVSRITWS

SEQ ID NO: 399 - DNA, 189 bp, Cannabis sativa
  source 1..189: mol_type = other DNA
gggcgtcgcaagggaatccacttaggcttagggaagataagagtttacaaaaaaggaataaaaaaaaaaaaaagaaacaaaggggatttattagcgaaggagggggggatcgaactaggtgtatgtaatctactcgctataagacaatttttgtggatgtttcaaagaatgattctagaatccgaatga

SEQ ID NO: 400 - protein, 62 aa, Cannabis sativa
GRRKGIHLGLGKIRVYKKGIKKKKRNKGDLLAKEGGIELGVCNLLAIRQFLWMFQRMILESE

SEQ ID NO: 401 - DNA, 1455 bp, Cannabis sativa
  source 1..1455: mol_type = other DNA
tgggaagttgcatctctacgatctggttttctttaccaatcaagaagatggcactcgaatactgcatctgcctttgcatcaattactgccagacttgagaagctccggagggaaaaggatggttctaaaaccgaaaagaacgggaacacttttcatgatgataataaagaaaaagacattgtggaggaagacttcaagcaggggcgaggccgcaagaattcccctagtttttacaagattagctctatcatcgataggaaacctggttcaattgacttaaatgaggcagaaccatttgtcatgaggctcagtgaggatcaaattgtacatttactctctgctttttggatacaagcttatctatcagacaatcttccctcgaatattgaatccattgctcattctttcattttaacacttatttcctcacgcttgaagaatccaacagacaaccttgtggtcaacttcttccagcttcttttgtctcttaggaaaatgtctcttgatcctaacaacgggatgttgacaccagcgggccagagatctgttttggtactatcaatgagcatgttgatgtttgcagctaagatatatcacattcctgaactgaatgattttctcaagtcattgattccatacgatattgacccctatcttggtattagcgatgatcttcaagtatatgtaaagcctgatgctgatgtaagagaattcggatctgcttctgataatcagctggctacatcaattctttccgagttgcgaaataaaatatatgacactgagaatatcgtaattgacatcttagttcaaaatctgacaaacattaccaagctggaggcggaagatgtgctaaagcaacttcaagaaccattcacacctgatgatacattctcatttggtccacaatctgcactggaccttgaccatgatcagatggcttcccactccaaagaatccttatcctttgatggggatttcccgacaaactctttagttgaggatgatgcgactagcgaattatccgttgcagatctctctcgtttcattcccagagtgccatcaacttcatctggctcacatattatcagcattggacagcttttagaatctgcacttgaggtagcaggtcacgtagcaggatcatccgtctctacatcacctctcccgtacaacgccatggccagccagtgtgaagcccttggcacaggcacaagaaagaagctctccaattggttggcccacgaaaattatcatggcaaagcagctgagaaatcttttccagcttttcccttcaataatagccgcagaactctccaccagataacaagtgagtgtgaaggtgaaagtagtggtcctggccagggagctgcatttccccaggatccatggttgtcgatgaggcttcctcctgctagtccctttgacaattttctcaaggcagctggctgttaa

SEQ ID NO: 402 - protein, 484 aa, Cannabis sativa
WEVASLRSGFLYQSRRWHSNTASAFASITARLEKLRREKDGSKTEKNGNTFHDDNKEKDIVEEDFKQGRGRKNSPSFYKISSIIDRKPGSIDLNEAEPFVMRLSEDQIVHLLSAFWIQAYLSDNLPSNIESIAHSFILTLISSRLKNPTDNLVVNFFQLLLSLRKMSLDPNNGMLTPAGQRSVLVLSMSMLMFAAKIYHIPELNDFLKSLIPYDIDPYLGISDDLQVYVKPDADVREFGSASDNQLATSILSELRNKIYDTENIVIDILVQNLTNITKLEAEDVLKQLQEPFTPDDTFSFGPQSALDLDHDQMASHSKESLSFDGDFPTNSLVEDDATSELSVADLSRFIPRVPSTSSGSHIISIGQLLESALEVAGHVAGSSVSTSPLPYNAMASQCEALGTGTRKKLSNWLAHENYHGKAAEKSFPAFPFNNSRRTLHQITSECEGESSGPGQGAAFPQDPWLSMRLPPASPFDNFLKAAGC

SEQ ID NO: 403 - DNA, 354 bp, Cannabis sativa
  source 1..354: mol_type = other DNA
gggtacttggttttgttgctgattattattaatattggtgtatcaagtaataatagtagatgtttgtgccttgctcaagacccttacggagattatggaaccccatacagaccccggacacccggaaaccgtaagactactactcaagctgctaccaacaccaacaccaacaccaatcatcctactttttacaaatctcccccacggcccagccaattaattaattgtatcactctctcgtacaaggatgaatcgcaatgtattcatcattctctcttaattggttggatggtaagtacgatgatttacttcacgagcgaggtctttatcaagctatacatcatctctaagtaa

SEQ ID NO: 404 - protein, 117 aa, Cannabis sativa
GYLVLLLIIINIGVSSNNSRCLCLAQDPYGDYGTPYRPRTPGNRKTTTQAATNTNTNTNHPTFYKSPPRPSQLINCITLSYKDESQCIHHSLLIGWMVSTMIYFTSEVFIKLYIISK

SEQ ID NO: 405 - DNA, 324 bp, Cannabis sativa
  source 1..324: mol_type = other DNA
tgggccagagagtgggtcgtagatcataagcttcgaactgtggggtctctttggctaagcggaatagcaggttcaatcgcatacaattggtcccgaccaaagaaaacaagtgtgaagattattcatgctaggttgcacgctcaggctctcaccttagctgcattagctggtgcagcagtggtggaatactatgaccacagaacaggcaaaaggctgatcagtacgctaagttcctcccaaactcctaccctcacaaggattgactgtgatattccatcaacaattgccatatgggagtatagaaaaagaggaaagaagttataa

SEQ ID NO: 406 - protein, 107 aa, Cannabis sativa
WAREWVVDHKLRTVGSLWLSGIAGSIAYNWSRPKKTSVKIIHARLHAQALTLAALAGAAVVEYYDHRTGKRLISTLSSSQTPTLTRIDCDIPSTIAIWEYRKRGKKL

SEQ ID NO: 407 - DNA, 732 bp, Cannabis sativa
  source 1..732: mol_type = other DNA
atgaagtcaaatttacttgataacatgccttggcttagaacccaaacaaacaaattgaacaatgatatggttgcatccattgaatccaatactagtgatgcactattagtagaagacacaaagcaaaagacttctcttgatgtaaaattttggaagtggtcttttatctcctttgttccatgggtgactccccatggtaaaaagacaccaactactataaatagagggctgagaaagcgtgccctgcctcgagggggagttgacaatgagcttcggggtgccccggtgcgctttaagccatatgtgtcgaaggtaccatggcatacaggcgtgagagcatttctttctcagctatttccccggtatgggcattattgtggacccaattggtcaagtgggaaggataatggatcgcttgtttgggacaaacgacctattgattggttggatttttgctgctactgccatgatattggttatgatactcatgatcaagctaagttattgaaggctgacttagcatttcttgagtgtcttgagagaaagaatatgactactaaaggagatgttcatgttgctcatctttacaggacaatgtgcattacaggcttaaggaatttactaattccataccgaactcacctactaaagctacaatcagcacaaccgctaattcagtttggatggctaagcaatgtaggttggaaaagttggagcgtccagaaatcctga

SEQ ID NO: 408 - protein, 243 aa, Cannabis sativa
MKSNLLDNMPWLRTQTNKLNNDMVASIESNTSDALLVEDTKQKTSLDVKFWKWSFISFVPWVTPHGKKTPTTINRGLRKRALPRGGVDNELRGAPVRFKPYVSKVPWHTGVRAFLSQLFPRYGHYCGPNWSSGKDNGSLVWDKRPIDWLDFCCYCHDIGYDTHDQAKLLKADLAFLECLERKNMTTKGDVHVAHLYRTMCITGLRNLLIPYRTHLLKLQSAQPLIQFGWLSNVGWKSWSVQKS

SEQ ID NO: 409 - DNA, 630 bp, Cannabis sativa
  source 1..630: mol_type = other DNA
tggctccccaccaccacccgtaaaatctccaccaccaccataccactacacgtccccaccaccaccatctccatcaccaccaccaccataccaatacagctctccaccaccaccagtgaagtctcctcctccaccataccactacacctctcctccaccaccaaagcacgaagaggagccaccataccactactcatccccaccaccaccatcaccgtcacctcctccaccataccactactcttccccaccaccaccaacaaagtctccacctcccccataccattacacctcacctcctccaccagtaaaattgccatcacctcccgcttacatttactcttccccaccacccccacctccatactaagctcaagaattttatcaccatacatacatggatcatgttctagattgaaaagtggaataaaagaagtttccatcaacgctcaccaagcctcgaatgaatgttcattattcaataagggtctgtcaattagcgatgacccacacatttcaacttcttttgtggtagcgttgcaaatgctttttctgattttaagttggagtggttatggacaaagcaaagtaagaagaagaatgtggagattggagattaagctaggataa

SEQ ID NO: 410 - protein, 215 aa, Cannabis sativa
LATIRLQLPTTTRKISTTTIPLHVPTTTISITTTTIPIQLSTTTSEVSSSTIPLHLSSTTKARRGATIPLLIPTTTITVTSSTIPLLFPTTTNKVSTSPIPLHLTSSTSKIAITSRLHLLFPTTPTSILSSRILSPYIHGSCSRLKSGIKEVSINAHQASNECSLFNKGLSISDDPHISTSFVVALQMLFLILSWSGYGQSKVRRRMWRLEIKLG

SEQ ID NO: 411 - DNA, 960 bp, Cannabis sativa
  source 1..960: mol_type = other DNA
atggttcaaaatttcatcgaggaaaccaacgagaggcaaccacctccacctccacccaaatgcggtcgtaaccgctgcaattgctttaacgcgaacagctacgatagctccgatgatgaattcgacgtcttcggcggtggtttcggagactccactacctccggatcttttggcggcgacgcctccgacatcctcaagagtctaattccttgcgcgagcgtagcagagagaaatctcttggccgatactgctaagatcgttgagaagaacaaaaatcacaaacgaaaagaagatttgataaagattgtaaccgaaggactctcatctatgggctacgattccaccatctgtaaatctaaatgggaaaaatcttcttcattcccagctggtgaatacgagtacatagatgtggtagtacaaggcgagaggttattgatagacatagatttcagatcagagtttgagatagctcgatcaaccggagcttacaaggctatcctccaatctctgccgtacatattcgtcggaaaatctgatcggcttggccaaatagtctccatcgtttctgaggcggccaaacagagtcttaagaagaaaggaatgcacttccctccatggcggaaggccgattacatgagagccaaatggctctctcctcactccagaaaagaccccatagccatcgctgttccagaatcggaatcggagaaggtcaagtctgacgttgaatctcaggacgacgacgggtttagcgattgcggtgaattggagctgattttcggtgagaagagcctgccgtcggagaacgatcccggcggatctgccgccgagaaagcttttccggcggatgagaaaaattcgtcggagccggtaacggcgtggcagccaccggcgattgaaccgaagagtgtagagagaggaactaaaatcgttaccggattagcttctctactcaaataa

SEQ ID NO: 412 - protein, 319 aa, Cannabis sativa
MVQNFIEETNERQPPPPPPKCGRNRCNCFNANSYDSSDDEFDVFGGGFGDSTTSGSFGGDASDILKSLIPCASVAERNLLADTAKIVEKNKNHKRKEDLIKIVTEGLSSMGYDSTICKSKWEKSSSFPAGEYEYIDVVVQGERLLIDIDFRSEFEIARSTGAYKAILQSLPYIFVGKSDRLGQIVSIVSEAAKQSLKKKGMHFPPWRKADYMRAKWLSPHSRKDPIAIAVPESESEKVKSDVESQDDDGFSDCGELELIFGEKSLPSENDPGGSAAEKAFPADEKNSSEPVTAWQPPAIEPKSVERGTKIVTGLASLLK

SEQ ID NO: 413 - DNA, 798 bp, Cannabis sativa
  source 1..798: mol_type = other DNA
atggtagttttcggtaactcagttcgtgaatccagttcttttgacactatcctctttgatttggatgatactttgtaccctggaaatactggattatctggtgctcttaagagaaacattgaagattttcttgtggagaaatgtggattctcagagactcaagctgcgactctcagagttgagcttttcaaaacttatggcagtactcttgctggtttaagggtgttgggatacaacgttgatgccgatgattatcatagtgtcgtacacggaagattgccgtatgatcttatcaaagcagaccctcaactacgaaacttgttaaacagtatctcccagagaaaaatcattttcacaaactcggatcggattcacgcgatgaaggccatggatcggcttggaatcgctgattgctttgaccaaatcatatgcttcgagaccatgaaccctaacttgtctaaagcgaccaggccagacgaattccctgtcgttctgaaaccttccatggaagccctgaaaattgctctctctgtcgctgaagtcgatcctcggcgtacgctgtttctcgacgacaatgttcgaaatatcgccgctggcaaagccgtgggtcttcgaacagctttggttggaaagaccatgaaaaccaaagaagccgattatgcattagagaaagtgaataatctggcacaagtgataccagaaatatgggtcaatggagttgaaaagggcgaaccaaggatcagccgcaccaacagtgagatcgagtcaagtcttgcaatcacagccgttggagcttga

SEQ ID NO: 414 - protein, 265 aa, Cannabis sativa
MVVFGNSVRESSSFDTILFDLDDTLYPGNTGLSGALKRNIEDFLVEKCGFSETQAATLRVELFKTYGSTLAGLRVLGYNVDADDYHSVVHGRLPYDLIKADPQLRNLLNSISQRKIIFTNSDRIHAMKAMDRLGIADCFDQIICFETMNPNLSKATRPDEFPVVLKPSMEALKIALSVAEVDPRRTLFLDDNVRNIAAGKAVGLRTALVGKTMKTKEADYALEKVNNLAQVIPEIWVNGVEKGEPRISRTNSEIESSLAITAVGA

SEQ ID NO: 415 - DNA, 282 bp, Cannabis sativa
  source 1..282: mol_type = other DNA
atggatgctctgagtagttttcaaggcagcaattcgcttgaggttaaaatggaagatagagctattgctcaacctgaatgtggacgcccaccaattgtgtctgaggcaaagagggctgctgtgcaggaagagctgaagagagttaataaacttccggcaacaagcacttatgcttctcatcgactgcgggttctcaataaaattctgcagcttttgtctgtccagagatcttcatcagaagaaatggagttggagttgctctttgctgggctgtctctgtga

SEQ ID NO: 416 - protein, 93 aa, Cannabis sativa
MDALSSFQGSNSLEVKMEDRAIAQPECGRPPIVSEAKRAAVQEELKRVNKLPATSTYASHRLRVLNKILQLLSVQRSSSEEMELELLFAGLSL

SEQ ID NO: 417 - DNA, 864 bp, Cannabis sativa
  source 1..864: mol_type = other DNA
ttgggagagagacctgaacctgcgccttatgggtttcagcctgaagctcagtatggctctgagagacctggatctgaagatggatctggatacggtcgtaagcctgagcctgaacctgaatcggaatatggatctggctatggaaagaagagtgagtttgaacagtcggaatatggatctggctacggtcgtaagcctaaacctgaacctgaatcggaatatggatctggctatggtagacagccagaacctgtgtcggagtatgggtctgggtatgggagacagccggaacctgtgtcggagtatgggtctgggtatgggagacagccggagccagtatcggaatacggatctggatatggtaggagacatgaatcagaggaaccgagttctgaatatggatctggttacggtcgaaagacagagtatgagagttctgggtatgtagcgtctgagtatgttaagccctcttatggtggtgatgatgagtcaccgaagaaaccaagctatgggaatccaagttacgggagtcaagaaggagaatacgggagatcaagttatgggaggtccgaggaagagggttatcgtaagcctagttatgagaggcgtgatgatgatgatgaggatcgtcccaagtacggttacagcgaggaacgccatggtcgcaagaaatatggtggtgatggctctgatgatgatgaagagaagaagcaatctcgctataagcagggttctgataatgaagatgatgaagagaagaggcgatctcgctacaacaagcaaggctctgatgatgatgaagagaataagcatcctcgctataagcaccaccaccaccaccgcaagagctacgacgacgaatga

SEQ ID NO: 418 - protein, 287 aa, Cannabis sativa
LGERPEPAPYGFQPEAQYGSERPGSEDGSGYGRKPEPEPESEYGSGYGKKSEFEQSEYGSGYGRKPKPEPESEYGSGYGRQPEPVSEYGSGYGRQPEPVSEYGSGYGRQPEPVSEYGSGYGRRHESEEPSSEYGSGYGRKTEYESSGYVASEYVKPSYGGDDESPKKPSYGNPSYGSQEGEYGRSSYGRSEEEGYRKPSYERRDDDDEDRPKYGYSEERHGRKKYGGDGSDDDEEKKQSRYKQGSDNEDDEEKRRSRYNKQGSDDDEENKHPRYKHHHHHRKSYDDE

SEQ ID NO: 419 - DNA, 489 bp, Cannabis sativa
  source 1..489: mol_type = other DNA
atggaaggagacagaagagttggggtggctgtggatttctctgagtgtagccgagaagccctgaaatggactctcaataacgttatccgagatggggatcaccttattctgatcagtgttcgtccagagggtaattatgaagatggggagatgcagctctgggcaaccactggttcacctttaattcctttgagtgagttctgtgatgctcacaccatgaaaaagtatggagtaactcctgaccctgcaacactagacattgttagcaccattgctaggcagaaagaggttgtggtgttaatgaagatctattggggtgatgcccgtgagaagatttgtgaagctattgataatattcctttgagctgccttgttattggtaacagaggccttggcaaactcaaaagggttgtccttgggagtgttagtaactatgtggtgaataatggctcatgcccagttactgtggtcaagcatggacaccattaa

SEQ ID NO: 420 - protein, 191 aa, Cannabis sativa
LGVFFGAYRRFRSKPRKEKGKKKKKKKKAMEGDRRVGVAVDFSECSREALKWTLNNVIRDGDHLILISVRPEGNYEDGEMQLWATTGSPLIPLSEFCDAHTMKKYGVTPDPATLDIVSTIARQKEVVVLMKIYWGDAREKICEAIDNIPLSCLVIGNRGLGKLKRVVLGSVSNYVVNNGSCPVTVVKHGHH

SEQ ID NO: 421 - DNA, 486 bp, Cannabis sativa
  source 1..486: mol_type = other DNA
atggctaccgctgagaaaccagtcatggtggttggaattgacgaaagcgaccacagctcttatgctctacaatggactctagaccgcttctttgctgcctatgctcccaactttccatttaagctcattcttgtccacgctaagcctactccaacctccgccgtcggtctcgctggccctggcgcggctgaggttttgccgtttgtcgatagtgatttgaggaggatcgctgcaagagtcgttgagaaagctaaggaagtttgcgccagtaaatcggtgaatgatgttttggttgaggttatggaaggtgatgctcggaatgtcctctgtgaaattgtagagaaacaccatgcatctgttttggttgtgggcagtcatggctatggagcaataaagagggctgttttgggcagtgtcagtgactattgtgctcaccatgctcactgctctgtgatgattgtgaagaagcctaaaaccaagcattaa

SEQ ID NO: 422 - protein, 161 aa, Cannabis sativa
MATAEKPVMVVGIDESDHSSYALQWTLDRFFAAYAPNFPFKLILVHAKPTPTSAVGLAGPGAAEVLPFVDSDLRRIAARVVEKAKEVCASKSVNDVLVEVMEGDARNVLCEIVEKHHASVLVVGSHGYGAIKRAVLGSVSDYCAHHAHCSVMIVKKPKTKH

SEQ ID NO: 423 - DNA, 303 bp, Cannabis sativa
  source 1..303: mol_type = other DNA
atgttgccacctcggatgaagcaggccatcacagataacccaaagaagctggcaaatttgattgatcttataaatcttcctacgccattaagggagttcatggggcagtctcagacttctcgtcttggctgtttcatgcgtgtctggtcatatatcaaggagaacaatcttcaggatcccaacaacaagaatgtggtcaattgtgatcataagttgaggagtattttattgggcaagcctcaagttgagttagctgatcttccttcattgatcaagcttcattttccaaagaaaacaagctaa

SEQ ID NO: 424 - protein, 100 aa, Cannabis sativa
MLPPRMKQAITDNPKKLANLIDLINLPTPLREFMGQSQTSRLGCFMRVWSYIKENNLQDPNNKNVVNCDHKLRSILLGKPQVELADLPSLIKLHFPKKTS

SEQ ID NO: 425 - DNA, 186 bp, Cannabis sativa
  source 1..186: mol_type = other DNA
atgtgtttggaagatatggttttacaattttataaccttgccaagtctcgtaggttagcagaaattcagcttgttttatattttcatgctgtacttttcaaatttcatagaaatttggagagaatgtttgcacagctgaaaatacttgttttggaattacattgcctaaagtaccaatcatgttga

SEQ ID NO: 426 - protein, 61 aa, Cannabis sativa
MCLEDMVLQFYNLAKSRRLAEIQLVLYFHAVLFKFHRNLERMFAQLKILVLELHCLKYQSC

SEQ ID NO: 427 - DNA, 660 bp, Cannabis sativa
  source 1..660: mol_type = other DNA
atggaaggagatgagaatgatcttacacccttttggctacaatcctcagatagtcgccggcagcggcggctgagccgttcatcatctctactcttcaactctagtgcttttctcatagtcttgattgttacaacacttgcatttatcttctttattattccaacgattctttccttcgcttctcaaatattcaaacctcagtctgtaaagaagagctgggattctctcaatctgattctagttctgttcgccattatttgtgggtttctaagcagaaacagtggtgagaacgctagcccttatgaagatcgaactgtttctaaatcttcttcaactcgagaggttgagaagtccaacccatcaagtcccaatcgatggtaccaatattcagatcggacactgtcatataacaatggaaatggacttactagactcagaaatagtagctcataccctgatctacggcaagaatcttcctgggttaccagggatgaacaatggcaattctacgatgatacccatgtctctaattaccgcccactcggctccgatgggctacaccaacgtcgccggccattgcaggaacccctcagccttctacgcagcttccgcctcagccacccccatttcctcctccgtcgccgcccacggctccggtga

SEQ ID NO: 428 - protein, 219 aa, Cannabis sativa
MEGDENDLTPFWLQSSDSRRQRRLSRSSSLLFNSSAFLIVLIVTTLAFIFFIIPTILSFASQIFKPQSVKKSWDSLNLILVLFAIICGFLSRNSGENASPYEDRTVSKSSSTREVEKSNPSSPNRWYQYSDRTLSYNNGNGLTRLRNSSSYPDLRQESSWVTRDEQWQFYDDTHVSNYRPLGSDGLHQRRRPLQEPLSLLRSFRLSHPHFLLRRRPRLR

SEQ ID NO: 429 - DNA, 321 bp, Cannabis sativa
  source 1..321: mol_type = other DNA
tggccaacttttttgtacaaagttgtccccccaacttttttgtacaaagttgtccccggccgcacaactttgtacaagaaagttgggttttttttttttttttttaatattttgctactacagtggggtttagaatcacctaacaaggttttgagattgctagggtttttggggggtgttagggttttctggtttttcttttttttagttgggttttattattatgagataatttcttgtcttttttttatgggtttattttttttttggttgcttatattttcccagtcttgtaaatctttgcagaggtcagctttttga

SEQ ID NO: 430 - protein, 106 aa, Cannabis sativa
WPTFLYKVVPPTFLYKVVPGRTTLYKKVGFFFFFFNILLLQWGLESPNKVLRLLGFLGGVRVFWFFFFLVGFYYYEIISCLFFMGLFFFWLLIFSQSCKSLQRSAF

SEQ ID NO: 431 - DNA, 999 bp, Cannabis sativa
  source 1..999: mol_type = other DNA
atgggaaatcctcttctcgacatttctgccgtcgtccctgaggaatttctgaaaaaatatgacgtcaagccaaacaatgctattcttgctgaggataagcacttgcccatgtatgatgaactggccaatgaatatgatgtggagtttattgctggaggtgctacacaaaattcaataaaagttgcccagtggatgctccaaattcctggtgcaactagttacatgggaagtattggaaaggataagtttggagaggagatgaagaaaaattgcaaacaagctggtgttaatgtgcactattatgaagatgaaactgcacctaccggaacttgtgctgtctgtgttcttggtggtgagaggtcacttattgcgaacttgtcagctgcaaattgttacaaaactgaacatctgaagcaaccagaaaactgggccctagtggaaaaggccaaatattactacattgctgggtttttcctcactgtgtctccagaatccattcaacttgttgctgcgcatgcagctgcaaacaacaaggtcttcacaatgaacctgtctgctccatttatttgtgaattcttcaaagatgcacaggagaaggctttgccgtacgtggattacgtttttggaaatgagactgaagcaagaacgtttgcaagggttcatggctgggagactgaaaatgttgaagagattgctgtcaagatttcccaattgcccaagcaatcaggtacacacaagaggacaactgtcattacccaaggtgcagatcctgttgtagttgctgaggatgggaaggtcaaattgttccctgttattttgttacccaaggagaagcttgttgacaccaatggagcaggggatgcatttgttggtggatttttgtcacagttagttaaggagaagccaattgaagattgtgtgaaagctgggtgttatgcagcaaatgttatcatccagaggtcaggatgcacataccctgaaaagccagatttcgaatag

SEQ ID NO: 432 - protein, 332 aa, Cannabis sativa
MGNPLLDISAVVPEEFLKKYDVKPNNAILAEDKHLPMYDELANEYDVEFIAGGATQNSIKVAQWMLQIPGATSYMGSIGKDKFGEEMKKNCKQAGVNVHYYEDETAPTGTCAVCVLGGERSLIANLSAANCYKTEHLKQPENWALVEKAKYYYIAGFFLTVSPESIQLVAAHAAANNKVFTMNLSAPFICEFFKDAQEKALPYVDYVFGNETEARTFARVHGWETENVEEIAVKISQLPKQSGTHKRTTVITQGADPVVVAEDGKVKLFPVILLPKEKLVDTNGAGDAFVGGFLSQLVKEKPIEDCVKAGCYAANVIIQRSGCTYPEKPDFE

SEQ ID NO: 433 - DNA, 654 bp, Cannabis sativa
  source 1..654: mol_type = other DNA
ggggggacaactttgtacaaaaaagttggtacagatcctgatgttgacatatttgatcgggaaaaggttttcattgaaactattttgcggcccttaatagagaggcttccacagctgaaagttgtgatggaacatgtaaccactttggatgctgttaggtttgtggagtcatgcaaagaaggctatgtggctgcaactgtcacaccacaacatcttctgcttaataggaactctatattccaaggaggattgcagcctcataattattgcctcccaatactcaaaagagagatccacagacaagctatagtgtctgctgtcaccagtggaagtaaaagatttttccttggaactgatagcgctcctcacgagagacgaagcaaagaatgttcttgtggttgtgctggtgtattcaatgcgcctgtggcactatcggcgtatgccaaagtctttgaagaggctggtgctcttgacaaactagaggcttttacaagctttaatgggccagatttctatgggatttccagaaacaactcaaaaattaaactaaggaaaaatccatggaaagttcctcattcttactcatattcttttggcgaactagtcccaatgtatgctggagaaacacttgattggaaaccatccttggactga

SEQ ID NO: 434 - protein, 217 aa, Cannabis sativa
GGTTLYKKVGTDPDVDIFDREKVFIETILRPLIERLPQLKVVMEHVTTLDAVRFVESCKEGYVAATVTPQHLLLNRNSIFQGGLQPHNYCLPILKREIHRQAIVSAVTSGSKRFFLGTDSAPHERRSKECSCGCAGVFNAPVALSAYAKVFEEAGALDKLEAFTSFNGPDFYGISRNNSKIKLRKNPWKVPHSYSYSFGELVPMYAGETLDWKPSLD

SEQ ID NO: 435 - DNA, 570 bp, Cannabis sativa
  source 1..570: mol_type = other DNA
atgggctttagagaagtacctaatgtgttggagaccaagtgtggagctgtttctgcctttgctattcaacaagaaggtggtggtttattagaagatagagaccagaaattcttgagtagggctgtagaggaggcttataaaggagttgaaagtggagatggagggccttttggagctgttattgtatgcaatgatgaagttgttgtaagttgtcataacatggttttgagatgcactgatcctactgctcatgctgaggttaatgccattagagaggcatgtaagaaactgaagaggattgagctatctgattgtgaaatctatgcatcatgtgagccttgccccatgtgctttggtgccatccacctttcaagacttaagaggctggtttatggagccaaagctgaagctgcaattgctattgggtttgatgatttcatagcagatgcattgagaggaactgggttctatcaaaaggctcacttggatattaaaaaggcagatggcaatgcagccattattgctgaacaagtctttgagaatactaaggctcatttccaaatctattaa

SEQ ID NO: 436 - protein, 189 aa, Cannabis sativa
MGFREVPNVLETKCGAVSAFAIQQEGGGLLEDRDQKFLSRAVEEAYKGVESGDGGPFGAVIVCNDEVVVSCHNMVLRCTDPTAHAEVNAIREACKKLKRIELSDCEIYASCEPCPMCFGAIHLSRLKRLVYGAKAEAAIAIGFDDFIADALRGTGFYQKAHLDIKKADGNAAIIAEQVFENTKAHFQIY

SEQ ID NO: 437 - DNA, 453 bp, Cannabis sativa
  source 1..453: mol_type = other DNA
tggcttcaacttgcactacttgaccgcgaatttgatgacttagattacgagaccttgagagcgctagatgcagataatgctccaacagctcattctatgacggaggaagagataaatgctcttcctgtccataaatacaaggttgctgatctacaaaatagtgcctcatctatgcaacaggcctcatcgtcagcatctgctgagaaacaaggatctaatgatgcagttgtgaaccccaaggcctcagaagatgaactgacttgcagtgtttgcctggagcaagtaaatgttggagaagtccttcgaagcttgccgtgcttgcatcagttccatgctaattgtattgatccttggctgaggcagcaaggaacttgtcctgtttgtaaattcaaggcaggatctgtgtggcaggaaagtggagaaggtgaagtggatgcttcttttatggtttag

SEQ ID NO: 438 - protein, 150 aa, Cannabis sativa
WLQLALLDREFDDLDYETLRALDADNAPTAHSMTEEEINALPVHKYKVADLQNSASSMQQASSSASAEKQGSNDAVVNPKASEDELTCSVCLEQVNVGEVLRSLPCLHQFHANCIDPWLRQQGTCPVCKFKAGSVWQESGEGEVDASFMV

SEQ ID NO: 439 - DNA, 354 bp, Cannabis sativa
  source 1..354: mol_type = other DNA
atgactaagtttctgtgtgttgttctcttagctcttcttagcatttccatggttgcaaaccaggttttggcaaaagaatcatctgagcacaacttagaaagtagaaactatggacctgcgagtcttaaaaagtaccaatgcccaggagaatgcacaagaagatgcagccaaacacaataccataaaccttgcatgttcttctgccaaaaatgctgcaacaagtgcctatgtgttcctcctggctattatggcaacaaagctgtatgcccctgctacaacaactggaaagaccaaggaaggaggacccaagtgcccttgagccattctattatatctattttagttaaattataa

SEQ ID NO: 440 - protein, 117 aa, Cannabis sativa
MTKFLCVVLLALLSISMVANQVLAKESSEHNLESRNYGPASLKKYQCPGECTRRCSQTQYHKPCMFFCQKCCNKCLCVPPGYYGNKAVCPCYNNWKDQGRRTQVPLSHSIISILVKL

SEQ ID NO: 441 - DNA, 1626 bp, Cannabis sativa
  source 1..1626: mol_type = other DNA
atggaagagatgccttcggcagttgcaatgacatttagcgtagataatcctatgtgtgataactctggaatcacaacccacgttgggttcactcagctcaagctcgtaactgaagctggaaatttaccattagatcctagtaagttggtgtctgctgagtctatttcatgtgctaatgttagttgtaatgctgcaaagaatacagttagcatggtgaccatgtcaactcaaggagagaatggaggtggagccgatttgtttactatattgcccgagaatggaaatatttgtgttaccagtgatgttatggtccaagttagcagtgatgatgaaatggtagcagtttgtgacaataataatggaattatcaaggaagagttggtagctttgggggtgggacctgaaataagcataccagatgttgtagagattgaaaatgttgaagatggtcagattgttgctaaggctattatcttggtggaatcaagcaatggtaaggtaccttctggtgaagttattgttgcagcagtgagtcctgcttcggatatttccagcaagtctgacttagtggcctccacactggttattcagtcacagggggagaggaacttcggtaaagggggcagccgaagtgttttggaacaggactgcatccccctttggggttctgtttctatttgtggaagaagaccggaaatggaagatgcagttgctgctatacctcggtgtataaaaattcctatcaaaatgcttttcggcaatcatatgctcaatgaaaccaaccagaatttgacccacctgaacagtcatttttatggtgtttatgatggccatggtggttctcaggttgctagttattgcagtgagcgtatccatttggctttggctgaggagatagaaatgtgtaatgatgatttaagcaatggagatgttggagaaaatcaacaagtactgtgggagaaagcctttacttcttgctttcaaaaggtagatgatgagattggtggaaaaattaatggagatgcttccgatgctacaattgagcctattgccccagaaactgttgggtctacagctgtggttgccttagtctgttcatcacacatcatagttgcaaactgtggagactcaagagcagtgctatgccgtgggaaagaacctgtcccgttgtcagtagatcataaacccaatagagaggatgaatatgctaggattgaggcatccggaggaaaggtcattcagtggaatggtcaccgagtttttggtgttcttgcaatgtcaaggtccataggagatagatatttaaaaccgtggataattccagaaccagaagttatgtttgtccctcgagccagagaggacgagtgccttatcttagccagtgatggtttatgggacgttatgacgaatgaagaaatttgtgaagtagcccgacggcggctactcctctggcacaagaagaacggtgtcacagctttggcagacaggggtttgggcgcggatctagcagcgcaagcagcagctgagtacctctcaatgctagcccttcaaaagggcagcaaggacaacatctctgtgattgtggtggatttgaaagctcatagaaaattcaagagcaaatcctaa

SEQ ID NO: 442 - protein, 541 aa, Cannabis sativa
MEEMPSAVAMTFSVDNPMCDNSGITTHVGFTQLKLVTEAGNLPLDPSKLVSAESISCANVSCNAAKNTVSMVTMSTQGENGGGADLFTILPENGNICVTSDVMVQVSSDDEMVAVCDNNNGIIKEELVALGVGPEISIPDVVEIENVEDGQIVAKAIILVESSNGKVPSGEVIVAAVSPASDISSKSDLVASTLVIQSQGERNFGKGGSRSVLEQDCIPLWGSVSICGRRPEMEDAVAAIPRCIKIPIKMLFGNHMLNETNQNLTHLNSHFYGVYDGHGGSQVASYCSERIHLALAEEIEMCNDDLSNGDVGENQQVLWEKAFTSCFQKVDDEIGGKINGDASDATIEPIAPETVGSTAVVALVCSSHIIVANCGDSRAVLCRGKEPVPLSVDHKPNREDEYARIEASGGKVIQWNGHRVFGVLAMSRSIGDRYLKPWIIPEPEVMFVPRAREDECLILASDGLWDVMTNEEICEVARRRLLLWHKKNGVTALADRGLGADLAAQAAAEYLSMLALQKGSKDNISVIVVDLKAHRKFKSKS

SEQ ID NO: 443 - DNA, 1053 bp, Cannabis sativa
  source 1..1053: mol_type = other DNA
atgggaatctatctcagttctccaaaaactgataaatcctcagaagatggagagaatgaaagacttcgatttggggtgtcttctatgcaagggtggcgggcatccatggaagatgctcattctgctcatccagatttggacacttcaacgtctctttttggtgtttatgacggtcatggaggtaaagcagttgcaaagttctgtgccaagtatcttcaccaacaggtgctcaagcatgaagattacatggctggagatataggctccgctttacagaaatgctttctaagaatggatgagatgatgcgtggccaaaaagggtggagggaattggcaatcttaggcgataatttagacaaggtttctggtgtgatagaagggttaatatggtctcctagaggtgggcaagctaagagaggcgaattaagtaaccatgttgaagattggccttctgaagagggacctcactcagattttgatggaccgaattgtggtagcacagcttgtgttgcagtcattcggaacaaacaacttgttgtagcaaatgccggtgattctcgttgtgtgatatccaggaagggtcaggcatataatttgtcgaaagatcataagcccgacttagaagttgagaaagataggattctgaaagccggtggatatatccaagttggtcggatcaatggaagtttgaacttgacaagagctataggagacgtggaattcaaacaaaacaaacatttgactgctgaaaaacagattgtaactgctaatccagacataaatactattgagctatgtgatgatgatgagtttctcgttttagcttgtgatgggatatgggattgcatgtctagccaacaacttgtggactacgtacgagaccagttaaaagaagaaactaagctctcggccatttgtgagaaagttttcgacagatgtgtggcaccaactgctggtggggaaggttgcgacaatatgactatgatcttggttcagtttaagaaaccgattaataatggagatgcttcagttgatgggcagactcctttatga

SEQ ID NO: 444 - protein, 350 aa, Cannabis sativa
MGIYLSSPKTDKSSEDGENERLRFGVSSMQGWRASMEDAHSAHPDLDTSTSLFGVYDGHGGKAVAKFCAKYLHQQVLKHEDYMAGDIGSALQKCFLRMDEMMRGQKGWRELAILGDNLDKVSGVIEGLIWSPRGGQAKRGELSNHVEDWPSEEGPHSDFDGPNCGSTACVAVIRNKQLVVANAGDSRCVISRKGQAYNLSKDHKPDLEVEKDRILKAGGYIQVGRINGSLNLTRAIGDVEFKQNKHLTAEKQIVTANPDINTIELCDDDEFLVLACDGIWDCMSSQQLVDYVRDQLKEETKLSAICEKVFDRCVAPTAGGEGCDNMTMILVQFKKPINNGDASVDGQTPL

SEQ ID NO: 445 - DNA, 879 bp, Cannabis sativa
  source 1..879: mol_type = other DNA
atggataatctatgttgcttcaattctgtctattctcaggttgttgggggacggtcttcatgtagctccgggaagggcaggagccatcaggcctcggtcaagtatgggttcagcctggttaaaggaaaagcaaatcatcccatggaagactatcatgtagccaaatttgttcaaatgcaaggacatgaacttggactatttgctatatatgatgggcatttgggagatagtgtacctgcttatctacaaaagaacttattttctaatatcttaaaggatgaagagttctggaccgatcccagcaggtccatttctaaggcctatgagagaacagaccaggctattctttctcacagcccagacttgggacgaggaggatccacggcagttactgccattttgatagacaaccgaaagttatggattgccaacgtcggagactcacgagcagttctctcaaagaaaggacaggcagttcagatgacaattgatcatgaacctaacactgagcgtggtagcattgagaaccgaggtggcttcgtctcaaacatgccaggagatgttgcacgggttaatgggcagctggcagtttctcgagcattcggagacaagaatcttaagtcacacttgcgctctgatcccgacataaaatctgaagatattaactcagacgccgactttctcatccttgcaagtgatggtttatggaaggttatgtctaaccaagaagcagttgatgttgcaaaaaagactaaagatccacagagggcagcgaaacaattagcagctgaggcactaaacaaagacagcaaagatgacatatcaatcgtcgtagttcgtttcaagggtgaaagacgaagaaaatatactatttactaa

SEQ ID NO: 446 - protein, 292 aa, Cannabis sativa
MDNLCCFNSVYSQVVGGRSSCSSGKGRSHQASVKYGFSLVKGKANHPMEDYHVAKFVQMQGHELGLFAIYDGHLGDSVPAYLQKNLFSNILKDEEFWTDPSRSISKAYERTDQAILSHSPDLGRGGSTAVTAILIDNRKLWIANVGDSRAVLSKKGQAVQMTIDHEPNTERGSIENRGGFVSNMPGDVARVNGQLAVSRAFGDKNLKSHLRSDPDIKSEDINSDADFLILASDGLWKVMSNQEAVDVAKKTKDPQRAAKQLAAEALNKDSKDDISIVVVRFKGERRRKYTIY

SEQ ID NO: 447 - DNA, 2184 bp, Cannabis sativa
  source 1..2184: mol_type = other DNA
atggatgggaattcagaacaagcttgctcagaaattgttgaagttgatcctactggaagatatggaaggtacaatgagattcttggtaaaggggcatcaaagacagtctatagagcctttgatgagtatgaagggattgaagtggcatggaaccaagttaaactctatgactttttgcaaaatcctgaagatcttgagagactctactctgaaattcatcttcttaagaccttgaaacacaaacatataatgaagttctacacttcttgggttgatactgctaacagaaacattaactttgtcactgagatgttcacctctggcactcttaggcagtataggctaaaacacaggagagttaacataagagcggttaagcattggtgcagacagattctgaaaggtcttctatatcttcacagccgtaacccccctgtgattcacagagatctcaagtgtgacaatattttcatcaatggaaaccaaggggaagttaagattggtgaccttggccttgctgctattctgagaaaatcccatgctgctcattgtgtaggaacaccagaatttatggctccagaagtgtatgaagaggaatacaacgaattagtagacatatatgcttttgggatgtgtatcttggaaatggttacctttgaatatccatacagtgaatgtacacaccctgctcagatttacaagaaagttatctctggtaaaaaaccagaagctttgtacaaagtgaaaaatcctgaggtgagacaattcgttgagaaatgcttagcaacagtgtcttgtaggtttactgctttagagcttctgaaagacccttttctccaagtggaggattatggatatgatatgagttcattggagtatgaaactgactatgatgatgtaggacctttctttaggcaacctcactatggacttcatcacagtaacagtaacagatatttgaacaatggttatgtctatgaacctgaaaatgacttggattgtcatcctgttattgagtatgaagcaaatgagattgacctcttctctggtcaagaggatgagcatgtgggagattttgacatcacaatcaaaggaaggagaaaagatggcgaggatggaatctttttaaggctcaggattgcagataaagagggccgaattcggaacatatatttcccatttgatattgagaatgatactgcgttgagcgttgctactgaaatggttaacgagcttgatatcactgaccaagatgtgatcaaaatagcagacatgatagataatgaaattgccattttggtaccagagtggaaaaggggattggccgtagaggaaaataagcattgctcagctacaagcttatgtcacaactgtgatacaaatggctccataatggattatgtaacatcagatagttcagctgccaagaaccttcaagtcctcgagtgttccaaacatggctctgcagccatccatggtcgatttgaagaaattacataccaagttgaagggtcttcagaacaatgccttgcagagggcgcagcagaacattctcgccactctcaaggcatccaattcgctgatatctgggcacagagagatgggccagaattacactcacaggggtcaaaagacatctactgcgacgatgcagataaatctttgatccttcctaaccctgatgagaaagaagagaagattatagttatggacaatcaaagcgagtctagcgctgcaaagttaagtccaccggcaaatcatgattctagttctgtttttgatgattatgagaatgacattagacaagagctgagatggctcaaagccaaatatcaaatgcaaatgagagggcttaaacaccaacagttaagagccaagaaaaagccatcaagcttagctccaagtcctgagaacttggaacaaaaggcagacagtaatggagaatcaatgtcttcagtctcacaccagctgaagaaagataatagaagacctccccttaaaacatccacctttgtaaaccatttttccaatgtggataatcaaaatgttcaaagcatggaggcaatgaagggttcatggagtccggaccaaatattcacggctaagagtttctacagtggatctatgttttcacagcctcttcatagagcaacttctcttccagtcgatgctatagatgcttaa

SEQ ID NO: 448 - protein, 727 aa, Cannabis sativa
MDGNSEQACSEIVEVDPTGRYGRYNEILGKGASKTVYRAFDEYEGIEVAWNQVKLYDFLQNPEDLERLYSEIHLLKTLKHKHIMKFYTSWVDTANRNINFVTEMFTSGTLRQYRLKHRRVNIRAVKHWCRQILKGLLYLHSRNPPVIHRDLKCDNIFINGNQGEVKIGDLGLAAILRKSHAAHCVGTPEFMAPEVYEEEYNELVDIYAFGMCILEMVTFEYPYSECTHPAQIYKKVISGKKPEALYKVKNPEVRQFVEKCLATVSCRFTALELLKDPFLQVEDYGYDMSSLEYETDYDDVGPFFRQPHYGLHHSNSNRYLNNGYVYEPENDLDCHPVIEYEANEIDLFSGQEDEHVGDFDITIKGRRKDGEDGIFLRLRIADKEGRIRNIYFPFDIENDTALSVATEMVNELDITDQDVIKIADMIDNEIAILVPEWKRGLAVEENKHCSATSLCHNCDTNGSIMDYVTSDSSAAKNLQVLECSKHGSAAIHGRFEEITYQVEGSSEQCLAEGAAEHSRHSQGIQFADIWAQRDGPELHSQGSKDIYCDDADKSLILPNPDEKEEKIIVMDNQSESSAAKLSPPANHDSSSVFDDYENDIRQELRWLKAKYQMQMRGLKHQQLRAKKKPSSLAPSPENLEQKADSNGESMSSVSHQLKKDNRRPPLKTSTFVNHFSNVDNQNVQSMEAMKGSWSPDQIFTAKSFYSGSMFSQPLHRATSLPVDAIDA

SEQ ID NO: 449 - DNA, 897 bp, Cannabis sativa
  source 1..897: mol_type = other DNA
atgggatctggtaatggggtttactcggttgacgagtttagtttagatgccaagtggttgatcgatccaaaacatctttttgttggaccgaggattggggaaggtgcacacgccaaagtgtacgaaggaaaatataagaatcagactgttgctgttaaaattgttcatagaggagatacaccagaagaaattgccaagagagaagggaggtttgcaagagaggttgcaatgttgtctaaagttcagcacaaaaatttagtgaagtttattggagcttgcaaggaacctgtcatggtcatagtgacagagcttctgttaggtgggacattgcggaaatacttgtttaatatgcggccaagatgcttggatacgcgtgtggcagtcgggtttgcgcttgatattgctcgtgcaatggaatgcttgcactctcatgggatcattcaccgtgacctcaaacccgagaacttgattttgactgcagaccataaaacggttaaactcgccgactttggcttagctagagaagagacactcacagagatgatgactgctgaaactgggacttatcggtggatggcaccagagctttacagcacggtcacattaaggcatggcgagaagaagcattacaatcacaaggtggatgcatacagctttgccattgttttatgggagctcgtccataataagttgccttttgaaggcatgtcgaatctacaagctgcatatgctgcagccttcaagaatgtgaggccaagtgctgagaacctccctgaggatttagcattgattgtaacttcatgttggaagaagatccaaatgacagacccaacttcagccaaattatacaaatgctcctgcactatctctcgacgatatcacctgcagagccggtaa

SEQ ID NO: 450 - protein, 298 aa, Cannabis sativa
MGSGNGVYSVDEFSLDAKWLIDPKHLFVGPRIGEGAHAKVYEGKYKNQTVAVKIVHRGDTPEEIAKREGRFAREVAMLSKVQHKNLVKFIGACKEPVMVIVTELLLGGTLRKYLFNMRPRCLDTRVAVGFALDIARAMECLHSHGIIHRDLKPENLILTADHKTVKLADFGLAREETLTEMMTAETGTYRWMAPELYSTVTLRHGEKKHYNHKVDAYSFAIVLWELVHNKLPFEGMSNLQAAYAAAFKNVRPSAENLPEDLALIVTSCWKKIQMTDPTSAKLYKCSCTISRRYHLQSR

SEQ ID NO: 451 - DNA, 819 bp, Cannabis sativa
  source 1..819: mol_type = other DNA
atggcattagtagaagtaagtgtaacccagatgtccagaacagattgtatgatattgacaaacacgcataaatgtgacattcatgggcagtattcggatcttttgaggctttttgagtacgggggttttcctcccaatgctaattatctattcttaggagactatgtcgaccgtggcaaacagagtttggaaactatttgcctcttgcttgcatataaaattaagtaccctgagaacttctttcttctaagaggaaatcatgaatgtgcttcgattaaccggatttatggattttatgatgaatgtaaacggcggttcaatgtcaggctttggaagacctttactgatagttttaactgtcttccagtagctgctctcatagatgataaaatattatgcatgcacggtggcctttctcctgatctaacacatttggatcagattaggaatttacctcgtccaactgatgttccagattctggtttgctttgcgatttactttggtcagatcctggtagagatgtcaaaggctggggaatgaatgaccgtggagtctcatacacttttggtgtggataaagttgcagagtttttaactaagcatgaaatggaccttgtttgtcgtgcccatcaggttgttgaggatgggtatgaattctttgctgacaggcagcttgttacaatattttctgctcctaattattgtggtgagttcgataatgctggtgcaatgatgagcgttgatgaaagcttaatgtgctctttccaaattctcaagccagctgaaaaaaaacctaggtttggttcaacaatgatgtga

SEQ ID NO: 452 - protein, 272 aa, Cannabis sativa
MALVEVSVTQMSRTDCMILTNTHKCDIHGQYSDLLRLFEYGGFPPNANYLFLGDYVDRGKQSLETICLLLAYKIKYPENFFLLRGNHECASINRIYGFYDECKRRFNVRLWKTFTDSFNCLPVAALIDDKILCMHGGLSPDLTHLDQIRNLPRPTDVPDSGLLCDLLWSDPGRDVKGWGMNDRGVSYTFGVDKVAEFLTKHEMDLVCRAHQVVEDGYEFFADRQLVTIFSAPNYCGEFDNAGAMMSVDESLMCSFQILKPAEKKPRFGSTMM

SEQ ID NO: 453 - DNA, 2283 bp, Cannabis sativa
  source 1..2283: mol_type = other DNA
atgaagtcagtggcagtggccgttgacggtggcaacggcagccaacgcgcagtcagctgggcggtggagaatctcatgccccaatctgcaagcttcgtcttgatccatgtcatgcccaaaatcacctctattcccactccatctggggattggattcccgtgtctgaactggacgcaaatatggttgctatgtatgttgaggagataaaatctaaggcggaagatgttttcattccatttaagaagctgtgcagaacgaaacagatggaaactttgattttagaggggaaagatccagcatccacgcttctaaaatatgttgctgatgcagggattgaatgcttagttctgggttcttcttcttcaaattttctaacaaggaaactgaagggaccaggggttccaacaagccttttaagttgtactaccaacccatgtgaaattcatgttgtatcgaagaagaaaataattacaaaggcagctaattcatcatcttcatatgagactagcaccgcacactgggtttatacttcaagagacaacagagaaggattcagtggttcagatgaacaattatccaggcgtcgattcttctcagtagagtcttcattaaatgaaagtcttggagcatcaataaatctggatcttaactacctaaactctgaagcatttcaacaaaatggttcttctgccaattctagttttcaagacttaaatctccaaatcttggaaaatagccttgaaactcttacattaaatggatgcaattatgcagattctttggatactgacaagttggactcacagactgaggttgcccgtgtgcgccaagaattgcaaaatactattgcaatgtataaaaaaacttgtgaagaattggtccatgcccaaaagaaggttcagttagtttcctctgcatgcctgaaagaaacaaacagagtcaatgctgctctcgtaagagaagaaactttgaagaaaattgcagccgaagaaaaagcaaatcatttggaagcggtaaaggaggttgagagggaaagagttttgcttgcaaaagaagcttatgaaagacagattgctgaagttaaggcctttgaagagtctttagagaaaagaaagcttgttgatgaactcttttctaatgataagaggtacaggagatacactaggaatgaaatagaaatagccacagatttcttttcagaggctaatgtgattggagaaggaggctatggaaaagtctacaagtgcagtcttgatcacatcccagtagctattaaggttcttcaaaatggtaaagatgacaagaaacatgaatttttgaaagaggttgaagttctaagtcagctacaccatccgaatattgttttattgcttggagcctgtcccgagaatggttgcctagtttaccaatatatggaaaatggaagcctagatgactatatctaccaacgtaatggaaaatcacaacttccatggttcataaggtttcgagtagcttttgaaatagcttgtggtcttgcattcttacacaatgcaaagccagagcctatagttcatcgagacctaaaaccgggaaatatcttgttagatagaaactatgtaagcaaaattggggatgttggtctagccaaaatcatttcggatgttgtgcctgacagcatcaccgagtacagagactcagttattgctggtacactcttctacttggacccggagtatcaaagaactggcacaatcagaccaaaatcagatttatatgcttttggcatcatagtcctccaattgttgacatttcgtcatccaaagggacttgttacaagtatcgagaatgctattcgacatggttccttctttgatattctagataattcagttacagattggccaatagaaacagaggaattagctcatattgctctgaagtgttcaaatgttagatgcagagacagacctgatcttgagactgaagttttgcctcttctcaaaagattggctgatcttgcaaatactaatgtagaagagaagaagaacatgcaggcactaagccactattattgtccaatccttcaggaaatcatggaggatccctatattgctgctgatggctttacttatgagtacagagcaattaagtcttggcttgagaaacacgatgtgtcaccaagtacaaagcaaaaattacagcattctatgttgataccagaccatacactccgatcagccataaatgattggaggtcatacgcctttgcgggtatccaaactaaaggaggtgacagttga

SEQ ID NO: 454 - protein, 760 aa, Cannabis sativa
MKSVAVAVDGGNGSQRAVSWAVENLMPQSASFVLIHVMPKITSIPTPSGDWIPVSELDANMVAMYVEEIKSKAEDVFIPFKKLCRTKQMETLILEGKDPASTLLKYVADAGIECLVLGSSSSNFLTRKLKGPGVPTSLLSCTTNPCEIHVVSKKKIITKAANSSSSYETSTAHWVYTSRDNREGFSGSDEQLSRRRFFSVESSLNESLGASINLDLNYLNSEAFQQNGSSANSSFQDLNLQILENSLETLTLNGCNYADSLDTDKLDSQTEVARVRQELQNTIAMYKKTCEELVHAQKKVQLVSSACLKETNRVNAALVREETLKKIAAEEKANHLEAVKEVERERVLLAKEAYERQIAEVKAFEESLEKRKLVDELFSNDKRYRRYTRNEIEIATDFFSEANVIGEGGYGKVYKCSLDHIPVAIKVLQNGKDDKKHEFLKEVEVLSQLHHPNIVLLLGACPENGCLVYQYMENGSLDDYIYQRNGKSQLPWFIRFRVAFEIACGLAFLHNAKPEPIVHRDLKPGNILLDRNYVSKIGDVGLAKIISDVVPDSITEYRDSVIAGTLFYLDPEYQRTGTIRPKSDLYAFGIIVLQLLTFRHPKGLVTSIENAIRHGSFFDILDNSVTDWPIETEELAHIALKCSNVRCRDRPDLETEVLPLLKRLADLANTNVEEKKNMQALSHYYCPILQEIMEDPYIAADGFTYEYRAIKSWLEKHDVSPSTKQKLQHSMLIPDHTLRSAINDWRSYAFAGIQTKGGDS

SEQ ID NO: 455 - DNA, 288 bp, Cannabis sativa
  source 1..288: mol_type = other DNA
atggagaaggttggttcaggtagtggaatgacttctcaacctgttatcattgaagattgtggacagttgaacttgagaactaatcttgtagaaattgttaaattaagattccaagcaacttgtattgttttttgttgcttttgcttgaggcctcccggggaaataatttatgtactaaatactaatgcaaagcctgaattacaggctgggtactccaatctcttaatatttgactctgtatattttatgctttgtgatgatactaaaataaaaatttctttattcatg

SEQ ID NO: 456 - protein, 96 aa, Cannabis sativa
MEKVGSGSGMTSQPVIIEDCGQLNLRTNLVEIVKLRFQATCIVFCCFCLRPPGEIIYVLNTNAKPELQAGYSNLLIFDSVYFMLCDDTKIKISLFM

SEQ ID NO: 457 - DNA, 642 bp, Cannabis sativa
  source 1..642: mol_type = other DNA
ggccaacttttttgtacaaagttgtcccctctttgtagccaccccgtagcgagacagtctgatcttcaggaactttttcttactgaaaaccaaaatatcacaaattggttttacaacatgtcgaaccccaaggtcttctttgacatccttattggaaagatgaaggctggccgagttgtaatggaattgtttgccgatgttacgccaaaaaccgctgaaaacttccgagctctatgcaccggagaaaaaggtcttggggtgtcagggaaggcgctgcattacaaaggctcagcctttcacaggatcatcccaaacttcatgtgtcaaggtggggactttaccagaggtaatgggaccggtggagagtcaatttatggtatgaagtttgaggatgagaacttcaagttgaagcacactggccctggaattctttcaatggctaattcgggaagaaacaccaatggctctcagttctttatatgcaccgacaagaccgactggcttgatgggaaacatgttgtttttgggaaagttgttgatggatatggcatagtcaaagaaatggagaaggttggttcaggtagtggaatgacttctcaacctgttatcattgaagattgtggacagttgactgagaactaa

SEQ ID NO: 458 - protein, 213 aa, Cannabis sativa
GQLFCTKLSPLCSHPVARQSDLQELFLTENQNITNWFYNMSNPKVFFDILIGKMKAGRVVMELFADVTPKTAENFRALCTGEKGLGVSGKALHYKGSAFHRIIPNFMCQGGDFTRGNGTGGESIYGMKFEDENFKLKHTGPGILSMANSGRNTNGSQFFICTDKTDWLDGKHVVFGKVVDGYGIVKEMEKVGSGSGMTSQPVIIEDCGQLTEN

SEQ ID NO: 459 - DNA, 435 bp, Cannabis sativa
  source 1..435: mol_type = other DNA
aggcctggaactggacccaagcccgtacctggtcgcaaggtcactgttcactgcactggttatggcaaaaatggtgatctctcccagaagttttggagcactaaagatcctgggcaggagccttttgcatttacaattggccaaggttctgttataaaaggatgggatgaaggtgttcttggaatgcaagtgggagaagttgctcgcctgcggtgcactccggattatgcttatggtccaagtggatttcctgcatggggaatcaaccaaattcttctttggttttcgagatcgaagtcctcaaggtggagtgaaatttggacagtaaaaagctatgcagaacaacttattgtgttcttgccaaacgtatctaaattattattatgtgatacgaataataatatctttggaggcttacttgatgagtacttataa

SEQ ID NO: 460 - protein, 144 aa, Cannabis sativa
RPGTGPKPVPGRKVTVHCTGYGKNGDLSQKFWSTKDPGQEPFAFTIGQGSVIKGWDEGVLGMQVGEVARLRCTPDYAYGPSGFPAWGINQILLWFSRSKSSRWSEIWTVKSYAEQLIVFLPNVSKLLLCDTNNNIFGGLLDEYL

SEQ ID NO: 461 - DNA, 672 bp, Cannabis sativa
  source 1..672: mol_type = other DNA
atggcagaaatggtgaagttttatggttctaggggaagcccttttagtaacagagtgaatgtagctctgaaactcaaaggtgttgaatacaaatactatgaagaagatctgaagaacaagagtgcttcgctcctcaaatacaacccaattcacaaaaaagtccctacctttgtccacaacgaaaagcccgtagcagagtcacttgtaattcttgaatacatcgatgagacatggaagactcaccccatcttgcctcaacacccttatgaaagagctcaagcccgtttctggagtcgtttcattgaagacaaggtcgtgccaactgcatggaaagctttctgggttaaggaggagcggaaaaaggcattagaagaagtaagtgagtatcttgaaattctagagaaagagttgaaaggtaggtactttggtggagaaagcattggattggtggatataacgggaaacttcatagcccaatggatcccagctgtacaagagcttgtgggagtagagatattgacagaggcaaagtttcccaagctctgtaaatggagccatgattttgccactcatcctatgatcaaggaagtatcaccttctaaagaagaactcattgctctcttcaagtctcgtttgaataccaccaacaaaaaaaatgatctagtttattag

SEQ ID NO: 462 - protein, 223 aa, Cannabis sativa
MAEMVKFYGSRGSPFSNRVNVALKLKGVEYKYYEEDLKNKSASLLKYNPIHKKVPTFVHNEKPVAESLVILEYIDETWKTHPILPQHPYERAQARFWSRFIEDKVVPTAWKAFWVKEERKKALEEVSEYLEILEKELKGRYFGGESIGLVDITGNFIAQWIPAVQELVGVEILTEAKFPKLCKWSHDFATHPMIKEVSPSKEELIALFKSRLNTTNKKNDLVY

SEQ ID NO: 463 - DNA, 555 bp, Cannabis sativa
  source 1..555: mol_type = other DNA
atggcggatttatctagttcgaacaacgatgagcgaaagggccggctttacaacccgtaccatgatctccatgtcccgatgcagaacctctacaatcttccaaccgcaccagaacacctctttatcgaagaagcctcaaagcctcatcgatcatggggtgagaaccttcaatactacaccggttgtggttacttgtccggtgcgatcatcggaggcaccaagggatccatagaaggtctcaaggcagctgaggcaggcgatacgctcaagctccgcgttaaccgtgttcttaattcgggcggctcactcggacggaggtacggaaacactcttggcattcttggattgatcttcgccggtttggaaagtggggtaattcaattaagaggtcaagatgatttgttgaatactgttgtggctggacttgggactggtgcgctttataaagcggcggcgggaccgaggtcggcggccattgctggtgccatcggagggattgcggcggcggctgcagttgcggggaagcaggctgtcaagagatacgttccgatatag

SEQ ID NO: 464 - protein, 184 aa, Cannabis sativa
MADLSSSNNDERKGRLYNPYHDLHVPMQNLYNLPTAPEHLFIEEASKPHRSWGENLQYYTGCGYLSGAIIGGTKGSIEGLKAAEAGDTLKLRVNRVLNSGGSLGRRYGNTLGILGLIFAGLESGVIQLRGQDDLLNTVVAGLGTGALYKAAAGPRSAAIAGAIGGIAAAAAVAGKQAVKRYVPI

SEQ ID NO: 465 - DNA, 1161 bp, Cannabis sativa
  source 1..1161: mol_type = other DNA
atgaagtttttagcccaagcagaggagattgcggataagtacaatgaaatggagctgaaagagaatgagaatccttacagaagggaagataggaagctatggaaatcggttcctcatgttcttgggcttgatggccggcctatgcctaggaaagccataaagaccagagaggaatctgatgataagttctgggattttgccaggcagttcttctttgggctttggggcttcagacagaggccttacccgcctgggcgccccattgatgttgctcaagctattgggtacaagagactcgagaagcgttactatgattttatcatgaggagtggtggattttactataaggatcggttgggccgtactagggggccttgtgaattgataaccctcaagactgcatggggtggtggcattattgataaggatactttcatttgggctgaagacatggatgaatgggcgccaattcacatggtttacggcatggagcgtgcgattgccacttgggaagttagatttggagctgctgctactgctttccttcacaaattacaaaaaggtatccctccttgggttcctctaaagggtcgtgagcctaagacttataagcaggctcaagaagaagcttatgaaagcaaaagacgtgatttagcggtactgaaagctaatgatgggatttggcctggtgttagaattccaagtcatgttctatttctctgggctggcggttctgaactcacgacaattcttgaagccgatcacatgccaaacaaatacattccaaaagatgttaggcgtcaattggctggaattatccctggtctaaggccatgggaggttctaagtgtagaacaagcaatggatcaaataacatatggcggggaatggtaccgtgaacctcttggttcatacaccacaggccctccatacattcaggagtggaacaaggaagtcatgagattgatagatgtttatacattgcttagcacccatgtttacgaaaaactgaaacagacaattcctggtttcgacactgtactcgaaagtgtccttgctgatgcttcagtgagggaaaagaaaaggaaggagaagaggatgagagagaagaaagaacaggaagagaaagaattgattcgccgacttgctcgtggtcggtag

SEQ ID NO: 466 - protein, 386 aa, Cannabis sativa
MKFLAQAEEIADKYNEMELKENENPYRREDRKLWKSVPHVLGLDGRPMPRKAIKTREESDDKFWDFARQFFFGLWGFRQRPYPPGRPIDVAQAIGYKRLEKRYYDFIMRSGGFYYKDRLGRTRGPCELITLKTAWGGGIIDKDTFIWAEDMDEWAPIHMVYGMERAIATWEVRFGAAATAFLHKLQKGIPPWVPLKGREPKTYKQAQEEAYESKRRDLAVLKANDGIWPGVRIPSHVLFLWAGGSELTTILEADHMPNKYIPKDVRRQLAGIIPGLRPWEVLSVEQAMDQITYGGEWYREPLGSYTTGPPYIQEWNKEVMRLIDVYTLLSTHVYEKLKQTIPGFDTVLESVLADASVREKKRKEKRMREKKEQEEKELIRRLARGR

SEQ ID NO: 467 - DNA, 789 bp, Cannabis sativa
  source 1..789: mol_type = other DNA
ttggcccaagagaggattgctgctcttgctgaaattgagaaagcgaaactggaatcacaaacgctggagtcattattggtttctggagatgataattttgtaattggcaagaatggttccaaggtacctatttctgaactagttggaaagaatgttgttctctatttctcagctcactggtgtccaccatgccgtcgattcacacctaagcttatcaagacatacaacgaaatcaaagagaaagacgatgcatttgaagtaatcttcatctctagcgactcagaccagtcctccttcgaggaatacttctccatcatgccttggttagcccttccatttggagattcaaggaagaaactcctgaaccgaacattcaaagtcgagggcattccaacagcggtagccattggtccatctggtcgaacagtgacaaaggatgttgtggaactcgtaatggatcatggggcaaaggcttatccatttacagaagagcagctgaaacatttggaggaacaagaagaagaaattgcaaaggggtggcctgagaaattgaaacatcatcttcatcctgaacatgagcttaagcgtgctcatcgcccaggatactgctgcgatgcatgtgaggaaatgggaaagggatggtctttctactgtgaggaatgtgactttgatcttcacccaaaatgtgctttggagaaggttgaagaagagggtaaaaatggtaatgatgctgctcagaatggaaaggaggggtttgtttgtgaaggtgatgtgtgccgccgggtttga

SEQ ID NO: 468 - protein, 262 aa, Cannabis sativa
LAQERIAALAEIEKAKLESQTLESLLVSGDDNFVIGKNGSKVPISELVGKNVVLYFSAHWCPPCRRFTPKLIKTYNEIKEKDDAFEVIFISSDSDQSSFEEYFSIMPWLALPFGDSRKKLLNRTFKVEGIPTAVAIGPSGRTVTKDVVELVMDHGAKAYPFTEEQLKHLEEQEEEIAKGWPEKLKHHLHPEHELKRAHRPGYCCDACEEMGKGWSFYCEECDFDLHPKCALEKVEEEGKNGNDAAQNGKEGFVCEGDVCRRV

SEQ ID NO: 469 - DNA, 747 bp, Cannabis sativa
  source 1..747: mol_type = other DNA
atgaccttaccctccgccgctttgttctcccaaaaccctgtcgtttctgatgtcgttgccactgccttgtccggctctgtcgccttctctttgctccaattgttgaaagagaccgccacacgtggtattttcgaccagaaacttaacaggaagcttgtgcatataagcattggcatagctttcatgctctgctggccacttttcagtcctgggtctcgtggagcatggttagcagcaactattccaggattgaacattatacggatgcttatcttaggacttggattatggaaagatgaggcaactgtgaaatcaatgagcagattcggagactacagggagcttctcaagggaccactgtactatgtcatagcaattgctcttgtttgtataatctattggcgaacttcccctatttcgattgttgcaatatgcaacctatgtgctggagatggtgtagcagacattataggaaggcggtttggtagtattaaaattccatacaacagaaacaaatctattgctggaagcattgcaatggtatctgctagtttcttagcgtctgtggggtttatgtactatttctccttatttgggtatatggaaagaagttgggaaatggtcttggggttttggattgttgctcttgcatcagcactggtggaatcactcccaataagcaccgaccttgatgataacctcacagttcccgcaacatccattttggtgggcattcttgtgttctaa

SEQ ID NO: 470 - protein, 248 aa, Cannabis sativa
MTLPSAALFSQNPVVSDVVATALSGSVAFSLLQLLKETATRGIFDQKLNRKLVHISIGIAFMLCWPLFSPGSRGAWLAATIPGLNIIRMLILGLGLWKDEATVKSMSRFGDYRELLKGPLYYVIAIALVCIIYWRTSPISIVAICNLCAGDGVADIIGRRFGSIKIPYNRNKSIAGSIAMVSASFLASVGFMYYFSLFGYMERSWEMVLGFWIVALASALVESLPISTDLDDNLTVPATSILVGILVF

SEQ ID NO: 471 - DNA, 741 bp, Cannabis sativa
  source 1..741: mol_type = other DNA
atggcgcctagagataagtcgtccaccgccaacaccgcctccgccgccgttaaacttaacggtaacgtcaaggaggtccatttcaggggagtcaggaagaggccatggggcaggtacgccgccgagatcagggacccaggcaagaagagccgggtctggctcggcaccttcgacacagctgaggaagccgcccgagcttatgacaccgccgcaagagagtttcgtggtgccaaggctaagaccaacttccctctcccttctgattgtatcaacgagaataatctcaacgttttgaatgttaacagaaagaatcttaaccagagcccgagccagagtagtacggtcgagtcctcgagccgggagccggctctgatggttgaatcctcgcctttagatcttaatcttggccacgggtttggctcggccaattcctccgccgtccggtttccgtttcaaccgctccctgctgtcggagccgtttatggtatgccgacgactaaccaacaagtcctgtatttcgatccgtacgttcatgctgggttggtgaatggtcatcatcatcagcagcttcaacggctgagatttgatcatcaggcggcccgtaactaccatgccgcttctttcgccggtggtgctcagagtgattcagattcatcctccgtcgtcgatctgaaccataacgatcaacctcgtagaatgttcgatctcgatctcaactttcctccgccagaagttgcgtaa

SEQ ID NO: 472 - protein, 246 aa, Cannabis sativa
MAPRDKSSTANTASAAVKLNGNVKEVHFRGVRKRPWGRYAAEIRDPGKKSRVWLGTFDTAEEAARAYDTAAREFRGAKAKTNFPLPSDCINENNLNVLNVNRKNLNQSPSQSSTVESSSREPALMVESSPLDLNLGHGFGSANSSAVRFPFQPLPAVGAVYGMPTTNQQVLYFDPYVHAGLVNGHHHQQLQRLRFDHQAARNYHAASFAGGAQSDSDSSSVVDLNHNDQPRRMFDLDLNFPPPEVA

SEQ ID NO: 473 - DNA, 909 bp, Cannabis sativa
  source 1..909: mol_type = other DNA
atgtatggaagaggagccatggcgaataataacatgcatagtggtggtggtggtcatggggatgaagaagatgacgaggtgaccggtgctgaagagtccattgataaccctcagctccggtatgaagatggtggatgtgttggagccatgaacggcgttcaagatgttccttcaaatgttctctacgtgcccaactctgaatatcctcccgttgccgaaaatggaggctctgatcagcttactctatcgtttcagggcgaggtttacgttttcgatgctgtttcgcctgataaggtgcaggcagtattgttacttctgggtggatatgaaatcccttctggtattcctgccatgggtacagcccctcttaatcaacgggctgtgaatcaatttgtttcaaagccggttcaaccacatagagctgcttctttaaatcggttccgggagaagaggaaagaacgttgttttgataaaaaaatccgttacactgtccgcaaggaagttgctatgaggatgcagcgtaagaagggtcagtttacatcatctaaggctagttccgatgaggcgggctcagctgtaattgtttggaatggcgcacagggctctggccaagatgagagcatgcaggaaacttcgtgtacaaactgcggaactagttcgaagtccacaccaatgatgcgtcgtgggcctgctggtgcaaggactttgtgtaacgcatgtgggcttaagtgggctaataagggagtcttaagagatcttaaggttccgaacggtggtgtccaggctgcttcaatgaaggaaaccgggcagagcgatggtgatgctaatgattccgaagctgtaaacacaacaaccaacatcgtctcttccaacgggcaccacgaccatatgactactgaaacaggatga

SEQ ID NO: 474 - protein, 311 aa, Cannabis sativa
MSESNQEASMYGRGAMANNNMHSGGGGHGDEEDDEVTGAEESIDNPQLRYEDGGCVGAMNGVQDVPSNVLYVPNSEYPPVAENGGSDQLTLSFQGEVYVFDAVSPDKVQAVLLLLGGYEIPSGIPAMGTAPLNQRAVNQFVSKPVQPHRAASLNRFREKRKERCFDKKIRYTVRKEVAMRMQRKKGQFTSSKASSDEAGSAVIVWNGAQGSGQDESMQETSCTNCGTSSKSTPMMRRGPAGARTLCNACGLKWANKGVLRDLKVPNGGVQAASMKETGQSDGDANDSEAVNTTTNIVSSNGHHDHMTTETG

SEQ ID NO: 475 - DNA, 345 bp, Cannabis sativa
  source 1..345: mol_type = other DNA
tggggggacaactttgtacaaaaaagttggcggccgcacaactttgtacaagaaagttgggttttggaacttgctgccggagtcatggacgagctttccaaccctaccggtcccagaaaggagttcgtcaacaaccattgccgcgagttcatgcaaatgatcaaggatatccaagtggcattgagagaagaaatcaagagtgcatgtgattatcgtccatttgagaagtgtgattacagttcgagaatagccaatgagatttgttgcaagaagctggattatgtggtgttgcagttggatcaaatgaaacaaaccattgatgattatcgtagtagtacactgtga

SEQ ID NO: 476 - protein, 114 aa, Cannabis sativa
WGDNFVQKSWRPHNFVQESWVLELAAGVMDELSNPTGPRKEFVNNHCREFMQMIKDIQVALREEIKSACDYRPFEKCDYSSRIANEICCKKLDYVVLQLDQMKQTIDDYRSSTL

SEQ ID NO: 477 - DNA, 819 bp, Cannabis sativa
  source 1..819: mol_type = other DNA
atgggaaggtctccttgttgtgagaaagctcacacaaacaaaggagcgtggactaaagaggaagatgatcgacttattgcatacataagggctcacggcgagggttgctggcgttcactacctaaagccgccggtctcctaaggtgtggcaagagttgtagacttcgttggattaattaccttagacctgacctcaaacgtggaaactttacagaagaagaagacgaactcatcatcaagctccatagtctccttggaaacaaatggtctttaatagctggaagactacctggaagaacagacaatgagataaagaactactggaacacccacataagaagaaagcttttgaacagaggaattgaccctgcaacacaccgaccactgaacgagtcaggtcaagagacgactaatacttcgaccaccacaagcgccaccgcagccaccaacaccactgccggtaccaacacaaccaccacaatatcatttggtgcttctgttgttaaagaagaagagaaaacgacaagtgttctgttgttaaacccagttcatcaagaacagtgtcctgacctgaaccttgagctaagaattagccctccttatccgccgcatcaacaacaacaacgccagcagcctgaccaattgaagagcggcggtgcttctctctgcttcgcctgtagtttgggtttgcagaacagtaaagagtgttgctgtacaatttcaagtatggatagcaataacccaagcaccagtgttggttatgatttcttgggtttgaaatctggtgttttggattacagaagcttggaaatgaaatag

SEQ ID NO: 478 - protein, 272 aa, Cannabis sativa
MGRSPCCEKAHTNKGAWTKEEDDRLIAYIRAHGEGCWRSLPKAAGLLRCGKSCRLRWINYLRPDLKRGNFTEEEDELIIKLHSLLGNKWSLIAGRLPGRTDNEIKNYWNTHIRRKLLNRGIDPATHRPLNESGQETTNTSTTTSATAATNTTAGTNTTTTISFGASVVKEEEKTTSVLLLNPVHQEQCPDLNLELRISPPYPPHQQQQRQQPDQLKSGGASLCFACSLGLQNSKECCCTISSMDSNNPSTSVGYDFLGLKSGVLDYRSLEMK

SEQ ID NO: 479 - DNA, 819 bp, Cannabis sativa; identical to SEQ ID NO: 477
  source 1..819: mol_type = other DNA

SEQ ID NO: 480 - protein, 272 aa, Cannabis sativa; identical to SEQ ID NO: 478

SEQ ID NO: 481 - DNA, 720 bp, Cannabis sativa
  source 1..720: mol_type = other DNA
atggatatggcttctaaccctcgaactgtggaggaaatcttcaaagattacagtgctcgtagagttggtctcgttcgtgctctcactaacgatgttgatgaattttacgcgctttgtgatccagagaaggagaatttgtgtctatatggacatccaaatgaaagctgggatgtggctctgcctgcagaagaagttccaccagagcttcctgagccagctcttggtatcaactttgctagagacggcatgaaccgcaaggactggctttcactagtagctgttcatagtgactcttggctgctttctgtggctttttactttggagctcgtcttaaccgcaatgagaggaagcgtctgttcagtatgatcaacgatctacctacagtctttgaagttgtcacagaaaggaagcccatcaaagaaaagcctagcattgatagcggaagcaaatctcgtggcagcacaaagagatccaatgatggtcaagtgaaaagcaatccgaagctagcagatgaaagtttcgaggaggatgaagacgagcacagcgaaaccctctgcggtagctgtggtggaaactacaacgcagacgagttttggatcggctgcgacatatgcgagaagtggttccacgggaaatgtgtgaagattacacctgctaaggccgagagcatcaagcaatacaaatgcccatcttgcagcatgaaaagaggcaggcagtag

SEQ ID NO: 482 - protein, 239 aa, Cannabis sativa
MDMASNPRTVEEIFKDYSARRVGLVRALTNDVDEFYALCDPEKENLCLYGHPNESWDVALPAEEVPPELPEPALGINFARDGMNRKDWLSLVAVHSDSWLLSVAFYFGARLNRNERKRLFSMINDLPTVFEVVTERKPIKEKPSIDSGSKSRGSTKRSNDGQVKSNPKLADESFEEDEDEHSETLCGSCGGNYNADEFWIGCDICEKWFHGKCVKITPAKAESIKQYKCPSCSMKRGRQ

SEQ ID NO: 483 - DNA, 1074 bp, Cannabis sativa
  source 1..1074: mol_type = other DNA
atggcggtggagctaatgaggttccccaaaatggaagatcagaaagagattcaagaagctgcgactcagggcttgaagagtatggagcacctgattcgatttctctctcacaatcaacagactactcaaatggactgctctgaccttacggatgtaactgtctctaagttcaggaaggtgatctctcttctcaaccggactggtcatgccaggttcagacgtgcgccggctcattcgtcatcttcttcttcgcctccctcgtcttcttcgtcctccggttcgctctcgcttcatcaacctcaggtacctaatacactggccttagctcccgcgtcgattgcttctcatccgacttacgtttctcccgcgccgatcgctcctccggtaaccgttcaggcggcgagtttcgcacaatctcagaagcagagcatgactctggattttactaaacctaacaataatatacttagctcgaacattcacaaggcctccgcggaggttgagttcacaaaagacagcttcagcgtttcttccagctcgtcgttcatgtcgtcggcgatcaccggagacggaagcgtctcgaacggaaagcaagggtcgatgttcttgactcctgcaccgactgtctccgtcggtaaaccacccctctcaacggcgccgtttaagaagaggtgccatgatcacgaacactccggcgatttctcaggcaaaatatcgggatctgcatccggatccggaaagtgccattgctcaaagagaaggaaaaatcgtgtgaagaagacaattagagtcccggcgattagttccaaagtcgctgatattccacaagacgagtattcgtggaggaagtatggacaaaaaccgataaaaggctctccttatccaagaggttactacaaatgtagtaccatgagaggttgcccggcgaggaaacacgtggagagggctcccgatgatccggctatgcttattgtaacctacgagggagagcaccgtcatcagcagccggacaccatggcgccaatgggtggtggtggaggagtctctttggtattcgactctaatcgagtgtga

SEQ ID NO: 484 - protein, 357 aa, Cannabis sativa
MAVELMRFPKMEDQKEIQEAATQGLKSMEHLIRFLSHNQQTTQMDCSDLTDVTVSKFRKVISLLNRTGHARFRRAPAHSSSSSSPPSSSSSSGSLSLHQPQVPNTLALAPASIASHPTYVSPAPIAPPVTVQAASFAQSQKQSMTLDFTKPNNNILSSNIHKASAEVEFTKDSFSVSSSSSFMSSAITGDGSVSNGKQGSMFLTPAPTVSVGKPPLSTAPFKKRCHDHEHSGDFSGKISGSASGSGKCHCSKRRKNRVKKTIRVPAISSKVADIPQDEYSWRKYGQKPIKGSPYPRGYYKCSTMRGCPARKHVERAPDDPAMLIVTYEGEHRHQQPDTMAPMGGGGGVSLVFDSNRV

SEQ ID NO: 485 - DNA, 561 bp, Cannabis sativa
  source 1..561: mol_type = other DNA
ttggccaacttttttgtacaaagttgtccccctcgtttcccatactggcacctcttctttttttcccgtttcccctttgtaactcggttaatgatggacccgatggatatcgtcgggaagtccaaagaggacgcttcgcttcctaaagccactatgacaaaaattattaaggagatgttgcctccggacgttcgtgttgcaagagatgctcaagatcttttgattgagtgttgtgtagagtttataaaccttatttcatcagaatctaatgaagtttgtggcagagaagaaaaaagaacaattgcaccagaacatgttctcaaagctttagaggtgcttggctttggtgagtatatagaagaagtttatgcagcatatgaacaacacaagcttgagaccatgcaggacttaagaagtggcaaatggagcaacggagccgaaatgacagaggaagaagcattggctgagcaacagcggatgtttgctgaggctcgtgcaagaatgaatggtggcactgctactccaaaacaaccagaacctgaccaaagtttagagagctaa

SEQ ID NO: 486 - protein, 186 aa, Cannabis sativa
LANFFVQSCPPRFPYWHLFFFSRFPFVTRLMMDPMDIVGKSKEDASLPKATMTKIIKEMLPPDVRVARDAQDLLIECCVEFINLISSESNEVCGREEKRTIAPEHVLKALEVLGFGEYIEEVYAAYEQHKLETMQDLRSGKWSNGAEMTEEEALAEQQRMFAEARARMNGGTATPKQPEPDQSLES

SEQ ID NO: 487 - DNA, 387 bp, Cannabis sativa
  source 1..387: mol_type = other DNA
ggaaaaattatcatccctgatcagaaaatctacaaaaacccttctctcaagatccgattcatatttaccatgactggaaaggcgaagcaaagaaacacacaacgaaggaaatcgccgctaagatcgatgcagcctacaaccaaccgcggcggcggcaaggcagggctagccgatcgctccggcatcgaaaagggtggtcacgctaagtttgagtgcccactctgtaagacgaccgtacctgatttgaaatcgatgcagattcatcacgaggctcgccacccgaagatcccattcgaagaggataagcttgtgaatcgtcactccaccgttcccgccgtcgagagctccaagccccgccctggtattcgtggcagtctcaagaagtaa

SEQ ID NO: 488 - protein, 128 aa, Cannabis sativa
GKIIIPDQKIYKNPSLKIRFIFTMTGKAKQRNTQRRKSPLRSMQPTTNRGGGKAGLADRSGIEKGGHAKFECPLCKTTVPDLKSMQIHHEARHPKIPFEEDKLVNRHSTVPAVESSKPRPGIRGSLKK

SEQ ID NO: 489 - DNA, 393 bp, Cannabis sativa
  source 1..393: mol_type = other DNA
gggttcttggtcttccttgctcttttgacctccgataaaaaatcaacaaaacccttctccttgatccgatcatatcaccatgacagggaaggcgaagccaagaagcacacagcgaaggagattgctgccaagattgatgcggcaaccaccaaccgcggcggcggaaaggctggcatggctgatcgcaccggcatcgagaaaggtggtcacgcaaaatacgagtgtccgcactgcaagacgaccgcacctgatttgaaatcgatgcagattcatcacgaggctcgacacccgaagcttccatacgaagaggacaagcttataaatcgtcacgcggcccttgccgtcgagacttctaagggtcgccctggtattcgtggtagcctcaagaagtaa

SEQ ID NO: 490 - protein, 130 aa, Cannabis sativa
GFLVFLALLTSDKKSTKPFSLIRSYHHDREGEAKKHTAKEIAAKIDAATTNRGGGKAGMADRTGIEKGGHAKYECPHCKTTAPDLKSMQIHHEARHPKLPYEEDKLINRHAALAVETSKGRPGIRGSLKK

SEQ ID NO: 491 - DNA, 855 bp, Cannabis sativa
  source 1..855: mol_type = other DNA
atgaactctaacccttccaataatccacaatcaacgacgcccgctgatggctcagccaagaaggtcagaaagccctatactataaccaagtccagagaaagctggacagaggaagaacacgacaaattcctcgaagctcttcaactgtttgatcgtgactggaaaaaaatcgaagattttgtgggttccaagacagtcattcagattaggagtcacgcccagaaatattttcagaaagtgcagaagaacgggactcttgctcatgttcctccaccacgcccgaagcgcaaagctgctcacccttaccctcaaaaagcatccaaaaatgttttggttccccttcaagcttccttagcttatccttcttcaatgaatcctatggtgcctgcgtattcaccatgggatgaagtttctttgttgataagtcccacatcaagtaaaataacatcccaggaagaatttggtaatcttcaagcaactgaagctgatattgcatcaaagggggctgcaaggaatagtaacaacagtatttgtggcattgcaagctcaagtagatcaatactaggttctgagataccaaagcatgggaaacaaggttctgtgctccatggacccgattttgctgaagtttatagctttattgggagtgtctttgatccagacactaatgaccatgtgcagaaactcaaggagatggatcccattaattttgagacagttttaatgttaatgagaaacctcaccttcaacttgtgtaacccagattttgagcctattagaaaggttatgtcatcgtatgacgtcaacgcaaaatcgatgggaattgctaagcagaactataatctgccagtttga

SEQ ID NO: 492 - protein, 284 aa, Cannabis sativa
MNSNPSNNPQSTTPADGSAKKVRKPYTITKSRESWTEEEHDKFLEALQLFDRDWKKIEDFVGSKTVIQIRSHAQKYFQKVQKNGTLAHVPPPRPKRKAAHPYPQKASKNVLVPLQASLAYPSSMNPMVPAYSPWDEVSLLISPTSSKITSQEEFGNLQATEADIASKGAARNSNNSICGIASSSRSILGSEIPKHGKQGSVLHGPDFAEVYSFIGSVFDPDTNDHVQKLKEMDPINFETVLMLMRNLTFNLCNPDFEPIRKVMSSYDVNAKSMGIAKQNYNLPV

SEQ ID NO: 493 - DNA, 750 bp, Cannabis sativa
  source 1..750: mol_type = other DNA
atggattggaatagcaacttaagacttgtttcaagacctgaatgttcttatggcttcctttacaactacaactatgatcagtaccagggtatggatatgaaacatcatcaagcagctttaatggcggagacaacacaagggttagggctacttggagttccatcaatggagaagatgaatggtgggggtagtagtggtaacactggtaacagtagttatgggatgatgatgagtaatcaggacaagaagaagagactaacgacagatcagttggagtctttggagagaagcttccaagaggagattaagcttgaccctgataggaagatgaagctgtctagagatttaggccttcagcctcgccaaatagctgtttggtttcagaataggcgtgctcgttggaaagctaagcagctcgaacgcttgtatgatgctcttaaacaagaacttgatgtcatgtctaaagagaaacagaagctccaagaagaggtaacgaaactgaaggggatgctaagagagcaaggaggtgggtggaagcaagtgtccaccacaggtggttacactgaaatttccggtgaagagacggtggaaagcacgtcggcgggaggtattcgaagcagtagcaagtcactaggaacgaaccccttgggtggtggtggcagcggcggtggtgagtgcagtcatcacattctcaatgtggaggaatacaaccctgtatcaccaccttactggctcccttcttatccttaa

SEQ ID NO: 494 - protein, 249 aa, Cannabis sativa
MDWNSNLRLVSRPECSYGFLYNYNYDQYQGMDMKHHQAALMAETTQGLGLLGVPSMEKMNGGGSSGNTGNSSYGMMMSNQDKKKRLTTDQLESLERSFQEEIKLDPDRKMKLSRDLGLQPRQIAVWFQNRRARWKAKQLERLYDALKQELDVMSKEKQKLQEEVTKLKGMLREQGGGWKQVSTTGGYTEISGEETVESTSAGGIRSSSKSLGTNPLAGGGGGGGECSHHILNVEEYNPVSPPYWLPSYP

SEQ ID NO: 495 - DNA, 480 bp, Cannabis sativa
  source 1..480: mol_type = other DNA
aattcatccgacagtgaaactcgaagtgaaaaccatgaccggtccaagagtacaacacaaaatgaccaaaacccgaaagatgaaacaacttcaactacttctactcttaaagaagaatccattaaagaaatgaaacagaacatggaggaaaacaggttctattacatgccgccgaggaaacaattgaaaaaatttgggtatctacattacgcgagaaagagggacgaaggctcgttgacttattctgttcatgcagattactacatcttgcttcgtgcttgtgctaaagttgtccaaattgaaactcggtatatgcatttcggggtgttgagtttcgagaaacgactggtttggactgagcacaaaatcaaccactgtttgcatatgactcctccaaatctctcttgtgaattttgtactaattccataagtggagatggcttagaagagtctcttggactttcaaatttgaatatttag

SEQ ID NO: 496 - protein, 159 aa, Cannabis sativa
NSSDSETRSENHDRSKSTTQNDQNPKDETTSTTSTLKEESIKEMKQNMEENRFYYMPPRKQLKKFGYLHYARKRDEGSLTYSVHADYYILLRACAKVVQIETRYMHFGVLSFEKRLVWTEHKINHCLHMTPPNLSCEFCTNSISGDGLEESLGLSNLNI

SEQ ID NO: 497 - DNA, 1359 bp, Cannabis sativa
  source 1..1359: mol_type = other DNA
atgacagttaaagaagtgacaaaattgtgggtttatcaagctcgagataggatacctatagctgaggctcctcctggttcttgggttctcattgaaggagtggatgcttcaatcatgaaaactgctactatttgcagtgagtattatgacgaagatgtatacatctttcgacctttgcaattcaatacccttccggtagtgaaaacggcgaccgagccattgaaccctagtgaattgccaaagatggtcgagggtcttaggaaaatcagcaagagctatcctcttgccattactaaagttgaggaatctggagaacacacaattttgggtacaggcgaactgtacttggattctattatgaaggatctaagagaactctattctgaagttgaagtcaaggtggcagatcctgttgtgtcattctgtgaaacagtggtggagtcttcatctatgaaatgttttgctgaaacacccaacaaaaagaataaaattaccatgattgcagaaccactggagaggggacttgcagaggacatagagaacagtgttgtaagccttgattggcctcggaaagatattggtaacttttttcagacaagatatgaatgggatgtgcttgctgcacggtccatatgggcatttggtcctgataagcaggggcctaatatcctgttggatgatacactttcaactgaagtcgacaagggcttgctaaatgcggtcaaagattccatagttcaaggatttcagtggggtgctcgtgaaggacctctctgcgatgagccaattagaaatgtaaagttcaagattgttgatgcaaaaattgcccccgagtcattgcatcggggatctggtcagatcattcccacagctcgacgtgttgcttattcatcttttctcatggcaaccccccgacttatggaacctgtatattatgtggagattcaaacgccaattgactgtgtctctgcaatttacacggtcttatctcgaaggcgtgggcatgttacatctgatgttcctcaaccaggaaccccatcttatattgtcaaggcatttttacctgtgatagaatcatttggttttgagacggacttaagatatcatacccaaggacaagcattctgcgtatcggtttttgatcactgggctatagttcctggggatccccttgacaagagtattgtcctgaggccacttgaacctgctcctatccagcaccttgctcgtgaatttatggtgaagacaaggcgtcgaaagggaatgagtgaggacgtgagcatcaacaaattctttgacgaggcaatgatggtggagctggctcagcaggcagctgatctacacctgcaaatgatatga

SEQ ID NO: 498 - protein, 452 aa, Cannabis sativa
MTVKEVTKLWVYQARDRIPIAEAPPGSWVLIEGVDASIMKTATICSEYYDEDVYIFRPLQFNTLPVVKTATEPLNPSELPKMVEGLRKISKSYPLAITKVEESGEHTILGTGELYLDSIMKDLRELYSEVEVKVADPVVSFCETVVESSSMKCFAETPNKKNKITMIAEPLERGLAEDIENSVVSLDWPRKDIGNFFQTRYEWDVLAARSIWAFGPDKQGPNILLDDTLSTEVDKGLLNAVKDSIVQGFQWGAREGPLCDEPIRNVKFKIVDAKIAPESLHRGSGQIIPTARRVAYSSFLMATPRLMEPVYYVEIQTPIDCVSAIYTVLSRRRGHVTSDVPQPGTPSYIVKAFLPVIESFGFETDLRYHTQGQAFCVSVFDHWAIVPGDPLDKSIVLRPLEPAPIQHLAREFMVKTRRRKGMSEDVSINKFFDEAMMVELAQQAADLHLQMI

SEQ ID NO: 499 - DNA, 663 bp, Cannabis sativa
  source 1..663: mol_type = other DNA
gttggggggacaactttgtacaaaaaagttggggccgcacaactttgtacaagaaagttgggtttttttttttttttttttttttttttttttctcctttttattatatttagggtttcctttttactcttccttctccaaatgggttccgccgacgtcgagtacagatgtttcgtcggagggctcgcatgggccaccgacagcaagtctctggaggatgctttctctacctttggcgaaatccttgaatcgaagatcattaacgatcgtgagactggaagatcaaggggattcggattcgttactttctctaacgaaaaggcgatgagagatgccatcgagggcatgaacggccaagatcttgatggacgtaatatcaccgttaacgaggcccaatcgcgcggaagcggtggtggcggcggcggtggtggttacagccgcggaggtggaggtggaggctacgggggtggtggtggctacagccgcgggggcggtggtggtggatacggtggtggccgtcgtgagggaggtggatacggaggtgagagtggtggtggatacggaggccgacgtgaaggtggtggctatggagatggtggtggttcccgttactccaacagaggaagtggtggtggcgccgccgaaggcaactggagaagttag

SEQ ID NO: 500 - protein, 220 aa, Cannabis sativa
VGGTTLYKKVGAAQLCTRKLGFFFFFFFFFFLLFIIFRVSFLLFLLQMGSADVEYRCFVGGLAWATDSKSLEDAFSTFGEILESKIINDRETGRSRGFGFVTFSNEKAMRDAIEGMNGQDLDGRNITVNEAQSRGSGGGGGGGGYSRGGGGGGYGGGGGYSRGGGGGGYGGGRREGGGYGGESGGGYGGRREGGGYGDGGGSRYSNRGSGGGAAEGNWRS

SEQ ID NO: 501 - DNA, 852 bp, Cannabis sativa
  source 1..852: mol_type = other DNA
atgtgtattgctgatccgctgccgcagcttccgcctgagaaatcccaacgacccgttgtgattcgatcggtgtgggagtataacctagagtccgagttcaagttgatacgatccgccatcgatgatttcccgcttatctccatggatacggagtttccgggcgtcgtgatccgaccctcggtagatccgacgctacgaacttcccaggccattgcgcactacgaggtcctcaaggccaacgtggatcgcttgaatctgatccagatcggtctcagcctttccgacgacgagggaaaccttcctgatctcggcactaacagcctctacatctgggaattcaacttcaaggacttcgacgtggcgcgtgacgcacacgctcacgattccgtggaattgctgaagaggcagggtattgatttcgagcggaatcgagaatctggtatcgattcgatccggttcgccgaactgatgatgtcctcggggctcgtctgcaacgatttggtgagttgggcgacgttccacagcgcctatgattttggttaccttgtcaaggtgttgactcggcgtcttttgcctgatgagttggctgattttctgaagctcgtgacggtgttcttcgggtctagggtcttcgatgtgaaacatctcatgagatactgcgcgagcctttacggtgggctagaccgggtctgtcaggtcctgaatgtgagtcgggtcattggaaagagccaccaggccgggtctgatagcttgctgaccttacatgccttccagaaaatcagagaggtctatttcaacaagaacgggcttgatcagtacgctggtgttttatatggtttagaaatttcgtga

SEQ ID NO: 502 - protein, 283 aa, Cannabis sativa
MCIADPLPQLPPEKSQRPVVIRSVWEYNLESEFKLIRSAIDDFPLISMDTEFPGVVIRPSVDPTLRTSQAIAHYEVLKANVDRLNLIQIGLSLSDDEGNLPDLGTNSLYIWEFNFKDFDVARDAHAHDSVELLKRQGIDFERNRESGIDSIRFAELMMSSGLVCNDLVSWATFHSAYDFGYLVKVLTRRLLPDELADFLKLVTVFFGSRVFDVKHLMRYCASLYGGLDRVCQVLNVSRVIGKSHQAGSDSLLTLHAFQKIREVYFNKNGLDQYAGVLYGLEIS

SEQ ID NO: 503 - DNA, 825 bp, Cannabis sativa
  source 1..825: mol_type = other DNA
atgtcgattctatcgaaaggtgattctattcacatcagagaagtatggaatgataatcttgacgaagagtttgcattaattcgtgatattgttgatcggttcaattatattgctatggatactgagtttcccggtgttgtacttcgccctgtgggaaatttcaagaacattaatgactataattaccagactctgaaagacaatgttgatatgttgaaattgattcaattgggtctcacattctctgacgaggatgggaaccttccaacttgcggaactgataagttttgcatttggcaatttaattttcgtgagttcaacataagcgaagatgtctttgctagtgattcgattgagttgttgcgtcagtgtgggattgatttcaagaagaacaacgagaagggtattgatgtgaatcgctttggtgagcttttgatgtcatctgggattgtgttaaacgatggtgttaattgggtgacatttcacagcgggtacgattttgggtacttgctcaagctattgacctgtagaagtttacccgattcacaagcagatttctttgatttgatcaatatctacttcccaatggtgtatgacatcaagcatttgatgaagttttgcaacagcctccatggtgggttgaataagcttgctgagttgttggatgtggaaagagttggtgtgtgccatcaagccggttctgatagtttgctcacagcttgcacattcaggaaattgagggaaacatttttcagtggctccactgagaaatatgcaggtgtgctatatggtcttggtgttgagaatggacagagtactaattga

SEQ ID NO: 504 - protein, 274 aa, Cannabis sativa
MSILSKGDSIHIREVWNDNLDEEFALIRDIVDRFNYIAMDTEFPGVVLRPVGNFKNINDYNYQTLKDNVDMLKLIQLGLTFSDEDGNLPTCGTDKFCIWQFNFREFNISEDVFASDSIELLRQCGIDFKKNNEKGIDVNRFGELLMSSGIVLNDGVNWVTFHSGYDFGYLLKLLTCRSLPDSQADFFDLINIYFPMVYDIKHLMKFCNSLHGGLNKLAELLDVERVGVCHQAGSDSLLTACTFRKLRETFFSGSTEKYAGVLYGLGVENGQSTN

SEQ ID NO: 505 - DNA, 834 bp, Cannabis sativa
  source 1..834: mol_type = other DNA
atgtcgctcttgccgaaatccgattcgattcatattcgcgaggtttgggatggaaatctagaggacgagttcgctttaattcgagaaatcgtggatgagtatccttacgttgctatggatacggagtttcccggaatcgttctccgtccagtgggaaatttcaagaacagttacgacttccattaccagactttgaaggacaacgttgatatgttgaagttaattcaattgggcttaacattttccgacgaggatggtaacttacccacttgcggaacagataagtattgcatttggcaattcaatttccgcgaattcaatgtcaatgaagatgttttcgccaacgattcgatcgagcttttgcgccaaagcggtattgatttcatgaagaacaacgaaaatggcattgatgttaagcgatttggggagcttctaatgtcatctgggattgtattgaatgacaatgttcattgggtaacttttcatagtggttatgattttgggtacttgcttaagctcttaacttgccaggatttaccagaaacccaattagggttcttcaacttaatcaacatgtacttcccaacagtttatgatatcaaacacttgatgaagttctgtaacagccttcatggtggattgaacaagctagctgagttattggaagtggaaagagttggcatatgtcatcaggcaggttcggatagtttgcttacttcttgtacattcaggaaacttaaagagaacttctttagtggttctatgacgaaatatgctggtgttttgtatggattaggagttgagaatggacagaatattactcagccttactga

SEQ ID NO: 506 - protein, 277 aa, Cannabis sativa
MSLLPKSDSIHIREVWDGNLEDEFALIREIVDEYPYVAMDTEFPGIVLRPVGNFKNSYDFHYQTLKDNVDMLKLIQLGLTFSDEDGNLPTCGTDKYCIWQFNFREFNVNEDVFANDSIELLRQSGIDFMKNNENGIDVKRFGELLMSSGIVLNDNVHWVTFHSGYDFGYLLKLLTCQDLPETQLGFFNLINMYFPTVYDIKHLMKFCNSLHGGLNKLAELLEVERVGICHQAGSDSLLTSCTFRKLKENFFSGSMTKYAGVLYGLGVENGQNITQPY

SEQ ID NO: 507 - DNA, 438 bp, Cannabis sativa
  source 1..438: mol_type = other DNA
atgcctaaagtcaaaacaaaccgtgttaattacccagaagggtgggaattgattgaacctactcttcgtgaactacaagcgaagatgagagaagctgagaatgatcctcatgatggcaagagaaagtgcgaggctttgtggcctatattcaaaattgctcatcagaagagcaggtacatttttgacctttaccatcggcgaaaggagatatctaaagaattgtttgagttctgccttgaccaaggatatgctgaccgaaatctcattgccaaatggaagaagcctggttatgaacgcctctgctgcttaaggtgcatgcagcctcgagatcataactttgcaactacgtgtgtttgtcgagtgccgaaacacctgagagaggagaaggttatagagtgtgttcattgtggttgtaagggctgcgctagtggagactga

SEQ ID NO: 508 - protein, 145 aa, Cannabis sativa
MPKVKTNRVNYPEGWELIEPTLRELQAKMREAENDPHDGKRKCEALWPIFKIAHQKSRYIFDLYHRRKEISKELFEFCLDQGYADRNLIAKWKKPGYERLCCLRCMQPRDHNFATTCVCRVPKHLREEKVIECVHCGCKGCASGD

SEQ ID NO: 509 - DNA, 930 bp, Cannabis sativa
  source 1..930: mol_type = other DNA
atgagtagccgtgcgagcagaaccctttacgttgggaatctcccaggtgatattcgtgagagggaagtggaagatttgttttacaagtatggacgcatagcccacattgacttgaaggtccctccaaggcctcctggttatgcttttgttgagtttgaggatgccagagatgctgaagatgcaattcgtggtcgtgatggctatgattttgacgggcatcgcttgcgggttgaacttgcgcatggtggacgtggacattcatcatcaactgatcgctatagtggccacagtggtggtggtggtgggggtggaggccgagggggacgaggcgtatcaaggcgctctgagtatcgtgtgatggtatccggattaccttcgtcggcttcatggcaagacctgaaggatcacatgcgtcgtgcaggggatgtttgtttctctcaagtgtttcgtggtagtagtggcacaacaggaatcgtggactacactaactatgaagacatgaagtatgcaataaaaaagcttgatgattctgagttccggaatgcattttctcggtcatatgttcgtgttagggaatatgattcaaagagagactcatctaggagccctagccctggtcgtggtcgttctcattcaaaggggaggagtggcagtcgcagtcggagccgtagttacagtaggagcagaagcaggagcaaatctccaaaagccaaagcttcacgtcgctcaccggctaaatctggatcaagatctaggtctcgttcacgctctcgctccttgtctggatctcggtcaagatctagatctccattaccgttgaaacgtacaagtgaaagcccaaaaaagcgcagcgtcagcagaagtcccagcagaagcagaagtaggagccgtagcaggagcaggagccctagcaagagcttatcccggtga

SEQ ID NO: 510 - protein, 309 aa, Cannabis sativa
MSSRASRTLYVGNLPGDIREREVEDLFYKYGRIAHIDLKVPPRPPGYAFVEFEDARDAEDAIRGRDGYDFDGHRLRVELAHGGRGHSSSTDRYSGHSGGGGGGGGRGGRGVSRRSEYRVMVSGLPSSASWQDLKDHMRRAGDVCFSQVFRGSSGTTGIVDYTNYEDMKYAIKKLDDSEFRNAFSRSYVRVREYDSKRDSSRSPSPGRGRSHSKGRSGSRSRSRSYSRSRSRSKSPKAKASRRSPAKSGSRSRSRSRSRSLSGSRSRSRSPLPLKRTSESPKKRSVSRSPSRSRSRSRSRSRSPSKSLSR

SEQ ID NO: 511 - DNA, 2343 bp, Cannabis sativa
  source 1..2343: mol_type = other DNA
atgggtttcttggacgaaaatcttcctgctcaacctcccttacctgaaaccctagaccaatccattgaaaagaccaataccgattccgtcgccgatgcggacaataccgctactgccgatgctgatgctgctcctactgagcctgctccggccgccgtaaagcctttaccgccgactctgcttttagagaatggcctcgcgcacagtggtacaggtactggtaatggtactgatagggattgctctggtggtgaggaagaaacgacgagtaagcgaaaacgtcggagtagatgggacccgcagcctgagattgataaccagacggctgctgctgccggttccggtgccggtggtgattcaggtagcggaccgcgaaagaggaagtctcgttgggctgatgatgatccgaagccggtgattcagttgcctgattttatgggaggtattgaatttgatcctgaaattcaagctttgaatagtagacttcttgagattagtagaatgttgtcttctggtttgcctttagatgatcgtcctgaaggggctaggtctccttcacctgaacctatatatgataatatgggaattaggattaatactagagagtatcgtgctcgtgagagattgaataaggagagacaggaaattatagctcagattattaagcgaaacccggcttttaaaccaccggcagattataggccacccaagcttcagaagaagctttatattcccatgaaagaatacccgggttataattttattgggctcattattggccctaggggtaatacccagaagagaatggagaaagaaaccggtgcgaaaatagtaattcgcggtaaaggttcggtgaaagagggtaggttgcagcagaagagagatttaaaacctgatccgtctgagaatgaggatttgcatgttttggtggaggcagacacacaagatgcgcttgatgctgcggcggggatggttgagaagctcttgcagcctgtagatgaggttttgaatgagcataagaggcagcagcttagggaacttgcagccttgaatggaacaataagggatgaagagtattgtagattgtgtggtgagccaggtcatcgccaatatgcttgtccttcgcgcacttcaacttttaagagtgatgttctttgtaaaatttgtggtgatggtggtcatccgactattgattgtccggtgaagggtacaaccgggaagaagatggatgatgagtaccagaacttcttggcagagttaggtgggacggttcccgagtctgccatcaagcaaactgctaccttagcaattggacctggtagtacaggaagtaatcctccgtgggctaacaataatagcagtgctgttagtggtgctattactgtttcttcgcatccaggtttggggtcatcacctgtgatgaagcctaaggaatatgatgatactaatttgtatattggatacttgccacctactcttgatgatgattctttaatccggttgttttcaccttttggtgatattgttatggcaaaggttataaaggaccgaattaccggattgagcaagggttatggctttgtgaaatactctgatgtacaaatggccaataccgcgattactagcatgaatggttatcgtctagagggccgaactattgctgttagagttgctggtaagcctccacagcctactgtgcctcctggaccaccggcatcagcaccatctgcatttcctgtttctagtcagcctatgggtgcttatccatcccagcaattcgcaccaggtggtcccctgggaaatgcaccacctcctagctattctggtactccagttccatggggaccccctgttccatctccatatgctggttacccgccacctcctcctggttcaaatatgtatcctcctatgcatggtcaacctatggccccttatggtgttcagtatcctccatcaatgcaggcaggcccccctggtgccccgccacagccgcctactacaagtgaagctcaacagagttaccctccaggtgtgcaatccgaaaatggtgcttctcaatctttaccaagcaatatgtatgggaactctgtagcaacaatgccacccaatggtcaacctgcatacccccctccagcttcttatggttattcatcttattacaccagcactcctccccctcctcccgcacccttacccggttctaatacagaccaatctcagagcattggaaatgttccttgggccacaaattcctctgttccacctcctcctcctccagcagagaagacagctttcggtgcagatgcggagtatgagaagtttatggcagagatgaaatga

SEQ ID NO: 512 - protein, 780 aa, Cannabis sativa
MGFLDENLPAQPPLPETLDQSIEKTNTDSVADADNTATADADAAPTEPAPAAVKPLPPTLLLENGLAHSGTGTGNGTDRDCSGGEEETTSKRKRRSRWDPQPEIDNQTAAAAGSGAGGDSGSGPRKRKSRWADDDPKPVIQLPDFMGGIEFDPEIQALNSRLLEISRMLSSGLPLDDRPEGARSPSPEPIYDNMGIRINTREYRARERLNKERQEIIAQIIKRNPAFKPPADYRPPKLQKKLYIPMKEYPGYNFIGLIIGPRGNTQKRMEKETGAKIVIRGKGSVKEGRLQQKRDLKPDPSENEDLHVLVEADTQDALDAAAGMVEKLLQPVDEVLNEHKRQQLRELAALNGTIRDEEYCRLCGEPGHRQYACPSRTSTFKSDVLCKICGDGGHPTIDCPVKGTTGKKMDDEYQNFLAELGGTVPESAIKQTATLAIGPGSTGSNPPWANNNSSAVSGAITVSSHPGLGSSPVMKPKEYDDTNLYIGYLPPTLDDDSLIRLFSPFGDIVMAKVIKDRITGLSKGYGFVKYSDVQMANTAITSMNGYRLEGRTIAVRVAGKPPQPTVPPGPPASAPSAFPVSSQPMGAYPSQQFAPGGPLGNAPPPSYSGTPVPWGPPVPSPYAGYPPPPPGSNMYPPMHGQPMAPYGVQYPPSMQAGPPGAPPQPPTTSEAQQSYPPGVQSENGASQSLPSNMYGNSVATMPPNGQPAYPPPASYGYSSYYTSTPPPPPAPLPGSNTDQSQSIGNVPWATNSSVPPPPPPAEKTAFGADAEYEKFMAEMK

SEQ ID NO: 513 - DNA, 483 bp, Cannabis sativa
  source 1..483: mol_type = other DNA
atgcctcggtattactgtgactattgcgacacttatctaacccatgattcgccatctgttagaaaacagcataatgctggctacaagcacaaggcaaatgtacgaacctactatcagcagttcgatgagcaacaaacccaaagtttgattgaccaaaggattaagaacaccttgaccaagctggagcttatcatcagtttggtgcggcttataatcaacatctacttgctcagaggcctcgtcttcctgttctaccaaacacctatgatgccacacatgcctggaggtggaccaccaatgctacccgggatgattagacctcctgttttgcctagaccaggtcctggtgctccaggaggctatggtcttcatccaaacatgccaccaatgctggctcctcccggtgctccacctggggctcctcctggtgctccatccatgcctggccaattaaaattctctcccgaggcccccaaccattaa

SEQ ID NO: 514 - protein, 160 aa, Cannabis sativa
MPRYYCDYCDTYLTHDSPSVRKQHNAGYKHKANVRTYYQQFDEQQTQSLIDQRIKNTLTKLELIISLVRLIINIYLLRGLVFLFYQTPMMPHMPGGGPPMLPGMIRPPVLPRPGPGAPGGYGLHPNMPPMLAPPGAPPGAPPGAPSMPGQLKFSPEAPNH

SEQ ID NO: 515 - DNA, 897 bp, Cannabis sativa
  source 1..897: mol_type = other DNA
atgactactcctttggatatgtcgcttgatgacatgataaagagcagcaagggtaatcgtgagagaattagagaaagaggtagagcccgtcgaggacgtggttcgcgtgggtcctttaatggtggtggtggtgctgctgctggaagaatgaatgggccagttcgtaggggccctctcagggtgaatgctcagccatctgcttacaccattgccaagtctatccgcagaaccaaggccttcccatggcagcatgatttgtttgaagatagtatcagagctgctggaataacaggcattgagattggcacaaaactatatgtctccaacttggacaatggagtgaccaatgaggatataagggaacttttctctgaaattggggacttaaaaagatatgcagtgcattatgacaaaactggtcgcccaagtggttcagctgaagtggtgtataccagaagaagtgatgcctttgcagcattgaagcgttacaataatgtactgctggatgggaagccaatgaagcttgagattgtgggtctcaattcagaagttcctttagctcgtgtgaatgttgctggattaaatggaaggaagaagaggactgtggttatgacgtcaggacctggtcgagctggtggccctcctatggttaatcgtggtcctggtgtttctggattcgccagtcaacgaggtcgtagtggacttaggaatggtcgtggccgtggtggtggtagtaatattggaggacgtggacgtggaggtggccgtgggcgtgggcgtggtggtagcattggtggtggtggcggcgctcgtggaaagaagaaaccagttgagaagtctgctgatgaccttgacaaagaactggatagttaccatgctgaagctatgcaaacatga

SEQ ID NO: 516 - protein, 298 aa, Cannabis sativa
MTTPLDMSLDDMIKSSKGNRERIRERGRARRGRGSRGSFNGGGGAAAGRMNGPVRRGPLRVNAQPSAYTIAKSIRRTKAFPWQHDLFEDSIRAAGITGIEIGTKLYVSNLDNGVTNEDIRELFSEIGDLKRYAVHYDKTGRPSGSAEVVYTRRSDAFAALKRYNNVLLDGKPMKLEIVGLNSEVPLARVNVAGLNGRKKRTVVMTSGPGRAGGPPMVNRGPGVSGFASQRGRSGLRNGRGRGGGSNIGGRGRGGGRGRGRGGSIGGGGGARGKKKPVEKSADDLDKELDSYHAEAMQT

SEQ ID NO: 517 - DNA, 1191 bp, Cannabis sativa
  source 1..1191: mol_type = other DNA
atggcaaggttggcccttgtacaacaacaacacaatcatccttctttctccatactcccttcttctctctctgatttcaatggcaccagactccactctcaaatccagttcaagaagaggatttctcaaccaaaaggtgcattacatgtttcagcatcaagtgagaaaaagattcttataatgggtggtactcggtttattggtatattcttgtccagactacttgtcaaagagggtcataaggtgactttgtttactagaggaaaagcacccattgcccaacaattgccaggggaatcagatgaagatttcagtgatttttcttccaagattttgcatctgaaaggagatagaaaggactatgattttgtgaaatctagcctctcagctgaaggttttgatgtagtttatgatataaatggaagagaggcagaggaagttgcacctatattggatgcactacccaatcttgaacagtacatatactgttcttcagctggtgtgtatctcaaatctgatctattacctcatttcgagaaagatgctgttgacccgaaaagcaggcacaaaggaaagctcgaaaccgagagcctgctagcgtctagggatgttaattggacttccataaggccagtctacatctatggacccttgaattacaatcccgtcgaagaatggttcttccaccggttgaaagctggtcgccctattccagttcccaattccggagtgcaaataacacaactcggtcatgttaaggacttagcaacagcttttattcaggtccttggtaatgaaaaagccagtagagaaatattcaacatatctggagaaaaatatgtcacatttgatggactagcaagagcatgtgcaaaggctggtggttttcccgagcctgagatcgttcactacaacccgaaagagtttgattttggcaaaaagaaagcgtttccattccgtgatcagcatttctttgcatcggtcgataaggcgaagagtgttcttggttggaaaccagaatttgatcttgtggaaggtcttactgactcctacaatttagactttggcagaggcacattcaggaaagaggctgatttttcaacagatgacattattctggaaagagtcttgttcttcaaagctagacatttcatcattttgctatttttcttgtatcttctttctttgtaa

SEQ ID NO: 518 - protein, 396 aa, Cannabis sativa
MARLALVQQQHNHPSFSILPSSLSDFNGTRLHSQIQFKKRISQPKGALHVSASSEKKILIMGGTRFIGIFLSRLLVKEGHKVTLFTRGKAPIAQQLPGESDEDFSDFSSKILHLKGDRKDYDFVKSSLSAEGFDVVYDINGREAEEVAPILDALPNLEQYIYCSSAGVYLKSDLLPHFEKDAVDPKSRHKGKLETESLLASRDVNWTSIRPVYIYGPLNYNPVEEWFFHRLKAGRPIPVPNSGVQITQLGHVKDLATAFIQVLGNEKASREIFNISGEKYVTFDGLARACAKAGGFPEPEIVHYNPKEFDFGKKKAFPFRDQHFFASVDKAKSVLGWKPEFDLVEGLTDSYNLDFGRGTFRKEADFSTDDIILERVLFFKARHFIILLFFLYLLSL

SEQ ID NO: 519 - DNA, 984 bp, Cannabis sativa
  source 1..984: mol_type = other DNA
atgtcagtgtctctcaataacttaaagatgcccgagctactatacaggtctctagcagcaaagcttgtcgtcgggatgccatttaaggatctggcaacggtagactccatcttattgagacaacttccacctattgacgatgacaacgctagactagctctcaaaagattgatagacataagtatgggggtcataactcctttgtcggagcagctaacaaagccattgccgaatgctatggttttggtaaatcttaaggagttatcatctggtgcacacaagcttttgccagaaggcacgcgtttggttgtatccttgcgcggtgatgaaccttacgaagaactggagattctcaaaggcgttgatgatgttgttatgattcttcatgatcttccgttcgatgaacataaaattagcagagtccactcagcaagaagattatttgagtatctatcagataattctcttaactttcctgtaatacaccacattcaatttccaaatggaatccacagggatgacttagtcatcggtgcaggtagcaacgctggtgcccttttagtagatggactcggggacggtatcctcttagaagccccagatcaggatttcgattttctcagaaatacttctttcaacctacttcaaggttgtagaatgcgaaatacaaagacggagtatgtctcgtgcccatcctgcggtagaactttgtttgaccttcaagaaattagcgcagagattcgagagaagacatcacacctgcccggtgtctcaattgcaatcatgggttgcattgttaatggacccggagagatggctgatgcagacttcggttatgtcggtggtgctcccggaaagattgacctttatgttggaaagacggtagtgcagcgtgcaatcgcaatggaacaagcgaccgatgcattgattcagctaataaaagatcatggccgatgggttgaaccaccctcggacgaagaatga

SEQ ID NO: 520 - protein, 742 aa, Cannabis sativa
MASGAVPASISCLKSRDSGLSFAKSSDFVRLSDLKRVGSSRTRVSVIRNSNPGSDIAELQPASKGSPLLVPRQKYCESLHKTVRRKTRTVMVGNVALGSEHPIRIQTMTTNDTKDVAGTVEEVMRIADKGADIVRITVQGRKEADACFEIKNSLVQKNYNIPLVADIHFAPPVALRVAECFDKIRVNPGNFADRRAQFETLEYTDDDYQKELEHIEQVFSPLVEKCKKYGRAMRIGTNHGSLSDRIMSYYGDSPRGMVESAFEFARICRKLDFHNFVFSMKASNPVVMVQAYRLLVAEMYVQGWDYPLHLGVTEAGEGEDGRMKSAIGIGTLLQDGLGDTIRVSLTEPPEEEIDPCRRLANLGTKAADLQQGVAPFEEKHRHYFDFQRRTGQLPLQKEGDEVDYRGALHRDGSVLMSVSLNNLKMPELLYRSLAAKLVVGMPFKDLATVDSILLRQLPPIDDDNARLALKRLIDISMGVITPLSEQLTKPLPNAMVLVNLKELSSGAHKLLPEGTRLVVSLRGDEPYEELEILKGVDDVVMILHDLPFDEHKISRVHSARRLFEYLSDNSLNFPVIHHIQFPNGIHRDDLVIGAGSNAGALLVDGLGDGILLEAPDQDFDFLRNTSFNLLQGCRMRNTKTEYVSCPSCGRTLFDLQEISAEIREKTSHLPGVSIAIMGCIVNGPGEMADADFGYVGGAPGKIDLYVGKTVVQRAIAMEQATDALIQLIKDHGRWVEPPSDEE

SEQ ID NO: 521 - DNA, 1221 bp, Cannabis sativa
  source 1..1221: mol_type = other DNA
atggctccagcagcagcttcttcagagtcagtaaagtcaagagatgtttgcattgttggtgtcgcgcgtacaccaatgggtggctttcttggtactttatcctctttacctgcaacaaaacttggatcaatagctattgaagctgcacttaaaagggctggtgttgatccttcacttgtacaagaagttatcttcggcaatgttcttagtgcaaatttaggtcaggctccagctagacaagctgcattaggtgcaggaatatctgatacagtaatctgtaccactgttaacaaagtctgtgcatcaggaatgaaagctacaatgcttgcagcacaaagcattcagttgggaattaatgatgttgttgtggctggtggcatggaaagcatgtctaatgtgcctaaatatattgctgaagcaagaaagggatctcgacttggacatgattctcttgttgatggaatgttgaaagatggtttgtgggatgtttataatgatgttggtatgggagtatgtgctgaaacatgtgcagaaaaccataaagtaacaagggaggagcaggacgattatgctattcagagctttgagcgtggtattgctgcccaagacagtggggcctttgtgtgggaaattgtaccggttgaagtttctggtggaaggggaaaaccagcaataattgttgacaaggatgaaggtctagggaagtttgatgctgggaaactaagaaagctccgtccaagtttcaaggcgaatggtggctcggtgactgctggaaatgcttccagcataagtgatggtgctgctgcattagtcttggtgagtggagaaacagcaataaagcttgggttgcaagttattgcaaagatctctggatatgctgatgctgctcaggcaccagaactatttactacagctccagcccttgcaatccccaaagctatttcaaatgctggcttgaatgctaatcaaattgatttctatgaaattaatgaagcttttgctgttgtggctcttgcaaatcagaaactgcttggactcaacacagaaaaagttaatgtgaacggcggggctgtagcgttgggacatcctctaggttgcagcggagctcgtatcttggtcacacttttgggggtattgaagcagaaaaatggtaaatatggtgttggtggcgtttgcaacggcggaggtggtgcttctgcacttgtggtagagctactttaa

SEQ ID NO: 522 - protein, 406 aa, Cannabis sativa
MAPAAASSESVKSRDVCIVGVARTPMGGFLGTLSSLPATKLGSIAIEAALKRAGVDPSLVQEVIFGNVLSANLGQAPARQAALGAGISDTVICTTVNKVCASGMKATMLAAQSIQLGINDVVVAGGMESMSNVPKYIAEARKGSRLGHDSLVDGMLKDGLWDVYNDVGMGVCAETCAENHKVTREEQDDYAIQSFERGIAAQDSGAFVWEIVPVEVSGGRGKPAIIVDKDEGLGKFDAGKLRKLRPSFKANGGSVTAGNASSISDGAAALVLVSGETAIKLGLQVIAKISGYADAAQAPELFTTAPALAIPKAISNAGLNANQIDFYEINEAFAVVALANQKLLGLNTEKVNVNGGAVALGHPLGCSGARILVTLLGVLKQKNGKYGVGGVCNGGGGASALVVELL

SEQ ID NO: 523 - DNA, 1227 bp, Cannabis sativa
  source 1..1227: mol_type = other DNA
ggcgatgttccaggtttgcttagcttgtatgaggcttcgcacttgagttatgttggagaaagcatactagatgaagcacttgctttcacaaccactcaccttaaggctattgtggcaaatagtaaagatcatccattatcacatcaaatatccatagtcctgcataggcctctaagaaagaccatagagaggcttcatgctaggttttacatctcaatctatgaaaaggatgcctctcataacaaactattgctagagcttgcaaagttagacttcaatctacttcaatgtttccacaaaaaggaacttagtgaaattacgaggtggtggaaggagcatgagtttgcaaagaaattcccttttgcaagagataggatggtggaactgtatttttggatattgggtgtatattatgaacccaaatactctcgagcaaggaagcttttaaccaaagtcattgcattgacctcaatcactgatgatatttatgatgcatatggtactattgatgagcttcagcttcttaccaaagcaattcaaaggtgggacataaattgtatggataaacttaagcaagaatatttaaagacatattataaggtaatgttggattcttatgaagaatttgaaaaggagcttaaaaaggaagaattatacaaacttgagtatgcaaaagaagagatgaaaagaattattggaggttattttgaagaagctcgatggttgaatgaaggatatttcccaagcttcgatgagcatttgagagtctcttatgtttcttctggtaacgttttgttgatagccacaagttttgtagggatgcatgatgttgtaacacatgaaactctagattggctctcgaaagaccctaaaattgtttcagcttctactctcctttctaggttcatggatgatattggttctcgcaagtttgagcaaaagagaaatcacataccatctacagtggattgttacatgaaacaatatggggtatcagaggaagaggcaattaaagaacttaataaaagagtggacacccactggaaagaaattaatgaagactttattaggccagcagttgtgccctttcctatcttagttcgtgttcttaattttacaaaaatagtagatcttctttacaaagagggcgatgatcaatacacaaatgttggaaaagtgctcaaagaaagcattgctgctttgcttatagattcaatcccattataa

SEQ ID NO: 524 - protein, 551 aa, Cannabis sativa
MSNIQVLASSQLSDKIIARPTTKFHPSIWGDRFLHYNISEQDLVCKQEKVEELIQVVKKEILSSNHDQLKLIDNLQRLGLSYHFESEIEKLLEQLSITHHQNHHDLQDASLWFRLLRQHGFNVSSSIFEKFKDEEGNFKESLITDVPGLLSLYEASHLSYVGESILDEALAFTTTHLKAIVANSKDHPLSHQISIVLHRPLRKTIERLHARFYISIYEKDASHNKLLLELAKLDFNLLQCFHKKELSEITRWWKEHEFAKKFPFARDRMVELYFWILGVYYEPKYSRARKLLTKVIALTSITDDIYDAYGTIDELQLLTKAIQRWDINCMDKLKQEYLKTYYKVMLDSYEEFEKELKKEELYKLEYAKEEMKRIIGGYFEEARWLNEGYFPSFDEHLRVSYVSSGNVLLIATSFVGMHDVVTHETLDWLSKDPKIVSASTLLSRFMDDIGSRKFEQKRNHIPSTVDCYMKQYGVSEEEAIKELNKRVDTHWKEINEDFIRPAVVPFPILVRVLNFTKIVDLLYKEGDDQYTNVGKVLKESIAALLIDSIPL

SEQ ID NO: 525 - DNA, 705 bp, Cannabis sativa
  source 1..705: mol_type = other DNA
atgggagactctgccgacgctggaatggacgctgtccagagacgccttatgtttgatgatgaatgcattctagtggatgagaatgaccgagttgttggtcatgatacaaaatataactgtcacttgatggaaaagattgaaaaggataatttgctacacagggctttcagtgtgttcttgttcaactcaaaatatgagttgcttcttcagcaacgttctgcaacaaaggtaacattccctcttgtgtggacaaacacctgttgtagccacccgctctaccgtgaatctgagcttatcgatgaggagtcccttggagcaaggaatgcagcacagagaaagcttttagatgagctgggtattcctgctgaagatgtgccagttgatcaatttaccccactaggcaggatgctgtacaaagctccttctgatggcaaatggggcgagcatgaacttgattacctgctcttcatcgtccgggatgttagtgtcaatccaaacccagatgaagtagctgatatcaagtatgtaaaccgggacgagttgaaagagttgttgaggaaagcagatgctggggaaggaggcttgaagctatccccttggttcagactggttgtggataatttcttgttcaagtggtgggaccatgttgagaaaggcacacttaaggaagttgctgatatgaaaaccattcacaagttgacttaa

SEQ ID NO: 526 - protein, 287 aa, Cannabis sativa
MTLLLNTTAKLYIAPRTLPFTSSSTFARSPFLRIPSLLKPLSPLTARVSLSSTMGDSADAGMDAVQRRLMFDDECILVDENDRVVGHDTKYNCHLMEKIEKDNLLHRAFSVFLFNSKYELLLQQRSATKVTFPLVWTNTCCSHPLYRESELIDEESLGARNAAQRKLLDELGIPAEDVPVDQFTPLGRMLYKAPSDGKWGEHELDYLLFIVRDVSVNPNPDEVADIKYVNRDELKELLRKADAGEGGLKLSPWFRLVVDNFLFKWWDHVEKGTLKEVADMKTIHKLT

SEQ ID NO: 527 - DNA, 981 bp, Cannabis sativa
  source 1..981: mol_type = other DNA
atggaacacaaaggagacagagccttaatatccactcaaggtgaactggctcatgcattgcgattcttggcctctgttgtatcacagagctttttagcttttggtgacattctcgaattgcatcggaagtttgttgaactttctggtggggtcaatcgaatttttgaacttgaagagcttctagatgctgcacagtccggagatcctaatgccaatgctggatcatcagctgaaaggagacatctttactctgaggatgccattagcttctctgaagttgacattgttacccctgggcagaaattgttggctcggaaattgacatgtgacatatcacctggaaaaagcctgcttgttactggtccaaacgggagtggaaaaagttctgttttcagagctcttagaggtctttggcccatcatgagtggaaggctcactcaaccatcccaacatgtcactgaagaggtcgggtcaggttgtggtgtgttctatgttcctcagcgaccatacacttgcttgggaacgcttagggatcaaataatatatcctctctcacagagggaagcagaattgagggcattgcagctctatgggaaaggtaaagagtctaccgacaccaaaaacattctggacatgcaattgaaaactattctagaaaatgttagattgaattatcttttggaaagggagggaagtggttgggatgctaacttgaattgggaggatattctttctcttggagaacaacagagattaggaatggcccgcttatttttccacaagcctaaatttggaatcctcgacgagtgtacaaacgccacgagtgtagacgttgaagaacatctttatagacttgcaaaggagatgggtattacagtagttacatcctcacaacgtcccgctttaataccattccactctgtggaattgcggcttatcgacggtgagggtaactgggaattacgttcaatcaagcaatga

SEQ ID NO: 528 - protein, 326 aa, Cannabis sativa
MEHKGDRALISTQGELAHALRFLASVVSQSFLAFGDILELHRKFVELSGGVNRIFELEELLDAAQSGDPNANAGSSAERRHLYSEDAISFSEVDIVTPGQKLLARKLTCDISPGKSLLVTGPNGSGKSSVFRALRGLWPIMSGRLTQPSQHVTEEVGSGCGVFYVPQRPYTCLGTLRDQIIYPLSQREAELRALQLYGKGKESTDTKNILDMQLKTILENVRLNYLLEREGSGWDANLNWEDILSLGEQQRLGMARLFFHKPKFGILDECTNATSVDVEEHLYRLAKEMGITVVTSSQRPALIPFHSVELRLIDGEGNWELRSIKQ

SEQ ID NO: 529 - DNA, 915 bp, Cannabis sativa
  source 1..915: mol_type = other DNA
atggtgaaaggaccaggtctctataccgatattggcaagaaagccagagatcttctgtacagagattatcaggctgatcataagtttactgtcactacctatacttctaacggagttgcaattacttcaactggagttaagaaaggcgagctatttttggcagatttgagcactcagttgaagaacaagaatatcacgactgacataaaagttgacactaactctaatcttatcaccaccatcaccgttgatgaaccagcacctggacttaagacaatctttagctttattgctcccgatcagagatctgggaaggttgagctccaatatcagcacgagtatgctgggatcagcactagcattggattgaccgcaaatcctagtgtcaacttttctggtgtgattggaaacaattctttggcagttggaacagatttgtcctttgatacagcatctgggaacttcaccaaatttaatacaggattgaatttcattcacaatgatctcattgcttccctgatattaaatgataaaggtgacacccttactgcatcgtactaccatactgtaagcccccttaccaacactaacgttggtgcagagttttcccatagcttttcaagcaatgaaaacactctcactattggcacacaacacgcacttgatcccttgaccaatttgaaagctagagtgaacaattatgggaaggcaagtgctcttattcaacatgagtggcgcccaaagtctctcttcaccatctcaggagaagtggataccagggcaattgaaaaagagtgcaaaggttggtctagctttggtactcaagccttgagactaattccattcgaaaggttggttttttctttgagcttcatggacaggaataagaggaagggactggaacatttccgatga

SEQ ID NO: 530 - protein, 304 aa, Cannabis sativa
MVKGPGLYTDIGKKARDLLYRDYQADHKFTVTTYTSNGVAITSTGVKKGELFLADLSTQLKNKNITTDIKVDTNSNLITTITVDEPAPGLKTIFSFIAPDQRSGKVELQYQHEYAGISTSIGLTANPSVNFSGVIGNNSLAVGTDLSFDTASGNFTKFNTGLNFIHNDLIASLILNDKGDTLTASYYHTVSPLTNTNVGAEFSHSFSSNENTLTIGTQHALDPLTNLKARVNNYGKASALIQHEWRPKSLFTISGEVDTRAIEKECKGWSSFGTQALRLIPFERLVFSLSFMDRNKRKGLEHFR

SEQ ID NO: 531 - DNA, 1182 bp, Cannabis sativa
  source 1..1182: mol_type = other DNA
atgcttggacgcattttcaatggttctggaaaacctattgataatgggcccccaattttgcctgaggcatacttggatatatcagggagttctatcaatcctagtgagagaacatatcctgaagaaatgattcagacggggatttcaacaattgatgtcatgaattccattgctagaggacagaagatcccacttttctctgctgctggtcttcctcacaatgaaattgctgctcaaatctgtcgtcaggctggtttggtcaagcgattggagaaaactgaaaatcttctcgagcaatctgaagaggacaattttgccattgtgtttgctgctatgggtgtcaacatggagacagcacaatttttcaaacgtgattttgaggaaaatggctcaatggagagagtgacacttttcttgaatctggcaaatgaccctacaattgaacgtatcatcactccccgtattgctcttaccactgctgagtatttggcatatgaatgcgggaagcacgtgcttgtcatccttacagatatgagttcttatgctgatgctcttcgtgaggtgtctgctgctagagaagaagtacccggtagacgtgggtatcctgggtatatgtataccgatcttgccacaatctatgagcgtgcaggaagaattgaagggcgaacaggttccatcacccagattccaattttaactatgcctaatgatgatattacccatcccacccctgatcttactggatatattaccgaggggcagatctacgttgataggcaactccaaaacagacagatataccctcccatcaacgtgcttccttccctctctcgtcttatgaagagtgctattggtgagggaatgactcgcagggatcatgctgatgtttccaaccagctctatgcaaattatgctattgggaaggatgtccaggccatgaaggctgtggttggagaagaagctctttcatctgaggatctgctatacttggaattcttggacaaattcgaaggaagtttgtcgctcaaggagcatacgacactcgcaacatcttccagtcacttgatttggcatggacactgcttcgcatcttcccccgagagcttcttcatcgtattccagccaagacccttgacctgtactacagccgagatgcagctaactaagtatgaagacacataa

SEQ ID NO: 532 - protein, 393 aa, Cannabis sativa
MLGRIFNGSGKPIDNGPPILPEAYLDISGSSINPSERTYPEEMIQTGISTIDVMNSIARGQKIPLFSAAGLPHNEIAAQICRQAGLVKRLEKTENLLEQSEEDNFAIVFAAMGVNMETAQFFKRDFEENGSMERVTLFLNLANDPTIERIITPRIALTTAEYLAYECGKHVLVILTDMSSYADALREVSAAREEVPGRRGYPGYMYTDLATIYERAGRIEGRTGSITQIPILTMPNDDITHPTPDLTGYITEGQIYVDRQLQNRQIYPPINVLPSLSRLMKSAIGEGMTRRDHADVSNQLYANYAIGKDVQAMKAVVGEEALSSEDLLYLEFLDKFEGSLSLKEHTTLATSSSHLIWHGHCFASSPESFFIVFQPRPLTCTTAEMQLTKYEDT

SEQ ID NO: 533 - DNA, 1014 bp, Cannabis sativa
  source 1..1014: mol_type = other DNA
atgggtgtcaaaggatttgttgaaggaggcatagcttcgatcgttgccggctgttcaacccacccaatcgacctcgtcaaggtccgtatgcagctccagggcgagtccgtcacccccaacccggcggcggttcaaaatctccgaccggctctttcttttcaccccaactcagctgctggatccgtccgtatcccaactccgcctcccgcgccgccggtgcgcgtcggcccaatcgccgtcggagttcgcattgtgcagcaagaaggaatggcgggtttgttctctggtgtctccgctactgttctcaggcagactctctactccaccacacgtatgggtctgtacgatatcttgaagcagaagtggaccgatcccaacacggggaacatgcccttgacccgcaaaatcacggctggtctgatagccggcgcgatcggagctgccgttggaaacccagctgatgtggcgatggttcgtatgcaagccgacggtcggcttcccatggctcagcgccgtaattacacgagcgtggtcgatgctctgacgcggatgtcgcgccaggaagggattcggagcttgtggcgcggctcgtcgatgacgatcaaccgagccatgctcgtgacggcttcgcagctggcttcgtacgaccagatcaaggagacgattctagaaaaaggggtcatgaaagacgggctcgggacccacgtgacggcgagctttgcagcggggtttgtggcatcggttgcgtcaaacccagttgacgttatcaaaaccagagtcatgaacatgaaggtggagcccggatcgcctccgccgtacaacggagccattgactgtgctctcaaaacggtgcgttcagagggtccctttgctctctacaaagggttttttgccaacgatctcgaggcaaggacctttcactgtgatgctgttcgtgacattggagcaggttcgaaagttgctcaaggatttctgattagaatgatgatgacgacaattaagactattaattaa

SEQ ID NO: 534 - protein, 337 aa, Cannabis sativa
MGVKGFVEGGIASIVAGCSTHPIDLVKVRMQLQGESVTPNPAAVQNLRPALSFHPNSAAGSVRIPTPPPAPPVRVGPIAVGVRIVQQEGMAGLFSGVSATVLRQTLYSTTRMGLYDILKQKWTDPNTGNMPLTRKITAGLIAGAIGAAVGNPADVAMVRMQADGRLPMAQRRNYTSVVDALTRMSRQEGIRSLWRGSSMTINRAMLVTASQLASYDQIKETILEKGVMKDGLGTHVTASFAAGFVASVASNPVDVIKTRVMNMKVEPGSPPPYNGAIDCALKTVRSEGPFALYKGFFANDLEARTFHCDAVRDIGAGSKVAQGFLIRMMMTTIKTIN

SEQ ID NO: 535 - DNA, 1116 bp, Cannabis sativa
  source 1..1116: mol_type = other DNA
atggccggctccatagtatttttcttcgttggggctcctctttacaatcacgtgaaaccagaaggcagcatattttccggtattgctcaggttttcgttgctgctttcaagaagcgtcgcctaatgattccggcacccactgataatgttgacggagatggctccctcttattttatgatcctccactcaatggaaatgtcttgtcaaaactccctcgaaccaatcagtttaggtattttgacaaagcagctgtaatattggaaaatgagataaacccaaacggcgatcctttgaacaagtggagattgtgcagtatccaacaggtagaggaagtgaagtgcttagcaagaatcacaccgatatgggcgtcaggaatcataagcttgatctccatagtccaacaagggactttcacagtctctcaagctctcaaaatggatcgtcacataggttccaattttgaggtcccagcaggttcaataggcgtcatctcactcatcaccatcggcatttggcttcctttctacgaccgcctactcgtcccagctctccgccgaatcaccaaccacgagaacggaatcacactcctccagagaatcggaatcggaaacattttctcaattctgtcaatggtagtagccggattagtcgaaaaggagagaagaagatgggccaactcagggatggcgtccatgtcggtgttctggttggccccacaattggtgttaatgggattttgtgaagcctttaacatgataggactaattgagtttttcaaccgccagtttccggatcacatgaggagcattggaaatgcgctcttctcttgctctttcgctggtgccagttacactagcagtatattggttactgtcctccatcgagttaccgctaccaatgaccgacccgattggttgactaatgatattaacaaggggaggctggactatttttactatcttttagctggtttgggtgtctttaatgttctttattttctctattgtgctagtcgttatcgttacaaaggggctgtcatagaattggagaacaaagctacaaatgtggtagatgtggagatgaattcggtgcttaataaagggctcagtacttga

SEQ ID NO: 536 - protein, 371 aa, Cannabis sativa
MAGSIVFFFVGAPLYNHVKPEGSIFSGIAQVFVAAFKKRRLMIPAPTDNVDGDGSLLFYDPPLNGNVLSKLPRTNQFRYFDKAAVILENEINPNGDPLNKWRLCSIQQVEEVKCLARITPIWASGIISLISIVQQGTFTVSQALKMDRHIGSNFEVPAGSIGVISLITIGIWLPFYDRLLVPALRRITNHENGITLLQRIGIGNIFSILSMVVAGLVEKERRRWANSGMASMSVFWLAPQLVLMGFCEAFNMIGLIEFFNRQFPDHMRSIGNALFSCSFAGASYTSSILVTVLHRVTATNDRPDWLTNDINKGRLDYFYYLLAGLGVFNVLYFLYCASRYRYKGAVIELENKATNVVDVEMNSVLNKGLST

SEQ ID NO: 537 - DNA, 672 bp, Cannabis sativa
  source 1..672: mol_type = other DNA
atggaagaaaaagtgaaagagattgtggagattaagcaaagaagcttggcagaggagaaccaaaaaacacttgaggtcctgaaagatagagagcagctgttgcaagatcaattacgacaagctaaagagagtgtttccaatatgcagaagttgcacgaacttgcacaaagccaaatgtttgagcttcgtgctcaatcagatgaagatagggcagcaaagcagtcagaggtcaatcttctgatggatgaagttgaacgggctcaaactcggcttcttagtcttgagagggaaaagggaattctacgcacccagttgcagtcagccaatgaagacactgataaaaagagtgatgatatagattcaaataccatccttgagaattcattgatagcaaaagagaagatcatttctgagctcaacatggaacttcacaatattgaaactaccttgtcaaatgagcgagaagaacacattaatgatatcaaaaaattgaatttcctgcttcatgaaaaggaagtagcccttgaggagatgaagaaagagattcaggcaaggccaacagaaaagttggttgatgatttgcgtaaaaaagtgaagatcttgcaggcagtaggttataattcaattgaggcagaagattgggaagttgctactagcggggaagagatgagc

SEQ ID NO: 538 - protein, 224 aa, Cannabis sativa
MEEKVKEIVEIKQRSLAEENQKTLEVLKDREQLLQDQLRQAKESVSNMQKLHELAQSQMFELRAQSDEDRAAKQSEVNLLMDEVERAQTRLLSLEREKGILRTQLQSANEDTDKKSDDIDSNTILENSLIAKEKIISELNMELHNIETTLSNEREEHINDIKKLNFLLHEKEVALEEMKKEIQARPTEKLVDDLRKKVKILQAVGYNSIEAEDWEVATSGEEMS

SEQ ID NO: 539 - DNA, 1926 bp, Cannabis sativa
  source 1..1926: mol_type = other DNA
atgtcgtgcttagccctctctcttcaacccgccaatggctccgacatccttctacagacccgagagtggttccctccggcacgagcccttgtcgcactctccgcctttcgccagactcgtctcgctttcggctccagcaagaacaacaacagcagcaacaaccagtctcagttcaccgattccgatccctccgttgattccataggcgatgatccactcgcagcttccagtggccagctcatcgtcggagtcgagagtcggtaccgagtcgtgtacagactcgtcaacagcatctatgttctcggaatcactacggccgatcacgacaacgcaatcaacgtcttcgaatgcatccacgtggtgaaccaagctgtgagcgtgatcgtcaccgcctgccgcggcgtcgacgtaacgcctgagaagctaggaagaaagtacgccgagatctacatggcgttggacattgttctcaggggagttagcaatatccgactcgcggccatgctcgcttcgttgcacggcgatagcattgcgaagatggtacattcggcgctcgattctgagaacaagatccgtggtgctgataattggaccagtgtggaggtgaattctgttaaacactcggcgaacattgaggtattttcccacgcttcgtttgaaatgccgccggagactattgctgccggagacgagatcgctgctactatcgctccggcgttaccgactgagcagttgcaagatcagaaggaggagatagaagagattgaagctgaaaaggatccttttgctgctagtgataagattaacgaaccggcggagcttgttgggggtttcaagaagaacagagacccttctgctactgatttgactaaagctttagctgggcttgaggtaactacgttgcctcctgcggaggcaacacaatcgactcatattgctgttgaagggtttgaaggtgagtatggtggaattgagtttgggaatgaacaagcttctcttggtgaaacttttgaagggtttagtgatgcttggggtggaggattggatccatcggaatttgttggaccaactaaaggtcctaagacacaaggtcttggaggactcgaattgcttcagactggtcctgataatgctcctaaagtggataatgctgcagccaccgggggtacaggtacgccgcttgatgaacttgttaagtctactgaaatgaaaggaccagagatgtatatatctgaacagattaatgttgagtttagagaatcattacttgctagagtaggattaatgggtactgtctatttgaaaactttgcctactaaaacatctggtgataaagaaactgaattttcattccgagttgagggcacaacggctgttaagagatttgttattcagagttctcgtgttagtagtttaggcaatggaatgttccatgttaggactgccgcttcggatgaacctttgccggttttgaagtatagtttggttcctagattaacaccattgcctctgaggattcgtcttataaaacgttttactggaaccttgctatctctgatgattcagtatgtatcgaaccctgagttgccggtgccattgaatgatgtaacatttgttttgaaactaccggtagacccttcattgttgcaagtttcgcctaaagcaatgcttaataggtctgagaaagaattgaaatggcatattgctgagattccacaaaagggatctcctggtaggttgagagcaaggatgcctgtggatttaaatgatggtgacgaagagatggatatcgttgtttatgtgaaattctctatacaaggaaagcaatcgctttcgggggtttgtctacggcctgccactgaggggaagactgacttctatgaggttaatcacaggtttgaaaatggagtatatatgtgcaattga

SEQ ID NO: 540 - protein, 641 aa, Cannabis sativa
MSCLALSLQPANGSDILLQTREWFPPARALVALSAFRQTRLAFGSSKNNNSSNNQSQFTDSDPSVDSIGDDPLAASSGQLIVGVESRYRVVYRLVNSIYVLGITTADHDNAINVFECIHVVNQAVSVIVTACRGVDVTPEKLGRKYAEIYMALDIVLRGVSNIRLAAMLASLHGDSIAKMVHSALDSENKIRGADNWTSVEVNSVKHSANIEVFSHASFEMPPETIAAGDEIAATIAPALPTEQLQDQKEEIEEIEAEKDPFAASDKINEPAELVGGFKKNRDPSATDLTKALAGLEVTTLPPAEATQSTHIAVEGFEGEYGGIEFGNEQASLGETFEGFSDAWGGGLDPSEFVGPTKGPKTQGLGGLELLQTGPDNAPKVDNAAATGGTGTPLDELVKSTEMKGPEMYISEQINVEFRESLLARVGLMGTVYLKTLPTKTSGDKETEFSFRVEGTTAVKRFVIQSSRVSSLGNGMFHVRTAASDEPLPVLKYSLVPRLTPLPLRIRLIKRFTGTLLSLMIQYVSNPELPVPLNDVTFVLKLPVDPSLLQVSPKAMLNRSEKELKWHIAEIPQKGSPGRLRARMPVDLNDGDEEMDIVVYVKFSIQGKQSLSGVCLRPATEGKTDFYEVNHRFENGVYMCN

SEQ ID NO: 541 - DNA, 1638 bp, Cannabis sativa
  source 1..1638: mol_type = other DNA
atgaattgctcagcattttccttttggtttgtttgcaaaataatatttttctttctctcattccatatccaaatttcaatagctaatcctcgagaaaacttccttaaatgcttctcaaaacatattcccaacaatgtagcaaatccaaaactcgtatacactcaacacgaccaattgtatatgtctatcctgaattcgacaatacaaaatcttagattcatctctgatacaaccccaaaaccactcgttattgtcactccttcaaataactcccatatccaagcaactattttatgctctaagaaagttggcttgcagattcgaactcgaagcggtggccatgatgctgagggtatgtcctacatatctcaagtcccatttgttgtagtagacttgagaaacatgcattcgatcaaaatagatgttcatagccaaactgcgtgggttgaagccggagctacccttggagaagtttattattggatcaatgagaagaatgagaatcttagttttcctggtgggtattgccctactgttggcgtaggtggacactttagtggaggaggctatggagcattgatgcgaaattatggccttgcggctgataatattattgatgcacacttagtcaatgttgatggaaaagttctagatcgaaaatccatgggagaagatctgttttgggctatacgtggtggtggaggagaaaactttggaatcattgcagcatggaaaatcaaactggttgctgtcccatcaaagtctactatattcagtgttaaaaagaacatggagatacatgggcttgtcaagttatttaacaaatggcaaaatattgcttacaagtatgacaaagatttagtactcatgactcacttcataacaaagaatattacagataatcatgggaagaataagactacagtacatggttacttctcttcaatttttcatggtggagtggatagtctagtcgacttgatgaacaagagctttcctgagttgggtattaaaaaaactgattgcaaagaatttagctggattgatacaaccatcttctacagtggtgttgtaaattttaacactgctaattttaaaaaggaaattttgcttgatagatcagctgggaagaagacggctttctcaattaagttagactatgttaagaaaccaattccagaaactgcaatggtcaaaattttggaaaaattatatgaagaagatgtaggagctgggatgtatgtgttgtacccttacggtggtataatggaggagatttcagaatcagcaattccattccctcatcgagctggaataatgtatgaactttggtacactgcttcctgggagaagcaagaagataatgaaaagcatataaactgggttcgaagtgtttataattttacgactccttatgtgtcccaaaatccaagattggcgtatctcaattatagggaccttgatttaggaaaaactaatcatgcgagtcctaataattacacacaagcacgtatttggggtgaaaagtattttggtaaaaattttaacaggttagttaaggtgaaaactaaagttgatcccaataatttttttagaaacgaacaaagtatcccacctcttccaccgcatcatcattaa

SEQ ID NO: 542 - protein, 545 aa, Cannabis sativa
MNCSAFSFWFVCKIIFFFLSFHIQISIANPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 543 - DNA, 1557 bp, Cannabis sativa
  source 1..1557: mol_type = other DNA
atgaatcctcgagaaaacttccttaaatgcttctcaaaacatattcccaacaatgtagcaaatccaaaactcgtatacactcaacacgaccaattgtatatgtctatcctgaattcgacaatacaaaatcttagattcatctctgatacaaccccaaaaccactcgttattgtcactccttcaaataactcccatatccaagcaactattttatgctctaagaaagttggcttgcagattcgaactcgaagcggtggccatgatgctgagggtatgtcctacatatctcaagtcccatttgttgtagtagacttgagaaacatgcattcgatcaaaatagatgttcatagccaaactgcgtgggttgaagccggagctacccttggagaagtttattattggatcaatgagaagaatgagaatcttagttttcctggtgggtattgccctactgttggcgtaggtggacactttagtggaggaggctatggagcattgatgcgaaattatggccttgcggctgataatattattgatgcacacttagtcaatgttgatggaaaagttctagatcgaaaatccatgggagaagatctgttttgggctatacgtggtggtggaggagaaaactttggaatcattgcagcatggaaaatcaaactggttgctgtcccatcaaagtctactatattcagtgttaaaaagaacatggagatacatgggcttgtcaagttatttaacaaatggcaaaatattgcttacaagtatgacaaagatttagtactcatgactcacttcataacaaagaatattacagataatcatgggaagaataagactacagtacatggttacttctcttcaatttttcatggtggagtggatagtctagtcgacttgatgaacaagagctttcctgagttgggtattaaaaaaactgattgcaaagaatttagctggattgatacaaccatcttctacagtggtgttgtaaattttaacactgctaattttaaaaaggaaattttgcttgatagatcagctgggaagaagacggctttctcaattaagttagactatgttaagaaaccaattccagaaactgcaatggtcaaaattttggaaaaattatatgaagaagatgtaggagctgggatgtatgtgttgtacccttacggtggtataatggaggagatttcagaatcagcaattccattccctcatcgagctggaataatgtatgaactttggtacactgcttcctgggagaagcaagaagataatgaaaagcatataaactgggttcgaagtgtttataattttacgactccttatgtgtcccaaaatccaagattggcgtatctcaattatagggaccttgatttaggaaaaactaatcatgcgagtcctaataattacacacaagcacgtatttggggtgaaaagtattttggtaaaaattttaacaggttagttaaggtgaaaactaaagttgatcccaataatttttttagaaacgaacaaagtatcccacctcttccaccgcatcatcattaa

SEQ ID NO: 544 - protein, 518 aa, Cannabis sativa
MNPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH